(19) United States
(12) Patent Application Publication
(10) Pub. No.: US 2026/0272879 A1
(43) Pub. Date: Sep. 17, 2026

(54) ANION-BINDING AROMATIC OLIGOAMIDE MACROCYCLES AND METHODS OF MAKING AND USING SAME

(71) Applicant: The Research Foundation for The State University of New York, Amherst, NY (US)

(72) Inventors: Bing GONG, Amherst, NY (US); Ruikai CAO, Amherst, NY (US)

(21) Appl. No.: 19/480,236

(22) PCT Filed: Apr. 30, 2024
(86) PCT No.: PCT/US2024/027117

§ 371 Date: Oct. 30, 2025

Related U.S. Application Data
(60) Provisional application No. 63/499,257, filed on Apr. 30, 2023.

(51) Int. Cl. A61K 31/395; C07D 259/00

(52) U.S. Cl. CPC A61K 31/395; C07D 259/00

(57) ABSTRACT

Provided are compounds having the following structure (I) or (II) The compounds may be used to sequester one or more hydrogen-bond acceptors and/or ions. Also provided are methods for making the compounds and methods for using the compounds for treating individuals diagnosed with or suspected of having an extracellular and/or intracellular anion imbalances and/or cystic fibrosis.

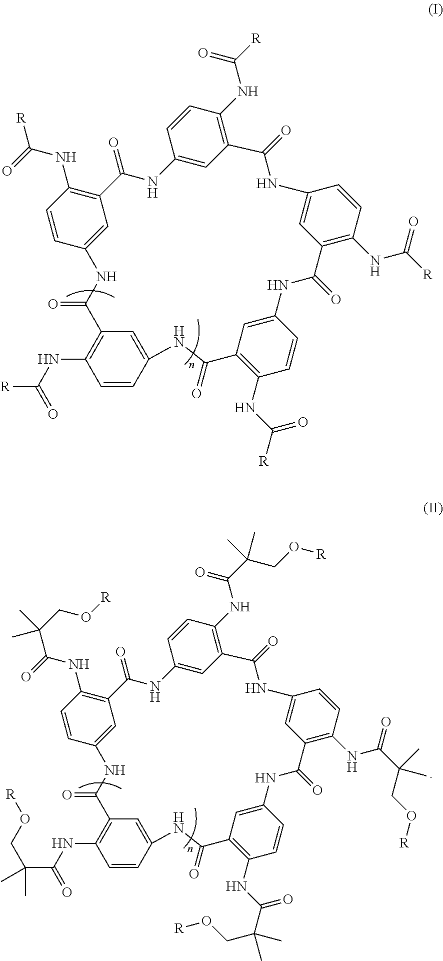

### CROSS REFERENCE TO RELATED APPLICATIONS

This application claims the benefit of U.S. Provisional Patent Application No. 63/499,257, filed on Apr. 30, 2023, the disclosure of which is incorporated herein by reference in its entirety.

### STATEMENT REGARDING FEDERALLY SPONSORED RESEARCH

This invention was made with government support under contract nos. CHE-1905094 & CHE-2108538 awarded by the National Science Foundation. The government has certain rights in the invention.

### BACKGROUND OF THE DISCLOSURE

Anion recognition, as a major theme of supramolecular chemistry, is attracting wide attention in a variety of areas because of the central role played by anions in biology. Anions are found in the anionic centers of most biomolecules. The majority of enzyme substrates are anionic. The maintenance of cellular functions relies on transmembrane anion transport. The development of anion binders and receptors has great potential for various biological activities. Many diseases are related to ion transport and anion channels. By promoting transmembrane transport of anions, either through anion carriers or anion channels, genetic conditions and diseases caused by defective anion channels could be remedied. For example, cystic fibrosis, a genetic disorder caused by a mutation in the gene cystic fibrosis transmembrane conductance regulator (CFTR) and disfunctioning chloride channel, is accompanied by long-term complications including difficulty breathing and coughing up mucus as a result of frequent lung infections. Other signs and symptoms related to malfunctioned channels may include sinus infections, poor growth, fatty stool, clubbing of the fingers and toes, and infertility in most males. Although there is no known cure for cystic fibrosis, synthetic anion carriers and channels could substitute the mutated CFTR chloride channels and restore transmembrane chloride conductance, which alleviates the symptoms of cystic fibrosis. For this reason, anion carriers and channels especially those for the chloride ion are being actively pursued. In comparison to the numerous synthetic cation binders and receptors known thus far, much less anion receptors have been reported. The design of anion binders or receptors typically requires the convergent arrangement of multiple hydrogen-bond donors which engage in multiple cooperative H-bonding interactions with an anion guest. The H-bond donors are based on the NH groups of amide, urea, or thiourea groups. However, due to the inherent structural limitation of oligoamides and oligoureas, arranging multiple NH groups convergently turns out to be a rather challenging task. As a result, the convergent placement of multiple NH groups has to rely on cyclic structures that may not be readily available synthetically. Besides, few examples of anion binders with systematically variable binding strength are known.

The convergent positioning of functional groups in biomacromolecules leads to astonishing binding, catalytic, and transport capabilities. In contrast, synthetic frameworks capable of convergently locking functional groups with minimized conformational uncertainty are highly desirable but rare.

Molecular and supramolecular structures with multiple functional groups being convergently placed at defined positions occur ubiquitously in nature. Amassing multiple noncovalent forces, such structures exhibit sophisticated functions from ligand binding and mass transport to signal transduction that are still beyond the reach of synthetic constructs. For example, with a “selectivity filter” defined by aligned polar groups and dipoles, the potassium channel KcsA or the water channel aquaporin 1 achieves astounding efficiency and exclusive selectivity in transporting potassium ions or water molecules. Anion channels such as the ClC Cl⁻ channels also contain selectivity filters defined by multiple NH and OH groups, along with CH groups, that converge toward the bound chloride. The remarkable power of convergent functional groups is also demonstrated by many ligand-binding proteins such as the sulfate-binding protein (SBP) from the gram-negative bacterium S. typhimurium. SBP binds the sulfate ion, an anion with a large hydration enthalpy (−258 kcal/mol), in high affinity (Kₐ=8.3×10⁶ M⁻¹ at pH 8.3) in water. By forming seven neutral hydrogen bonds with the sulfate ion, five involving the main chain peptide NH groups, one with a serine OH and another with the indole NH of a tryptophan residue, SBP demonstrates that neutral hydrogen bonds, if properly placed, can result in extraordinary binding affinity for an anion.

Anions have attracted wide interest in molecular recognition because of their importance in biology, medicine, and the environment. In contrast to cations, anions, with a variety of sizes and shapes, are challenging targets that remained largely elusive for synthetic receptors until recent years. Anion recognition requires the convergent alignment of binding groups, especially hydrogen-bond donors. Typical anion receptors include linear or branched molecules carrying hydrogen-bond donors based on the NH groups of amides, ureas, pyrroles, amines, and ammoniums. More recently, systems based on halogen bonds were reported. With such hosts, the entropic costs for hydrogen-bond donors to converge on anions have to be compensated by the enthalpic gains from multiple non-covalent interactions.

Considerable effort has been devoted to arrange convergent functional groups with synthetically accessible structures. A promising strategy for converging multiple functional groups is based on rigid structures such as molecular clefts, foldamers, and macrocycles. Such structures exhibit unique, often remarkable properties including selective and tight binding of guest species.

In this regard, shape-persistent macrocycles are especially powerful in converging functional groups and generating non-deformable cavities with defined sizes. Flood et al. demonstrated the formation of stable 2:1 sandwich complexes of large anions with a rigid macrocycle having convergent cyanostilbene and phenyl CH groups, and another rigid macrocycle having convergent triazole and phenyl CH groups for binding the chloride ion in a 1:1 stoichiometry. These systems, along with examples such as those reported by Anslyn, Sessler, and Gale, demonstrate that rigid cyclic hosts with convergently organized hydrogen-bond donors, even those involving aromatic CH groups which are regarded as “weak” donors, could achieve strong overall binding for anions.

Among hydrogen-bond donors, the amide NH functionality, with its exceptional hydrogen-bonding capability and synthetic accessibility, is much preferred in the design of anion receptors. Aromatic oligoamide macrocycles, with rigid residues linked by amide linkages, have persistent shapes that organize multiple binding elements together in a rigid relationship amenable to strong anion binding. Ideally, the backbones of such oligoamide macrocycles should be rigid and fully constrained, i.e., even the rotation around each backbone aryl-amide single bonds is hindered, so that the ambiguity in local orientation associated with the amide groups could be strongly restricted or even eliminated. However, due to the structural limitations of the amide group, most known amide-based anion receptors have global to local conformational flexibility, along with unhindered rotation around the aryl-amide single bonds, that has compromised both binding affinity and selectivity. In addition, incorporating amide NH units into anion receptors inevitably brings along amide C═O groups that may result in undesired hydrogen-bonding interactions, leading to self-aggregation and poor solubility. As a result, few anion binders based on shape-persistent oligoamide macrocycles are known to date.

Hamilton et al. reported their seminal work on a rigid aromatic triamide macrocycle that projects three NH groups in a ~5-Å cavity and binds dihydrogen phosphate strongly (Kₐ≈10+M⁻¹ in DMSO-d₆). This macrocycle, however, has a backbone in which the rotation of aryl-amide single bonds is not restricted. While there can be aromatic oligoamide macrocycles with backbone amide C═O groups rigidly placed into a convergent orientation, the inherent limitation of the amide group precludes the creation of rigid aromatic oligoamide macrocycles capable of convergently locking NH groups for anion-binding. In fact, the design of rigid hosts with preorganized, convergently placed amide NH groups for anion binding remains a daunting conceptual and synthetic challenge with few feasible solutions.

### BRIEF SUMMARY OF THE DISCLOSURE

The present disclosure provides linear and cyclic oligoamides. The present disclosure also provides methods of making and uses of linear and cyclic oligoamides.

In an aspect, the present disclosure provides compounds. The compounds are linear or cyclic oligoamides. A compound, which may be a linear or cyclic macrocyclic oliogamide (which may also be referred to as a macrocyclic compound/oligoamide), may comprise one or more aromatic substituents. In the case where a compound comprises a plurality of aromatic substituents, adjacent aromatic substituents are linked by at least one amide group. Non-limiting examples of linear and cyclic/macrocyclic oligoamides are provided herein.

In various examples, a compound of the present disclosure has the following structure:

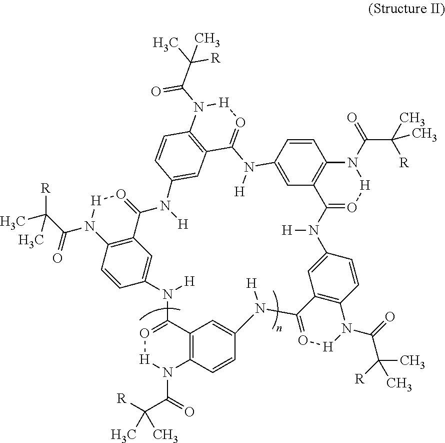

where n is 1 or 2. R is independently at each occurrence chosen from:

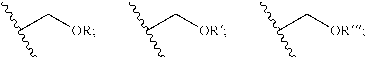

linear aliphatic groups (which may be C₁-C₂₀ linear aliphatic groups (e.g., linear alkyl groups, such as, for example, methyl, ethyl, propyl, butyl, pentyl, hexyl, heptyl, octyl groups, and the like, which may be C₁-C₂₀ linear alkyl groups));
branched aliphatic groups (which may be C₁-C₂₀ branched aliphatic groups (e.g., branched alkyl groups, such as, for example, isopropyl, isobutyl, t-butyl, neopentyl, isopentyl groups, and the like, which may be C₁-C₂₀ branched alkyl groups));
fluorinated linear aliphatic groups (which may be C₁-C₂₀ fluorinated linear aliphatic groups (e.g., fluorinated linear alkyl groups, such as, for example, fluoromethyl, fluoroethyl, fluoropropyl, fluorobutyl, fluoropentyl, fluorohexyl, fluoroheptyl, fluorooctyl groups, and the like), which comprise one or more fluorine group(s) (e.g., a fluorinated linear aliphatic group (such as, for example, a fluorinated linear alkyl group or the like) is a perfluorinated linear aliphatic group (e.g., alkyl and the like)));
fluorinated branched aliphatic groups (which may be C₁-C₂₀ fluorinated branched alkyl groups (e.g., fluorinated branched alkyl groups, such as, for example, branched derivatives of fluoropropyl, fluorobutyl, fluoropentyl, fluorohexyl, fluoroheptyl, fluorooctyl groups, and the like) and the like, which comprise one or more fluorine group(s) (e.g., a fluorinated branched aliphatic group (such as, for example, a fluorinated branched alkyl group or the like) is a perfluorinated branched aliphatic group (e.g., alkyl or the like))); ether groups (e.g., —(CH₂)₂OCH₃, —(CH₂)₂OCH₂CH₃, —(CH₂)₂OCH₂CH(CH₃)₂, and —(CH₂)₂O(CH₂)₂CH(CH₃)₂, fluorinated analogs thereof, and the like) (which may comprise one or two group(s) chosen from linear aliphatic groups (e.g., linear alkyl groups and the like); branched aliphatic groups (e.g., branched alkyl groups and the like); fluorinated linear aliphatic groups (e.g., fluorinated linear alkyl groups and the like); fluorinated branched aliphatic groups (e.g., fluourinated branched alkyl groups and the like); and oligoether groups (e.g.,

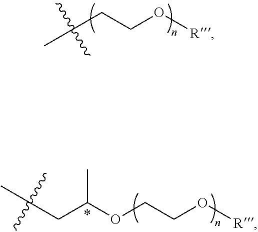

fluorinated analogs thereof, and the like, where the asterisk denotes a stereogenic carbon (i.e., a carbon having R or S stereochemistry, n is 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 8, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100)) (which may comprise one or more group(s) chosen from linear aliphatic groups (e.g., linear alkyl groups and the like), branched aliphatic groups (e.g., branched alkyl groups and the like), fluorinated linear aliphatic groups (e.g., fluorinated linear alkyl groups and the like) (e.g., ethyl groups, propyl groups, and the like and combinations thereof and/or may comprise one or more fluorine groups (e.g., the ether group may be perfluorinated)); and
R′″ is a linear or branched aliphatic group (e.g., alkyl group and the like) (e.g., methyl, ethyl, propyl, isopropyl, butyl, isobutyl, t-butyl, pentyl, neopentyl, isopentyl, and the like), which may comprise one or more fluorine groups (e.g., the oligoether group may be perfluorinated)), and combinations thereof.

In various examples, when a compound of the present disclosure is cyclic, n is 1. In various other examples, when a compound of the present disclosure is cyclic n is 2. In various examples, when a compound of the present disclosure is cyclic, R is

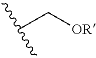

and R′ is —(CH₂)₇CH₃, —(CH₂CH₂O)₃CH₃, —CH₂CH═CH₂, methyl groups, ethyl groups, linear and branched propyl groups, linear and branched butyl groups, linear and branched pentyl groups, linear and branched hexyl groups, linear and branched heptyl groups, linear and branched octyl groups, linear and branched nonyl groups, unsaturated analogs thereof (e.g., linear and branched propenyl groups, linear and branched butenyl groups, linear and branched pentenyl groups, linear and branched hexenyl groups, linear and branched heptenyl groups, linear and branched octenyl groups, and linear and branched nonenyl groups), or

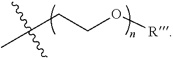

In various embodiments, R is independently

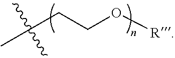

at each occurrence and R′ at each occurrence is independently chosen from

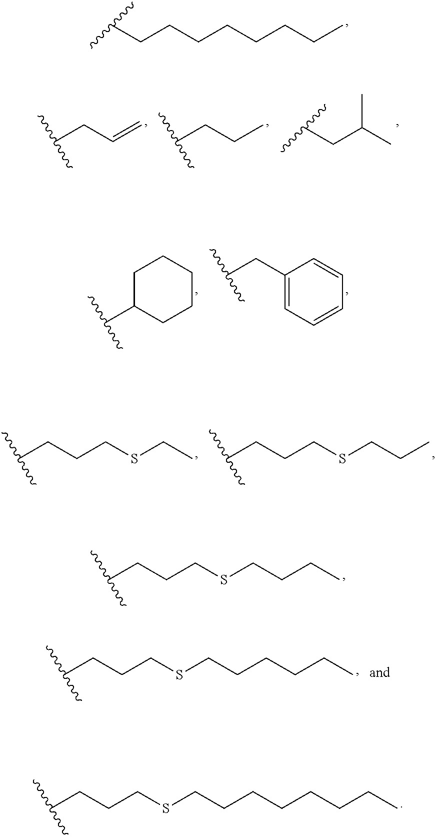

In various embodiments, the compound comprises one or more of aromatic substituents, wherein adjacent aromatic substituents are linked by at least one amide group and the compound has the following structure:

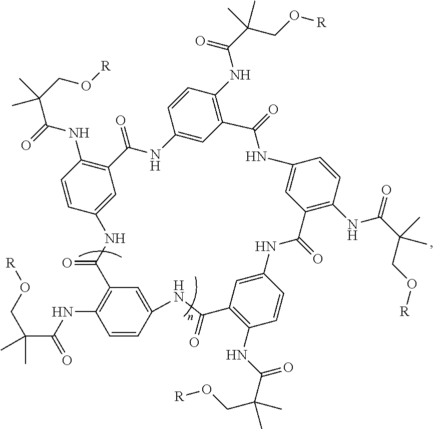

wherein n is 1 or 2, and wherein R is independently at each occurrence chosen from substituted or unsubstituted aliphatic groups, substituted or unsubstituted cyclic aliphatic groups, and substituted or unsubstituted aryl groups. The substituted or unsubstituted aliphatic groups may have one or more heteroatoms (e.g., O, S, or N) in its longest linear chain. In some embodiments, n is 1. In some embodiments, n is 2.

In various embodiments, R is independently at each occurrence chosen from

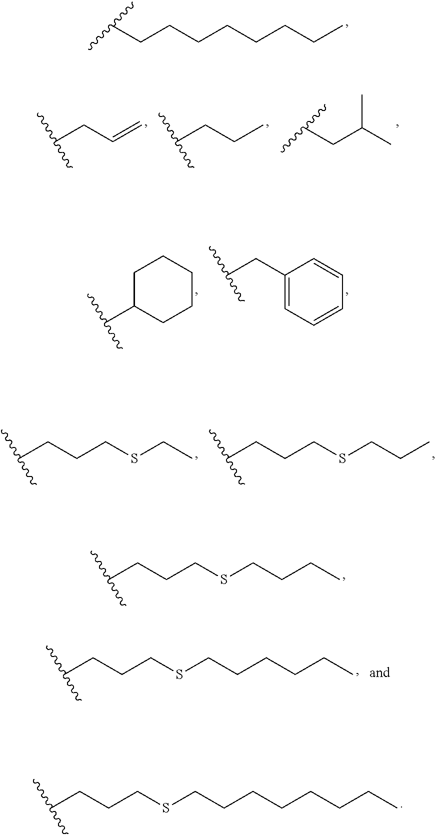

In an aspect, the present disclosure provides compositions. A composition may comprise one or more linear and/or one or more cyclic oligoamide of the present disclosure. A composition may be a pharmaceutical composition. A composition may be suitable for administration to an individual. Non-limiting examples of compositions are provided herein.

In an aspect, the present disclosure provides method of making linear and cyclic oligoamides. In various examples, a linear or cyclic amide of the present disclosure is made by a method of the present disclosure. Non-limiting examples of methods of making linear and cyclic oligoamides are provided herein.

In an aspect, the present disclosure provides uses of linear and cyclic oliogoamides. The linear and/or cyclic oliogoamides may be used in methods such as, for example, forming transmembrane pores for transmembrane transport of hydrogen-bond acceptors and/or ions, sequestering hydrogen-bond acceptors and/or ions (e.g., anions), or the like. Compounds of the present disclosure may be used to enrich materials with ions (e.g., lithium). Non-limiting examples of uses of linear and/or cyclic oligoamides are provided herein.

In an aspect, the present disclosure provides a method of treating an individual diagnosed with or suspected of having cystic fibrosis comprising administering to the individual one or more compound(s) of the present disclosure such that one or more symptom(s) related to the cystic fibrosis is at least partially or completely alleviated.

### BRIEF DESCRIPTION OF THE FIGURES

For a fuller understanding of the nature and objects of the disclosure, reference should be made to the following detailed description taken in conjunction with the accompanying figures.

FIG. 1A shows previously developed aromatic oligoamide foldamer A has a fully constrained backbone. Oligoamide H, with the orientation of its backbone amide groups (red) being inverted relative to those of A, has multiple amide NH groups available for H-bonding.

FIG. 1B shows instead of adopting multiple conformations as represented by H′, oligoamide H, upon binding a guest via H-bonding, will be driven to adopt a crescent conformation in which all backbone amide protons and “internal” aromatic protons, are convergently arranged.

FIG. 2 shows a reactions scheme for the conversion of B1 to B2.

FIG. 3A shows X-ray structure of compound B2b.

FIG. 3B shows X-ray structure of compound 2b′ reveal two conformations related by rotation around the single bonds as indicated by arrows.

FIG. 4A shows two outcomes for the ring-opening reactions of (4H)-3, 1-benzoxazine-4-one C with a nucleophile such as an amine.

FIG. 4B shows treating n-octylamine with compounds B2a and B2b, respectively, gives products B3 and 1b.

FIG. 5A shows a reaction scheme of (a) 1b, c to 1-5.

FIG. 5B shows 2c′ and 4c-NH₂ to form 6c.

FIG. 6A shows partial NOESY spectra of 4c (5 mM) in CDCl₃ containing 5% DMSO-d₆ (500 MHz, 25° C., mixing time=0.4 s). The observed NOEs are indicated by double-headed arrows.

FIG. 6B shows partial NOESY spectra of 4c (5 mM) and tetrabutylammonium iodide (5 mM) in CDCl₃ containing 5% DMSO-d₆ (500 MHz, 25° C., mixing time=0.4 s). The observed NOEs are indicated by double-headed arrows.

FIG. 7 shows (left) a reaction scheme of the precursors to a compound of the present disclosure and (right) a schematic depicting the binding of a polar species such as an anion or a negatively charged group.

FIG. 8 shows ¹H NMR spectra of B2a, B2b, and B2c. (400 MHz, 25° C., 10 mM of corresponding benzoxazinone derivatives in CDCl₃).

FIG. 9A shows mass spectra (ESI-MS) of B2a (m/z [M+H]⁺ Calcd for C₁₇H₂₃N₂O₄ 319.37; Found 319.24). Acetonitrile was used as the mobile phase, since benzoxazinone derivatives (B2a) could undergo the ring-opening reaction with methanol and give corresponding methyl esters in the presence of methanol.

FIG. 9B shows mass spectra (ESI-MS) of B2b (m/z [M+H]⁺ Calcd for C₁₂H₁₃N₂O₄ 249.24; Found 249.06). Acetonitrile was used as the mobile phase, since benzoxazinone derivatives (B2b) could undergo the ring-opening reaction with methanol and give corresponding methyl esters in the presence of methanol.

FIG. 9C shows mass spectra (ESI-MS) of B2c (m/z [M+H]′ Calcd for C₂₀H₂₉N₂O₅ 377.45; Found 377.12). Acetonitrile was used as the mobile phase, since benzoxazinone derivatives (B2c) could undergo the ring-opening reaction with methanol and give corresponding methyl esters in the presence of methanol.

FIG. 10A shows mass spectra (ESI-MS) of 1b (m/z [M+H]⁺ Calcd for C₂₀H₃₂N₃O₄ 378.48; Found 378.11; m/z [M+Na]⁺ Calcd for C₂₀H₃₁N³NaO₄ 400.46; Found 400.18). Methanol was used as the mobile phase.

FIG. 10B shows mass spectra (ESI-MS) of B3 (m/z [M+H]⁺ Calcd for C₂₅H₄₀N₃O₃ 430.60; Found 430.36). Methanol was used as the mobile phase.

FIG. 11 shows ¹H NMR spectra of 1b and B3. (400 MHz, 25° C., 20 mM of corresponding materials in CDCl₃).

FIG. 12 shows ¹H NMR spectra of 2b′ and 2c′. (400 MHz, 25° C., 15 mM of corresponding materials in CDCl₃).

FIG. 13 shows partial stacked ¹H NMR spectra of 4c with 0.0 to 2.0 equiv of TBACl (top) and TBAI (bottom) (400 MHz, 25° C., 5 mM of 4c in 10% CD₃CN/90% CDCl₃. The peak of TMS was set to 0.0 ppm. The chemical shift of corresponding amide protons are shown with dash lines).

FIG. 14 shows partial stacked ¹H NMR spectra of 6c with 0.0 to 2.0 equiv of TBACl (top) and TBAI (bottom) (400 MHz, 25° C., 2 mM of 6c in 5% DMSO-d₆/95% CDCl₃. The peak of TMS was set to 0.0 ppm. The chemical shifts of corresponding amide protons are shown with dashed lines).

FIG. 15 shows partial stacked ¹H NMR spectra of 4c with 0 to 2 equiv of TBAPh₄B (400 MHz, 25° C., 5 mM of 4c in 10% CD₃CN/90% CDCl₃. The peak of TMS was set to 0.0 ppm. The chemical shift of corresponding amide protons are shown with dashed lines).

FIG. 16 shows NOESY of 4c showing NOE interaction between protons d and 4. (500 MHz, 25° C., 5 mM of 4c in 5% DMSO-d₆/95% CDCl₃, mixing time=300 ms).

FIG. 17 shows NOESY spectrum of 4c with 1 equiv of TBAI showing NOE interaction between protons d and 4. (500 MHz, 25° C., 5 mM in 5% DMSO-d₆/95% CDCl₃, mixing time=300 ms).

FIG. 18 shows NOESY spectrum of 4c with 1 equiv of TBACl showing NOE interaction between amide NH protons a, b, c and aromatic CH protons 1, 2, 3, 4. (500 MHz, 25° C., 5 mM in 5% DMSO-d₆/95% CDCl₃, mixing time=300 ms).

FIG. 19 shows partial NOESY spectra of 6c (2 mM) showing NOE interactions between amide NH protons a, b, e, f and aromatic CH protons 1, 2, 3, 5, 6. (500 MHz, 25° C., 5% DMSO-d₆/95% CDCl₃, mixing time=300 ms). The NH protons c and d are overlapped and their NOE interactions with aromatic CH protons cannot be assigned accurately. The important NOE interactions were shown with double-head arrows.

FIG. 20 shows labeled partial ¹H NMR spectra of 6c and equimolar amount of TBACl. (500 MHz, 25° C., 2 mM in the 5% DMSO-d₆/95% CDCl₃).

FIG. 21 shows partial NOESY spectra of 6c with equimolar amount of TBACl showing NOE interaction between amide NH protons a, b, c, d, e, f and aromatic CH protons 1. 2, 3, 4, 5, 6. (500 MHz, 25° C., 2 mM in 5% DMSO-d₆/95% CDCl₃, mixing time=300 ms). The important NOE interactions were shown with double-head arrows.

FIG. 22 shows partial ¹H NMR of 4c. (500 MHz, 25° C., 5 mM in the 5% DMSO-d₆/95% CDCl₃).

FIG. 23 shows partial 2-D gCOSY of 4c. (500 MHz, 25° C., 5 mM in the 5% DMSO-d₆/95% CDCl₃, mixing time=300 ms, and interactions highlighted with double-head arrows).

FIG. 24 shows partial ¹H NMR of 1:1 complex of 4c·TBACl. (500 MHz, 25° C., 5 mM in the 5% DMSO-d₆/95% CDCl₃).

FIG. 25 shows partial 2-D gCOSY of 1:1 complex of 4c·TBACl. (500 MHz, 25° C., 5 mM in the 5% DMSO-d₆/95% CDCl₃, mixing time=300 ms and interactions highlighted with double-head arrows).

FIG. 26 shows partial ¹H-NMR of 1:1 complex of 4c·TBAI. (500 MHz, 5 mM in the 5% DMSO-d₆/95% CDCl₃).

FIG. 27 shows partial 2-D gCOSY of 1:1 complex of 4c·TBAI. (500 MHz, 5 mM in the 5% DMSO-d₆/95% CDCl₃, 25° C., mixing time=300 ms, and interactions highlighted with double-head arrows).

FIG. 28 shows partial ¹H NMR of 4c (400 MHz, 4 mM in different mixed solvents, 25° C.). The amide protons of interest were shown using dashed lines. The peak of TMS was set to 0.0 ppm. In ¹H NMR of 4c in pure DMSO-do, the DMSO-do residue was set to 2.49 ppm. In ¹H NMR of 4c in pure CDCl₃, the proton peaks showed significant line broadening and no accurate assignment was obtained.

FIG. 29 shows partial ¹H NMR of 6c. (500 MHz, 25° C., 2 mM in the 5% DMSO-d₆/95% CDCl₃).

FIG. 30A shows chemical shift of amide NH protons of 4c with increasing amount of TBACl ((a), Kₐ=860±40 M⁻¹) and corresponding fitting output from BindFit v0.5. (5 mM of 4c in 10% CD₃CN/90% CDCl₃, 25° C., 400 MHz).

FIG. 30B shows chemical shift of amide NH protons of 4c with increasing amount of TBAI ((b), Kₐ=400±10 M⁻¹) and corresponding fitting output from BindFit v0.5. (5 mM of 4c in 10% CD₃CN/90% CDCl₃, 25° C., 400 MHz).

FIG. 31A shows chemical shift of amide NH protons of 6c with increasing amount of TBACl (Kₐ=2240±250 M⁻¹) and corresponding fitting output from Bindfit v0.5. (2 mM of 6c in 5% DMSO-d₆/95% CDCl₃, 25° C., 400 MHz).

FIG. 31B shows chemical shift of amide NH protons of 6c with increasing amount of TBAI (Kₐ=2340±120 M⁻¹) and corresponding fitting output from Bindfit v0.5. (2 mM of 6c in 5% DMSO-d₆/95% CDCl₃, 25° C., 400 MHz).

FIG. 32 shows ¹H NMR of compound 1 (CDCl₃, 25° C., 500 MHz).

FIG. 33 shows ¹³C NMR of compound 1 (CDCl₃, 25° C., 75 MHz).

FIG. 34 shows HRMS-ESI spectrum of compound 1 (positive mode).

FIG. 35 shows ¹H NMR of compound 2 (CDCl₃, 25° C., 400 MHz).

FIG. 36 shows ¹³C NMR of compound 2 (CDCl₃, 25° C., 75 MHz).

FIG. 37 shows HRMS-ESI spectrum of compound 2 (positive mode).

FIG. 38 shows ¹H NMR of compound B2a (CDCl₃, 25° C., 400 MHz).

FIG. 39 shows ¹³C NMR of compound B2a (CDCl₃, 25° C., 75 MHz).

FIG. 40 shows ¹H NMR of compound B2b (CDCl₃, 25° C., 400 MHz).

FIG. 41 shows ¹³C NMR of compound B2b (CDCl₃, 25° C., 75 MHz).

FIG. 42 shows ¹H NMR of compound B2c (CDCl₃, 25° C., 400 MHz).

FIG. 43 shows ¹³C NMR of compound B2c (CDCl₃, 25° C., 75 MHz).

FIG. 44 shows ¹H NMR of compound B3 (CDCl₃, 25° C., 400 MHz).

FIG. 45 shows ¹³C NMR of compound B3 (CDCl₃, 25° C., 75 MHz).

FIG. 46 shows HRMS-ESI spectrum of compound B3 (positive mode).

FIG. 47 shows 1HNMR of compound 1b (CDCl₃, 25° C., 500 MHz).

FIG. 48 shows ¹³C NMR of compound 1b (CDCl₃, 25° C., 75 MHz).

FIG. 49 shows HRMS-ESI spectrum of compound 1b (positive mode).

FIG. 50 shows ¹H NMR of compound 1c (CDCl₃, 25° C., 400 MHz).

FIG. 51 shows ¹³C NMR of compound 1c (CDCl₃, 25° C., 75 MHz).

FIG. 52 shows HRMS-ESI spectrum of compound 1c (positive mode).

FIG. 53 shows ¹H NMR of compound 7b (CDCl₃, 25° C., 500 MHz).

FIG. 54 shows ¹³C NMR of compound 7b (DMSO-d₆, 25° C., 75 MHz).

FIG. 55 shows HRMS-ESI spectrum of compound 7b (positive mode).

FIG. 56 shows ¹H NMR of compound 7c (CDCl₃, 25° C., 400 MHz).

FIG. 57 shows ¹³C NMR of compound 7c (CDCl₃, 25° C., 75 MHz).

FIG. 58 shows HRMS-ESI spectrum of compound 7c (positive mode).

FIG. 59 shows ¹H NMR of compound 8b (CDCl₃, 25° C., 500 MHz).

FIG. 60 shows ¹³C NMR of compound 8b (DMSO-d₆, 25° C., 75 MHz).

FIG. 61 shows HRMS-ESI spectrum of compound 8b (positive mode).

FIG. 62 shows ¹H NMR of compound 8c (CDCl₃, 25° C., 400 MHz).

FIG. 63 shows ¹³C NMR of compound 8c (CDCl₃, 25° C., 75 MHz).

FIG. 64 shows HRMS-ESI spectrum of compound 8c (positive mode).

FIG. 65 shows ¹H NMR of compound 9b (CDCl₃, 25° C., 500 MHz).

FIG. 66 shows ¹³C NMR of compound 9b (DMSO-d₆, 25° C., 75 MHz).

FIG. 67 shows HRMS-ESI spectrum of compound 9b (positive mode).

FIG. 68 shows ¹H NMR of compound 9c (CDCl₃, 25° C., 400 MHz).

FIG. 69 shows ¹³C NMR of compound 9c (CDCl₃, 25° C., 75 MHz).

FIG. 70 shows HRMS-ESI spectrum of compound 9c (positive mode).

FIG. 71 shows ¹H NMR of compound 2b′ (CDCl₃, 25° C., 500 MHz).

FIG. 72 shows ¹³C NMR of compound 2b′ (DMSO-d₆, 25° C., 75 MHz).

FIG. 73 shows ¹H NMR of compound 2c′ (CDCl₃, 25° C., 400 MHz).

FIG. 74 shows ¹³C NMR of compound 2c′ (CDCl₃, 25° C., 75 MHz).

FIG. 75 shows ¹H NMR of compound 2b (CDCl₃, 25° C., 500 MHz).

FIG. 76 shows ¹³C NMR of compound 2b (CDCl₃, 25° C., 75 MHz).

FIG. 77 shows HRMS-ESI spectrum of compound 2b (positive mode).

FIG. 78 shows ¹H NMR of compound 2c (CDCl₃, 25° C., 400 MHz).

FIG. 79 shows ¹³C NMR of compound 2c (CDCl₃, 25° C., 75 MHz).

FIG. 80 shows HRMS-ESI spectrum of compound 2c (positive mode).

FIG. 81 shows ¹H NMR of compound 3b (CDCl₃, 25° C., 500 MHz).

FIG. 82 shows ¹³C NMR of compound 3b (CDCl₃, 25° C., 75 MHz).

FIG. 83 shows HRMS-ESI spectrum of compound 3b (positive mode).

FIG. 84 shows ¹H NMR of compound 3c (10% DMSO-d₆/90% CDCl₃, 25° C., 400 MHz).

FIG. 85 shows ¹³C NMR of compound 3c (10% DMSO-d₆/90% CDCl₃, 25° C., 75 MHz).

FIG. 86 shows HRMS-ESI spectrum of compound 3c (positive mode).

FIG. 87 shows ¹H NMR of compound 4c (10% DMSO-d₆/90% CDCl₃, 25° C., 400 MHz).

FIG. 88 shows ¹³C NMR of compound 4c (10% DMSO-d₆/90% CDCl₃, 25° C., 75 MHz).

FIG. 89 shows HRMS-ESI spectrum of compound 4c (positive mode).

FIG. 90 shows ¹H NMR of compound 5c (10% DMSO-d₆/90% CDCl₃, 25° C., 400 MHz).

FIG. 91 shows ¹³C NMR of compound 5c (10% DMSO-d₆/90% CDCl₃, 25° C., 75 MHz).

FIG. 92 shows HRMS-ESI spectrum of compound 5c (positive mode).

FIG. 93 shows ¹H NMR of compound 6c (10% DMSO-d₆/90% CDCl₃, 25° C., 400 MHz).

FIG. 94 shows ¹³C NMR of compound 6c (10% DMSO-d₆/90% CDCl₃, 25° C., 75 MHz).

FIG. 95 shows HRMS-MALDI spectrum of compound 6c (positive mode). In the HRMS-MALDI spectrum, the monomer fragment (m/z: 347.2344) is also observed, indicating that 6c is not stable in the HRMS-MALDI.

FIG. 96 shows a copy of a thermal ellipsoid plot for the crystal structure of B2b which was drawn at 30% probability.

FIG. 97 shows a copy of a thermal ellipsoid plot for the crystal structure of 2b′ which was drawn at 30% probability.

FIG. 98A shows the general structure of previously developed aromatic oligoamide foldamer AO with a backbone fully constrained by three-center intramolecular H-bonds. Inverting the orientation of the backbone amide groups of AO gives aromatic oligoamide OA.

FIG. 98B shows the population of crescent conformation OA, in which all backbone amide protons and the “inner” aromatic protons are convergently arranged, will be increased upon binding to a guest (sphere) via H-bonding interactions (dashed lines).

FIG. 99A shows design of aromatic oligoamide macrocycles cOA as cyclic anion binders.

FIG. 99B shows an energy-minimized structure of the cyclic 5mer (R=methyl) with an inner cavity of ~6.8 Å (van der Waals 4.4 Å) across.

FIG. 100 shows an X-ray structure of compound A2b.

FIG. 101A shows two outcomes for the ring-opening reactions of (4H)-3, 1-benzoxazine-4-one B with a nucleophile such as an amine.

FIG. 101B shows treating n-octylamine with compounds A2a and A2b, respectively, gives products C and 1b.

FIG. 102A shows the chemical structure of 2b′ and 2c′.

FIG. 102B shows a structure of compound 2b′ revealing two conformations that are related by rotation around the amide-aryl bond indicated by arrows.

FIG. 103 shows binding constants Kₐ (M⁻¹) of amide hosts with anions. The titrations were performed in mixed solvent containing 10% CD₃CN in CDCl₃ at room temperature.

FIG. 104 shows binding constants Kₐ (M⁻¹) of 7c with anions.

FIG. 105 shows the general structure (top) and crystal structure (bottom) of a cyclic compound of the present disclosure. In the top image, R=Sidechain. The property of the sidechain determines the compatibility of the macrocycle (MC) and anion-MC complex(es) with different solvents. The bottom image shows the persistent shape of the macrocyclic backbone; and the rigidly held, multiple NH and CH hydrogen bond donors that orient toward the center of the inner cavity which is predisposed for binding anions

FIG. 106 shows a schematic of an anion binding to a cyclic compound of the present disclosure. Specifically, binding to the c5mer wraps an anion in a layer of “cloth”, making the anion compatible with (soluble in) a solvent (e.g., a hydrophobic one) in which a naked anion cannot go into. The shaded circle can represent an ion, such as, for example, chloride (Cl⁻), bromide (Br⁻), iodide (I⁻), or other anion. For example, the compound may have high affinity for the binding of anions. Binding constants (Kₐ)>10⁷ M⁻¹ for Cl⁻, Br⁻, I⁻ in chloroform (CHCl₃). Order of binding strength: Cl⁻<Br⁻<I⁻. Binding of environmentally important anions (Kₐ's measured in chloroform with 5% methanol): sulfate (SO₄²⁻): 4×10⁵ M⁻¹, perchlorate (ClO₄₋): 3×10⁶ M⁻¹, and nitrate (NO₃): 2×10⁶ M⁻¹

FIG. 107 shows a schematic for assessment of the transport of halides across the cell membranes (e.g., lipid bilayers). Lipids (X⁻) are hydrophilic and cannot cross cell membranes. Upon being bound to c5mer, an anion is carried (shuttled) by the macrocycle to cross the membrane and delivered into liposomes (or large unilamellar vesicles (LUVs)). The internalized anions (and protons), are detected by the quenched emission of HPTS.

FIG. 108 shows c5mer-mediated transport of halides across cell membranes and the results from vesicle (LUV)-based assays. The efficient and selective transport of the chloride ion across cell membranes by the c5mer suggests that the macrocycle could serve as an effective carrier to restore chloride transport to cells with defect anion transport, a hallmark of the genetic disease cystic fibrosis (CF).

FIG. 109 shows the transport of anions (via ion pairs) across bulk organic phase. Organic cations have been used as the counterions to assist the transport of anions (e.g., Cl⁻) across the organic phase.

FIG. 110 shows enrichment of lithium ions with liquid membranes. The top layer is a solution (mixture) of LiCl, NaCl, and KCl in water. The middle layer shows a liquid membrane (LM). The solubilization of LiCl into the LM, assisted by an organic anion binder (e.g., the c5mer) for chloride and an organic binder specific for the lithium ion. The bottom layer shows the second (or n^{th}) aqueous phase. LiCl is release from the LM into the second aqueous phase.

FIG. 111 shows the general structure of cyclic compound of the present disclosure and possible synthetic routes.

FIG. 112 shows a synthetic route for a cyclic compound of the present disclosure with six repeat units.

FIG. 113A shows linear oligoamide 1 and macrocycles c5 consisting of basic residues derived from 5-amino-N-acylanthranilic acid. Linear oligoamides 1 with its energetically unfavorable crescent conformation 1crs, and the overall electrical dipole moments of amides 1a and 1b in the depicted conformations from DFT calculations performed with the ADF software package. The backbones of the linear and cyclic oligoamides are highlighted with thick bonds.

FIG. 113B shows linear oligoamide 1 and macrocycles c5 consisting of basic residues derived from 5-amino-N-acylanthranilic acid. Synthesis of macrocycles c5a-c based on a one-pot macrocyclization process. The backbones of the linear and cyclic oligoamides are highlighted with thick bonds.

FIG. 113C shows linear oligoamide 1 and macrocycles c5 consisting of basic residues derived from 5-amino-N-acylanthranilic acid. Crystal structure of c5c in which two of the five side chains are disordered with water molecules being hydrogen-bonded to the backbone NH groups.

FIG. 114A shows binding stoichiometry of macrocycle c5 with anions. a, Changes in the 330-nm absorbance of c5a (20 μM) titrated with 0-2.5 equiv of TBA⁺Cl⁻ in CH₃OH:CHCl₃ (5:95, v/v). b, Changes in the chemical shifts of endocyclic protons a of c5a titrated with 0-5 equiv of TBA⁺Cl⁻ in DMSO-d₆:CDCl₃ (1:99, v/v). c, Optimized structures of the 1:1 complexes of c5 with Cl⁻, Br⁻, I⁻, NO₃⁻, ClO₄⁻, and DPP⁻. In each complex, the CH/NH protons and the anion that are within van der Waals contact distances (d_{H . . . O}≤2.8 Å; d_{H . . . CI-}≤3.0 Å; d_{H . . . Br-}≤3.1 Å; d_{H . . . I-}≤3.3 Å) are indicated by red dashed lines. The structures were computationally optimized in vacuum.

FIG. 114B shows binding stoichiometry of macrocycle c5 with anions. Changes in the chemical shifts of endocyclic protons a of c5a titrated with 0-5 equiv of TBA⁺Cl⁻ in DMSO-d₆:CDCl₃ (1:99, v/v).

FIG. 114C shows binding stoichiometry of macrocycle c5 with anions. Optimized structures of the 1:1 complexes of c5 with Cl⁻, Br⁻, I⁻, NO₃, ClO₄, and DPP⁻. In each complex, the CH/NH protons and the anion that are within van der Waals contact distances (d_{H . . . O}≤2.8 Å; d_{H . . . CI-}≤3.0 Å; d_{H . . . Br-}≤3.1 Å; d_{H . . . I-}≤3.3 Å) are indicated by red dashed lines. The structures were computationally optimized in vacuum.

FIG. 115A shows ¹H NMR spectra and association constants (log Kₐ) of c5a. Top: ¹H NMR spectra of c5a (0.1 mM) titrated with 0 to 5 equiv. of TBA⁺I⁻ in DMSO-d₆:CDCl₃ (15:85, v/v); bottom: The solvated cavity of c5 with DMSO molecule(s) hydrogen-bonded to amide protons b (left) and the desolvated cavity of c5 occupied by an anion (X−) which forms hydrogen bonds with amide protons b and phenyl protons a (right).

FIG. 115B shows ¹H NMR spectra and association constants (log Kₐ) of c5a. Top: ¹H NMR spectra of c5a (0.1 mM) titrated with 0 to 2 equiv. of TBA⁺HSO₄⁻ in DMSO-d₆:CDCl₃ (15:85, v/v); bottom: structures of three oxoanions.

FIG. 115C shows ¹H NMR spectra and association constants (log Kₐ) of c5a. c, Association constants (log Kₐ) of c5a with the anions in CH₃CN:CHCl₃ (5:95).

FIG. 115D shows ¹H NMR spectra and association constants (log Kₐ) of c5a. Partial 2D (ROESY) spectra of the 1:1 mixture of c5a and TBA·MPP in DMSO-d₆ (20 mM, 500 MHz, mixing time=0.4 s, 25° C.).

FIG. 116A shows transmembrane transport of halide ions promoted by macrocycle c5a. Dissipation of the pH gradient quenches the emission of the pH-sensitive dye HPTS entrapped in 1-palmitoyl-2-oleoyl-sn-glycero-3-phosphocholine (POPC) LUVs.

FIG. 116B shows transmembrane transport of halide ions promoted by macrocycle c5a. Normalized ratio of time-lapse fluorescence emission intensities of Cl⁻, Br⁻, and I⁻ transport facilitated by c5a, along with the control curves (in blue) measured with vesicles in the absence of c5a. The transport of halides was examined by measuring the fluorescence excitation/emission wavelengths of HPTS, with excitation at 405 nm and emission at 510 nm, and excitation at 450 nm and emission at 510 nm. The transmembrane anion permeability of vesicles with and without c5a was determined by numerically fitting (black lines) the ratio of emission intensities corresponding to the 450 nm/405 nm excitations over the time-lapse. The total anion permeability (ΔP_{X-}) of each halide ion elicited by c5a is shown. (Intensity ratio, F450/F405=fluorescence emission intensities at excitation wavelengths of 450 nm and 405 nm).

FIG. 116C shows transmembrane transport of halide ions promoted by macrocycle c5a. Normalized ratio of time-lapse fluorescence emission intensities of Cl⁻, Br⁻, and I⁻ transport facilitated by c5a, along with the control curves (in blue) measured with vesicles in the absence of c5a. The transport of halides was examined by measuring the fluorescence excitation/emission wavelengths of HPTS, with excitation at 405 nm and emission at 510 nm, and excitation at 450 nm and emission at 510 nm. The transmembrane anion permeability of vesicles with and without c5a was determined by numerically fitting (black lines) the ratio of emission intensities corresponding to the 450 nm/405 nm excitations over the time-lapse. The total anion permeability (ΔP_{X-}) of each halide ion elicited by c5a is shown. (Intensity ratio, F450/F405=fluorescence emission intensities at excitation wavelengths of 450 nm and 405 nm).

FIG. 116D shows transmembrane transport of halide ions promoted by macrocycle c5a. Normalized ratio of time-lapse fluorescence emission intensities of Cl⁻, Br⁻, and I⁻ transport facilitated by c5a, along with the control curves (in blue) measured with vesicles in the absence of c5a. The transport of halides was examined by measuring the fluorescence excitation/emission wavelengths of HPTS, with excitation at 405 nm and emission at 510 nm, and excitation at 450 nm and emission at 510 nm. The transmembrane anion permeability of vesicles with and without c5a was determined by numerically fitting (black lines) the ratio of emission intensities corresponding to the 450 nm/405 nm excitations over the time-lapse. The total anion permeability (ΔP_{X}⁻) of each halide ion elicited by ca is shown. (Intensity ratio, F450/F405=fluorescence emission intensities at excitation wavelengths of 450 nm and 405 nm).

FIG. 117A shows macrocycle c5a restores ASL volume hemostasis in CF airway epithelial cultures. a, Illustration of normal and CF human airway cells in profile showing the effect of reduced CFTR-mediated Cl⁻ secretion combined with unregulated sodium absorption (via ENaC) on the ASL volume thickness (blue region). b, Representative XZ-confocal microscopy images of human airway epithelial cultures (labeled with Texas Red-dextran; red) showing the steady-state ASL thickness in non-CF (i.e., normal) (left) and CF (center; control) airways and the effect of treating CF cultures with c5a (right). The location of the unlabeled airways cells is shown for reference. Bar=7 μm, the length of extended cilia. c, Summary of the average thickness of the ASL layers of the control and treated CF cell cultures based on 12 independent measurements (***p<0.05).

FIG. 117B shows macrocycle c5a restores ASL volume hemostasis in CF airway epithelial cultures. a, Illustration of normal and CF human airway cells in profile showing the effect of reduced CFTR-mediated Cl⁻ secretion combined with unregulated sodium absorption (via ENaC) on the ASL volume thickness (blue region). b, Representative XZ-confocal microscopy images of human airway epithelial cultures (labeled with Texas Red-dextran; red) showing the steady-state ASL thickness in non-CF (i.e., normal) (left) and CF (center; control) airways and the effect of treating CF cultures with c5a (right). The location of the unlabeled airways cells is shown for reference. Bar=7 μm, the length of extended cilia. c, Summary of the average thickness of the ASL layers of the control and treated CF cell cultures based on 12 independent measurements (***p<0.05).

FIG. 117C shows macrocycle c5a restores ASL volume hemostasis in CF airway epithelial cultures. a, Illustration of normal and CF human airway cells in profile showing the effect of reduced CFTR-mediated Cl⁻ secretion combined with unregulated sodium absorption (via ENaC) on the ASL volume thickness (blue region). b, Representative XZ-confocal microscopy images of human airway epithelial cultures (labeled with Texas Red-dextran; red) showing the steady-state ASL thickness in non-CF (i.e., normal) (left) and CF (center; control) airways and the effect of treating CF cultures with c5a (right). The location of the unlabeled airways cells is shown for reference. Bar=7 μm, the length of extended cilia. c, Summary of the average thickness of the ASL layers of the control and treated CF cell cultures based on 12 independent measurements (***p<0.05).

FIG. 118A shows top (left) and side (right) views of energy-minimized structures of the five-residue macrocycle c5.

FIG. 118B shows top (left) and side (right) views of energy-minimized structures of the six-residue macrocycle c6.

FIG. 119 shows formation energy for ‘pentamer L5’→‘macrocycle c5’+‘CH₄’. The energy of a CH₄ molecule was added to the energy of the macrocycle c5 to keep the atom type and atom count equal for comparing the energies of macrocycle c5 (left) and linear pentamer L5 (right). The formation of the macrocycle c5 and a methane molecule is calculated to be 5.93 kcal/mol endothermic with respect to the linear pentamer L5.

FIG. 120A shows crystal structure of macrocycle c5a. A topo view is shown. Most of the sidechains are disordered. Two DMSO molecules are H-bonded (dashed lines) are shown.

FIG. 120B shows crystal structure of macrocycle c5a. A side view is shown. Most of the sidechains are disordered. Two DMSO molecules are H-bonded (dashed lines) are shown.

FIG. 121A shows top view packing of a top view of macrocycle c5c in the solid state. The two residues that are involved in stacking are shown as sticks and the rest of the bonds are shown as wire. Hydrogen atoms, except for those of amide groups, are omitted for clarity.

FIG. 121B shows side view packing of two adjacent molecules of macrocycle c5c in the solid state. The two residues that are involved in stacking are shown as sticks and the rest of the bonds are shown as wire. Hydrogen atoms, except for those of amide groups, are omitted for clarity.

FIG. 122 shows ¹H NMR spectra of a, c5a; b, c5b; and c, c5c in CDCl₃ (0.2 mM, 400 MHz, 25° C.).

FIG. 123 shows ¹H NMR spectra of c5a in mixed solvent of DMSO-d₆ and CDCl₃ (1.0 mM, 400 MHz, 25° C.).

FIG. 124A shows UV-vis titration of c5a (20 μM) with TBA⁺Br⁻, in MeOH:CHCl₃ (5:95, v/v) at 25° C.

FIG. 124B shows UV-vis titration of c5a (20 μM) with TBA⁺I⁻, in MeOH:CHCl₃ (5:95, v/v) at 25° C.

FIG. 124C shows UV-vis titration of c5a (20 μM) with TBA⁺BF₄⁻, in MeOH:CHCl₃ (5:95, v/v) at 25° C.

FIG. 124D shows UV-vis titration of c5a (20 μM) with TBA⁺ClO₄⁻, in MeOH:CHCl₃ (5:95, v/v) at 25° C.

FIG. 124E shows UV-vis titration of c5a (20 μM) with TBA⁺IO₄⁻, in MeOH:CHCl₃ (5:95, v/v) at 25° C.

FIG. 124F shows UV-vis titration of c5a (20 μM) with TEA⁺PF₆⁻, in MeOH:CHCl₃ (5:95, v/v) at 25° C.

FIG. 124G shows UV-vis titration of c5a (20 μM) with TBA NO₃⁻, in MeOH:CHCl₃ (5:95, v/v) at 25° C.

FIG. 124H shows UV-vis titration of c5a (20 μM) with TBA⁺HSO₄⁻, in MeOH:CHCl₃ (5:95, v/v) at 25° C.

FIG. 124I shows UV-vis titration of c5a (20 μM) with TBA⁺MPP⁻, in MeOH:CHCl₃ (5:95, v/v) at 25° C.

FIG. 124J shows UV-vis titration of c5a (20 μM) with, TMA⁺DPP⁻, in MeOH:CHCl₃ (5:95, v/v) at 25° C.

FIG. 125A shows ¹H NMR titration of c5a (0.25 mM) with TBA⁺Cl⁻, in DMSO-d₆:CDCl₃ (1:99, v/v) at 25° C.

FIG. 125B shows ¹H NMR titration of c5a (0.25 mM) with TBA⁺Br⁻, in DMSO-d₆:CDCl₃ (1:99, v/v) at 25° C.

FIG. 125C shows ¹H NMR titration of c5a (0.25 mM) with TBA⁺I⁻, in DMSO-d₆:CDCl₃ (1:99, v/v) at 25° C.

FIG. 125D shows ¹H NMR titration of c5a (0.25 mM) with TBA⁺ClO₄⁻, in DMSO-d₆:CDCl₃ (1:99, v/v) at 25° C.

FIG. 126A shows high-resolution mass spectra (negative ion mode) the 1:1 complexes of macrocycle c5c with TBA⁺Cl⁻. If [2×c5c+Cl⁻]⁻ existed, a peak at m/z=2778.6037 would be observed.

FIG. 126B shows high-resolution mass spectra (negative ion mode) the 1:1 complexes of macrocycle c5c with TBA⁺Br⁻. If [2×c5c+Br]⁻ existed, peak at m/z=2823.0547 would be observed.

FIG. 126C shows high-resolution mass spectra (negative ion mode) the 1:1 complexes of macrocycle c5c with TBA⁺I⁻. If [2×c5c+I]⁻ existed, peak at m/z=2870.0552 would be observed.

FIG. 126D shows high-resolution mass spectra (negative ion mode) the 1:1 complexes of macrocycle c5c with TBA⁺BF₄⁻. If [2×c5c+BF₄]⁻ existed, a peak at m/z=2828.3209 would be observed.

FIG. 126E shows high-resolution mass spectra (negative ion mode) the 1:1 complexes of macrocycle c5c with TBA⁺ClO₄⁻. If [2×c5c+ClO₄]⁻ existed, a peak at m/z=2946.0635 would be observed. The peak at m/z=1416.1518 corresponds to the 1:1 complex of c5c and formate—formic acid was added in the mobile phase. The peak at m/z=2156.4422 cannot be assigned to any complex of c5a and ClO₄⁻ and is most likely from impurity/contaminant from the equipment. The peak at m/z=2101.9665 cannot be assigned to any complex of c5a and ClO₄⁻ and is most likely from impurity/contaminant from the equipment.

FIG. 126F shows high-resolution mass spectra (negative ion mode) the 1:1 complexes of macrocycle c5c with TBA⁺IO₄⁻. If [2×c5c+IO₄]⁻ existed, a peak at m/z=2934.1838 would be observed. The peak at m/z=1416.6587 corresponds to the 1:1 complex of c5c and formate—formic acid was added in the mobile phase.

FIG. 126G shows high-resolution mass spectra (negative ion mode) the 1:1 complexes of macrocycle c5c with TEA⁺PF₆⁻. (If [2×c5c+PF₆]⁻ existed, a peak at m/z=2888.1149 would be observed. The peak at m/z=1416.6529 corresponds to the 1:1 complex of c5c and formate—formic acid was added in the mobile phase).

FIG. 126H shows high-resolution mass spectra (negative ion mode) the 1:1 complexes of macrocycle c5c with TBA⁺NO₃⁻. If [2×c5c+NO₃]⁻ existed, a peak at m/z=2803.3058 would be observed. The peak at m/z=1416.6607 corresponds to the 1:1 complex of c5c and formate—formic acid was added in the mobile phase. The peaks at m/z=2110-2119 cannot be assigned and are likely from impurity/contaminant from the equipment.

FIG. 126I shows high-resolution mass spectra (negative ion mode) the 1:1 complexes of macrocycle c5c with TBA⁺HSO₄⁻. If [2×c5c+HSO₄]⁻ existed, a peak at m/z=2840.2207. would be observed. The peak at m/z=1419.1239 corresponds to [2×c5c+SO₄²⁻].

FIG. 126J shows high-resolution mass spectra (negative ion mode) the 1:1 complexes of macrocycle c5c with TMA DPP⁻. If [2×c5c+DPP]⁻ existed, a peak at m/z=2992.3299 would be observed. The peak at m/z=1416.6491 corresponds to the 1:1 complex of c5c and formate—formic acid was added in the mobile phase.

FIG. 126K shows high-resolution mass spectra (negative ion mode) the 1:1 complexes of macrocycle c5c with TBA⁺MPP⁻. If [2×c5c+MPP]⁻ existed, a peak at m/z=2914.2611 would be observed. The peak at m/z=1416.6499 corresponds to the 1:1 complex of c5c and formate—formic acid was added in the mobile phase.

FIG. 127 shows optimized structures of the 1:1 complexes of c5 with HSO₄⁻ and MPP⁻. In each complex, the CH/NH protons and the anion that are within van der Waals contact distances (d_{H . . . O}≤2.8 Å) are indicated by red dashed lines. The structures were computationally optimized in vacuum.

FIG. 128A shows partial 1H NMR spectra of c5a (0.1 mM) titrated with 0 to 5 equiv of TBA⁺Cl⁻, in DMSO-d6:CDCl₃ (15:85, v/v).

FIG. 128B shows partial 1H NMR spectra of c5a (0.1 mM) titrated with 0 to 5 equiv of TBA⁺Br⁻, in DMSO-d6:CDCl₃ (15:85, v/v).

FIG. 128C shows partial 1H NMR spectra of c5a (0.1 mM) titrated with 0 to 5 equiv of TBA⁺ClO₄⁻, in DMSO-d6:CDCl₃ (15:85, v/v).

FIG. 128D shows partial 1H NMR spectra of c5a (0.1 mM) titrated with 0 to 2 equiv. of TMA⁺DPP⁻, in DMSO-d6:CDCl₃ (15:85, v/v).

FIG. 128E shows partial 1H NMR spectra of c5a (0.1 mM) titrated with 0 to 2 equiv of TBA⁺MPP⁻, in DMSO-d6:CDCl₃ (15:85, v/v).

FIG. 129A shows binding isotherms of c5a or c5b (0.1-0.2 mM) titrated with 0-5 equiv of TBA⁺Cl⁻ in DMSO-_{d6}:CDCl₃ (15:85, v/v). In each case, the fitting output is based on the chemical shift changes of CH protons a and NH protons b (unless indicated otherwise) using the online fitting tool BindFit (v0.5).

FIG. 129B shows binding isotherms of c5a or c5b (0.1-0.2 mM) titrated with 0-5 equiv of TBA⁺I⁻ in DMSO-_{d6}:CDCl₃ (15:85, v/v). In each case, the fitting output is based on the chemical shift changes of CH protons a and NH protons b (unless indicated otherwise) using the online fitting tool BindFit (v0.5).

FIG. 129C shows binding isotherms of c5a or c5b (0.1-0.2 mM) titrated with 0-5 equiv of TBA⁺NO₃⁻ in DMSO-_{d6}:CDCl₃ (15:85, v/v). In each case, the fitting output is based on the chemical shift changes of CH protons a and NH protons b (unless indicated otherwise) using the online fitting tool BindFit (v0.5).

FIG. 129D shows binding isotherms of c5a or c5b (0.1-0.2 mM) titrated with 0-5 equiv of TBA⁺IO₄⁻ in DMSO-_{d6}:CDCl₃ (15:85, v/v). In each case, the fitting output is based on the chemical shift changes of CH protons a and NH protons b (unless indicated otherwise) using the online fitting tool BindFit (v0.5).

FIG. 129E shows binding isotherms of c5a or c5b (0.1-0.2 mM) titrated with 0-5 equiv of TBA⁺Br⁻ in DMSO-_{d6}:CDCl₃ (15:85, v/v). In each case, the fitting output is based on the chemical shift changes of CH protons a and NH protons b (unless indicated otherwise) using the online fitting tool BindFit (v0.5).

FIG. 129F shows binding isotherms of c5a or c5b (0.1-0.2 mM) titrated with 0-5 equiv of TBA⁺BF₄⁻ in DMSO-_{d6}:CDCl₃ (15:85, v/v). In each case, the fitting output is based on the chemical shift changes of CH protons a and NH protons b (unless indicated otherwise) using the online fitting tool BindFit (v0.5).

FIG. 129G shows binding isotherms of c5a or c5b (0.1-0.2 mM) titrated with 0-5 equiv of TBA⁺ClO₄⁻ in DMSO-_{d6}:CDCl₃ (15:85, v/v). In each case, the fitting output is based on the chemical shift changes of CH protons a and NH protons b (unless indicated otherwise) using the online fitting tool BindFit (v0.5).

FIG. 129H shows binding isotherms of c5a or c5b (0.1-0.2 mM) titrated with 0-5 equiv of TBA⁺Cl⁻ in CD₃CN:CDCl₃ (80:20, v/v). In each case, the fitting output is based on the chemical shift changes of CH protons a and NH protons b (unless indicated otherwise) using the online fitting tool BindFit (v0.5).

FIG. 129I shows binding isotherms of c5a or c5b (0.1-0.2 mM) titrated with 0-5 equiv of TBA⁺I⁻ in CD₃CN:CDCl₃ (80:20, v/v). In each case, the fitting output is based on the chemical shift changes of CH protons a and NH protons b (unless indicated otherwise) using the online fitting tool BindFit (v0.5).

FIG. 129J shows binding isotherms of c5a or c5b (0.1-0.2 mM) titrated with 0-5 equiv of TBA⁺NO₃ in CD₃CN:CDCl₃ (80:20, v/v). In each case, the fitting output is based on the chemical shift changes of CH protons a and NH protons b (unless indicated otherwise) using the online fitting tool BindFit (v0.5).

FIG. 129K shows binding isotherms of c5a or c5b (0.1-0.2 mM) titrated with 0-5 equiv of TBA⁺IO₄⁻ in CD₃CN:CDCl₃ (80:20, v/v). In each case, the fitting output is based on the chemical shift changes of CH protons a and NH protons b (unless indicated otherwise) using the online fitting tool BindFit (v0.5).

FIG. 129L shows binding isotherms of c5a or c5b (0.1-0.2 mM) titrated with 0-5 equiv of TBA⁺Br⁻in CD₃CN:CDCl₃ (80:20, v/v). In each case, the fitting output is based on the chemical shift changes of CH protons a and NH protons b (unless indicated otherwise) using the online fitting tool BindFit (v0.5).

FIG. 129M shows binding isotherms of c5a or c5b (0.1-0.2 mM) titrated with 0-5 equiv of TBA⁺BF₄⁻ in CDsCN:CDCl₃ (80:20, v/v). In each case, the fitting output is based on the chemical shift changes of CH protons a and NH protons b (unless indicated otherwise) using the online fitting tool BindFit (v0.5).

FIG. 129N shows binding isotherms of c5a or c5b (0.1-0.2 mM) titrated with 0-5 equiv of TBA⁺ClO₄⁻ in CD₃CN:CDCl₃ (80:20, v/v). In each case, the fitting output is based on the chemical shift changes of CH protons a and NH protons b (unless indicated otherwise) using the online fitting tool BindFit (v0.5).

FIG. 129O shows binding isotherms of c5a or c5b (0.1-0.2 mM) titrated with 0-5 equiv of TBA⁺PF₆⁻ in CD₃CN:CDCl₃ (80:20, v/v). In each case, the fitting output is based on the chemical shift changes of CH protons a and NH protons b (unless indicated otherwise) using the online fitting tool BindFit (v0.5).

FIG. 129P shows binding isotherms of c5a or c5b (0.1-0.2 mM) titrated with 0-5 equiv of TBA HSO₄⁻ in DMSO-_{d6}:CDCl₃ (70:30, v/v). In each case, the fitting output is based on the chemical shift changes of CH protons a and NH protons b (unless indicated otherwise) using the online fitting tool BindFit (v0.5).

FIG. 129Q shows binding isotherms of c5a or c5b (0.1-0.2 mM) titrated with 0-5 equiv of TBA⁺MPP⁻ in DMSO-_{d6}:CDCl₃ (70:30, v/v). In each case, the fitting output is based on the chemical shift changes of CH protons a and NH protons b (unless indicated otherwise) using the online fitting tool BindFit (v0.5).

FIG. 129R shows binding isotherms of c5a or c5b (0.1-0.2 mM) titrated with 0-5 equiv of TBA⁺DPP⁻ in DMSO-_{d6}:CDCl₃ (70:30, v/v). In each case, the fitting output is based on the chemical shift changes of CH protons a and NH protons b (unless indicated otherwise) using the online fitting tool BindFit (v0.5).

FIG. 129S shows binding isotherms of c5a or c5b (0.1-0.2 mM) titrated with 0-5 equiv of TBA⁺DPP⁻ in D₂O:acetone-_{d6} (20:80, v/v). In each case, the fitting output is based on the chemical shift changes of CH protons a and NH protons b (unless indicated otherwise) using the online fitting tool BindFit (v0.5).

FIG. 129T shows binding isotherms of c5a or c5b (0.1-0.2 mM) titrated with 0-5 equiv of TBA⁺MPP⁻ in D₂O:acetone-d₆ (20:80, v/v). In each case, the fitting output is based on the chemical shift changes of CH protons a and NH protons b (unless indicated otherwise) using the online fitting tool BindFit (v0.5).

FIG. 129U shows binding isotherms of c5a or c5b (0.1-0.2 mM) titrated with 0-5 equiv of TBA⁺DPP⁻ in D₂O:acetone-d₆ (60:40, v/v). In each case, the fitting output is based on the chemical shift changes of CH protons a and NH protons b (unless indicated otherwise) using the online fitting tool BindFit (v0.5).

FIG. 130A shows ITC titration curves (upper panel) and binding isotherms (lower panel) for the complexation of c5a (0.5 mM) with the TBA⁺ salts (5 mM each) of Cl⁻, Br⁻, I⁻, and NO₃⁻, in DMSO:CHCl₃ (5:95, v/v) at 25° C. Concentrations of c5a (in the cell) used in the titrations: 0.075 mM with TBA⁺Br⁻ (1 mM); 0.1 mM with TBA⁺Cl⁻ (1 mM); 0.5 mM with TBA⁺NO₃⁻ (5 mM); and TBA⁺ClO₄⁻ (5 mM). For two-step competition titration experiments, Cl⁻ (2.5 mM) served as the competitor for the titration of c5a with I⁻, PF₆⁻, served as the competitor for the titration of c5a with NO₃⁻ (PF₆⁻: 1 mM) and ClO₄⁻ (PF₆⁻: 0.5 mM).

FIG. 130B shows ITC titration curves (upper panel) and binding isotherms (lower panel) for the complexation of c5a (0.5 mM) with the TBA⁺ salts (5 mM each) of I⁻, BF₄⁻, IO₄⁻, and PF₆⁻ in DMSO:CHCl₃ (5:95, v/v) at 25° C. Concentrations of c5a (in the cell) used in the titrations: 0.09 mM with TBA⁺ IO₄⁻ (0.9 mM); and 0.5 mM with TBA⁺I⁻ (1 mM) and TBA BF₄⁻ (5 mM), For two-step competition titration experiments, Cl⁻ (2.5 mM) served as the competitor for the titration of c5a with I⁻, and PF₆⁻ served as the competitor for the titration of c5a with BF₄⁻ (PF₆⁻: 1 mM).

FIG. 130C shows ITC titration curves (upper panel) and binding isotherms (lower panel) for the complexation of c5a with various anions in CH₃CN:CHCl₃ (5:95, v/v). Concentrations of c5a (in the cell) used in the titrations: 0.075 mM with TBA⁺Br⁻ (1 mM); 0.1 mM with TBA⁺Cl⁻ (1 mM); 0.5 mM with TBA⁺NO₃⁻ (5 mM) and TBA⁺ClO₄⁻ (5 mM). For two-step competition titration experiments, PF₆⁻ served as the competitor for the titration of c5a with NO₃⁻ (PF₆⁻: 1 mM) and ClO₄⁻ (PF₆⁻: 0.5 mM).

FIG. 130D shows ITC titration curves (upper panel) and binding isotherms (lower panel) for the complexation of c5a with various anions in CH₃CN:CHCl₃ (5:95, v/v). Concentrations of c5a (in the cell) used in the titrations: 0.09 mM with TBA⁺IO₄⁻ (0.9 mM); and 0.5 mM with TBA⁺I⁻ (1 mM), TBA BF₄⁻ (5 mM), and TBA⁺ClO₄⁻ (5 mM). For two-step competition titration experiments, Cl⁻ (2.5 mM) served as the competitor for the titration of c5a with I⁻, PF₆⁻ served as the competitor for the titration of c5a with BF₄⁻ (PF₆⁻: 1 mM).

FIG. 130E shows ITC titration curves (upper panel) and binding isotherms (lower panel) for the complexation of c5a with various anions in CH₃CN:CHCl₃ (5:95, v/v). Concentrations of c5a (in the cell) used in the titrations: 0.5 mM with TBA⁺HSO₄⁻ (5 mM), TMA⁺DPP⁻ (5 mM), and TBA⁺MPP⁻ (4 mM). For two-step competition titration experiments, PF₆⁻ served as the competitor for the titration of c5a with HSO₄⁻ (PF₆⁻: 2.8 mM), DPP⁻ (PF₆⁻: 5 mM), and MPP⁻ (PF₆⁻: 2.5 mM).

FIG. 131A shows halide transport into POPC LUVs mediated by c5a. Inside vesicles: 10 mM HEPES, 100 mM NaNO₃, 1 mM lucigenin. Outside vesicles: 10 mM HEPES, 100 mM NaCl. The emission of the intravesicular lucigenin was monitored at 505 nm (excitation wavelength: 430 nm). After adding c5a (final concentration: 2 μM) and monitoring the emission of lucigenin for 200 seconds, the vesicles were lysed by adding 0.1% of Triton X-100.

FIG. 131B shows halide transport into POPC LUVs mediated by c5a. Inside vesicles: 10 mM HEPES, 100 mM NaNO₃, 1 mM lucigenin. Outside vesicles: 10 mM HEPES, 100 mM NaBr. The emission of the intravesicular lucigenin was monitored at 505 nm (excitation wavelength: 430 nm). After adding c5a (final concentration: 2 μM) and monitoring the emission of lucigenin for 200 seconds, the vesicles were lysed by adding 0.1% of Triton X-100.

FIG. 131C shows halide transport into POPC LUVs mediated by c5a. Inside vesicles: 10 mM HEPES, 100 mM NaNO₃, 1 mM lucigenin. Outside vesicles: 10 mM HEPES, 100 mM NaI. The emission of the intravesicular lucigenin was monitored at 505 nm (excitation wavelength: 430 nm). After adding c5a (final concentration: 2 μM) and monitoring the emission of lucigenin for 200 seconds, the vesicles were lysed by adding 0.1% of Triton X-100.

FIG. 132A shows dose-response curves and half-maximal effective concentration (EC50) values for the transport of Cl-mediated by macrocycle c5a (0.05 to 10 μM, final concentration) reflected by the emission of HPTS (1 mM) entrapped in POPC LUVs.

FIG. 132B shows dose-response curves and half-maximal effective concentration (EC50) values for the transport of Br-mediated by macrocycle c5a (0.05 to 10 μM, final concentration) reflected by the emission of HPTS (1 mM) entrapped in POPC LUVs.

FIG. 132C shows fluorescence trace of I— transport mediated by c5a at 10 μM over 200 s. The EC50 for I— transport by c5a, which cannot be determined due to the low transport activity, is estimated to be greater than 10 μM. (emission wavelength: 510 nm; normalized intensity ratio F450/F405:=ratio of fluorescence emission intensities at excitation wavelengths of 450 nm and 405 nm).

FIG. 132D shows fluorescence trace of Cl— transport mediated by c5a at different concentrations from 0.05 μM to 10 μM over 200 s. (emission wavelength: 510 nm; normalized intensity ratio F450/F405:=ratio of fluorescence emission intensities at excitation wavelengths of 450 nm and 405 nm).

FIG. 132E shows fluorescence trace of Br— transport mediated by c5a different concentrations from 0.05 μM to 10 μM over 200 s. (emission wavelength: 510 nm; normalized intensity ratio F450/F405:=ratio of fluorescence emission intensities at excitation wavelengths of 450 nm and 405 nm).

FIG. 133 shows ion transport into POPC LUVs mediated by c5a at 25° C. Inside vesicles: 10 mM HEPES, 100 mM MCI (M=Li, Na, K, Rb), 1 mM HPTS, pH 7.0. Outside vesicles: 10 mM HEPES, 100 mM MCI, pH 7.0. A base pulse (20 μL of 0.5 M NaOH) was applied to each vesicular solution at 50 s, followed by adding 20 μL of c5a in DMF at 100 s. For each group, the vesicles were lysed after 200 s by adding 0.1% Triton X-100 to obtain the maximum emission intensity for normalization. The emission intensities of the intravesicular HPTS at 510 nm, I₄₀₅ and I₄₅₀, corresponding to two excitation wavelengths (405 and 450 nm) were monitored simultaneously, based on which the normalized intensity ratios (I₄₀₅/I₄₅₀) of the two emision intensities were obtained.

FIG. 134A shows chloride transport into DPPC LUVs mediated by c5a at 25° C. Inside vesicles: 10 mM HEPES, 100 mM NaCl, 1 mM HPTS, pH 7.0. Outside vesicles: 10 mM HEPES, 100 mM NaCl, pH 7.0. To ensure proper insertion of c5a, the LUVs were incubated with c5a (final concentration: 2 μM) at 45° C. for 3 min before being cooled down to 25° C. The LUVs for the control groups do not contain c5a. A base pulse (20 μL of NaOH) was applied to each vesicular solution at 25° C. For each group, the vesicles were lysed after 200 s by adding 0.1% Triton X-100 to obtained the maximum emission intensity for normalization. The emission of the intravesicular HPTS was monitored at 505 nm (excitation wavelengths: 405 and 450 nm).

FIG. 134B shows chloride transport into DPPC LUVs mediated by c5a at 45° C. Inside vesicles: 10 mM HEPES, 100 mM NaCl, 1 mM HPTS, pH 7.0. Outside vesicles: 10 mM HEPES, 100 mM NaCl, pH 7.0. To ensure proper insertion of c5a, the LUVs were incubated with c5a (final concentration: 2 μM) at 45° C. for 3 min before being kept at 45° C. The LUVs for the control groups do not contain c5a. A base pulse (20 μL of NaOH) was applied to each vesicular solution at 45° C. For each group, the vesicles were lysed after 200 s by adding 0.1% Triton X-100 to obtained the maximum emission intensity for normalization. The emission of the intravesicular HPTS was monitored at 505 nm (excitation wavelengths: 405 and 450 nm).

FIG. 135A shows top (left) and side (right) views of the energy-minimized structures of the 1:1 complex of macrocycle c5 and a horizontally placed pyrophosphate ion.

FIG. 135B shows top (left) and side (right) views of the energy-minimized structures of the 1:1 complex of macrocycle c5 and a vertically placed pyrophosphate ion.

FIG. 135C shows top (left) and side (right) views of the energy-minimized structures of the 2:1 complex of macrocycle c5 and a vertically placed pyrophosphate ion.

FIG. 136 shows an ¹H NMR spectrum of compound L3-NO₂ (CDCl₃, 400 MHz, 25° C.).

FIG. 137 shows a ¹³C NMR spectrum of compound L3-NO₂ (CDCl₃, 75 MHz, 25° C.).

FIG. 138 shows an HRMS-ESI spectrum of compound L3-NO₂ (positive mode).

FIG. 139 shows an ¹H NMR spectrum of compound L5-NO₂ (DMSO-d₆, 400 MHz, 25° C.).

FIG. 140 shows a ¹³C NMR spectrum of compound L5-NO₂ (DMSO-d₆, 75 MHz, 25° C.).

FIG. 141 shows an HRMS-ESI spectrum of compound L5-NO₂ (positive mode)

FIG. 142 shows an ¹H NMR spectrum of compound L5-COOH (DMSO-d₆, 400 MHz, 25° C.).

FIG. 143 shows a ¹³C NMR spectrum of compound L5-COOH (DMSO-d₆, 75 MHz, 25° C.).

FIG. 144 shows an HRMS-ESI spectrum of compound L5-COOH (positive mode).

FIG. 145 shows an ¹H NMR spectrum of compound ₂a (CDCl₃, 400 MHz, 25° C.).

FIG. 146 shows a ¹³C NMR spectrum of compound 2a (CDCl₃, 75 MHz, 25° C.).

FIG. 147 shows an MS-ESI spectrum of compound 2a (positive mode).

FIG. 148 shows an ¹H NMR spectrum of compound c5a (DMSO-d₆, 400 MHz, 25° C.).

FIG. 149 shows a ¹³C NMR spectrum of compound c5a (DMSO-d₆, 75 MHz, 25° C.).

FIG. 150 shows an HRMS-ESI spectrum of compound c5a (positive mode).

FIG. 151 shows an ¹H NMR spectrum of compound 2b-SC (CDCl₃, 400 MHz, 25° C.).

FIG. 152 shows a ¹³C NMR spectrum of compound 2b-SC (CDCl₃, 75 MHz, 25° C.).

FIG. 153 shows an HRMS-ESI spectrum of compound 2b-SC (negative mode).

FIG. 154 shows an ¹H NMR spectrum of compound 2b-NO₂ (CDCl₃, 400 MHz, 25° C.).

FIG. 155 shows a ¹³C NMR spectrum of compound 2b-NO₂ (CDCl₃, 75 MHz, 25° C.).

FIG. 156 shows an MS-ESI spectrum of compound 2b-NO₂ (positive mode).

FIG. 157 shows an ¹H NMR spectrum of compound 2b (CDCl₃, 400 MHz, 25° C.).

FIG. 158 shows a ¹³C NMR spectrum of compound 2b (CDCl₃, 75 MHz, 25° C.).

FIG. 159 shows an MS-ESI spectrum of compound 2b (positive mode).

FIG. 160 shows an ¹H NMR spectrum of compound c5b (DMSO-d₆, 75 MHz, 25° C.).

FIG. 161 shows a ¹³C NMR spectrum of compound c5b (DMSO-d₆, 75 MHz, 25° C.).

FIG. 162 shows an HRMS-ESI spectrum of compound c5b (positive mode).

FIG. 163 shows an ¹H NMR spectrum of compound 2c-SC (CDCl₃, 400 MHz, 25° C.).

FIG. 164 shows a ¹³C NMR spectrum of compound 2c-SC (CDCl₃, 75 MHz, 25° C.).

FIG. 165 shows an HRMS-ESI spectrum of compound 2c-SC (negative mode).

FIG. 166 shows an ¹H NMR spectrum of compound 2c-1 (CDCl₃, 400 MHz, 25° C.).

FIG. 167 shows a ¹³C NMR spectrum of compound 2c-1 (CDCl₃, 75 MHz, 25° C.).

FIG. 168 shows an HRMS-ESI spectrum of compound 2c-1 (negative mode).

FIG. 169 shows an ¹H NMR spectrum of compound 2c (CDCl₃, 400 MHz, 25° C.).

FIG. 170 shows a ¹³C NMR spectrum of compound 2c (CDCl₃, 75 MHz, 25° C.).

FIG. 171 shows an MS-ESI spectrum of compound 2c (positive mode).

FIG. 172 shows an ¹H NMR spectrum of compound c5c (DMSO-d₆, 400 MHz, 25° C.).

FIG. 173 shows a ¹³C NMR spectrum of compound c5c (DMSO-d₆, 75 MHz, 25° C.).

FIG. 174 shows an HRMS-ESI spectrum of compound c5c (positive mode)

FIG. 175 shows a 2D ROESY spectrum of the 1:1 mixture of c5a and TBA. MPP (DMSO-d₆, 20 mM, 500 MHz, mixing time=0.4 s, 25° C.).

FIG. 176 shows a thermal ellipsoid plot for the crystal structure of c5c drawn at 30% probability.

FIG. 177 shows a cartoon depicting how cystic fibrosis is defined by a loss of chloride transport.

FIG. 178 shows the general design of non-cyclic oligomaides and their binding kinetics.

FIG. 179 shows the design of macrocyclic oligomaides.

FIG. 180 shows the binding of anions to c5mer.

FIG. 181 shows a cartoon depicting transport of halides across a lipid bilayer (cell membrane) for in vitro drug screening.

FIG. 182 shows results for c5a-mediated transport of halides across a cell membrane.

FIG. 183 shows data for macrocycle c5a, which restores ASL volume hemostatis in CF airway epithelial cultures.

FIG. 184 shows cytotoxicity of c5mer derivatives.

FIG. 185 shows derivatives of c5mer.

FIG. 186 shows comparison of the c5mers with previous binding systems.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

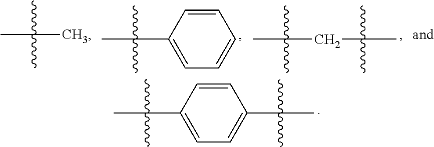

As used herein, unless otherwise indicated, the term “aliphatic groups” refers to branched or unbranched hydrocarbon groups that, optionally, contain one or more degrees of unsaturation.Degrees of unsaturation include, but are not limited to, alkenyl groups, alkynyl groups, and aliphatic cyclic groups. Aliphatic groups may be a C₁ to C₂₀ aliphatic group, including all integer numbers of carbons and ranges of numbers of carbons therebetween (e.g., C₁, C₂, C₃, C₄, C₅, C₆, C₁, C₈, C₉, C₁₀, C₁₁, C₁₂, C₁₃, C₁₄, C₁₅, C₁₆, C₁₇, C₁₈, C₁₉, and C₂₀). The aliphatic groups may include heteroatoms (e.g., —O—, —S—, or —N—) within the chains such that the aliphatic groups may have ethers chains, thioethers chains, or secondary amines chains. Aliphatic groups may be unsubstituted or substituted with one or more substituents. Examples of substituents include, but are not limited to, heteroatoms (e.g., —O, —S, —N, —P), halogens (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, alkoxide groups, amine groups, nitro groups, carboxylate groups, carboxylic acids, ether groups, thioether (sulfide) groups, alcohol groups, alkyne groups (e.g., acetylenyl groups and the like), and the like, and combinations thereof. Aliphatic groups may be alkyl groups, alkenyl groups, alkynyl groups, or carbocyclic groups, and the like.

As used herein, unless otherwise indicated, the term “alkyl group” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, the alkyl group is C₁ to C₂₀, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). The alkyl group may be unsubstituted or substituted with one or more substituents. Examples of substituents include, but are not limited to, various substituents such as, for example, halogens (—F, —Cl, —Br, and —I), aliphatic groups (e.g., alkyl groups, alkenyl groups, alkynyl groups, and the like), aryl groups, alkoxide groups, carboxylate groups, carboxylic acids, ether groups, amine groups, and the like, and combinations thereof.

As used herein, unless otherwise indicated, the term “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). An aryl group may also be referred to as an aromatic group. The aryl groups may comprise polyaryl groups such as, for example, fused rings, biaryl groups, or a combination thereof. The aryl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

The present disclosure provides linear and cyclic oligoamides. The present disclosure also provides methods of making and uses of linear and cyclic oligoamides.

In an aspect, the present disclosure provides compounds. The compounds are linear or cyclic oligoamides. A compound, which may be a linear or cyclic macrocyclic oliogamide (which may also be referred to as a macrocyclic compound/oligoamide), may comprise one or more aromatic substituents. In the case where a compound comprises a plurality of aromatic substituents, adjacent aromatic substituents are linked by at least one amide group. Non-limiting examples of linear and cyclic/macrocyclic oligoamides are provided herein.

A linear oligoamide or cyclic oligoamide may a curved backbone. Not intending to be bound by any particular theory, the curved backbone is largely due to intramolecular hydrogen bonds that rigidify the amide linkage of each amide group to each aromatic substituent and at least in part to an interaction between the aromatic substituents (e.g., π-π interactions), whereby the curved backbone is stabilized.

A linear compound/oligoamide may exhibit guest-dependent folding of linear oligoamides. A guest may be a hydrogen-bond donor, such as, for example, an anion, a polar molecule, or the like. Scheme 1 shows an illustration of an example of guest-dependent folding of linear oligoamides.

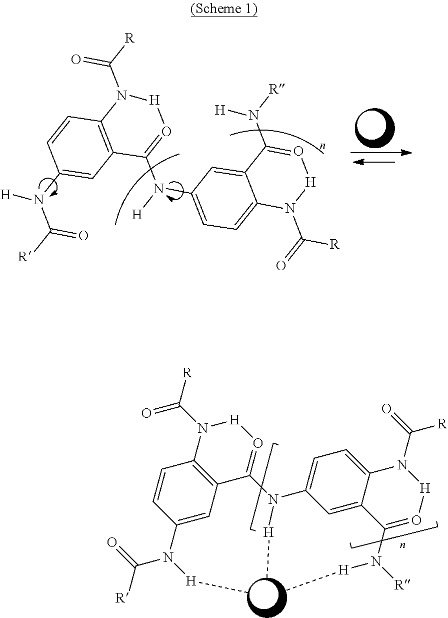

Structure on the left shows that, in the absence of a guest species capable of contributing hydrogen-bond donor(s), an oligoamide is not folded (left), i.e., the oligoamide adopts random, multiple conformations. Upon encountering a hydrogen-bond donor (e.g., anion, hydrophilic guest atom or molecule, etc.) (sphere), the backbone NH groups of the oligoamide forms hydrogen bonds with, for example, a hydrogen-bond donor (e.g., anion, hydrophilic guest atom or molecule, etc.), which “ties up” the oligoamide and forces it to adopt a crescent conformation for an oligoamide with ≤5 residues, or a helical conformation for an oligoamide with >5 residues. In various examples, by binding one or more hydrogen-bond donor(s) (e.g., anion(s), hydrophilic guest atom(s) or molecule(s), etc.) a linear oligoamide adopts a defined, folded conformation.

In various examples, a linear compound of the present disclosure has the following structure:

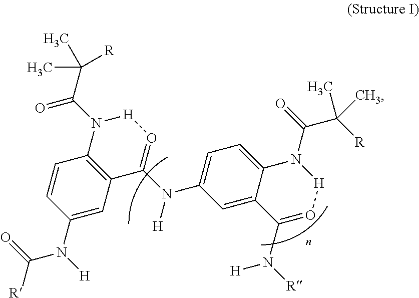

where n is 0 to 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50). R is independently at each occurrence chosen from
   linear aliphatic groups (which may be C₁-C₂₀ linear aliphatic groups (e.g., linear alkyl groups, such as, for example, methyl, ethyl, propyl, butyl, pentyl, hexyl, heptyl, octyl groups, and the like, which may be C₁-C₂₀ linear alkyl groups));
   branched aliphatic groups (which may be C₁-C₂₀ branched aliphatic groups (e.g., branched alkyl groups, such as, for example, isopropyl, isobutyl, t-butyl, neopentyl, isopentyl groups, and the like, which may be C₁-C₂₀ branched alkyl groups));

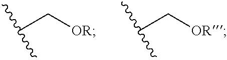

fluorinated linear aliphatic groups (which may be C₁-C₂₀ fluorinated linear aliphatic groups (e.g., fluorinated linear alkyl groups, such as, for example, fluoromethyl, fluoroethyl, fluoropropyl, fluorobutyl, fluoropentyl, fluorohexyl, fluoroheptyl, fluorooctyl groups, and the like), which comprise one or more fluorine group(s) (e.g., a fluorinated linear aliphatic group (such as, for example, a fluorinated linear alkyl group or the like) is a perfluorinated linear aliphatic group (e.g., alkyl and the like)));
fluorinated branched aliphatic groups (which may be C₁-C₂₀ fluorinated branched alkyl groups (e.g., fluorinated branched alkyl groups, such as, for example, branched derivatives of fluoropropyl, fluorobutyl, fluoropentyl, fluorohexyl, fluoroheptyl, fluorooctyl groups, and the like) and the like, which comprise one or more fluorine group(s) (e.g., a fluorinated branched aliphatic group (such as, for example, a fluorinated branched alkyl group or the like) is a perfluorinated branched aliphatic group (e.g., alkyl or the like)));
ether groups (e.g., —(CH₂)₂OCH₃, —(CH₂)₂OCH₂CH₃, —(CH₂)₂OCH₂CH(CH₃)₂, and —(CH₂)₂O(CH₂)₂CH(CH₃)₂, fluorinated analogs thereof, and the like)) (which may comprise one or two group(s) chosen from linear aliphatic groups (e.g., linear alkyl groups and the like), branched aliphatic groups (e.g., branched alkyl groups and the like), fluorinated linear aliphatic groups (e.g., fluorinated linear alkyl groups and the like), fluorinated branched aliphatic groups (e.g., fluourinated branched alkyl groups and the like); and oligoether groups (e.g.,

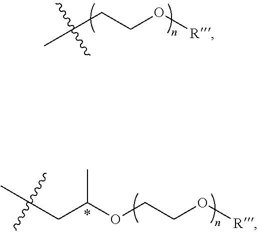

fluorinated analogs thereof, and the like, where the asterisk denotes a stereogenic carbon (i.e., a carbon having R or S stereochemistry, n is 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 8, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100)) (which may comprise one or more group(s) chosen linear aliphatic groups (e.g., linear alkyl groups and the like), branched aliphatic groups (e.g., branched alkyl groups and the like), fluorinated linear aliphatic groups (e.g., fluorinated linear alkyl groups and the like) (e.g., ethyl groups, propyl groups, and the like and combinations thereof and/or may comprise one or more fluorine groups (e.g., the ether group may be perfluorinated))); and
R″ is a linear or branched aliphatic group (e.g., alkyl group and the like) (e.g., methyl, ethyl, propyl, isopropyl, butyl, isobutyl, t-butyl, pentyl, neopentyl, isopentyl, and the like), which may comprise one or more fluorine groups (e.g., the oligoether group may be perfluorinated)), and combinations thereof.

In various examples, when the linear compound has structure I, n is 0, 1, 2, 3, or 4; R′ is chosen from methyl, ethyl, propyl, butyl, pentyl, hexyl, heptyl, octyl, and nonyl; R″ is chosen from methyl, ethyl, propyl, butyl, pentyl, and hexyl; R is
CH₂O(CH₂)₇CH₃ or —CH₂OR″′, where R″′ is chosen from methyl groups, ethyl groups, linear and branched propyl groups, linear and branched butyl groups, linear and branched pentyl groups, linear and branched hexyl groups, linear and branched heptyl groups, linear and branched octyl groups, and linear and branched nonyl groups.

In various examples, a compound of the present disclosure has the following structure:

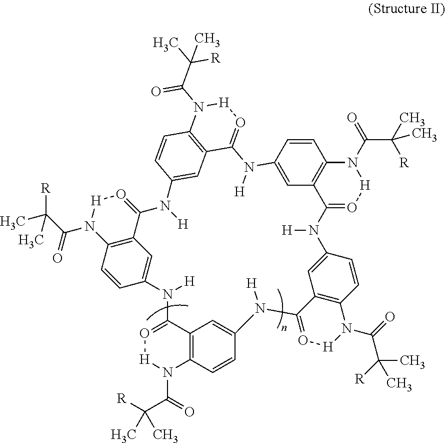

where n is 1 or 2. R is independently at each occurrence chosen from:

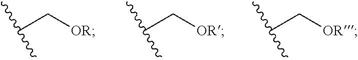

linear aliphatic groups (which may be C₁-C₃₀ linear aliphatic groups (e.g., linear alkyl groups, such as, for example, methyl, ethyl, propyl, butyl, pentyl, hexyl, heptyl, octyl groups, and the like, which may be C₁-C₃₀ linear alkyl groups));
branched aliphatic groups (which may be C₁-C₃₀ branched aliphatic groups (e.g., branched alkyl groups, such as, for example, isopropyl, isobutyl, t-butyl, neopentyl, isopentyl groups, and the like, which may be C₁-C₃₀ branched alkyl groups));
fluorinated linear aliphatic groups (which may be C₁-C₃₀ fluorinated linear aliphatic groups (e.g., fluorinated linear alkyl groups, such as, for example, fluoromethyl, fluoroethyl, fluoropropyl, fluorobutyl, fluoropentyl, fluorohexyl, fluoroheptyl, fluorooctyl groups, and the like), which comprise one or more fluorine group(s) (e.g., a fluorinated linear aliphatic group (such as, for example, a fluorinated linear alkyl group or the like) is a perfluorinated linear aliphatic group (e.g., alkyl and the like)));
fluorinated branched aliphatic groups (which may be C₁-C₃₀ fluorinated branched alkyl groups (e.g., fluorinated branched alkyl groups, such as, for example, branched derivatives of fluoropropyl, fluorobutyl, fluoropentyl, fluorohexyl, fluoroheptyl, fluorooctyl groups, and the like) and the like, which comprise one or more fluorine group(s) (e.g., a fluorinated branched aliphatic group (such as, for example, a fluorinated branched alkyl group or the like) is a perfluorinated branched aliphatic group (e.g., alkyl or the like)));
ether groups (e.g., —(CH₂)₂OCH₃, —(CH₂)₂OCH₂CH₃, —(CH₂)₂OCH₂CH(CH₃)₂, and —(CH₂)₂O(CH₂)₂CH(CH₃)₂, fluorinated analogs thereof, and the like) (which may comprise one or two group(s) chosen from linear aliphatic groups (e.g., linear alkyl groups and the like); branched aliphatic groups (e.g., branched alkyl groups and the like); fluorinated linear aliphatic groups (e.g., fluorinated linear alkyl groups and the like); fluorinated branched aliphatic groups (e.g., fluourinated branched alkyl groups and the like); and oligoether groups (e.g.,

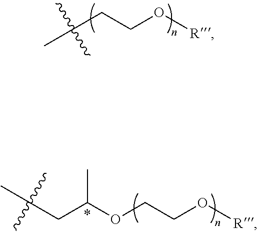

fluorinated analogs thereof, and the like, where the asterisk denotes a stereogenic carbon (i.e., a carbon having R or S stereochemistry, n is 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 8, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100)) (which may comprise one or more group(s) chosen from linear aliphatic groups (e.g., linear alkyl groups and the like), branched aliphatic groups (e.g., branched alkyl groups and the like), fluorinated linear aliphatic groups (e.g., fluorinated linear alkyl groups and the like) (e.g., ethyl groups, propyl groups, and the like and combinations thereof and/or may comprise one or more fluorine groups (e.g., the ether group may be perfluorinated)); and
R″′ is a linear or branched aliphatic group (e.g., alkyl group and the like) (e.g., methyl, ethyl, propyl, isopropyl, butyl, isobutyl, t-butyl, pentyl, neopentyl, isopentyl, and the like), which may comprise one or more fluorine groups (e.g., the oligoether group may be perfluorinated)), and combinations thereof.

In various examples, R, R′, and R′″ may comprise 1 to 30 carbon atoms. In various other examples, aliphatic group may be interrupted by one or more oxygen and/or sulfur atoms such that the aliphatic group comprises an ether or thioether moiety.

In various examples, when a compound of the present disclosure is cyclic, n is 1. In various other examples, when a compound of the present disclosure is cyclic n is 2. In various examples, when a compound of the present disclosure is cyclic, R is

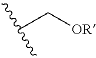

and R′ is —(CH₂)₇CH₃, —(CH₂CH₂O)₃CH₃, —CH₂CH═CH₂, methyl groups, ethyl groups, linear and branched propyl groups, linear and branched butyl groups, linear and branched pentyl groups, linear and branched hexyl groups, linear and branched heptyl groups, linear and branched octyl groups, linear and branched nonyl groups, unsaturated analogs thereof (e.g., linear and branched propenyl groups, linear and branched butenyl groups, linear and branched pentenyl groups, linear and branched hexenyl groups, linear and branched heptenyl groups, linear and branched octenyl groups, and linear and branched nonenyl groups), or

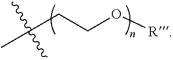

A cyclic compound/oligoamide may have the same backbone as a linear compound/oligoamide of the present disclosure. Scheme 2 shows an illustration of formation of a cyclic compound/oligoamide from a linear compound/oligoamide.

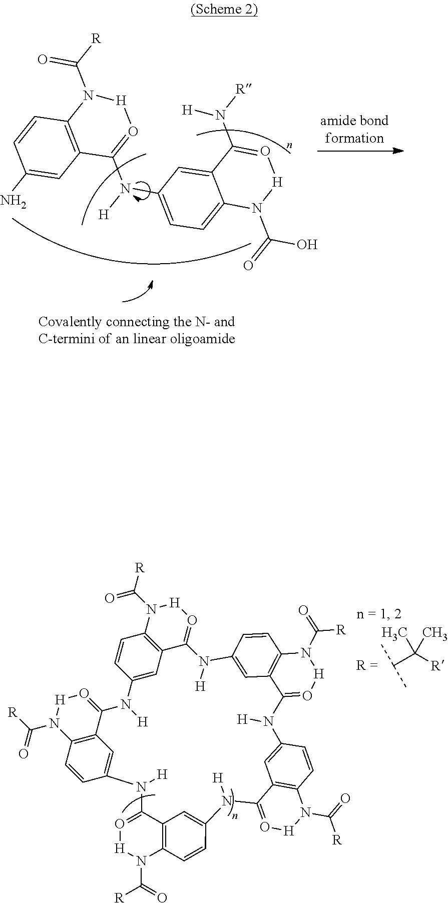

Linear oligoamide(s) may form a helical structure in the presence of one or more hydrogen-bond accepting atoms, molecules, etc. A helix can be right-handed or left-handed.

In an example, a helix comprises a compound having 5 residues (e.g., a residue is an aromatic substituent) per turn. A helix can comprise a compound having a pitch of about 3.6 Å per turn. The pitch and number of residues per turn are determined by the bond angles of the aromatic substituents. Not intending to be bound by any particular theory, these bond angles can change by several degrees, for example, depending on the temperature. As such, it is expected that the number of residues per turn and the pitch will not be exactly 5 residues and 3.6 Å, respectively, but rather the number of residues per turn and pitch will be a range surrounding these base values. For example, a helix can have 5±0.2 residues per turn, including all 0.1 residue values and ranges between 0 and 1. In another example, the helix has a pitch of 3.6±0.2 Å, including all 0.1 residue values and ranges between 0 and 1.

A helix of the present disclosure has an interior and an exterior portion. In an example, the interior of the helix is a hollow, tubular cavity comprising electropositive, hydrophilic NH groups. In an example, the exterior of the helix comprises hydrophobic groups.

The interior of the helix or the interior of the cyclic compound has a widest inner linear dimension (e.g., an inner diameter). The widest inner linear dimension of the interior is 6.4 Å (H to H), including all 0.1 Å values and ranges therebetween.

In an example, the widest inner linear dimension (e.g., an inner diameter) can vary in a compound of the present disclosure. In such an example, the helix can comprise different segments, each segment having a different widest inner linear dimension.

In an example, a helix has a longest linear dimension (e.g., a length). The longest linear dimension is 3.5 to 100 Å, including all 0.1 Å values and ranges therebetween. In another example, the longest linear dimension is 4 to 100 Å, including all 0.1 Å values and ranges therebetween.

It is desirable that one or more substituent(s) on the aromatic substituents are moderately hydrophilic. In an example, a compound of the present disclosure is soluble in a polar, aprotic solvent (e.g., N,N-dimethylformamide (DMF), dimethylsulfoxide (DMSO), and the like). For example, a compound of the present disclosure maintains solubility at a millimolar concentration (e.g., soluble at a concentration 0.1 to 10 mM, including all 0.1 mM values and ranges therebetween).

In various embodiments, the compound comprises one or more of aromatic substituents, wherein adjacent aromatic substituents are linked by at least one amide group and the compound has the following structure:

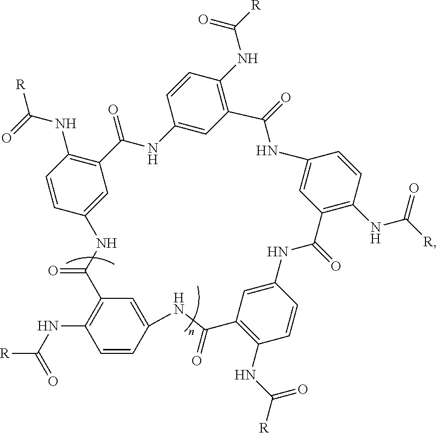

wherein n is 1 or 2, and wherein R is independently at each occurrence chosen from substituted or unsubstituted aliphatic groups, substituted or unsubstituted cyclic aliphatic groups, and substituted or unsubstituted aryl groups. The substituted or unsubstituted aliphatic groups may have one or more heteroatoms (e.g., O, S, or N) in its longest linear chain. In some embodiments, n is 1. In some embodiments, n is 2.

In various embodiments, R is independently at each occurrence chosen from

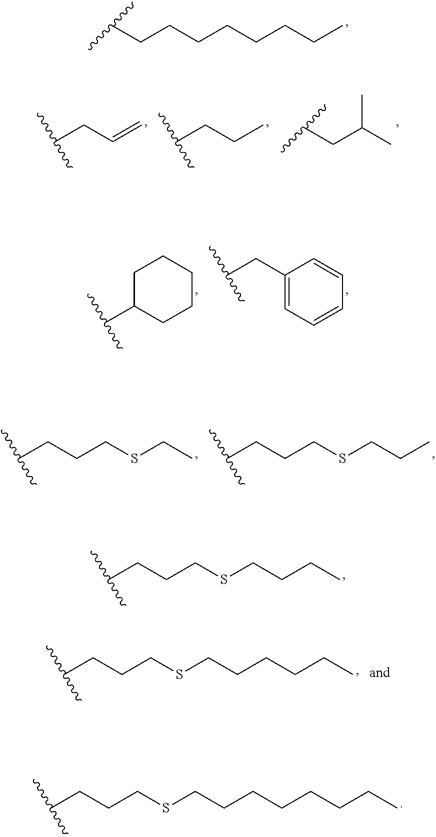

In various embodiments, the compound comprises one or more of aromatic substituents, wherein adjacent aromatic substituents are linked by at least one amide group and the compound has the following structure:

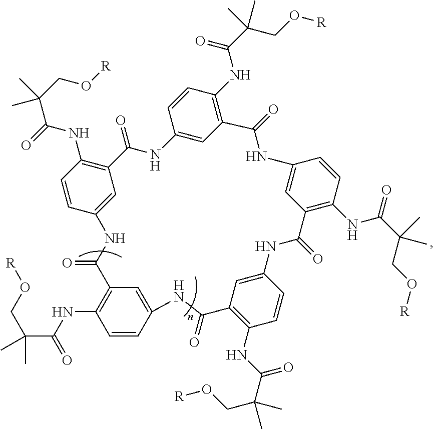

wherein n is 1 or 2, and wherein R is independently at each occurrence chosen from substituted or unsubstituted aliphatic groups, substituted or unsubstituted cyclic aliphatic groups, and substituted or unsubstituted aryl groups. The substituted or unsubstituted aliphatic groups may have one or more heteroatoms (e.g., O, S, or N) in its longest linear chain. In some embodiments, n is 1. In some embodiments, n is 2.

In various embodiments, R is independently at each occurrence chosen from

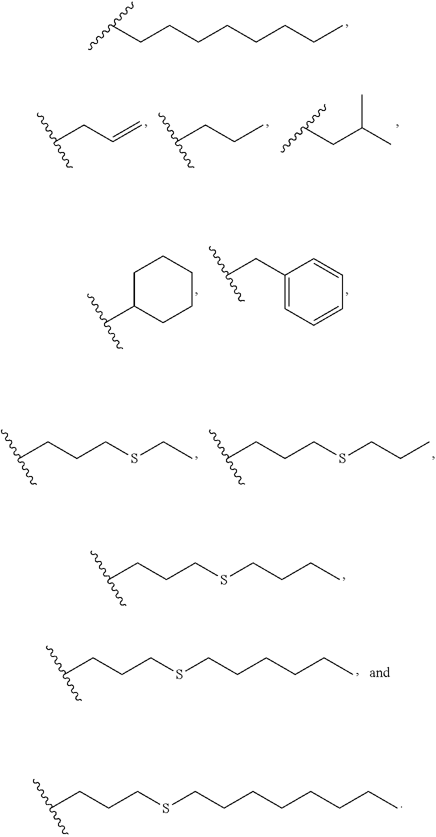

In an aspect, the present disclosure provides compositions. A composition may comprise one or more linear and/or one or more cyclic oligoamide of the present disclosure. A composition may be a pharmaceutical composition. A composition may be suitable for administration to an individual. Non-limiting examples of compositions are provided herein.

A composition may comprise one or more compound(s) or one or more complex(es) comprising one or more compound(s) and one or more hydrogen-bond acceptors and/or ions (e.g., anion(s)). In various non-limiting examples, a composition comprises one or more complexes formed by one or more compound(s) and one or more hydrogen-bond acceptors and/or ions (e.g., anion(s)). Without intending to be bound by any particular theory, it is considered that a complex is formed from (e.g., one or more interaction(s) between (e.g., one or more non-covalent interactions, such as, for example, one or more hydrogen bond(s), is formed between) the compound(s) and the hydrogen-bond acceptors and/or ions (e.g., anion(s))

In an aspect, the present disclosure provides method of making linear and cyclic oligoamides. In various examples, a linear or cyclic amide of the present disclosure is made by a method of the present disclosure. Non-limiting examples of methods of making linear and cyclic oligoamides are provided herein.

In an aspect, the present disclosure provides uses of linear and cyclic oliogoamides. The linear and/or cyclic oliogoamides may be used in methods such as, for example, forming transmembrane pores for transmembrane transport of hydrogen-bond acceptors and/or ions, sequestering hydrogen-bond acceptors and/or ions (e.g., anions), or the like. Compounds of the present disclosure may be used to enrich materials with ions (e.g., lithium). Non-limiting examples of uses of linear and/or cyclic oligoamides are provided herein.

Linear and cyclic oligoamides may be used in environmental applications. Oligoamides are expected to bind various ions (e.g., anions). The removal of sulfate from nuclear-waste media is of both environmental and economic importance. However, due to its very high hydration energy, the sulfate ion is one of the most difficult to be extracted from aqueous media. The macrocycles, with the predisposed, multiple amide NH groups, offer a sufficiently large inner cavity that can easily accommodate the sulfate ions by forming multiple hydrogen bonds that can replace the hydration shell of this anion. It is expected that oligoamides of the present disclosure can be used in extracting sulfate ions from aqueous solution.

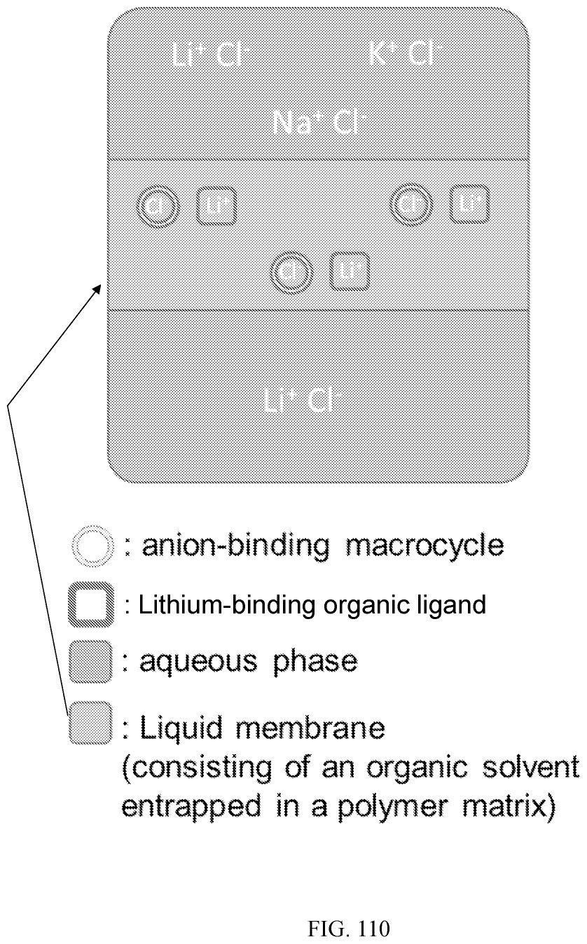

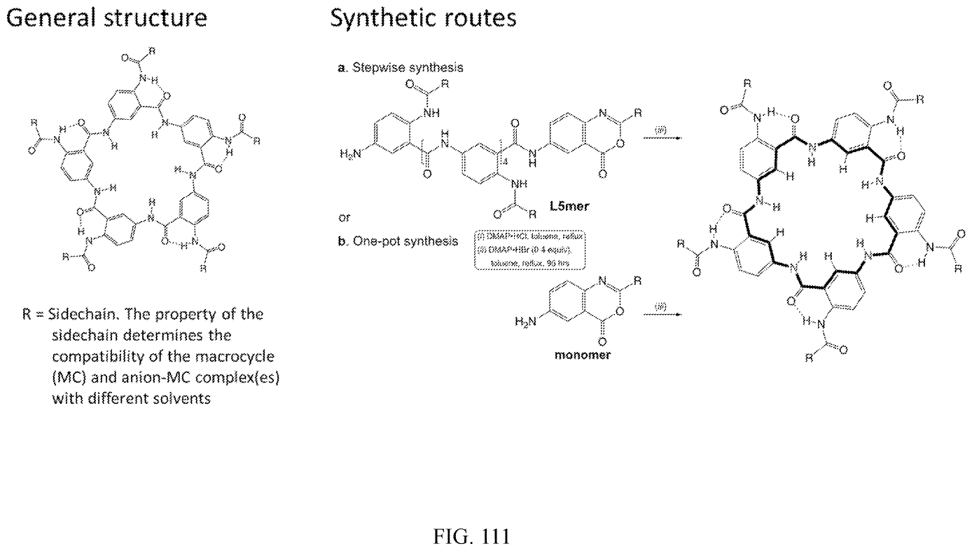

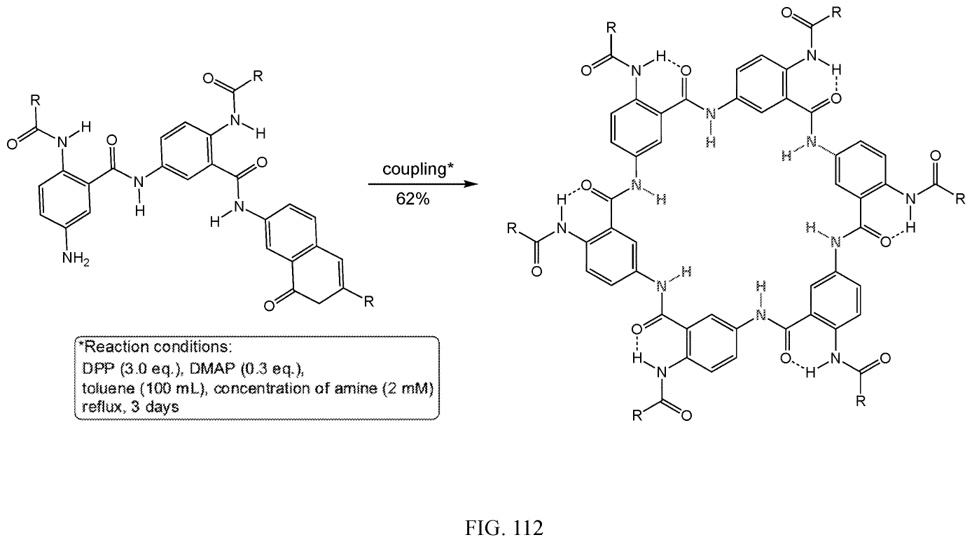

In various examples, compounds of the present disclosure are used for the enrichment of various ions, such as, for example, lithium. For example, organic binders, which are insoluble in water, are confined in the LM and serve as carriers that are not consumed. An anion binder such as the c5mer, and an organic binder specific for the lithium ion, can transfer LiCl from the first aqueous phase to the LM. A concentration gradient will drive the release of the bound LiCl in LM to the second aqueous phase as free LiCl. By selecting binders, such as crown ethers of various sizes or other reported organic cation binders that are specific for other cations, different metal ions can also be separated or enriched. This is displayed in FIG. 110, where the “lithium-binding organic ligand” should follow the second organic binder, i.e., the “square” below the “circle” shown as the “anion-binding macrocycle.”

Compound(s) and/or composition(s) can be used to sequester various hydrogen-bond acceptors and/or ions (e.g., anions). materials. In various non-limiting examples, one or more compound(s) is/are used to sequester one or more anions(s) (such as, for example, are halide ion(s) (fluoride ion(s), chloride ion(s), bromide ion(s), iodide ion(s), or a combination thereof), nitrate ions, carbonate ions, phosphate ions, sulfate ions, oxo anions, and the like, and combinations thereof.

The hydrogen-bond acceptors and/or ions (e.g., anion(s)) may be present in an aqueous sample, in a solid sample (such as, for example, a soil sample), in a gas sample, or the like. An aqueous sample may be derived (e.g., via extraction or other methods to isolate the anion(s)) from a solid sample. An aqueous sample may be a wastewater sample (e.g., a municipal wastewater sample, industrial wastewater sample, and the like), an industrial water sample (e.g., water used to make a commercial product, such as, for example, a reagent, a solvent, or the like), a municipal water sample, or the like.

After a hydrogen-bond acceptors and/or ions binds to a compound of the present disclosure, the compound bound to the hydrogen-bond acceptors and/or ions may be referred to as a “complex.” The complexes may be removed from the aqueous sample, the solid sample, the gas sample, or the like. In various examples, the anion(s) are removed from the aqueous sample, the solid sample, the gas sample, or the like using a solid surface with one or more of the compound(s) disposed thereon.

Linear and cyclic oligoamides may be used in biological applications. In various examples, linear and cyclic oligoamides may serve as binders (receptors) that bind (sequester) hydrogen-bond acceptors and/or ions (e.g., anions) in different affinities. For example, upon wrapping around or encircling an anion, the resultant complex becomes compatible (i.e., soluble) in non-polar media and can permeate the hydrophobic interior of cell membranes, facilitating (transporting) the otherwise hydrophilic, membrane-impermeable anion to cross cell membrane. An important application of anion binders is the binding of the chloride ion. Effective binders of chloride ion can help re-balance chloride gradient across cell membranes and thus provide therapeutics for diseases such as, for example, cystic fibrosis and the like.

In an aspect, the present disclosure provides a method of treating an individual diagnosed with or suspected of having cystic fibrosis comprising administering to the individual one or more compound(s) of the present disclosure such that one or more symptom(s) related to the cystic fibrosis is at least partially or completely alleviated.

In various embodiments, the compound forms a complex with a chloride anion. In embodiments, the compound of the present disclosure may transport the chloride anion across a cell membrane.

Linear oligoamide(s) and/or cyclic oligoamide(s) or one or more composition(s) may be used to treat an individual having an undesirable anion concentration (e.g., undesirable intra and/or extra cellular anion concentration, or the like). Compound(s) and/or compositions of the present disclosure can be administered to any human or non-human animal in need of therapy or prophylaxis for one or more condition(s) for which transport of one or more anion(s) is intended to provide a prophylactic or therapeutic benefit. Thus, the individual can be diagnosed with, suspected of having, or be at risk for developing any of a variety of conditions for which a reduction in severity would be desirable. Non-limiting examples of such conditions include cystic fibrosis, and other diseases known to be associated with defects in anion channels. Additional examples of such diseases may be found in C. A. Hubner, and T. J. Jentsch, Human Molecular Genetics, 2002, Vol. 11, 2435-2445), the relevant portions thereof are incorporated herein by reference.

In an example, one or more composition(s) of the present disclosure are delivered (e.g., administered) to an individual. Methods of administration are known in the art and non-limiting examples of which are described herein.

As described herein, compounds and/or compositions of the present disclosure can be provided in pharmaceutical compositions for administration by combining them with any suitable pharmaceutically acceptable carriers, excipients, stabilizers, or a combination thereof. Examples of pharmaceutically acceptable carriers, excipients, and stabilizers can be found in Remington: The Science and Practice of Pharmacy (2005) 21st Edition, Philadelphia, PA. Lippincott Williams & Wilkins. For example, suitable carriers include excipients and stabilizers which are nontoxic to recipients at the dosages and concentrations employed, and include buffers such as, for example, acetate, Tris, phosphate, citrate, and other organic acids; antioxidants including ascorbic acid and methionine; preservatives such as, for example, octadecyldimethylbenzyl ammonium chloride; hexamethonium chloride; benzalkonium chloride, benzethonium chloride; phenol, butyl or benzyl alcohol; alkyl parabens such as, for example, methyl or propyl paraben; catechol; resorcinol; cyclohexanol; 3-pentanol; and m-cresol; amino acids such as, for example, glycine, glutamine, asparagine, histidine, arginine, or lysine; monosaccharides, disaccharides, and other carbohydrates including glucose, mannose, or dextrins; chelating agents such as, for example, EDTA; tonicifiers such as, for example, trehalose and sodium chloride; sugars such as, for example, sucrose, mannitol, trehalose or sorbitol; surfactant such as, for example, polysorbate; salt-forming counter-ions such as, for example, sodium; and/or non-ionic surfactants such as, for example, Tween or polyethylene glycol (PEG). Additional examples of pharmaceutically acceptable carriers include, but are not limited to, sugars, such as lactose, glucose, and sucrose; starches, such as corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The pharmaceutical compositions may comprise other therapeutic agents. The present compositions can be provided as single doses or in multiple doses covering the entire or partial treatment regimen. The compositions can be provided in liquid, solid, semi-solid, gel, aerosolized, vaporized, or any other form from which it can be delivered to an individual.

The compositions may be administered parenterally. Administration of formulations comprising compounds and/or compositions as described herein can be carried out using any suitable route of administration known in the art. For example, the formulations comprising compounds and/or compositions of the present disclosure are administered via intravenous, intramuscular, intraperitoneal, intracerobrospinal, subcutaneous, intra-articular, or intrasynovial, routes. The compositions may be introduced as a single administration or as multiple administrations or may be introduced in a continuous manner over a period of time. For example, the administration(s) can be a pre-specified number of administrations or daily, weekly or monthly administrations, which may be continuous or intermittent, as may be clinically needed and/or therapeutically indicated.

Methods of the present disclosure may be used on various individuals. In various examples, an individual is a human or non-human mammal. Examples of non-human mammals include, but are not limited to, farm animals, such as, for example, cows, hogs, sheep, and the like, as well as pet or sport animals such as, for example, horses, dogs, cats, and the like. Additional non-limiting examples of individuals include, but are not limited to, rabbits, rats, mice, and the like.

In an aspect, the present disclosure provides methods of making compounds of the present disclosure. The method of making may be a one-pot method.

For example, the method comprises formation of a macrocycle of the present disclosure via a macrocyclization reaction. The macrocyclization may comprises self-condensation of the following compound:

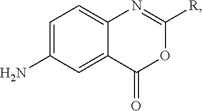

where R is independently at each occurrence chosen from substituted or unsubstituted aliphatic groups, substituted or unsubstituted cyclic aliphatic groups, and substituted or unsubstituted aryl groups. In various examples, R is

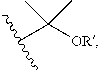

where R is chosen from:

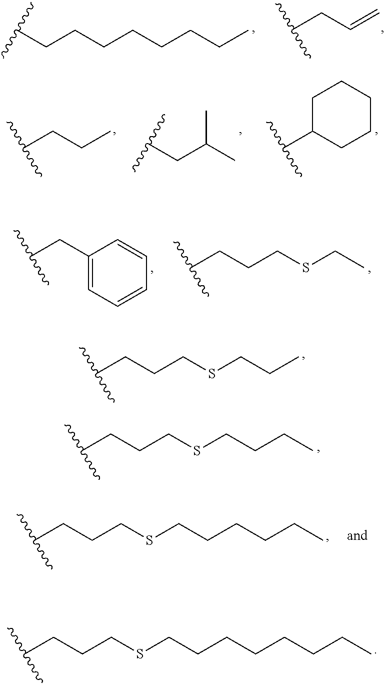

The self-condensation may occur via refluxing of a reaction mixture comprising

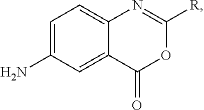

a nucleophilic catalyst, an acid, and a solvent. Following reflux, a macrocycle of the present disclosure is formed.

In various examples,

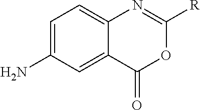

may be formed in situ via catalytic hydrogenation (e.g., reaction with H₂/Pd—C in a solvent, such as, for example, dichloromethane) of

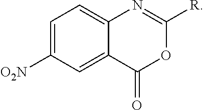

Various nucleophilic catalysts may be used. In various examples, the nucleophilic catalyst is 4-dimethylaminopyridine (DMAP).

Various acids may be used. In various examples, the acid is phosphoric acid.

Various solvents may be used. In various examples, the solvent is aromatic. In various examples, the solvent is toluene.

The steps of the methods described in the various embodiments and examples disclosed herein are sufficient to produce a polymer of the present disclosure or carry out a method of the present disclosure. Thus, in various embodiments, a method consists essentially of a combination of the steps of the methods disclosed herein. In various other embodiments, a method consists of such steps.

The following Examples describe various examples and embodiments of the present disclosure.
Example 1. A compound comprising one or more aromatic substituents, wherein in the case of a plurality of aromatic substituents, adjacent aromatic substituents are linked by at least one amide group, wherein the compound has the structure:
(which may be referred to a linear or helical compound or a linear or helical oligoamide)

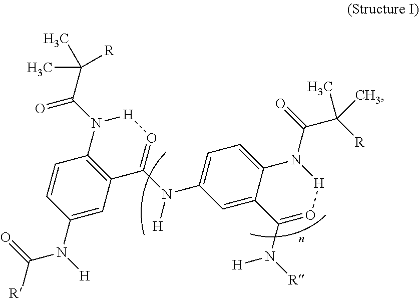

wherein n is 0 to 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) or
(which may be referred to as a cyclic compound or a cyclic oligoamide)

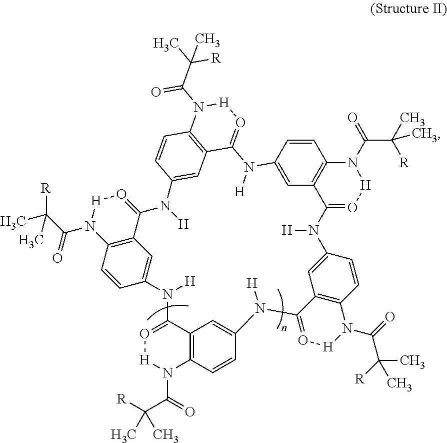

wherein n is 1 or 2, and
wherein R is independently at each occurrence chosen from:

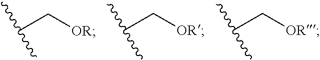

linear aliphatic groups, which may be C₁-C₂₀ linear aliphatic groups (e.g., linear alkyl groups, such as, for example, methyl, ethyl, propyl, butyl, pentyl, hexyl, heptyl, octyl groups, and the like, which may be C₁-C₂₀ linear alkyl groups);
branched aliphatic groups, which may be C₁-C₂₀ branched aliphatic groups (e.g., branched alkyl groups, such as, for example, isopropyl, isobutyl, t-butyl, neopentyl, isopentyl groups, and the like, which may be C₁-C₂₀ branched alkyl groups);
fluorinated linear aliphatic groups, which may be C₁-C₂₀ fluorinated linear aliphatic groups (e.g., fluorinated linear alkyl groups, such as, for example, fluoromethyl, fluoroethyl, fluoropropyl, fluorobutyl, fluoropentyl, fluorohexyl, fluoroheptyl, fluorooctyl groups, and the like, and the like), which comprise one or more fluorine group(s) (e.g., a fluorinated linear aliphatic group (such as, for example, a fluorinated linear alkyl group or the like) is a perfluorinated linear aliphatic (e.g., alkyl and the like) group);
fluorinated branched aliphatic groups, which may be C₁-C₂₀ fluorinated branched alkyl groups (e.g., fluorinated branched alkyl groups, such as, for example, branched derivatives of fluoropropyl, fluorobutyl, fluoropentyl, fluorohexyl, fluoroheptyl, fluorooctyl groups, and the like) and the like, which comprise one or more fluorine group(s) (e.g., a fluorinated branched aliphatic group (such as, for example, a fluorinated branched alkyl group or the like) is a perfluorinated branched aliphatic (e.g., alkyl or the like) group);
ether groups (e.g., —(CH₂)₂OCH₃, —(CH₂)₂OCH₂CH₃, —(CH₂)₂OCH₂CH(CH₃)₂, and —(CH₂)₂O(CH₂)₂CH(CH₃)₂, fluorinated analogs thereof, and the like) (which may comprise one or two group(s) chosen from linear aliphatic groups (e.g., linear alkyl groups and the like), branched aliphatic groups (e.g., branched alkyl groups and the like), fluorinated linear aliphatic groups (e.g., fluorinated linear alkyl groups and the like), fluorinated branched aliphatic groups (e.g., fluourinated branched alkyl groups and the like); and oligoether groups (e.g., the ether group may be perfluorinated)) (e.g.,

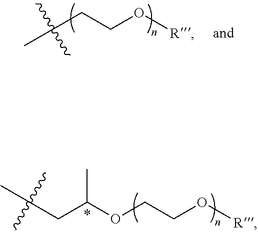

fluorinated analogs thereof, and the like, wherein the asterisk denotes a stereogenic carbon (i.e., a carbon having R or S stereochemistry), n is 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 8, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100) (which may comprise one or more group(s) chosen linear aliphatic groups (e.g., linear alkyl groups and the like), branched aliphatic groups (e.g., branched alkyl groups and the like), fluorinated linear aliphatic groups (e.g., fluorinated linear alkyl groups and the like) (e.g., ethyl groups, propyl groups, and the like and combinations thereof and/or may comprise one or more fluorine groups, and
R′″ is a linear or branched aliphatic group (e.g., alkyl group and the like) (e.g., methyl, ethyl, propyl, isopropyl, butyl, isobutyl, t-butyl, pentyl, neopentyl, isopentyl, and the like), which may comprise one or more fluorine groups (e.g., the oligoether group may be perfluorinated), and combinations thereof; and
wherein R′ and R″ are independently chosen from linear or branched aliphatic groups, aryl group (e.g., phenyl groups, substituted aryl groups, such as, for example, o-, m- or p-tolyl groups, o-, m-, or p-halo groups, methoxyphenyl groups, and the like)). Upon binding an anion, the compound may have a curved or helical backbone also due at least in part to intramolecular hydrogen bonds (e.g., between the sidechain amide NH groups and the backbone amide carbonyl groups) that contribute to rigidify the amide linkage of each amide group to each aromatic substituent and at least in part to an interaction between the aromatic substituents, whereby the curved backbone is stabilized along with intermolecular hydrogen bonds between the backbone NH groups and the anion. In various examples, when the compound has Structure I, n is 0, 1, 2, 3, or 4; the R′ group is a linear or branched methyl group, linear or branched ethyl group, linear or branched propyl groups, linear or branched butyl groups, linear or branched pentyl groups, linear or branched hexyl groups, linear or branched heptyl groups, linear or branched octyl groups, or linear or branched nonyl groups; the R″ group is a methyl group, ethyl group, linear or branched propyl groups, linear or branched butyl groups, linear or branched pentyl groups, or linear or branched hexyl groups; and the R group is —CH₂O(CH₂)₇CH₃ or —CH₂OR″′, where R″′ is chosen from methyl groups, ethyl groups, linear and branched propyl groups, linear and branched butyl groups, linear and branched pentyl groups, linear and branched hexyl groups, linear and branched heptyl groups, linear and branched octyl groups, and linear and branched nonyl groups. In various examples, when the compound has structure II, n is 1, R is

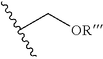

and R″′ is —(CH₂)₇CH₃, —(CH₂CH₂O)₃CH₃, —CH₂CH═CH₂, methyl groups, ethyl groups, linear and branched propyl groups, linear and branched butyl groups, linear and branched pentyl groups, linear and branched hexyl groups, linear and branched heptyl groups, linear and branched octyl groups, linear and branched nonyl groups, unsaturated analogs thereof (e.g., linear and branched propenyl groups, linear and branched butenyl groups, linear and branched pentenyl groups, linear and branched hexenyl groups, linear and branched heptenyl groups, linear and branched octenyl groups, and linear and branched nonenyl groups). In various examples, when the compound has structure II, n is 2, R is

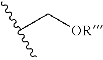

and R″′ is —(CH₂)₇CH₃, —(CH₂CH₂O)₃CH₃, —CH₂CH═CH₂, methyl groups, ethyl groups, linear and branched propyl groups, linear and branched butyl groups, linear and branched pentyl groups, linear and branched hexyl groups, linear and branched heptyl groups, linear and branched octyl groups, linear and branched nonyl groups, unsaturated analogs thereof (e.g., linear and branched propenyl groups, linear and branched butenyl groups, linear and branched pentenyl groups, linear and branched hexenyl groups, linear and branched heptenyl groups, linear and branched octenyl groups, and linear and branched nonenyl groups).
Example 2. A composition comprising one or more compound(s) of the present disclosure (e.g., one or more compound(s) of according to Example 1) and one or more hydrogen-bond acceptor(s) (e.g., polar guest molecule(s), anion(s), or the like), wherein the backbone of the compound has a crescent conformation or helical conformation (e.g., form a left-handed or right-handed helix) (e.g., extending longitudinally in the direction of a longitudinal axis). In the absence of an anion or a polar molecule providing hydrogen bond acceptors, a compound (i.e., a non-cyclic or linear oligoamide) adopts random (i.e., multiple) conformations. The backbone of the compound (a linear oligoamide) folds in the presence of an anion or a polar molecule offering hydrogen-bond acceptor(s) that form(s) one or more hydrogen bond(s) with the backbone NH groups of the compound.
Example 3. A composition according to Example 2, wherein the hydrogen-bond acceptor(s) and one or more anion(s) (such as, for example, halide ion(s) (fluoride ion(s), chloride ion(s), bromide ion(s), iodide ion(s), or a combination thereof), nitrate ions, carbonate ions, phosphate ions, sulfate ions, oxo anions, and the like, and combinations thereof).
Example 4. A composition according to Examples 2 or 3, wherein the compound has a linear structure that adopts a crescent or helical conformation has an interior and an exterior of the crescent or helix, and intramolecular hydrogen bonds are on the exterior of the helix and intermolecular hydrogen bonds are on the interior of the crescent or helix.
Example 5. A composition according to any one of Examples 2-4, wherein the helix has about ~5 residues per turn and/or a pitch of about 3.6 Å per turn.
Example 6. A composition according to any one of Examples 2-5, wherein the interior of a helical conformation defines a hollow tubular cavity that is parallel to the longitudinal axis.
Example 7. A composition according to any one of Examples 2-6, wherein the interior has an inner diameter of ~6.5 Å, including all 0.1 Å value and range therebetween.
Example 8. A composition according to any one of Examples 2-7, wherein the compound has a length (e.g., a length along the longitudinal axis) of 3.5 to 100 Å, including all 0.1 Å value and range therebetween.
Example 9. A composition according to any one of Examples 2-8, wherein the interior (also called the inner pore) is electrostatically positive and hydrophilic, and the exterior is hydrophobic.
Example 10. A composition according to any one of the preceding Examples, wherein the composition comprises a plurality of compounds that all have the same structure (e.g., share the same type of oligoamide backbone), at least one of the compounds has a different structure, or all of the compounds are different.
Example 11. A method of sequestering one or more anion(s) comprising: contacting one or more anion(s) with one or more compound(s) of the present disclosure (e.g., one or more compounds of Example 1), wherein at least a portion or all of the anion(s) are sequestered by the compound(s). The anion(s) may independently by bound by a compound. The compound(s) may be disposed on a substrate (e.g., conjugated to a substrate).
Example 12. A method of sequestering one or more anion(s) according to Example 11, wherein during the contacting the anion(s) are present in a sample.
Example 13. A method of sequestering one or more anion(s) according to Examples 11 or 12, wherein the sample is an organic or aqueous solution. In various examples, the anion(s) are halide ion(s) (fluoride ion(s), chloride ion(s), bromide ion(s), iodide ion(s), or a combination thereof), nitrate ions, carbonate ions, phosphate ions, sulfate ions, oxo anions, and the like, and combinations thereof.
Example 14. A method of sequestering one or more anion(s) according to any one of Examples 11-13, wherein the sample is a solution such as a wastewater sample (e.g., a municipal wastewater sample, industrial wastewater sample, and the like), an industrial water sample (e.g., water used to make a commercial product, such as, for example, a reagent, a solvent, or the like), a municipal water sample, a solution in organic solvent, or the like.
Example 15. A method of sequestering one or more anion(s) according to any one of Examples 11-14, wherein a complex is formed from (e.g., one or more interaction(s) between (e.g., one or more non-covalent bond(s) is/are formed between) the compound(s) and anion(s).
Example 16. A method of sequestering one or more anion(s) according to any one of Examples 11-15, herein the sequestered anions(s) (e.g., complex) is/are isolated (e.g., removed from the sample or the like).
Example 17. A method of treating an individual diagnosed with or suspected of having an extracellular and/or intracellular anion imbalance comprising: administering to the individual one or more compound(s) of the present disclosure (e.g., one or more compound(s) according to Example 1) to the individual. In an example, the individual is in need of treatment for an indication where the intracellular and/or extracellular concentration of an anion (typically, chloride ion) is unbalanced (e.g., physiologically undesirable) (e.g., the individual has been diagnosed with cystic fibrosis or the like). Without intending to be bound by any particular theory, it is considered that compound(s) of the present disclosure binds anions and facilitate transport of the bound anions across cell membranes, which results in correction of imbalanced extracellular and/or intracellular anion concentration gradients (e.g., the extracellular and/or intracellular anion imbalance is adjusted).
Example 18. The method of Example 17, wherein the physiological gradient of anion concentration in the individual is at least partially or completely restored.
Example 19. The method of Examples 17 or 18, wherein one or more symptom(s) related to the extracellular and/or intracellular anion imbalance in the individual is at least partially or completely alleviated.
Example 20. A method of sequestering one or more anion(s) according to any one of Examples 17-19, wherein the individual is a human or a non-human animal (e.g., a human mammal, a non-human mammal, or the like).
Example 21. A method of sequestering one or more anion(s) according to any one of Examples 17-20, herein the sequestered anions(s) (e.g., complex) is/are isolated (e.g., removed from the sample or the like).
Example 22. A method of sequestering one or more hydrogen-bond acceptors and/or ions comprising: contacting the one or more hydrogen-bond acceptors and/or ions with one or more compound(s) according to Example 1, wherein at least a portion or all of the one or more hydrogen-bond acceptors and/or ions are sequestered by the compound(s).
Example 23. A method according to Example 22, wherein the compound(s) are disposed on a substrate.
Example 24. A method according to Examples 22 or 23, wherein a sample comprises the one or more hydrogen-bond acceptors and/or ions and the sample is an organic or aqueous solution.
Example 25. A method according to Example 24, wherein the sample is a wastewater sample, an industrial water sample, a municipal water sample, or a solution in organic solvent.
Example 26. A method according to any one of Examples 22-25, wherein a complex is formed from the compound(s) and one or more hydrogen-bond acceptors and/or ions.
Example 27. A method according to any one of Examples 22-26, wherein the sequestered one or more hydrogen-bond acceptors and/or ions is/are isolated.
Example 28. A method of treating an individual diagnosed with or suspected of having an extracellular and/or intracellular anion imbalance comprising: administering to the individual one or more compound(s) according to Example 1 to the individual.
Example 29. A method according to Example 28, wherein the individual has been diagnosed with cystic fibrosis.
Example 30. A method according to Examples 28 or 29, wherein the physiological gradient of anion concentration in the individual is at least partially or completely restored.
Example 31. A method according to any one of Examples 28-30, wherein one or more symptom(s) related to the extracellular and/or intracellular anion imbalance in the individual is at least partially or completely alleviated.
Example 32. A method according to any one of Example 28-31, wherein the individual is a human or a non-human animal.
Example 33. A method according to any one of Example 28-32, wherein the sequestered anions(s) is/are isolated.
Example 34. A compound comprising one or more of aromatic substituents, wherein adjacent aromatic substituents are linked by at least one amide group and the compound has the following structure:

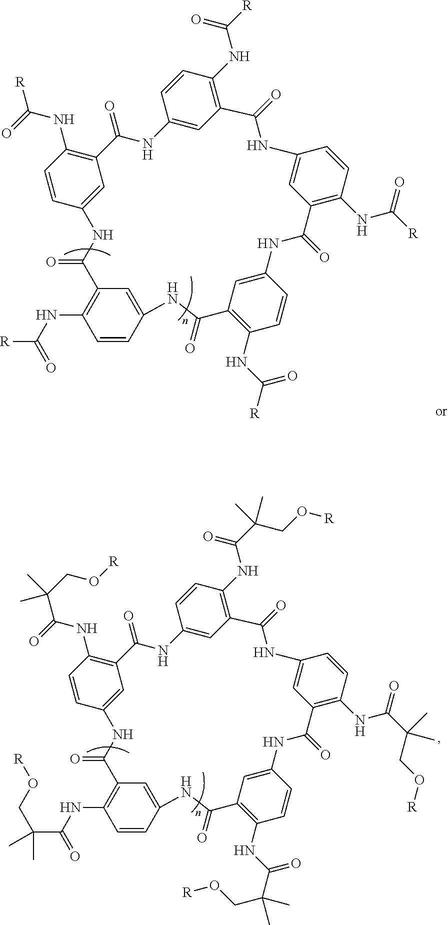

wherein n is 1 or 2, and wherein R is independently at each occurrence chosen from substituted or unsubstituted aliphatic groups, substituted or unsubstituted cyclic aliphatic groups, and substituted or unsubstituted aryl groups.
Example 35. A compound according to Example 34, wherein the compound has the following structure:

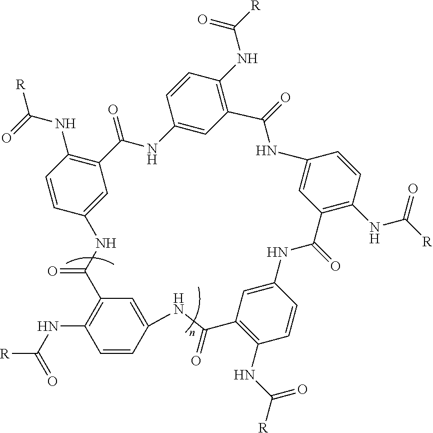

and each R is

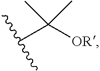

wherein each R′ is independently chosen from:

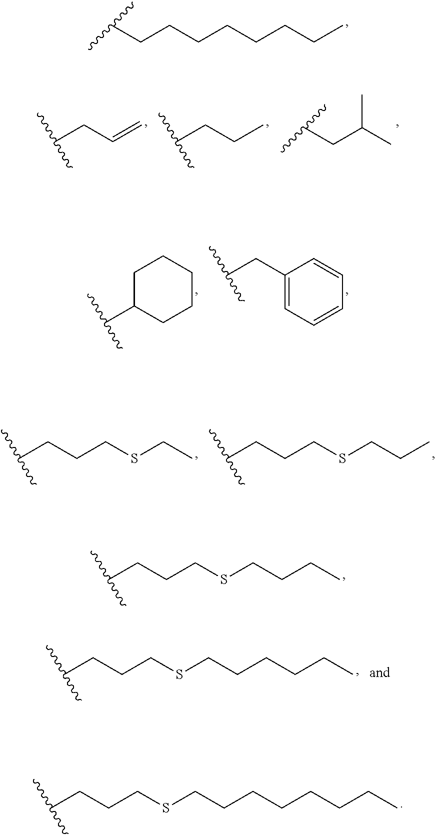

Example 35A. A compound according to Example 34, wherein R is independently at each occurrence chosen from

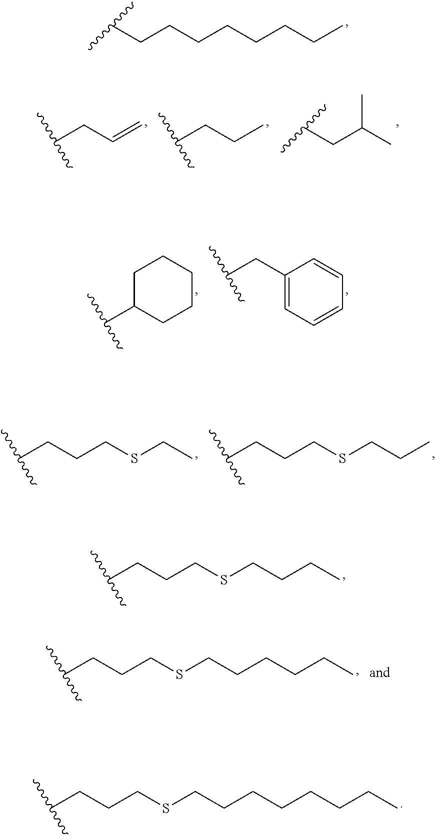

Example 36. A compound according to Example 34 or Example 35 or Example 35A, wherein n is 1.
Example 37. A compound according to Example 34 or Example 35 or Example 35A, wherein n is 2.
Example 38. A composition comprising one or more compound(s) according to any one of Examples 34-37.
Statement 39. A composition according to Statement 38, further comprising one or more hydrogen-bond acceptors and/or ions.
Example 40. A composition according to Example 39, wherein the hydrogen-bond acceptors and/or ions are polar guest molecules, anions, cations, or a combination thereof.
Example 41. A composition according to Example 40, wherein the anion is chosen from halide ions, nitrate ions, carbonate ions, phosphate ions, sulfate ions, oxo anions, and the like, and combinations thereof.
Example 42. A compound according to any one of Examples 34-37, wherein the compound has an inner diameter of 6.8-7.2 Å.
Example 43. A composition according to Examples 38-41, wherein the composition comprises a plurality of compounds that all have the same structure or a plurality of compounds wherein at least one of the compounds has a different structure.
Example 44. A method of sequestering one or more hydrogen-bond acceptors and/or ions comprising: contacting the one or more hydrogen-bond acceptors and/or ions with one or more compound(s) according to any one of Examples 34-37, wherein at least a portion or all of the one or more hydrogen-bond acceptors and/or ions are sequestered by the compound(s).
Example 45. A method according to Example 44, wherein a complex is formed from the compound(s) and one or more hydrogen-bond acceptors and/or ions.
Example 46. A method according to Example 45, wherein the sequestered one or more hydrogen-bond acceptors and/or ions is/are transported across a membrane.
Example 47. A method according to Example 46, wherein the membrane is a cell membrane.
Example 48. A method of treating an individual diagnosed with or suspected of having an extracellular and/or intracellular anion imbalance comprising: administering to the individual one or more compound(s) according to any one of Examples 34-37, such that the extracellular and/or intracellular anion imbalance is adjusted.
Example 49. A method according to Example 48, wherein the individual has been diagnosed with cystic fibrosis or cancers. Without intending to be bound by any particular theory, it is considered the method may be used to interrupt the pH balance inside cells and thus could be as an anticancer drug.
Example 50. A method of treating an individual diagnosed with or suspected of having cystic fibrosis comprising: administering to the individual one or more compound(s) according to any one of Examples 34-37, such that one or more symptom(s) related to the cystic fibrosis is at least partially or completely alleviated.
Example 51. A method according to Example 49, wherein the physiological gradient of anion concentration in the individual is at least partially or completely restored.
Example 52. A method according to Examples 49 or 51, wherein one or more symptom(s) related to the extracellular and/or intracellular anion imbalance in the individual is at least partially or completely alleviated.
Example 53. A method according to any one of Examples 49-52, wherein the individual is a human or a non-human animal.
Example 54. A method according to any one of Examples 49-53, wherein the compound forms a complex with a chloride anion.
Example 55. A method for making a compound according to any one Examples 34-37, comprising: forming a reaction mixture comprising nucleophilic catalyst, an acid, a solvent, and a compound having the following structure:

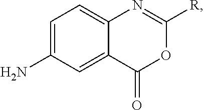

wherein R is independently at each occurrence chosen from substituted or unsubstituted aliphatic groups, substituted or unsubstituted cyclic aliphatic groups, and substituted or unsubstituted aryl groups, refluxing the reaction mixture, wherein following refluxing, the compound according to any one of Examples 34-37 is formed.
Example 56. A method according to Example 55, wherein R is

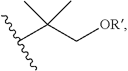

wherein R′ is chosen from:

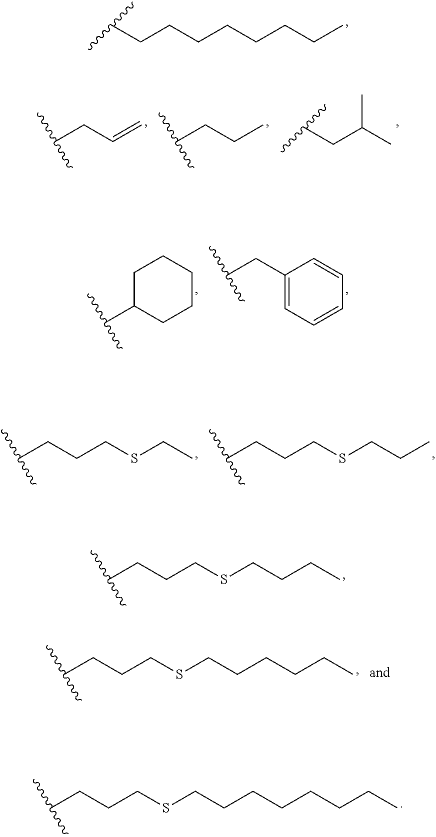

Example 57. A method according to Examples 55 or 56, wherein the acid is phosphoric acid.
Example 58. A method according to any one of Examples 55-57, wherein the nucelophic catalyst is 4-dimethylaminopydridine (DMAP).
Example 59. A method according to any one of Examples 55-58, wherein the solvent is toluene.
Example 60. A method according to any one of Examples 55-59, wherein the method is a one-pot method.
Example 61. A method according to any one of Examples 55-60, further comprising forming

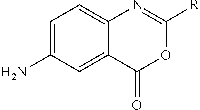

from

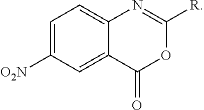

Example 62. A method according to any one of Examples 55-, wherein

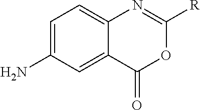

is formed via catalytic hydrogen of

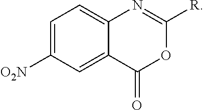

The following additional examples are presented to illustrate the present disclosure. The examples are not intended to be limiting in any matter.

### Example 1

This example provides a description of compounds of the present disclosure.

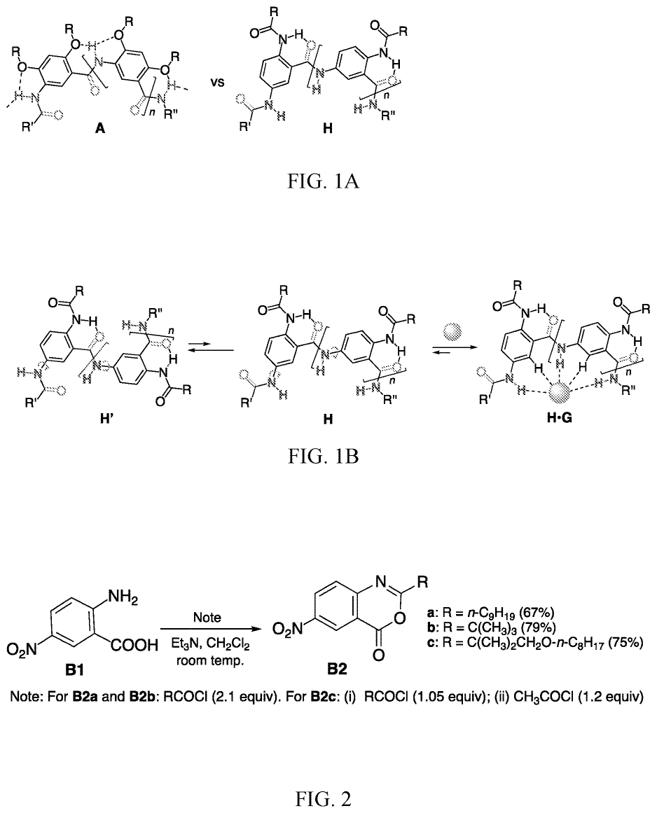

Described herein is the design, synthesis, and study of a hitherto unknown series of aromatic oligoamides that fold upon binding anions. As shown in FIG. 1A, inverting the orientation of the backbone amide groups of A leads to oligoamide H in which a six-membered intramolecular H-bond is introduced between each backbone amide oxygen and the amide proton of the adjacent acylamino sidechain. Such an intramolecular H-bond keeps the backbone amide oxygen atom from engaging in additional H-bonding and, more importantly, frees each backbone NH group to engage in H-bonding with other guest species.

Unlike that of A, the backbone of oligoamide H is partially constrained. Around each backbone amide group of H, the rotation of the aryl-CO single bond is limited by an intramolecular H-bond, while the rotation of the C(O)NH-aryl bond remains unhindered. It is thus expected that H will adopt multiple conformations which is entropically favorable (FIG. 1B). Upon adding a guest, such as an anion that forms strong H-bonds with the backbone amide protons, the entropic barrier for adopting a single conformation is overcome by the enthalpic contribution from the multiple H-bonds between the guest and the oligoamide host. As a result, the equilibrium is shifted toward complex H·G, resulting in a folded comformation for H.

In contrast to A and other aromatic oligoamide foldamers, which can be synthesized based on established amide chemistry, oligoamides H, which comprise 5-amino-N-acylanthranilic acid residues, cannot be obtained by using known methods. It was reported that, when being treated with acylating or coupling reagents, anthranilic acid and its N-acylated derivatives self-cyclize into derivatives having a (4H)-3, 1-benzoxazin-4-one (or benzoxazinone) core, which prevents amide coupling from happening.

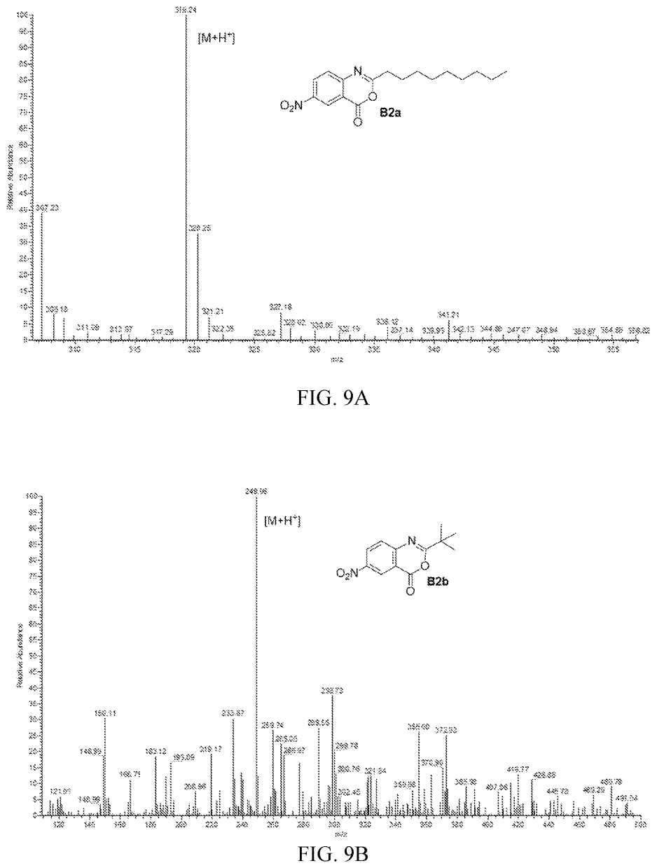

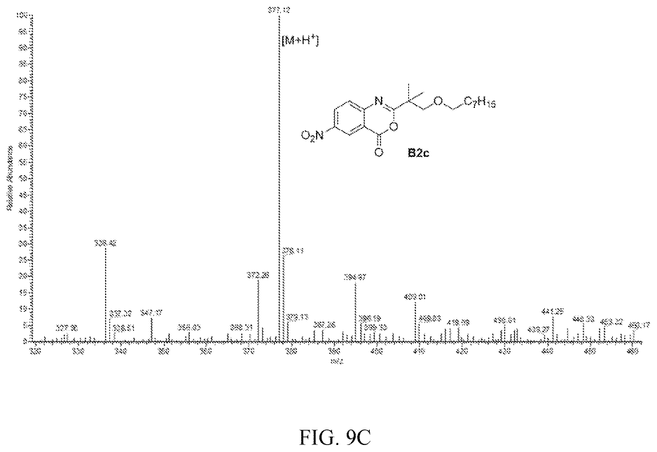

Indeed, treating 5-nitro-anthranilic acid B1 with two or more equivalents of decanoyl or trimethylacetyl chloride gave benzoxazinone derivatives B2a or B2b (Scheme 1). Similarly, compound B2c was obtained by converting B1 into the corresponding 5-nitro-N-acylanthranilic acid which was then treated with acetyl chloride. The ¹H NMR spectra of B2a-c reveal the same benzoxazinone core (FIG. 8). The identities of these compounds are also verified with ESI-MS spectra (FIG. 9).

Single crystals of B2b were obtained from hexane/ethyl acetate (1/2, v/v) by slow evaporation of solvents at room temperature. The X-ray structure of B2b confirms that acid B1 was converted into the benzoxazinone derivative by treating with trimethylacetyl chloride (FIG. 2).

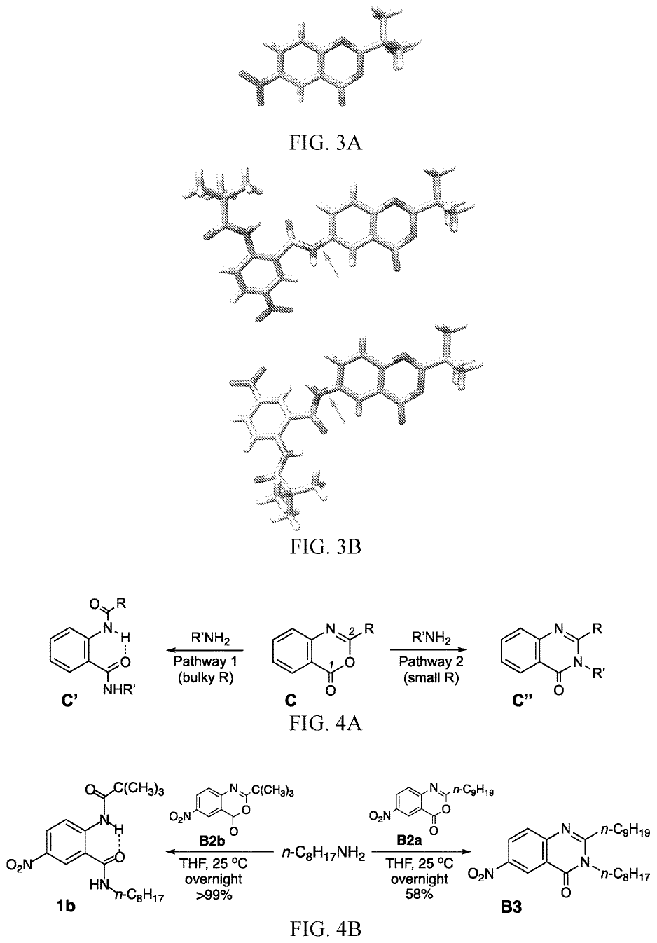

While anthranilic acid or its N-acyl derivatives cannot directly couple with other amines due to self-cyclization, benzoxazinone C and its derivatives do react with amines via two routes (FIG. 3A). One involves nucleophilic attack at carbon C-1, leading to ring-opening product C′; the other involves attacking C-2 to give quinazolinone C″. Steric factors determine the outcome of a reaction. With bulky R and/or R′, the desired ring-opening of C happens, forming product C′ having an acylamino (RCONH—) sidechain and a new amide bond; with small or slim R and/or R′ that do not impose significant steric hindrance, carbon C-2 is attacked to give C″ with no amide bond.

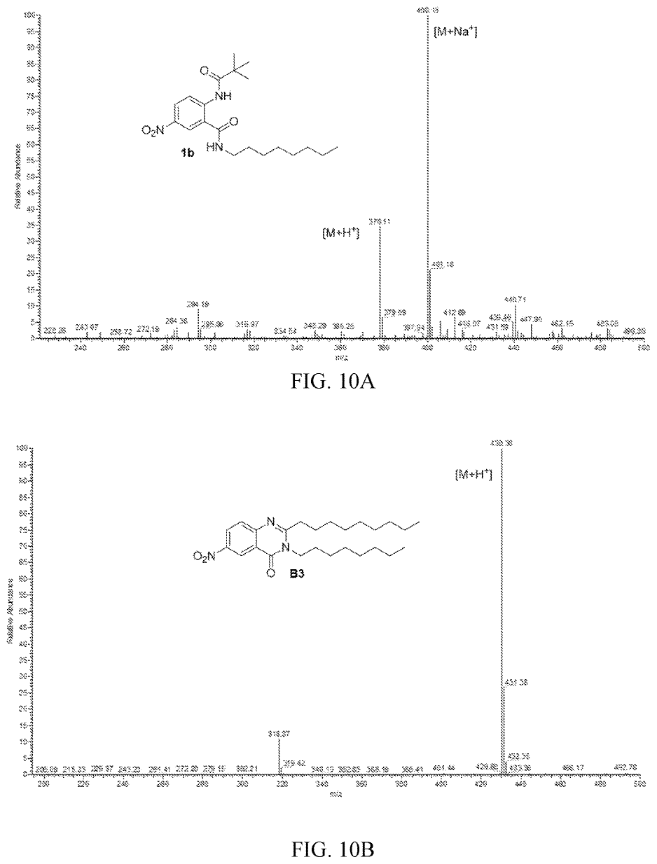

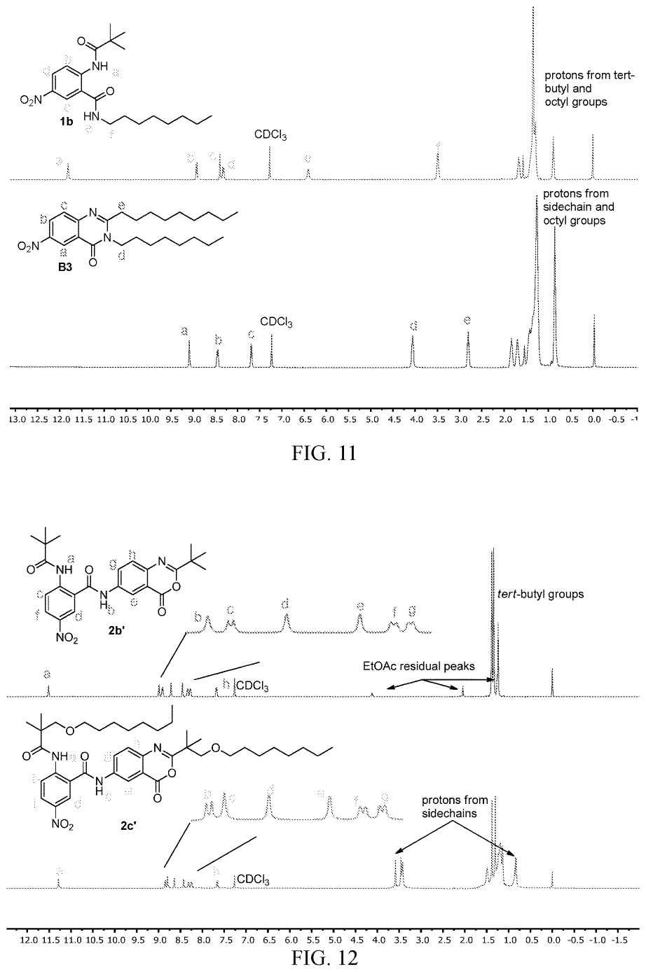

Compound B2a and B2b carrying an n-octyl and a t-butyl side chains, respectively, were treated with n-octylamine (FIG. 3B). The reaction of B2a and n-octylamine afforded quinazolinone B3 in 58% yield, while the reaction of B2b and n-octylamine led to the ring-opening product 1b nearly quantitatively. The molecular weights of B3 and 1b are confirmed by mass spectra (FIG. 10). ¹H NMR spectra (FIG. 11) reveal two amide signals at 6.33 and 11.79 ppm for 1b, with no amide signal observed with B3. Thus, to ensure the formation of the amide bond and the release of the acylamino side chain, as observed with amide 1b, a bulky R group, like that of B2b, with a tertiary α-carbon should be present.

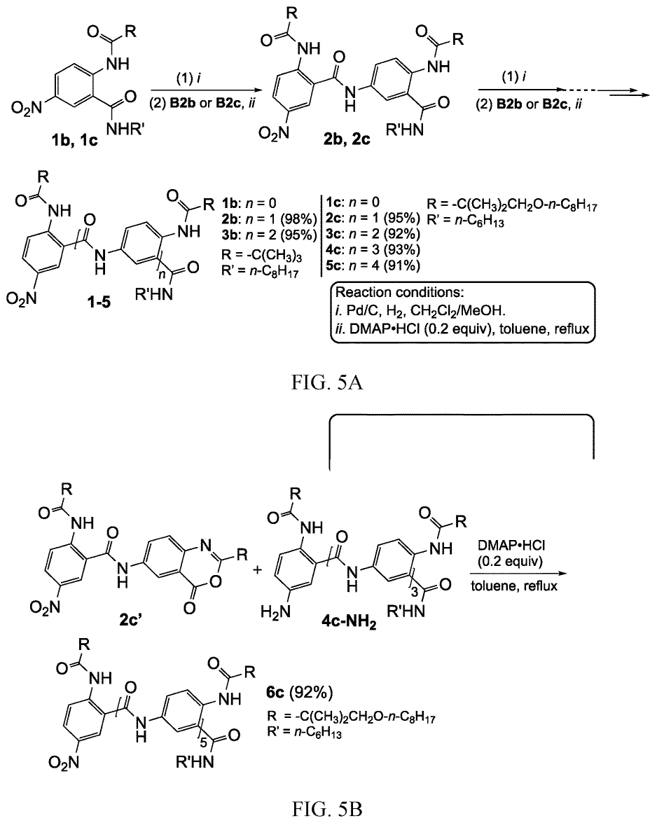

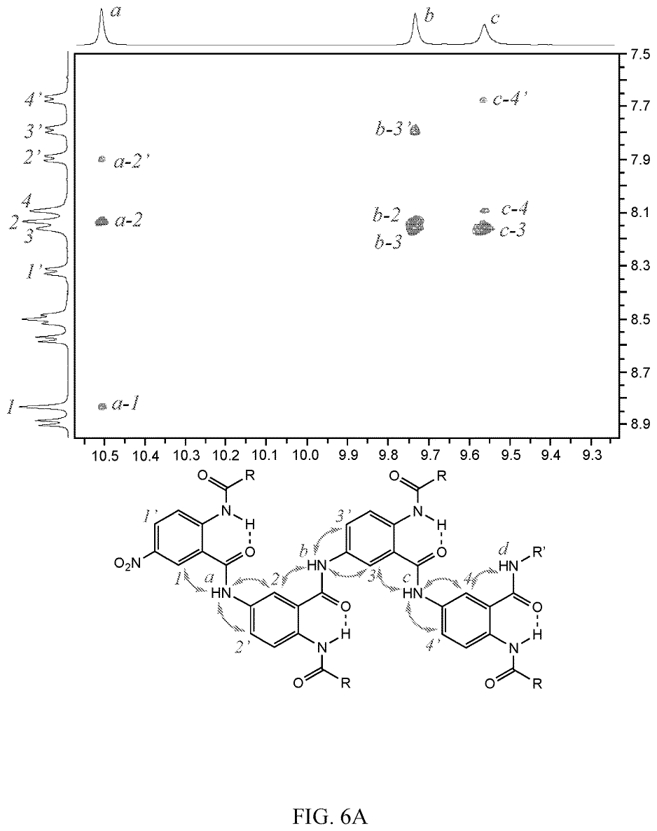

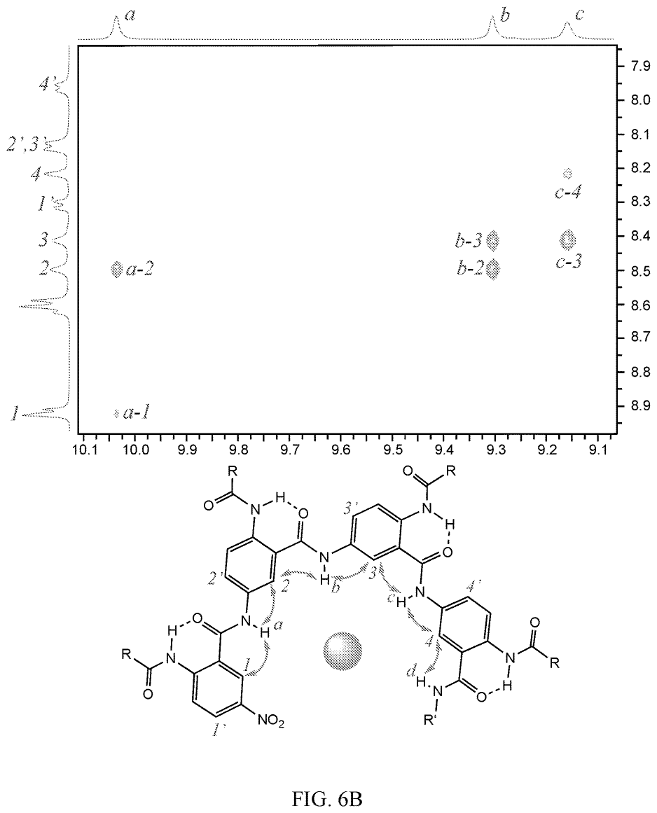

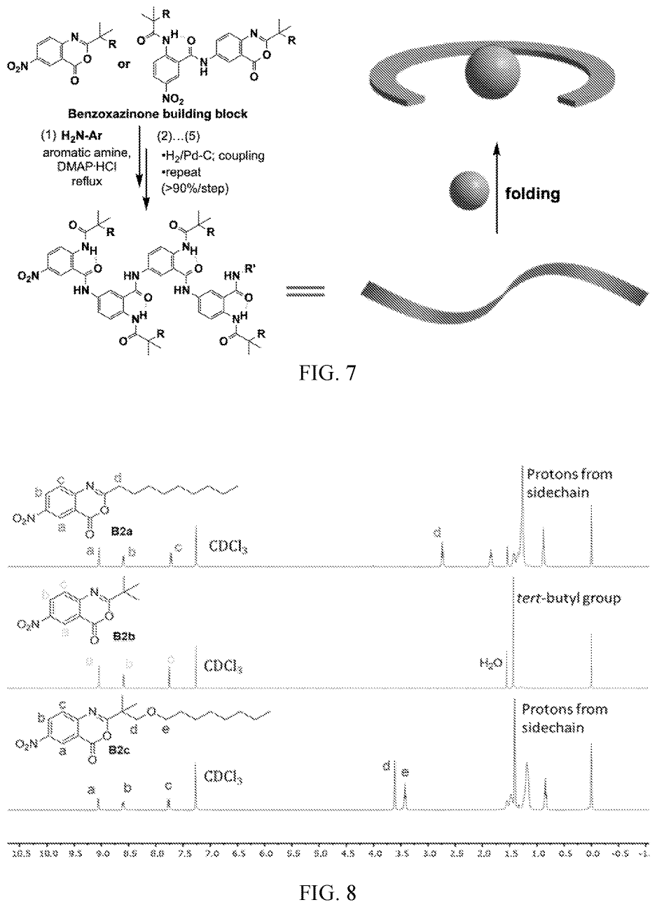

Based on the observation made with B2b, the synthesis of oligoamides having the backbone of general structure H was probed (FIG. 5A). Treating B2b and the amine derived from 1b in the presence of 4-dimethylaminopyridine hydrochloride (DMAP·HCl) gave dimer 2b in 98% yield. Reducing 2b to its corresponding amine followed by coupling with B2b gave trimer 3b in 95% yield. However, coupling B2b to the amine from trimer 3b led to a poorly soluble product, presumably the corresponding tetramer, that defied characterization. Dimer 2c to pentamer 5c carrying sidechains having a quaternary α-carbon and an n-octyloxy tail were obtained >90% yields by stepwise coupling of B2c to the amine precursors under the same conditions. Oligoamides 2c to 5c showed good solubility in solvents including chloroform and DMSO.

Dimeric 2b′ and 2c′, each having a benzoxazinone moiety, were also prepared (below) and characterized (FIG. 12). The solid-sate structure of 2b′ confirms the presence of the benzoxazinone moiety (FIG. 2), in which two conformations related by ~180° rotation around the single bond between the benzoxazinone unit and the rest of the molecule, are revealed.

Under the same conditions for coupling B2b or B2c, treating 2c′ and tetramer amine 4c-NH₂ gave hexamer 6c in 92% yield (FIG. 5B). The synthesis of 2b, 3b, and 2c-6c indicates that the adopted strategy, which involves repetitive coupling of benzoxazinone monomers B2b and B2c to a growing oligomer chain, or the coupling of oligomers 2c′ and 4c-NH₂, represents a new, highly efficient method for forming amide bonds. This method, which involves refluxing without any coupling reagent, is straightforward and convenient for synthesizing these new aromatic oligoamides.

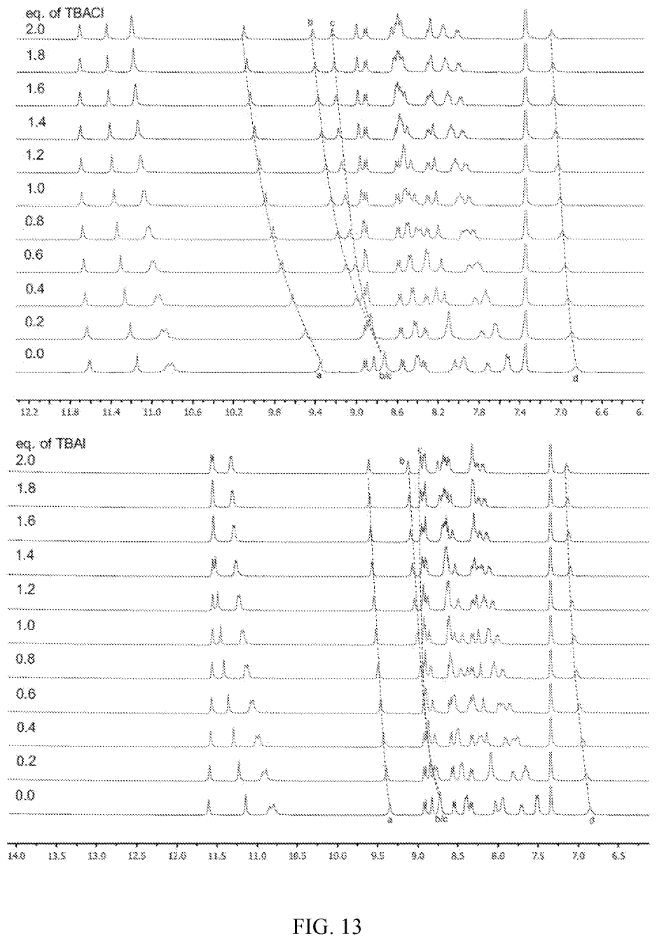

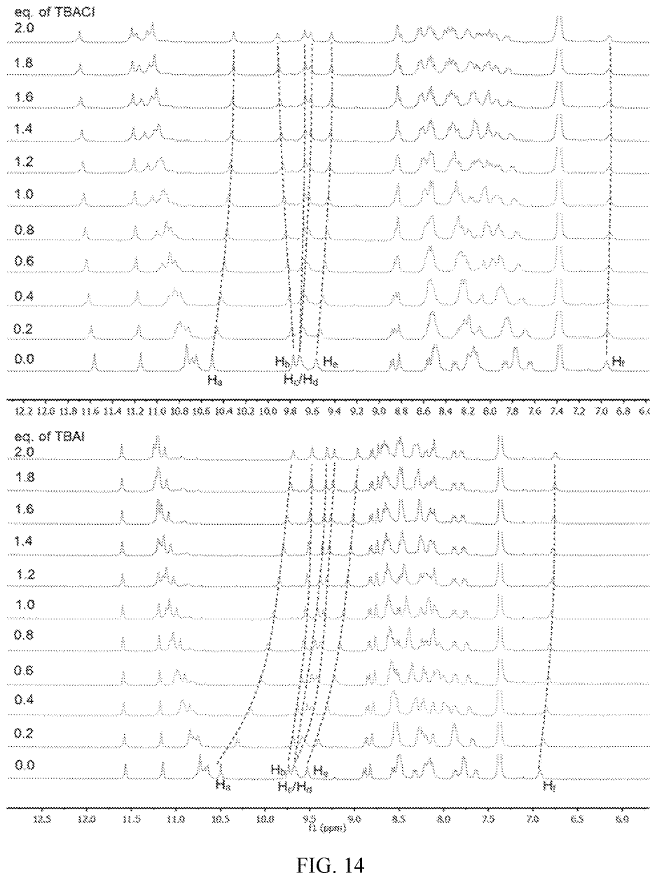

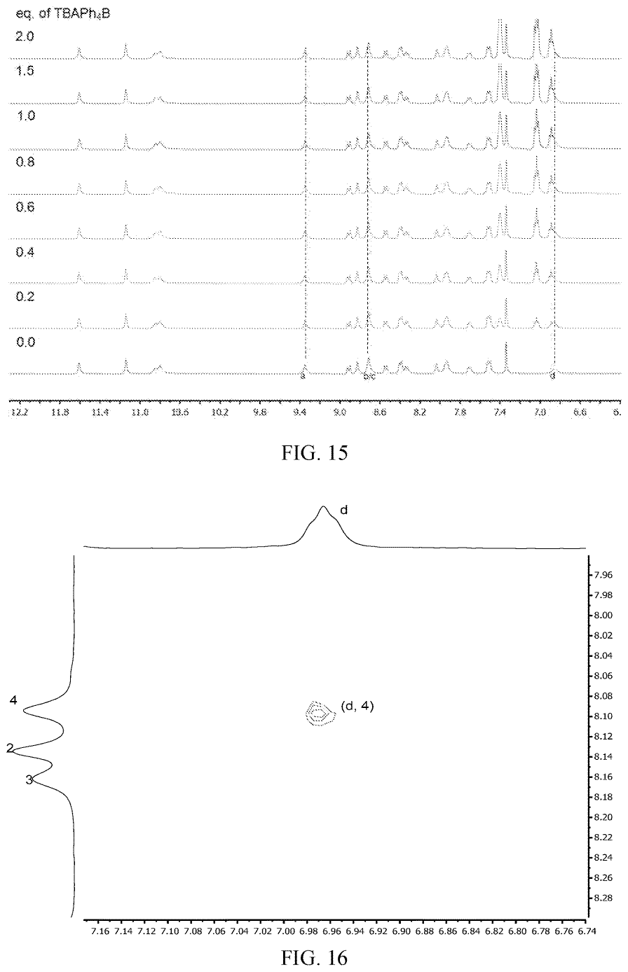

The interaction of the obtained oligoamides with anions was explored by titrating 4c (5 mM) with tetra-n-butylammonium (n-Bu₄N⁺) chloride or iodide. In CD₃CN/CDCl₃ (1/9, v/v), the signals of the backbone amide protons of 4c exhibit downfield shifts with 0 to 2.0 equivalents of n-Bu₄N⁺Cl⁻ (FIG. 13) or n-Bu₄N⁺I⁻ (FIG. 14), indicating that the chloride or iodide ions engage in H-bonding interactions with these amide protons. In contrast, titrating 4c (5 mM) with 0 to 2 equivalents of tetra-n-butylammonium tetraphenyl borate (n-Bu₄N⁺Ph₄B⁻), which cannot form H-bond, did not lead to any shift in the amide proton resonances (FIG. 15). These results demonstrate that 4c indeed bind anions like chloride and iodide.

The conformational change of tetramer 4c upon binding anions was probed by comparing the 2D (NOESY) spectrum of 4c in the absence and presence of one equivalent of n-Bu₄N⁺I⁻. In CDCl₃ containing 5% DMSO-d₆, the NOESY spectrum of free 4c reveals three NOEs involving each of amide protons a, b, and c and its neighboring aromatic protons (FIG. 4A). Among the NOEs, those involving protons a and 1, b and 2, and c and 3, along with that between protons d and 4 (FIG. 16), are expected because of the presence of the intramolecular H-bond between each backbone amide carbonyl group and the adjacent NH group of the sidechain. In addition, two NOEs involving amide proton a, b, or c, and the two “outer” (protons 1-3′) and “inner” (protons 1-3) aromatic protons are observed, consistent with the expected rotation of each (CO)NH-aryl single bond in 4c (FIG. 4A). These observed NOEs indicate that, due to the rotation of the (CO)NH-aryl bonds, oligoamide 4c most likely adopes random conformations roughly represented by the zig-zagged structure in FIG. 5A.

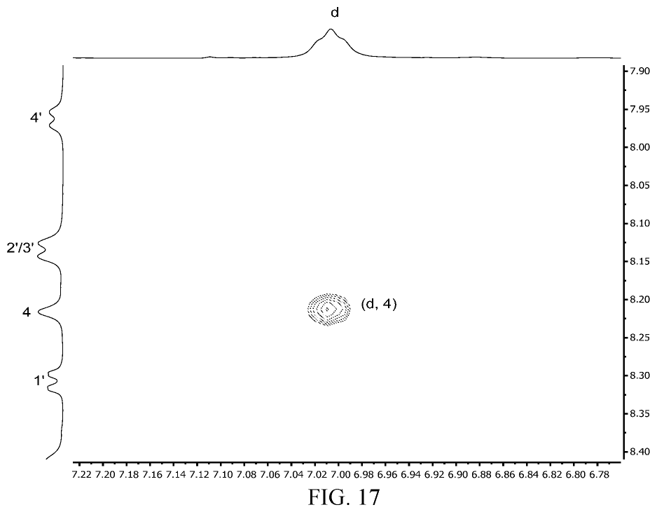

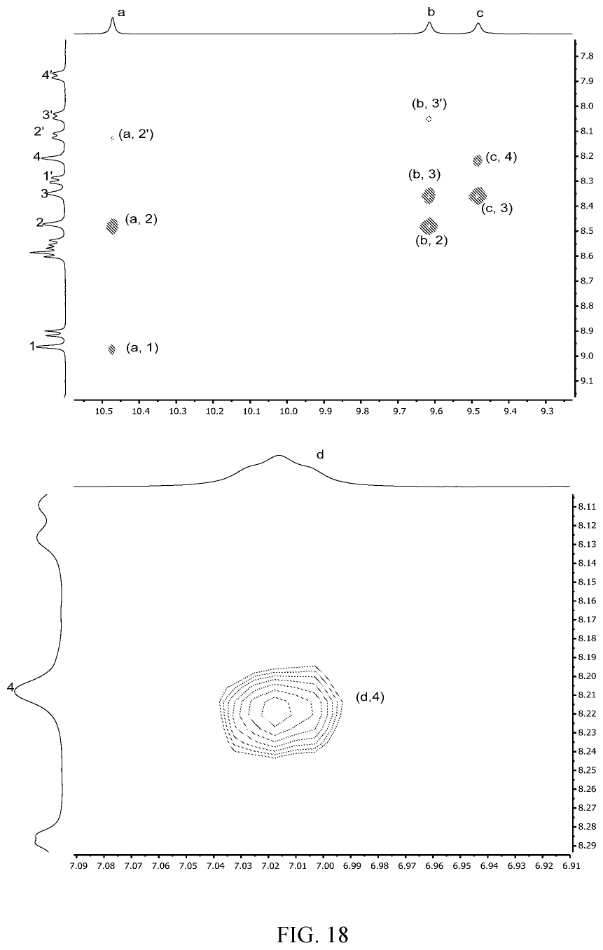

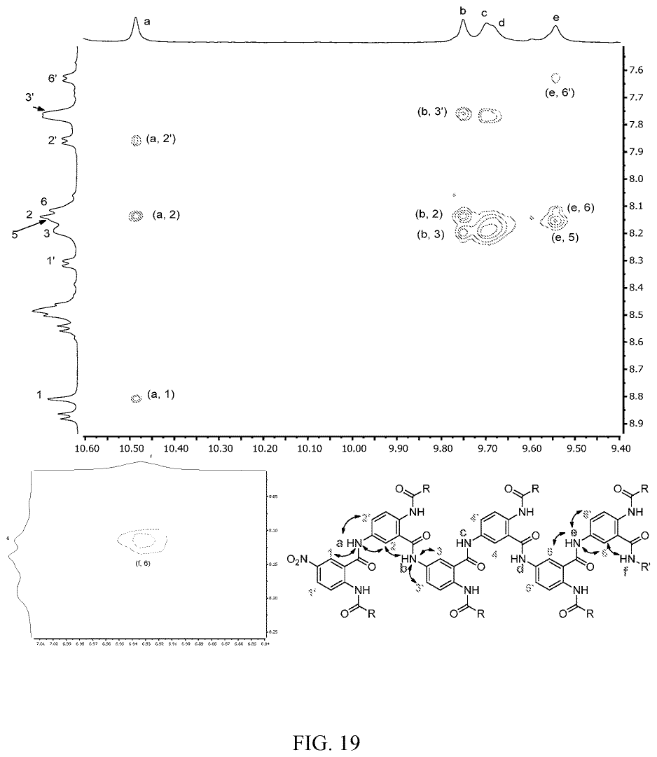

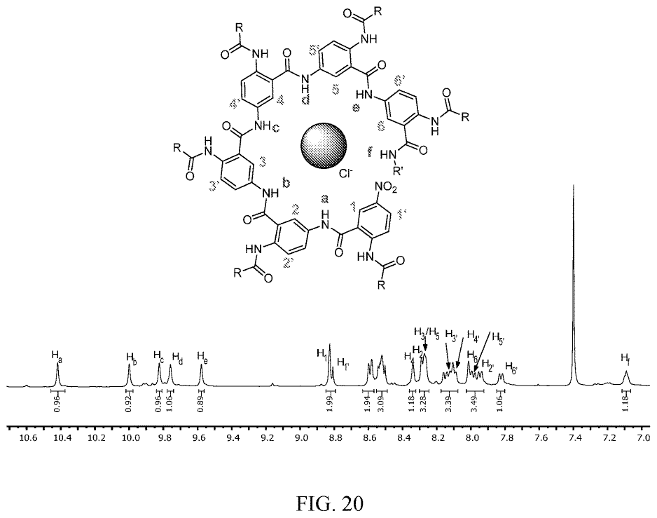

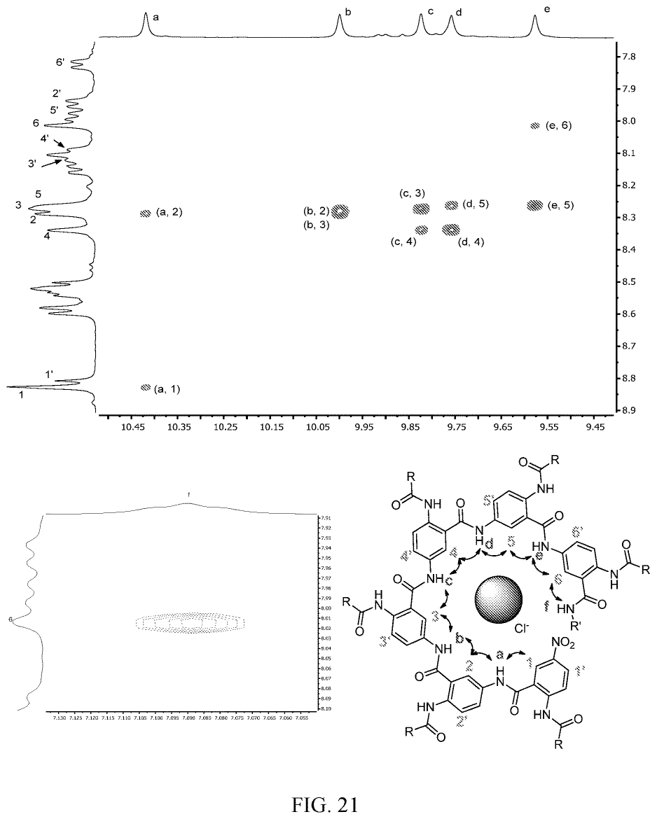

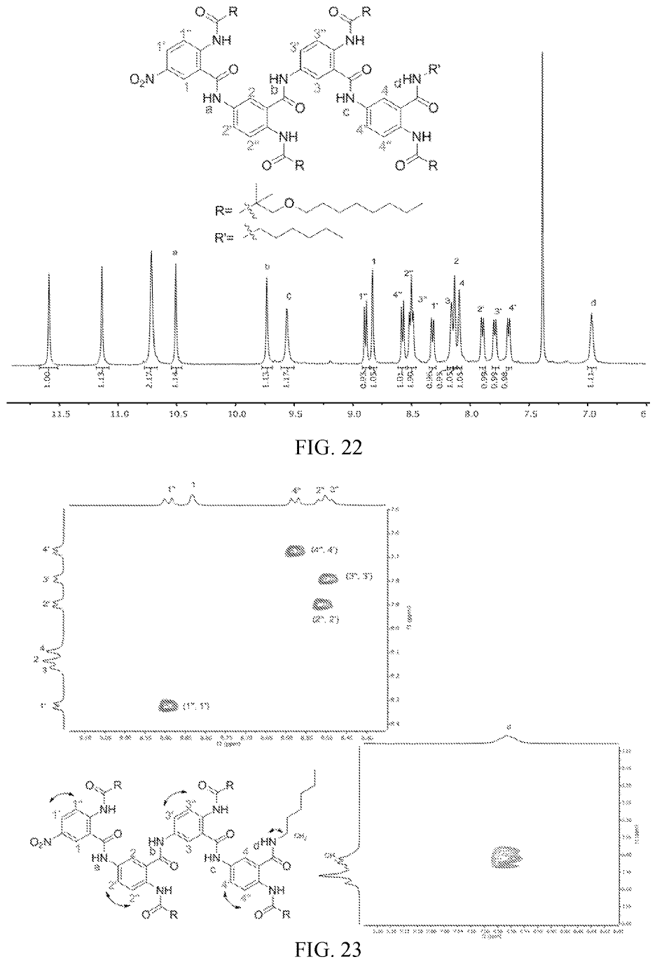

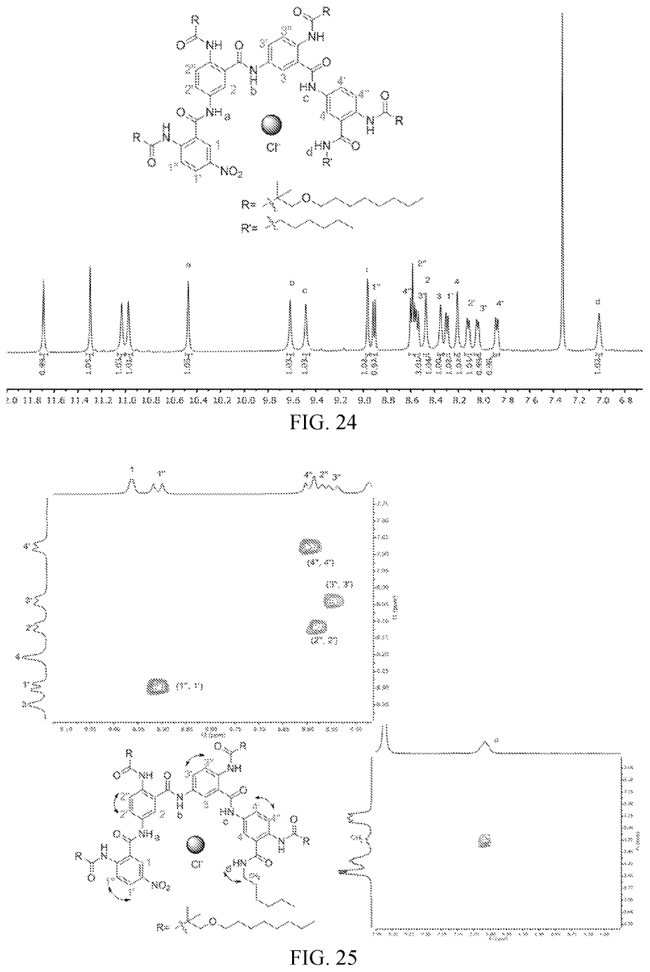

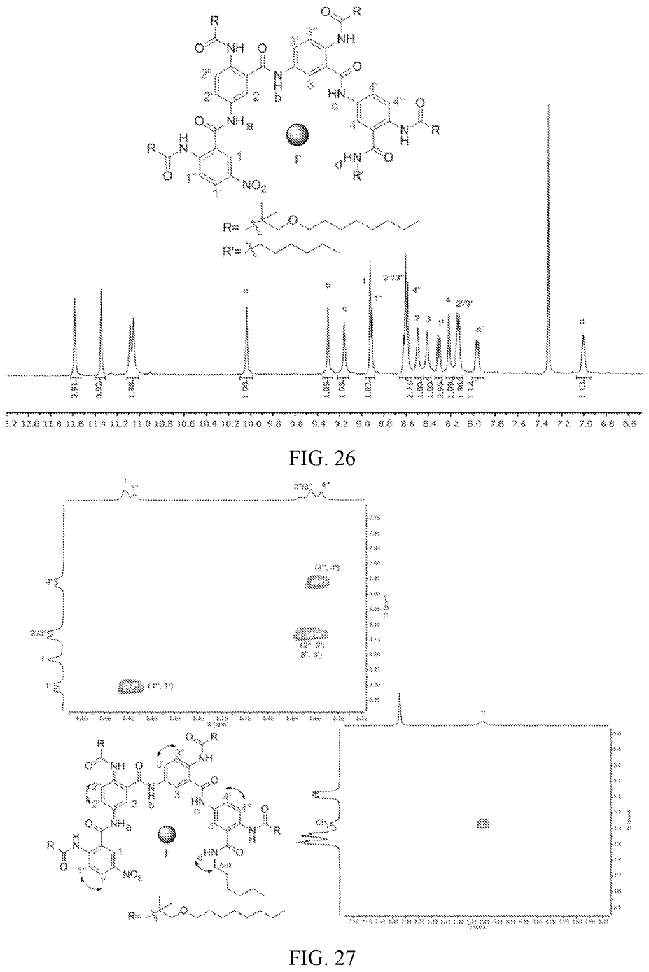

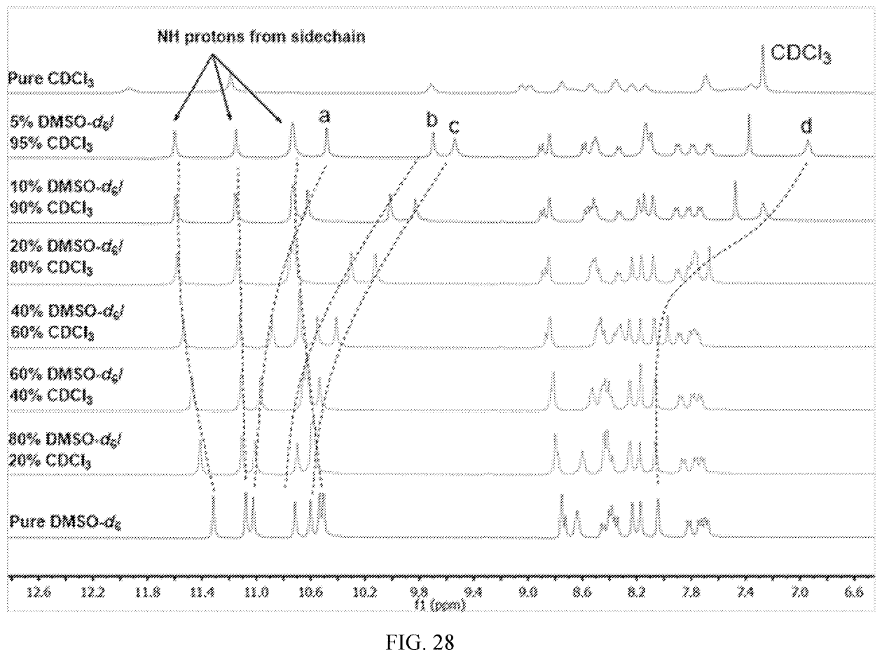

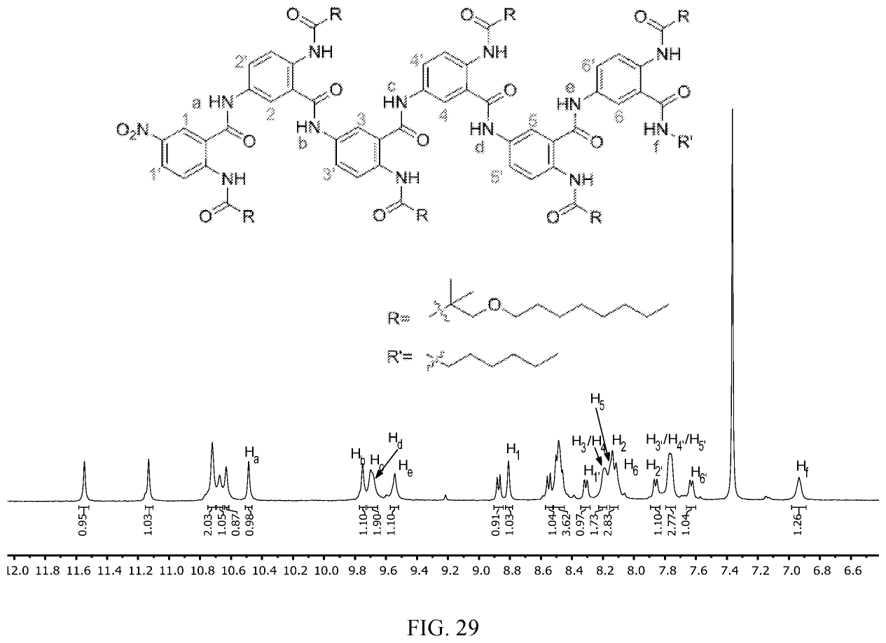

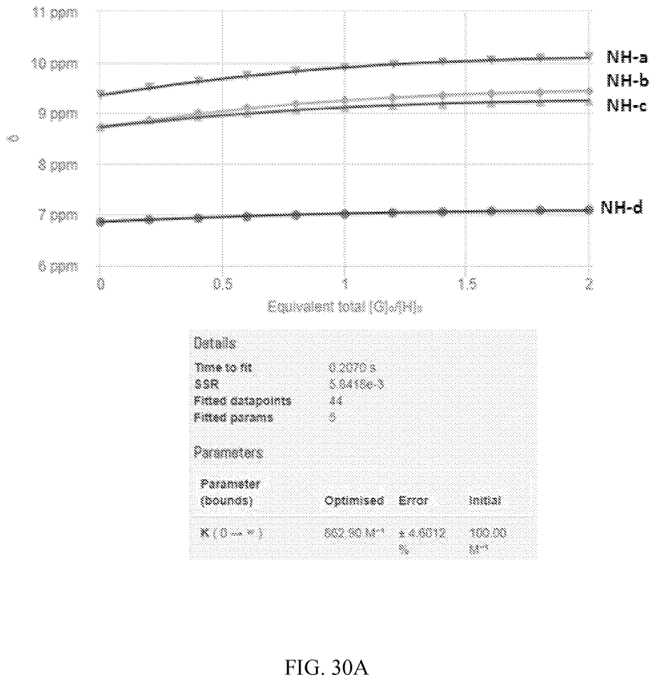

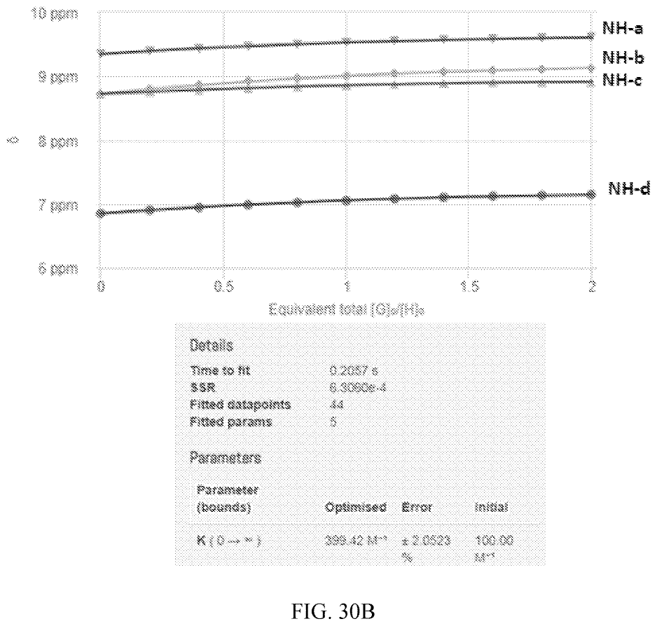

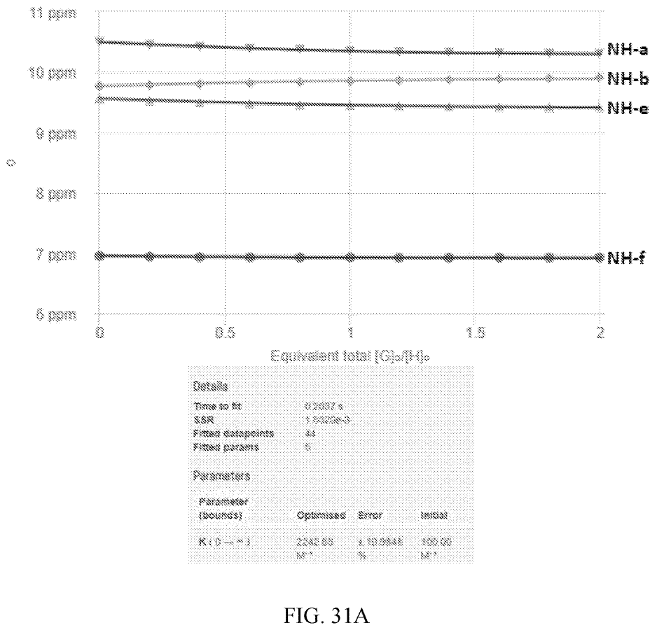

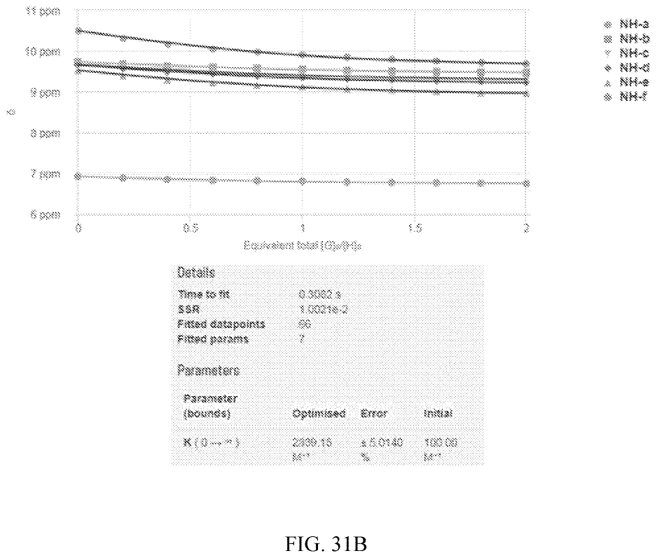

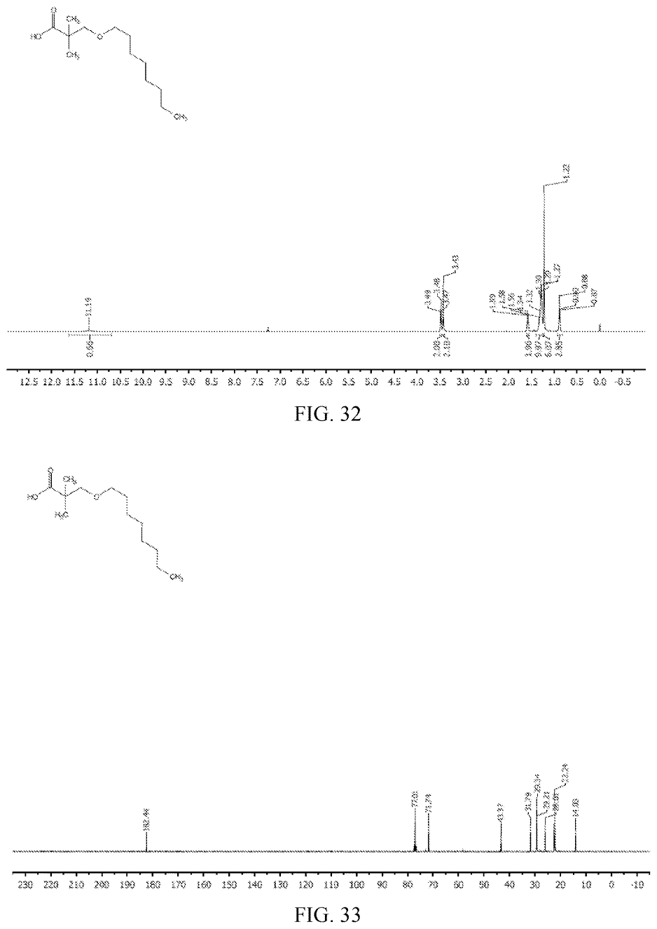

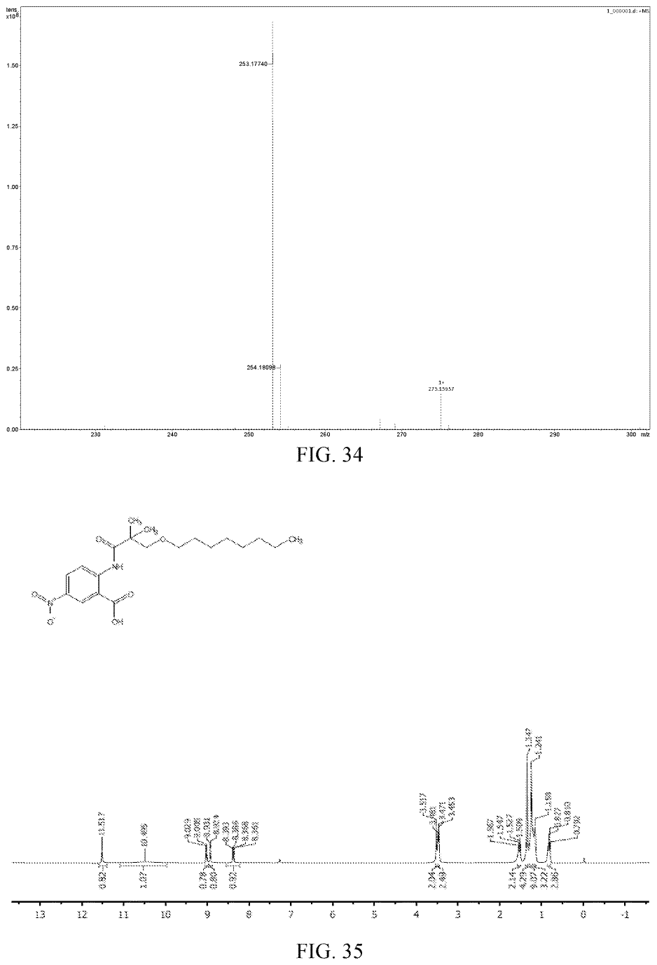

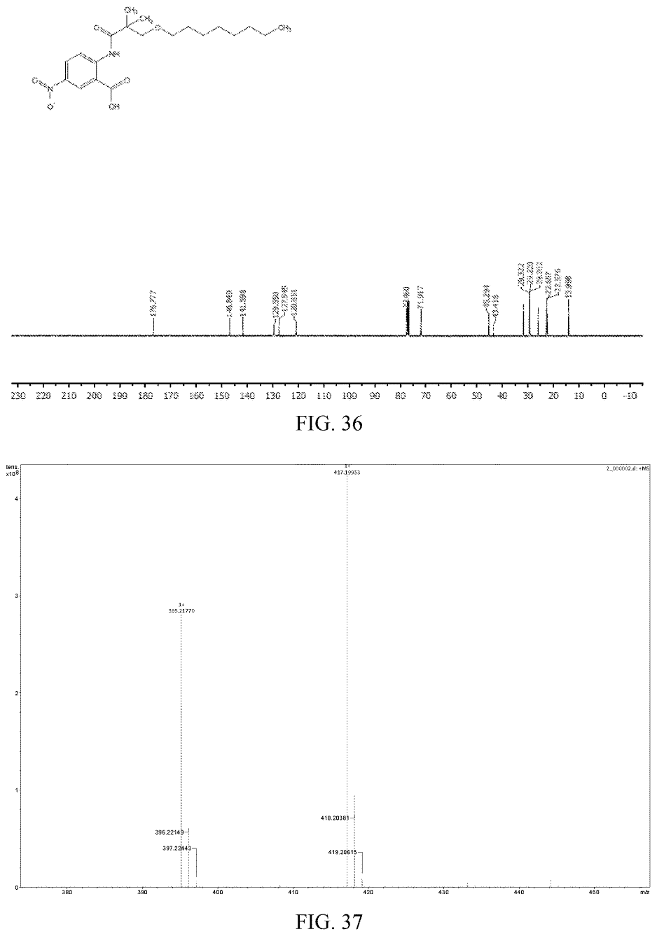

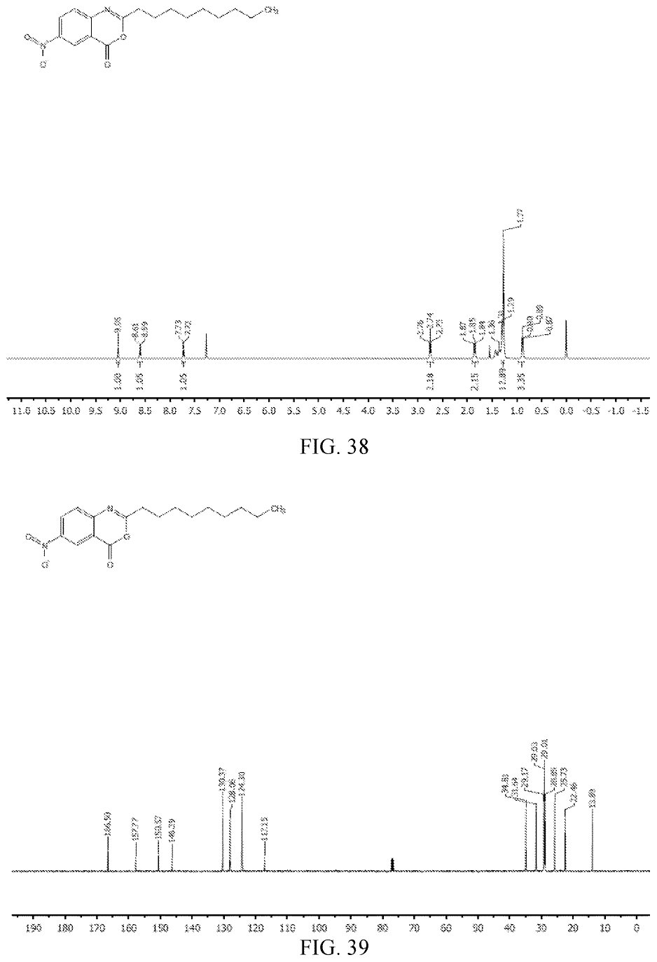

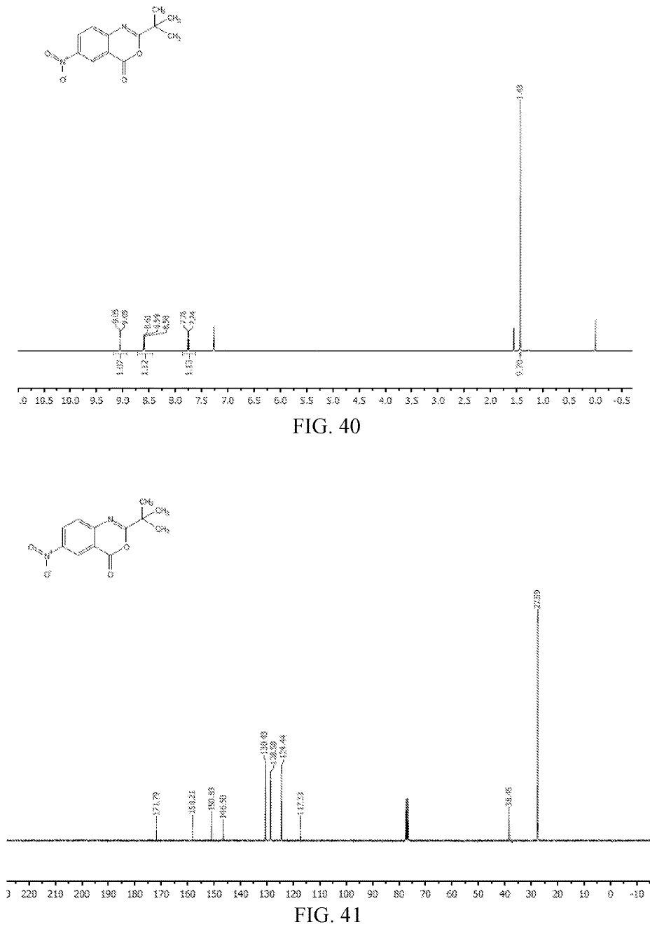

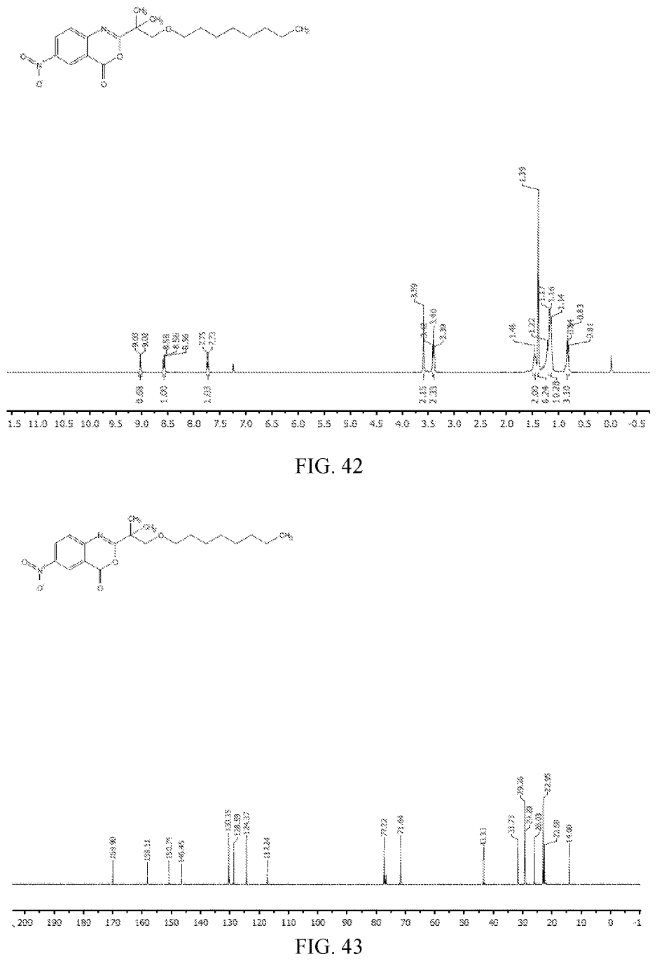

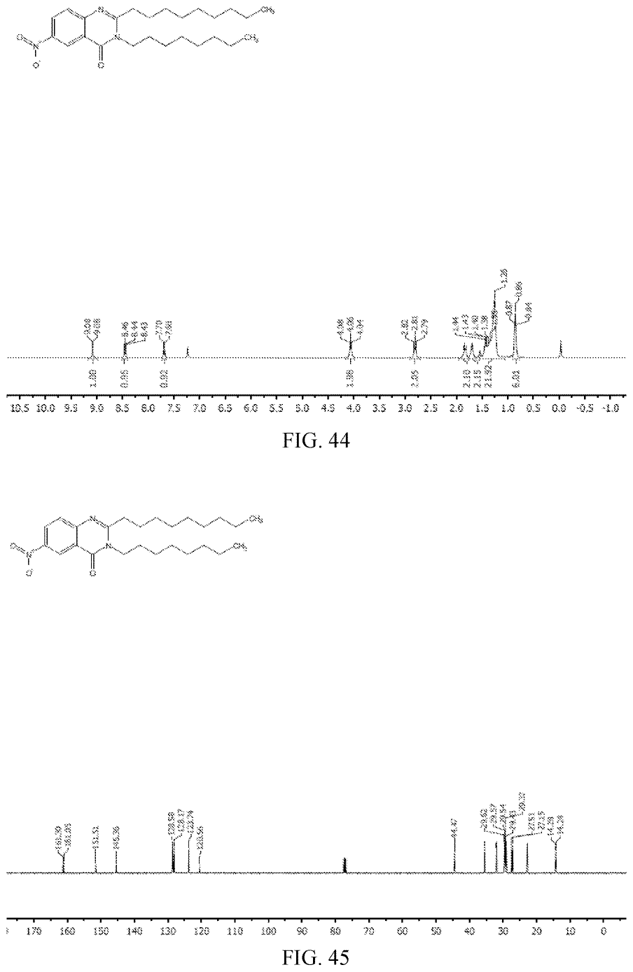

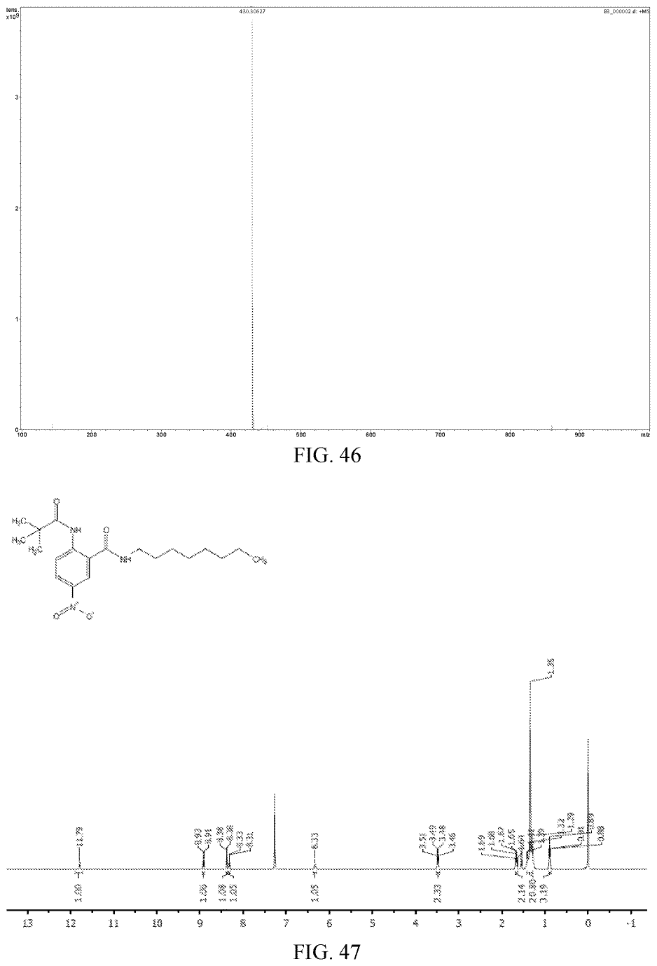

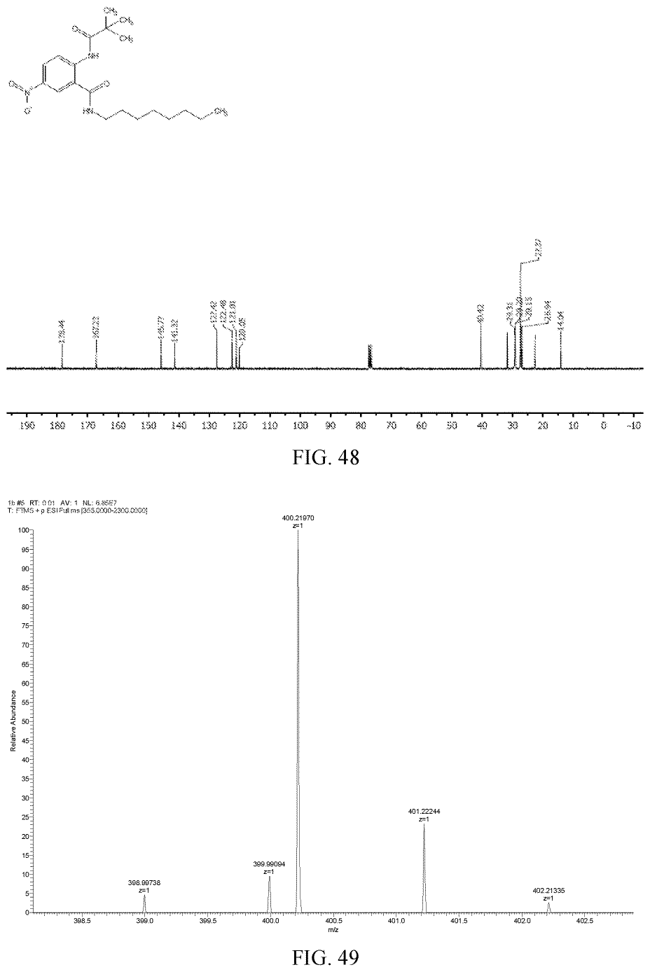

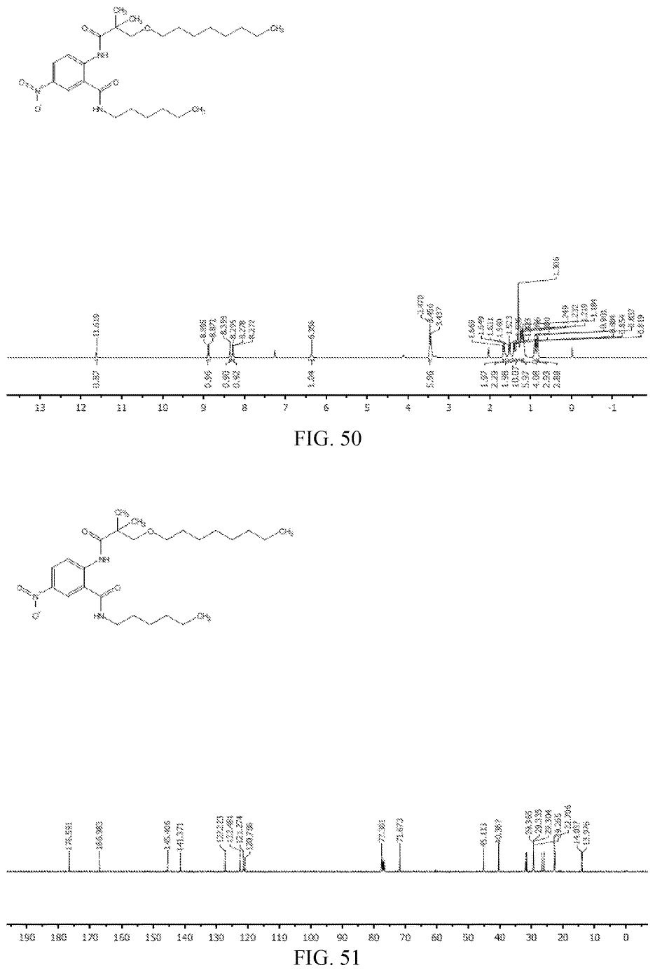

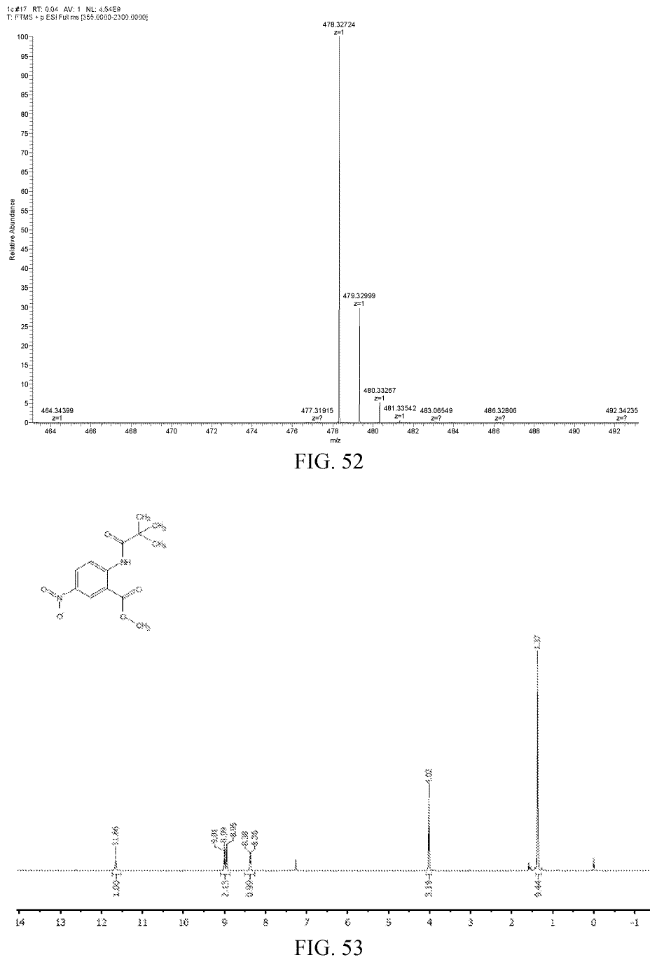

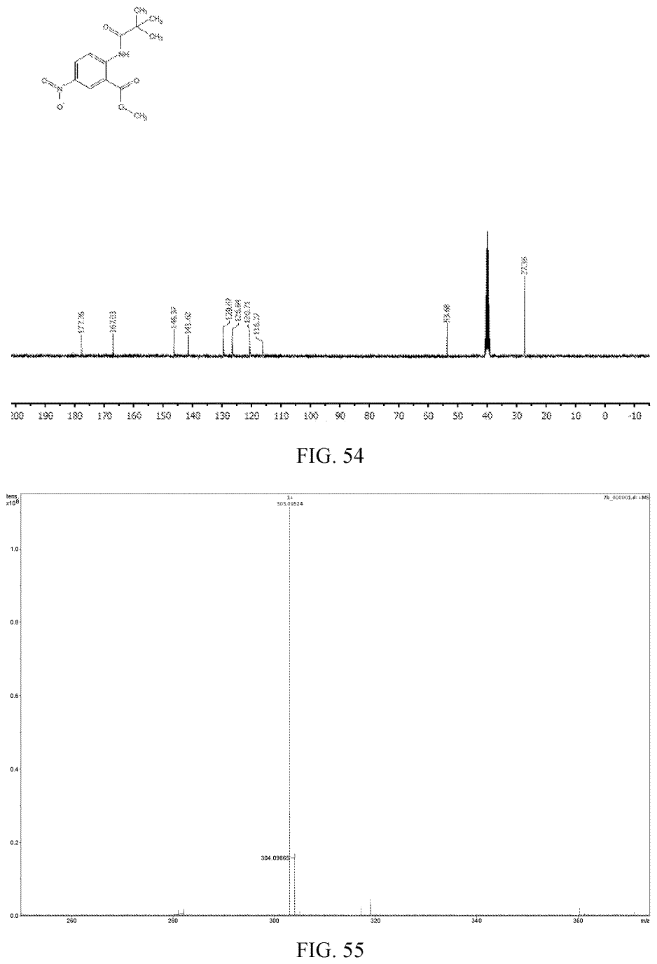

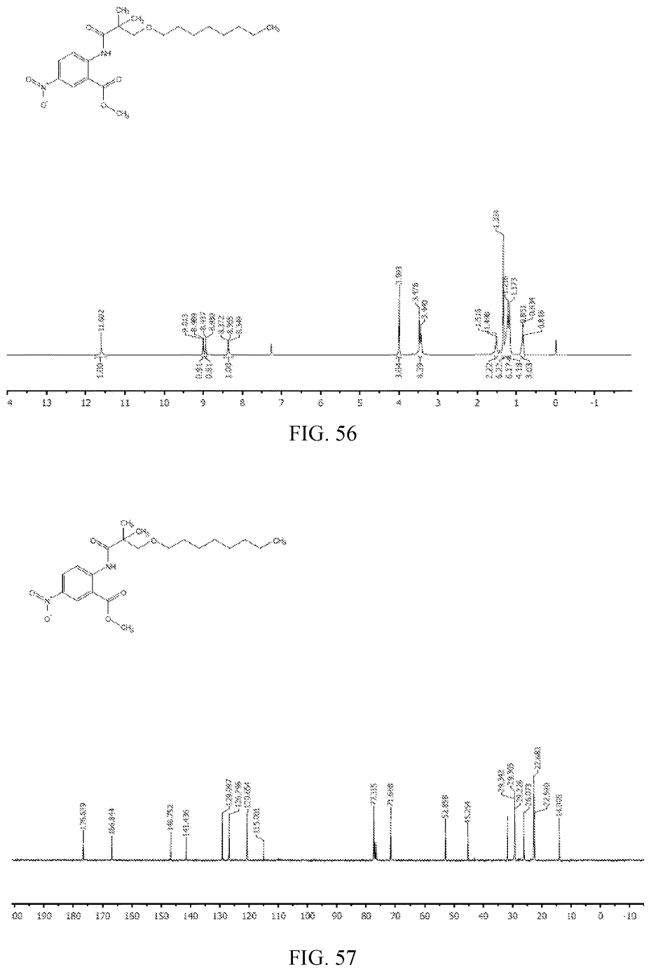

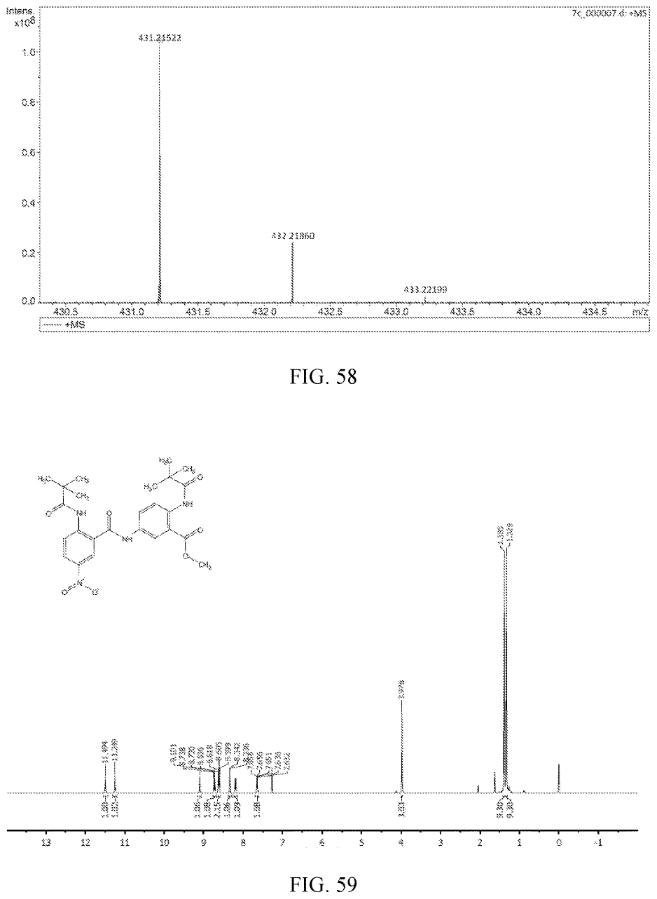

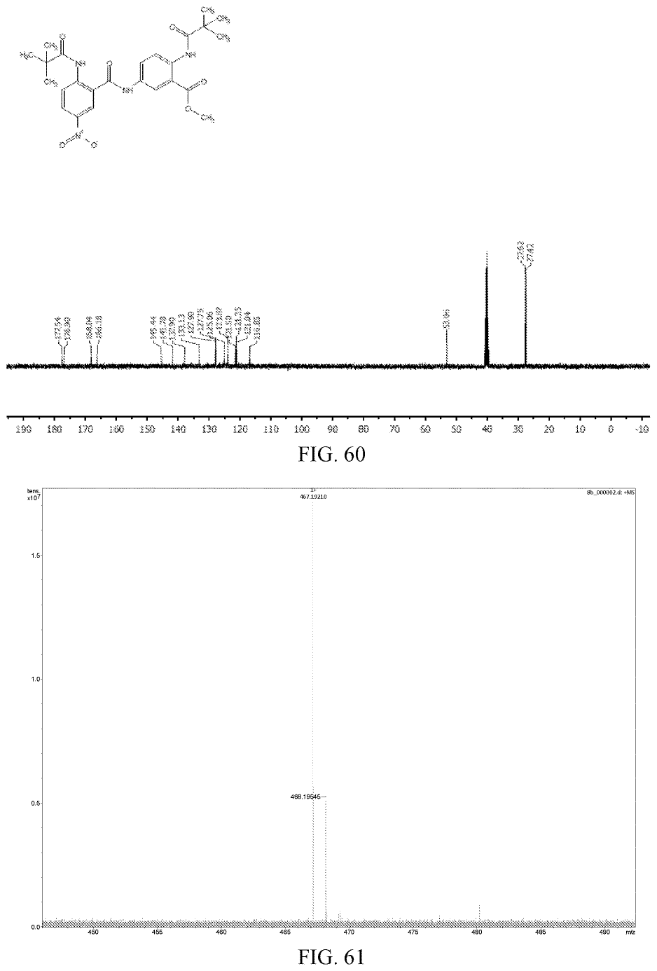

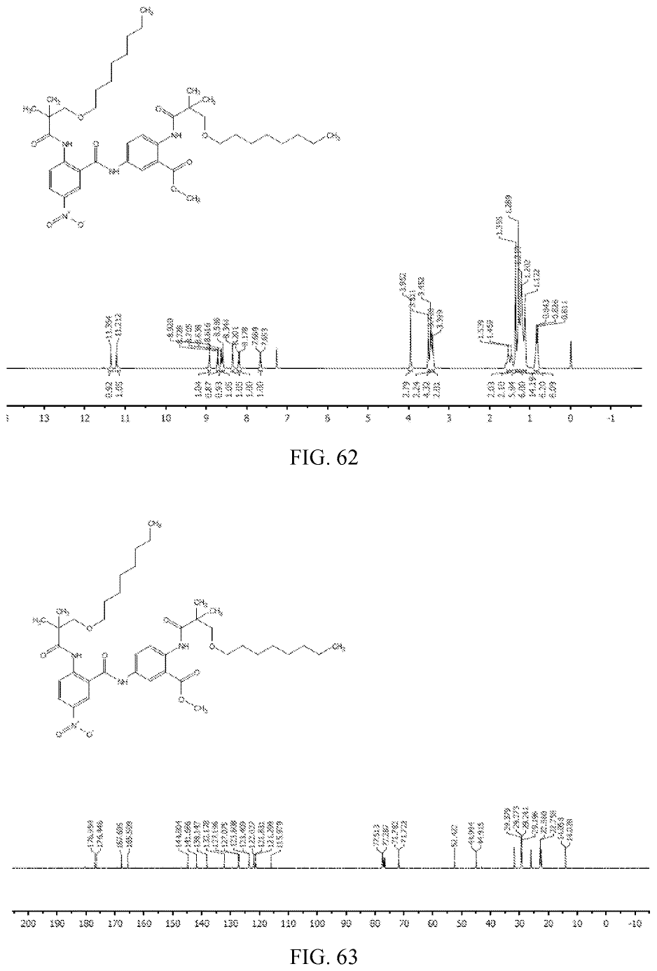

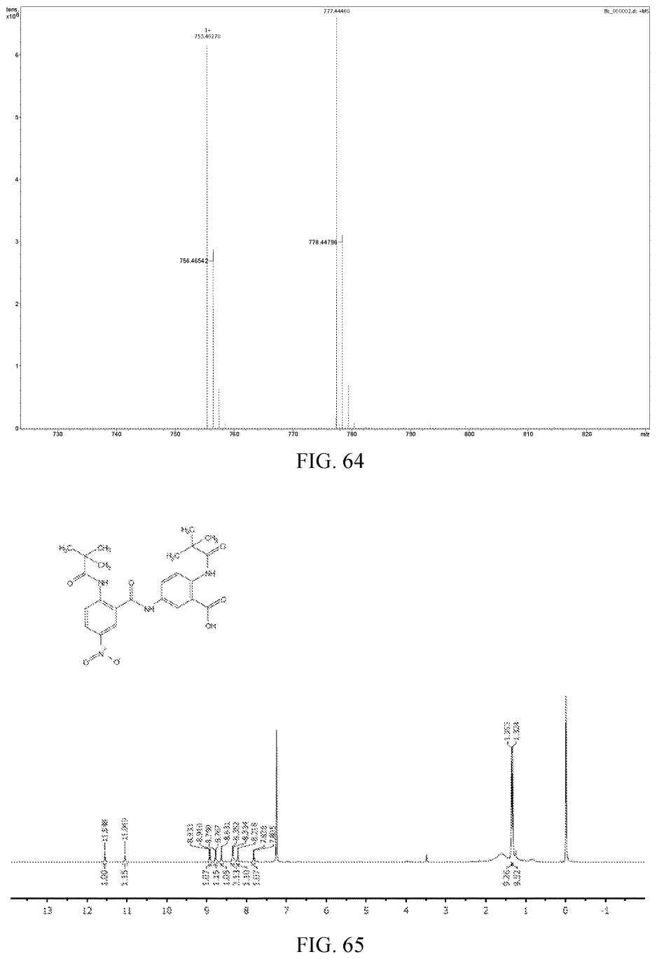

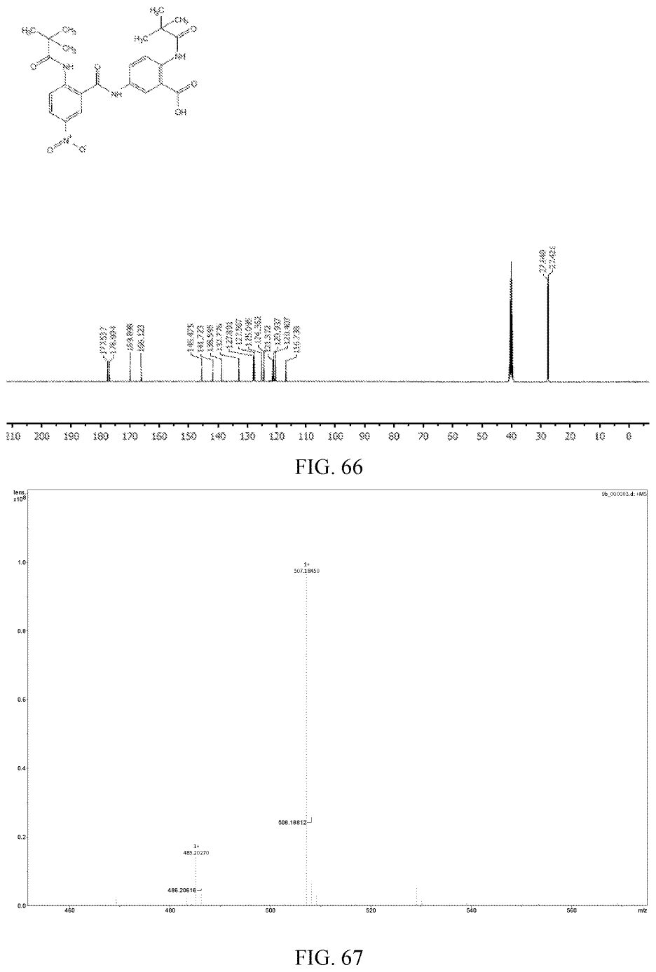

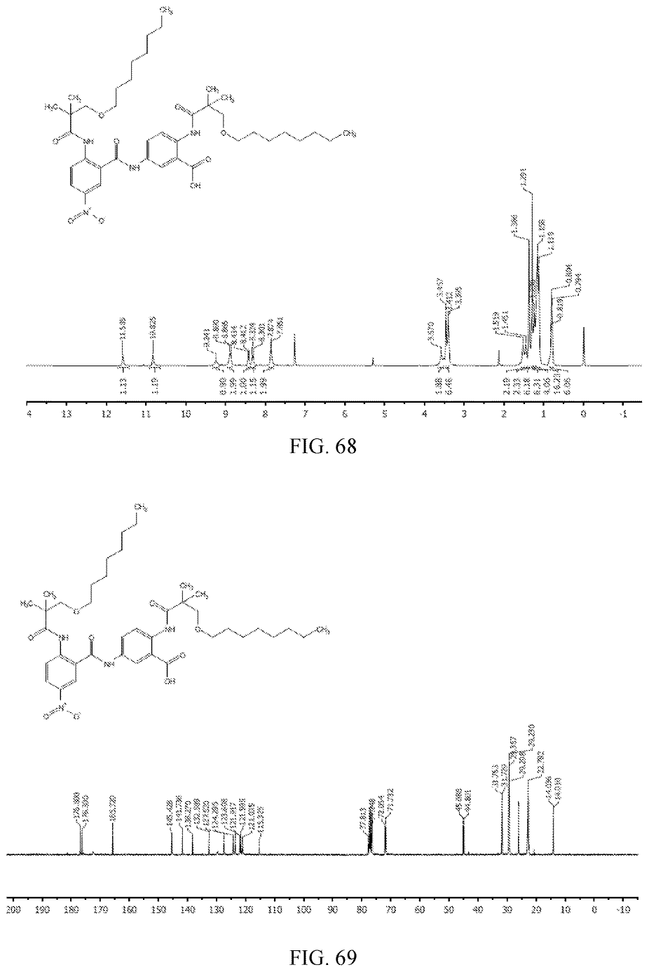

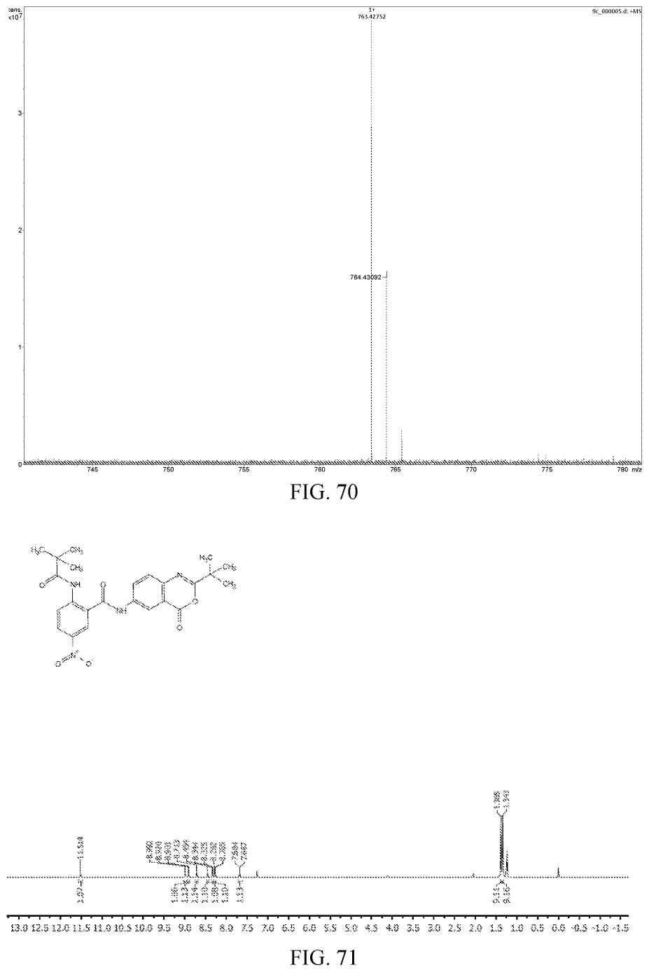

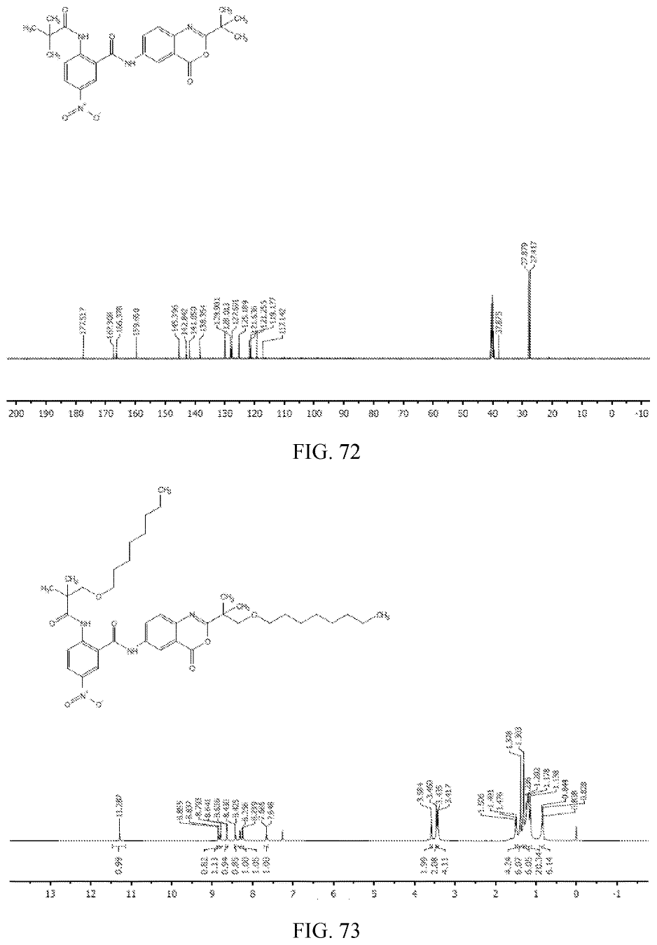

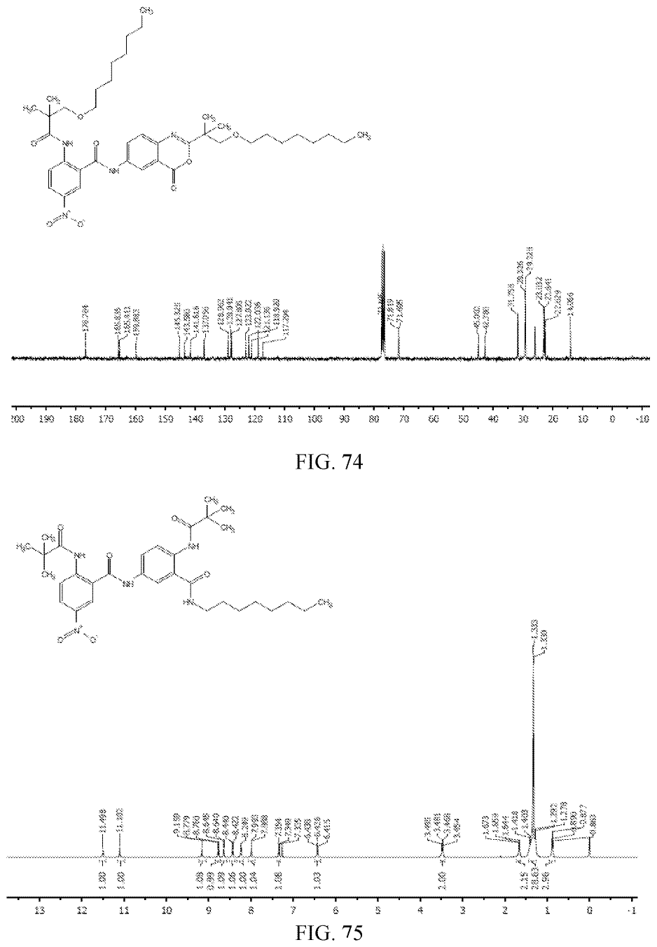

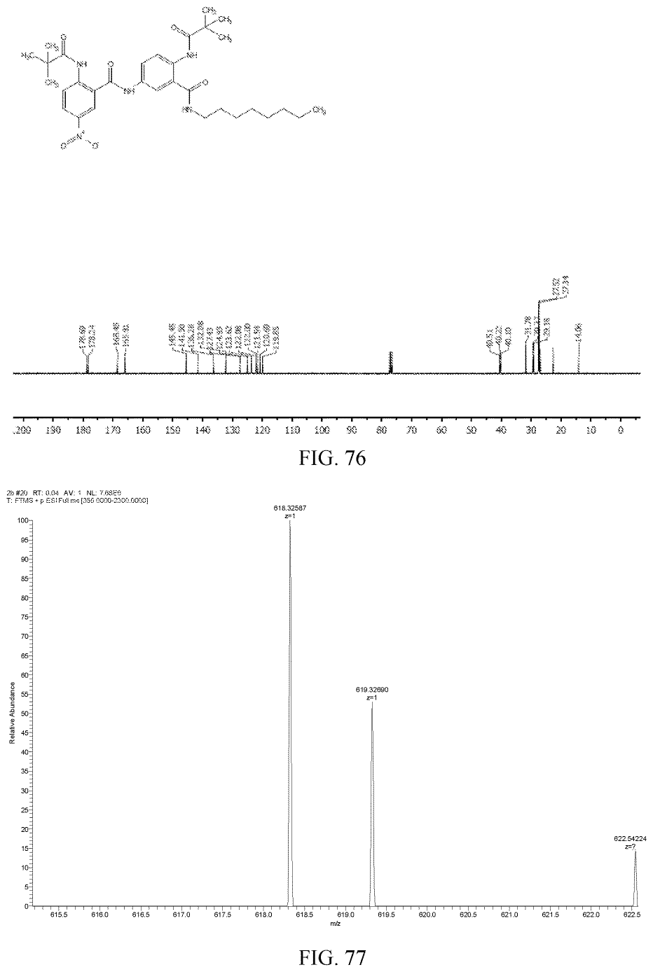

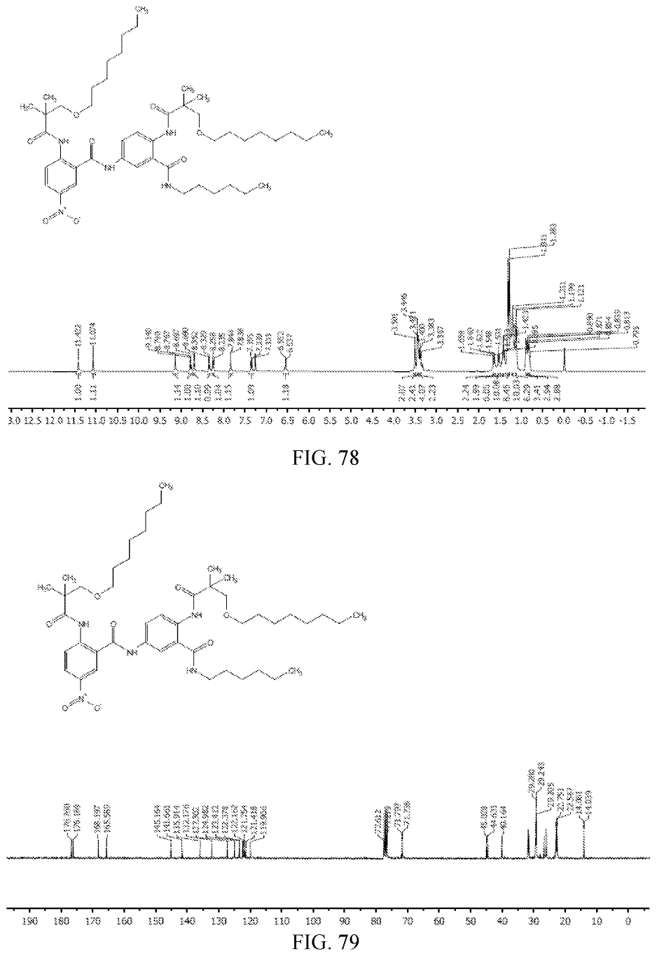

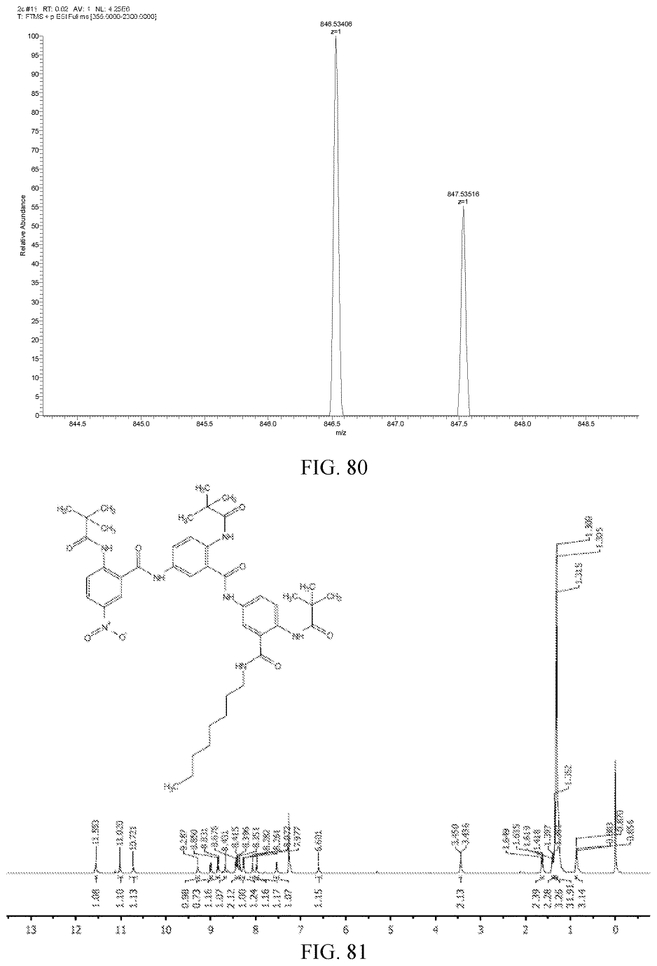

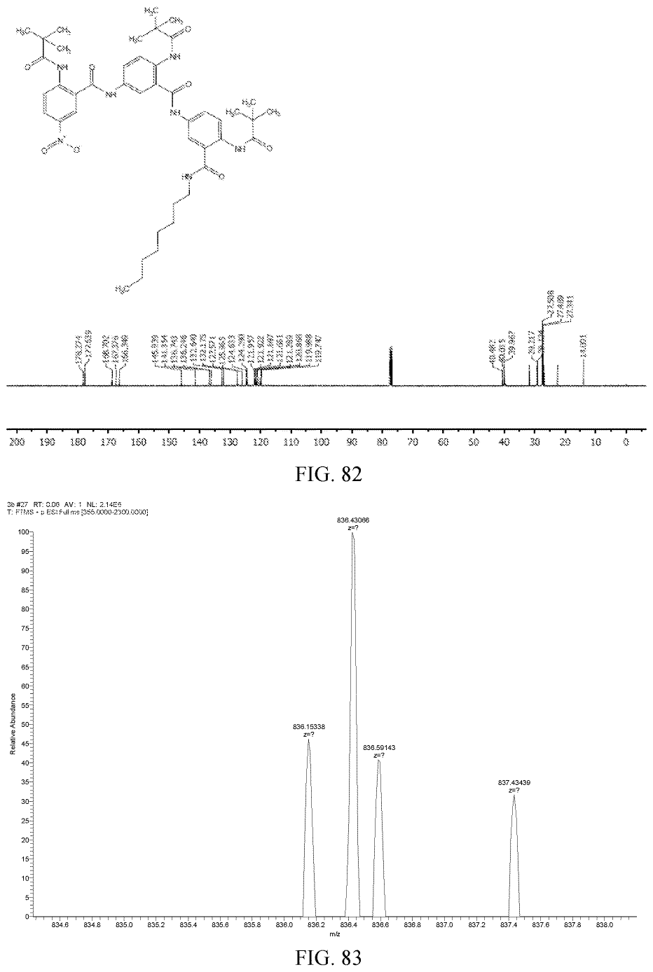

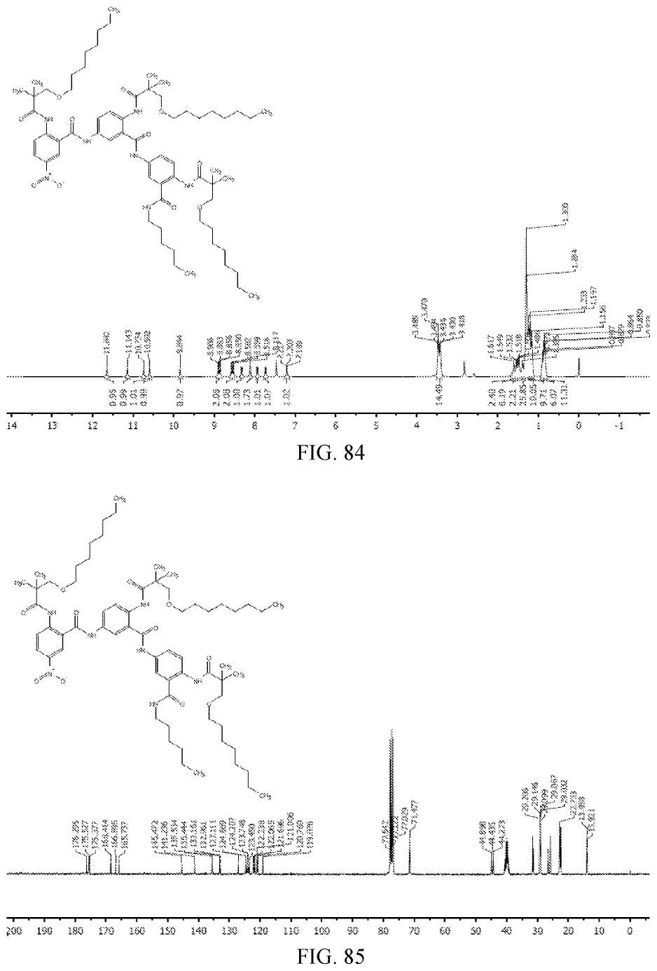

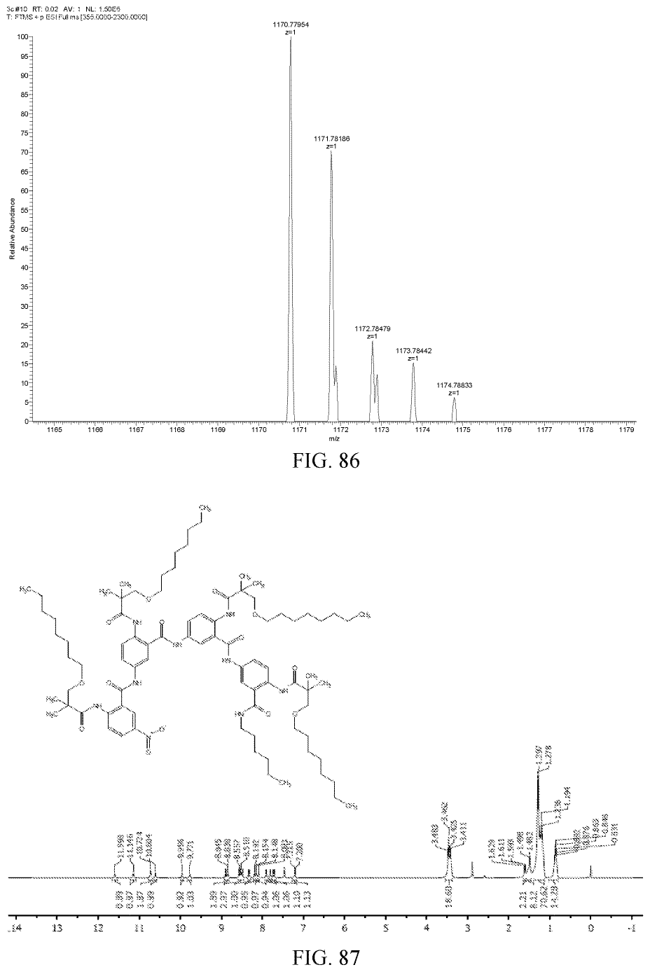

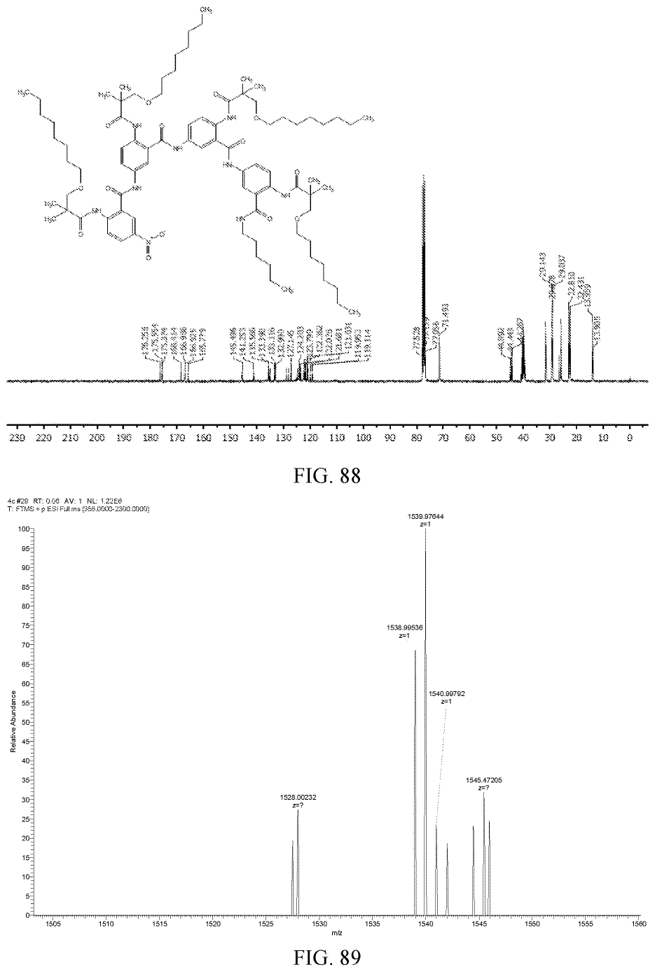

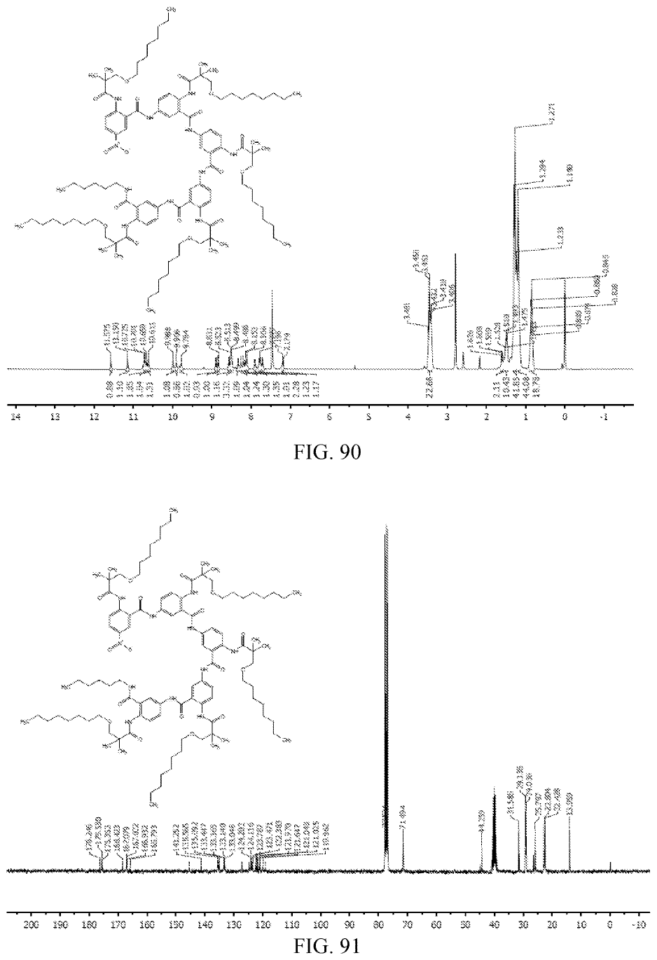

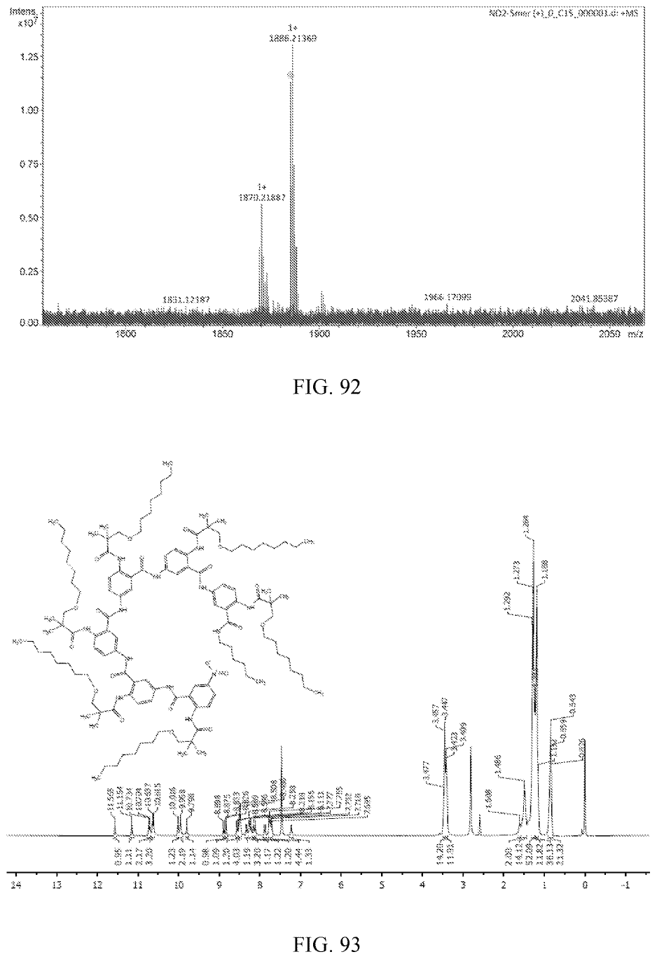

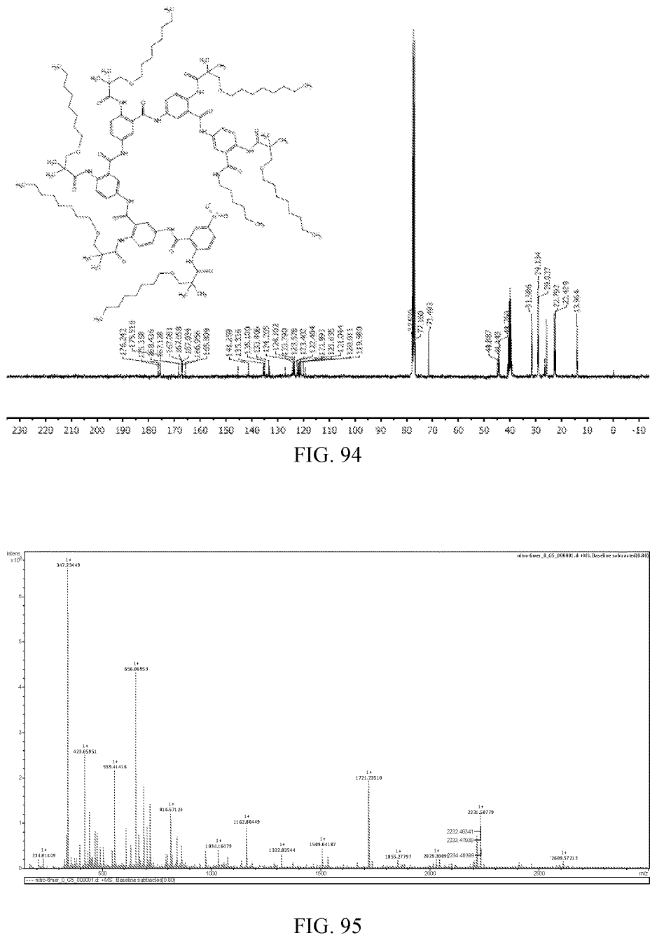

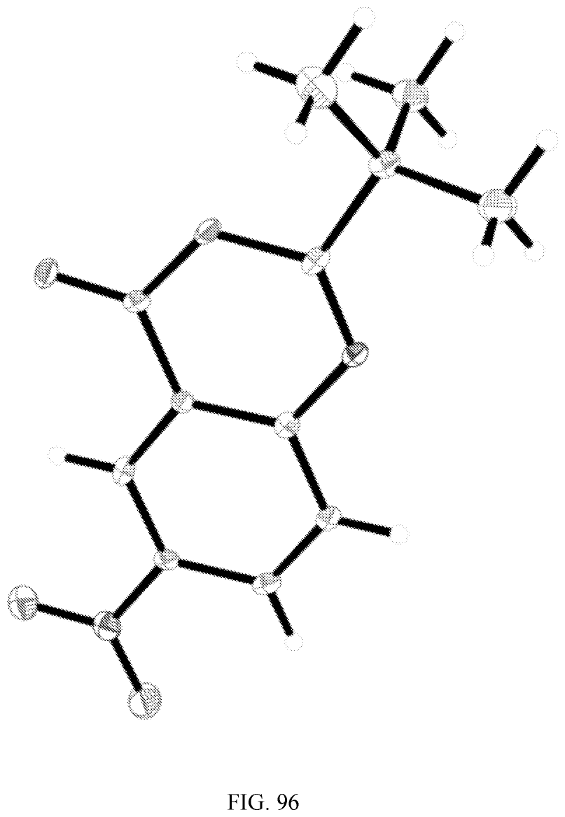

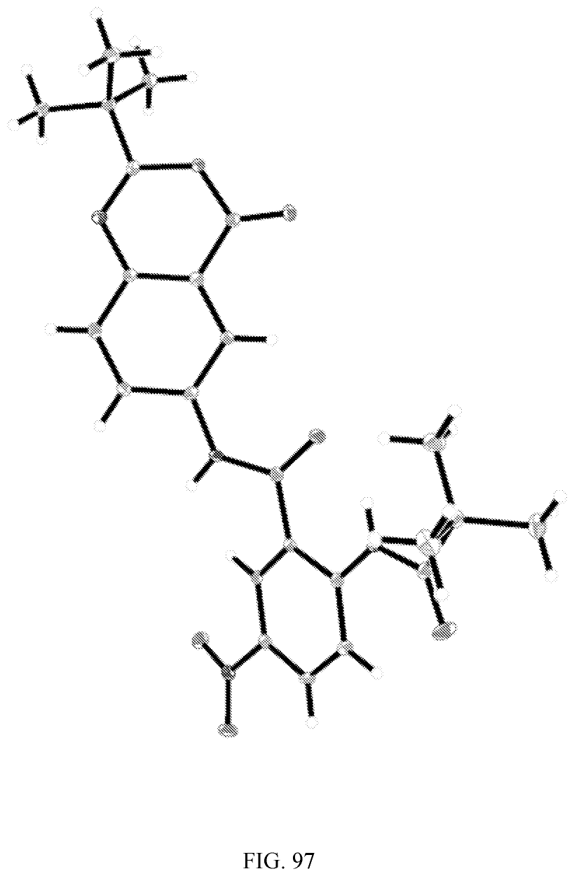

Like that of free 4c, the NOESY spectrum of 4c with 1 equiv. of n-Bu₄N⁺I⁻ (FIG. 4B) also contains NOEs between protons a and 1, b and 2, c and 3, along with d and 4 (FIG. 17), consistent with the presence of the intramoleculart H-bonds. The NOEs involving amide protons a, b, and c, and the “outer” proton 2′, 3′, and 4′, which are observed with free 4c, are absent in the presence of n-Bu₄N⁺I⁻. In contrast, a strong NOEs involving each of these amide protons and the “inner” aromatic proton 2, 3, or 4 is detected. Similar to n-Bu₄N⁺I⁻, mixing n-BuN⁺Cl⁻ with 4c also results in significant weakening or disappearance of NOEs involving the backbone amide and outer aromatic protons, while NOEs involving backbone amide protons and “inner” aromatic protons are strengthened (FIG. 18). The observed NOEs between the amide and inner aromatic protons in the presence of iodide or chloride ion indicate that anion-binding and anion-induced folding is a general behavior of 4c and, similarly, its homologous oligoamides.

The fact that only NOEs between amide protons a, b, and c and inner aromatic protons 1, 2, 3, and 4 are evident in the presence of iodide or chloride ion demonstrates that, upon binding an anion, oligoamide 4c adopts a conformation in which its backbone amide NH groups point convergently, i.e., being placed on the same side as the inner aromatic protons. This is consistent with the adoption of the crescent conformation shown in FIG. 4B. The entropic cost for adopting such a conformation is compensated by the enthalpic contribution from the multiple H-bonding interactions between the anion guest and the backbone amide and aromatic protons.

A new, highly efficient amide bond formation strategy has been established. Repetitive coupling of readily available building blocks derived from 5-aminoanthranilic acid led to oligo (5-amino-N-acylaminoanthranilic acids) with defined lengths in high yields. These are hitherto unknown aromatic oligoamides due to the unavailability of methods for their synthesis. The stepwise coupling of monomeric or oligomeric units allows oligoamides of defined lengths to be prepared under simple conditions that involve refluxing without any coupling reagent. Initial studies indicate that the resultant aromatic oligoamides, with multiple amide NH groups, undergo anion-dependent folding. Given their ready synthetic availability and tunable oligomer length, these novel aromatic oligoamides with multiple H-bond donors, provide a new, versatile platform for developing guest-dependent foldamers or binders of anions and other polar guests, with adjustable affinity and specificity.

TABLE 1
Summary of relative intensity of important NOE interactions between protons of interest.
The intensity of NOE interaction (a, 1) was set to be 1.00 as an internal standard

| NOE interactions | (a, 1) | (a, 2) | (a, 2′) | (b, 2) | (b, 3) | (b, 3′) | (c, 3) | (c, 4) | (c, 4′) | (d, 4) |
|---|---|---|---|---|---|---|---|---|---|---|
| 4c | 1.00 | 2.47 | 0.58 | 5.32 | 4.15 | 1.57 | 5.91 | 0.59 | 0.36 | 0.24 |
| 4c•TBACl | 1.00 | 5.79 | 0.13 | 9.56 | 5.79 | 0.39 | 9.22 | 2.04 | NA | 3.46 |
| 4c•TBAl | 1.00 | 6.40 | NA | 11.73 | 9.19 | NA | 12.70 | 2.96 | NA | 7.48 |

Materials and instruments. Chemicals were purchased from commercial sources and used as received. Silica gel for analytical thin layer chromatography (TLC) and column chromatography (mesh 230~400) were purchased from Sorbent Technologies Inc. 1H NMR spectra were recorded at 400 MHz on Bruker-400 and 500 MHz on Bruker-500, 13C NMR spectra were recorded at 75 MHz on Bruker-300 spectrometers, at ambient temperature using CDCl₃ or DMSO-d₆ as solvents (Cambridge Isotope Laboratories, Inc.). Chemical shifts are reported in parts per million (ppm) downfield from TMS (tetramethylsilane) or the deuterated solvents. Coupling constant in ¹H-NMR were expressed in Hertz (Hz). Regular mass spectra (MS-ESI) were recorded on a Thermo Finnegan LCQ Advantage MS spectrometer. High-resolution electrospray ionization mass spectra (HRMS-ESI) and Matrix-assisted laser desorption/ionization (HRMS-MALDI) were recorded on a Bruker SolariX 12 T Fourier Transform Mass Spectrometer.

Unless otherwise specified, all solvents, including high boiling point solvents, were removed under vacuum with a rotary evaporator. Anhydrous toluene was applied for the coupling reactions.

Preparation of 4-(N, N-dimethylamino)pyridine hydrochloride salt for the amide coupling reactions. 4-(N,N-dimethylamino)pyridine hydrochloride salt (DMAP·HCl) was prepared via the published method without any modifications. For DMAP. HCl mediated-ring opening reaction of benzoxazinone derivatives, 20 mol % of DMAP·HCl and anhydrous toluene were applied to coupling reactions. The reaction suspended solution containing equimolar amounts of benzoxazinone derivatives and amine and 20 mol % DMAP. HCl was heated under reflux with an oil bath. The reaction progress was always monitored with TLC plates. Upon the complete consumption of starting material, the workup was carried out which could be different, largely depending on properties of target molecules.

Hydrogenation reaction. The reduction of nitro compounds to their corresponding amine were carried out in a mixed solvent of 20% methanol and 80% CH₂Cl₂, in the presence of a catalytic amount of Pd/C and pressured hydrogen gas. The resulting reaction solution was stirred at room temperature for 2-8 hours. Upon the completion of reduction reactions, developed TLC plates were stained with a solution of ninhydrin in ethanol and the spot of aromatic amines always turned to be from red to purple. Pd/C was then filtered out and the filtrate was concentrated to afford the corresponding amines which were pure enough and applied directly to the coupling reaction without any further purifications. No analytical data is available for aromatic amines, as they were typically considered to be unstable under the ambient environment.

¹H NMR Titration methods. Titrations of 4c and 6c with anions as their tetra-n-butyl ammonium (TBA) salts were carried out by ¹H NMR (400 MHz on Bruker-400) at room temperature (298 K). 0.5 mL of receptor 4c (5.0 mM) or 6c (2.0 mM) were prepared in the rubber-cap NMR tubes. Aliquots of concentrated TBACl or TBAI solution (50 mM for the titration of 4c and 20 mM for the titration of 6c) containing the same amount of receptors were added into the receptor solution using 20 microliter pipette. An initial ¹H NMR spectrum was collected and additional spectra were obtained after each injection of TBACl or TBAI solution. All titration ¹H NMR spectra were stacked together and the chemical shifts of amide protons were then fitted to the proposed 1:1 binding model using BindFit v0.5 (http://app.supramolecular.org/bindfit/).

### Synthesis and Characterization

### Synthesis of Benzoic Acid.

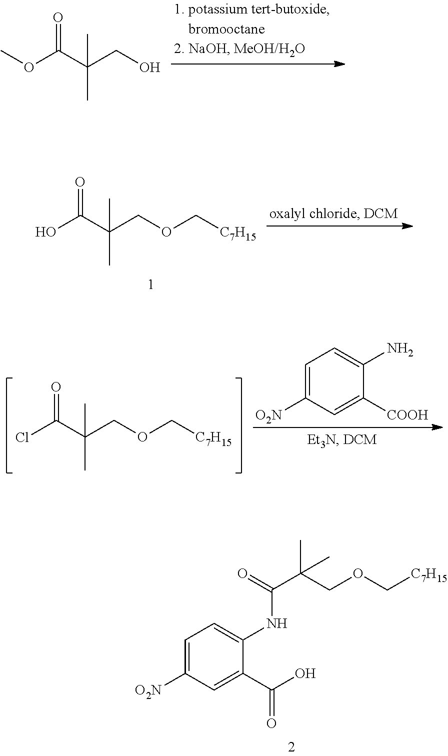

Compound 1: To a 250 mL of round flask were added bromooctane (26.4 g, 200 mmol) and methyl 3-hydroxy-2,2-dimethylpropanoate (38.4 g, 200 mmol). The mixture was stirred vigorously at 0° C. with an ice bath, while potassium t-butoxide (24.7 g, 220 mmol) was added portionwise into the mixture. The reaction mixture was heated to 40° C. overnight. The reaction mixture was poured into icy water and aqueous solution was extracted with ethyl acetate (300 mL). The organic layer was then washed with saturated brine (100 mL×2 times) and dried over anhydrous Na₂SO₄. Ethyl acetate was removed and the resulting residue was dissolved in methanol (250 mL) to give a clean solution to which an aqueous NaOH solution (32 g, 0.8 mol in 50 mL of H₂O) was added dropwise. The reaction solution was stirred at room temperature overnight. Methanol was removed and the resulting aqueous solution was extracted with ethyl acetate (300 mL). The organic layer was acidified with dilute HCl aqueous solution and washed with saturated brine (100 mL×2 times) solution. The organic layer was then dried over anhydrous Na₂SO₄ and removed under vacuum to afford pure acid 1 as a colorless oil (41.2 g, 88% for two steps). ¹H NMR (500 MHz, CDCl₃): δ 11.19 (broad, 1H), 3.48 (t, J=5.0 Hz, 2H), 3.47 (s, 2H), 1.56 (m, 2H), 1.29 (m, 10H), 1.22 (s, 6H), 0.88 (t. J=7.5 Hz, 3H). ¹³C NMR (75 MHz, CDCl₃): δ 182.4, 77.0, 71.7, 43.3, 31.7, 29.3, 29.2, 26.0, 22.6, 22.2, 14.0. HRMS (ESI) m/z: [M+Na]⁺ Calcd for C₁₃H₂₆NaO₃ 253.1780; Found 253.1774.

Compound 2: The preparation of corresponding acid chloride from acid 1 was accomplished by adding oxalyl chloride (9.3 g, 73.2 mmol) to a solution of acid 1 (16 g, 69.5 mmol) in dry CH₂Cl₂ (120 mL) followed with a drop of dry DMF as an initiator. The resulting reaction solution was stirred at room temperature for 6 h before removing CH₂Cl₂. The freshly prepared acid chloride was applied to the next step without any purification. To a solution of 2-amino-5-nitro benzoic acid (12.0 g, 66.0 mmol) and triethylamine (8.63 g, 79.2 mmol) in CH₂Cl₂ (150 mL) was added the freshly prepared acid chloride (16.9 g, 68.0 mmol) dropwise. The reaction solution was stirred at room temperature overnight. Then, the organic layer was washed with saturated NaHCO₃ aqueous solution (50 mL×2 times), brine (50 mL×2 times) and diluted HCl solution (50 mL), and dried over anhydrous Na₂SO₄. The concentration of the reaction solution afforded the pure acid 2 (20.6 g, 79%) as a yellowish oil. ¹H NMR (400 MHz, CDCl₃): δ 11.51 (s, 1H), 10.49 (broad, 1H), 9.02 (d, J=9.6 Hz, 1H), 8.93 (d, J=2.8 Hz, 1H), 8.39 (dd, J=9.6, 2.8 Hz, 1H), 3.51-3.45 (m, 4H), 1.57 (m, 2H), 1.34 (m, 4H), 1.24 (m, 9H), 1.15 (m, 3H), 0.81 (t, J=7.2 Hz, 3H). ¹³C NMR (75 MHz, CDCl₃): δ 167.7, 146.8, 141.6, 129.5, 127.5, 120.8, 77.4, 45.2, 43.4, 31.7, 29.2, 29.2, 25.9, 22.6, 22.5, 22.3, 14.0. HRMS (ESI) m/z: [M+H]⁺ Calcd for C₂₀H₃₁N₂O₆ 395.2182; Found 395.2177.

### Synthesis of Benzoxazinone Derivatives B2a, B2b and B2c

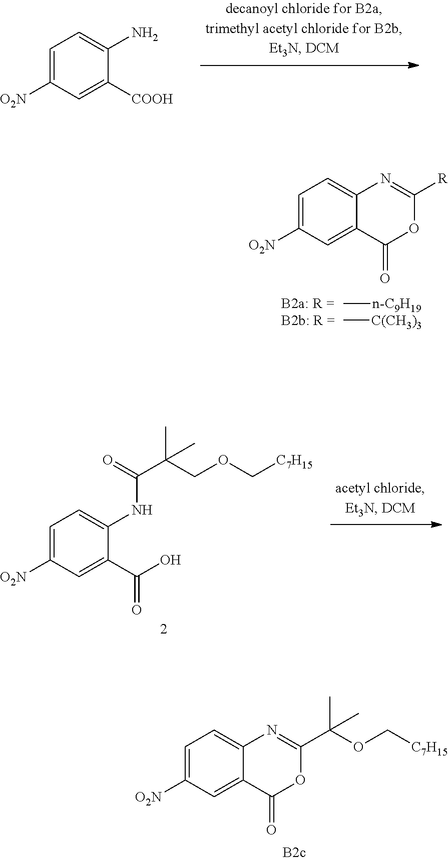

Compound B2a: To a solution of 2-amino-5-nitro benzoic acid (8.0 g, 30 mmol), triethylamine (7.8 g, 72 mmol) in CH₂Cl₂ was added decanoyl chloride (12.0 g, 63 mmol) dropwise. The reaction solution was then stirred at room temperature overnight. After filtering out any solid, the filtrate was concentrated and the residue was subject to silica gel column (hexane/CH₂Cl₂, from 2/1 to 1/1) to afford the pure benzoxazinone B2a as a white solid (6.39 g, 67%). ¹H NMR (400 MHz, CDCl₃): δ 9.05 (s, 1H), 8.61 (d, J=8.0 Hz, 1H), 7.73 (d, J=4.0 Hz, 1H), 2.74 (t, J=12.0 Hz, 2H), 1.85 (m, 2H), 1.36-1.29 (m, 12H), 0.89 (t, J=12.0 Hz, 3H). ¹³C NMR (75 MHz, CDCl₃): δ 116.6, 157.9, 150.7, 146.4, 130.5, 128.2, 124.4, 117.2, 34.9, 31.7, 29.3, 29.2, 29.1, 29.0, 25.9, 22.6, 14.0. MS (ESI) m/z: [M+H]⁺ Calcd, for C₁₇H₂₃N₂O₄ 319.2; Found 319.2.

Compound B2b: To a solution of 2-amino-5-nitro benzoic acid (8.0 g, 30 mmol) and triethylamine (7.8 g, 72 mmol) in dry CH₂Cl₂ (100 mL) was added trimethyl acetyl chloride (7.60 g, 63 mmol) dropwise. The resulting reaction solution was stirred at room temperature overnight. After filtering out any solid, the reaction solution was then concentrated and the residue was subject to silica gel column (hexane/CH₂Cl₂, from 2/1 to 1/1) to afford the pure benzoxazinone B2b as an off-white solid (5.88 g, 79%). ¹H NMR (400 MHz, CDCl₃): δ 9.05 (d, J=5.0 Hz, 1H), 8.61 (dd, J=10.0 and 5.0 Hz, 1H), 7.75 (d, J=10.0 Hz, 1H), 1.43 (s, 9H). ¹³C NMR (75 MHz, CDCl₃): δ 171.8, 158.2, 50.8, 146.5, 130.4, 128.5, 124.4, 117.2, 38.4, 27.6. MS (ESI) m/z: [M+H]⁺ Calcd, for C₁₂H₁₃N₂O₄ 249.1; Found 249.1.

Compound B2c: To a solution of acid 2 (19.7 g, 50 mmol) and triethyl amine (6.1 g, 60 mmol) in dry CH₂Cl₂ (250 mL) was added acetyl chloride (4.8 g, 60 mmol). The obtained reaction solution was stirred at room temperature overnight. After filtering out any solid, the reaction solution was concentrated and the residue was subject to silica gel column (hexane/CH₂Cl₂, from 2/1 to 1/1) to afford the pure benzoxazinone B2c as a yellowish oil (14.1 g, 75%). ¹H NMR (400 MHz, CDCl₃): δ 9.03 (d, J=5.0 Hz, 1H), 8.56 (dd, J=10.0 and 5.0 Hz., 1H), 7.75 (d, J=10.0 Hz, 1H), 3.59 (s, 2H), 3.40 (t, J=7.5 H7.5, 2H), 1.46 (m, 2H), 1.39 (s, 6H), 1.16 (m, 10H), 0.83 (t, J=7.5 Hz, 3H). ¹³C NMR (75 MHz, CDCl₃): δ 169.9, 158.1, 150.7, 146.4, 130.3, 128.6, 124.4, 117.2, 77.2, 71.6, 43.3, 31.7, 29.3, 29.2, 26.0, 22.9, 14.0. MS (ESI) m/z: [M+H]⁺ Calcd, for C₂₀H₂₉N₂O₅ 377.2; Found 377.1.

Products of Ring-Opening of Benzoxazinone Derivatives with Amines.

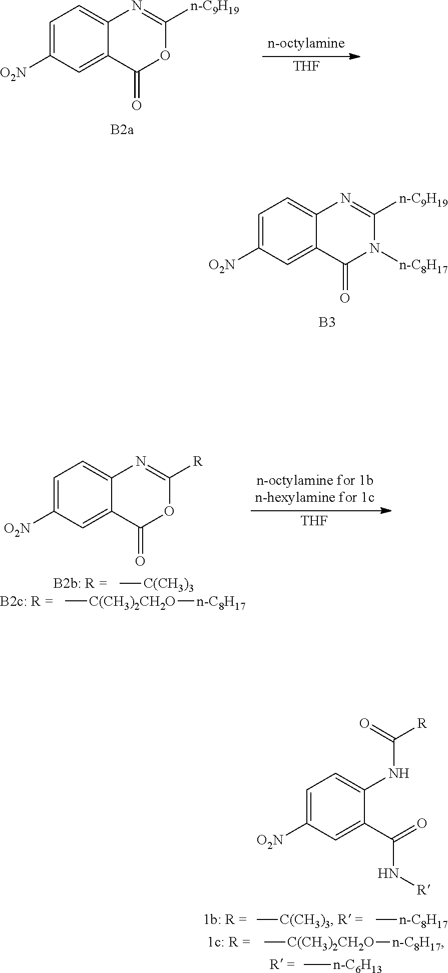

Compound B3: 1.59 g of benzoxazinone B2a (5.0 mmol) and n-octylamine (0.65 g, 5.0 mmol) was dissolved in dry THF (25 mL). The reaction solution was stirred at room temperature overnight. Upon the removal of THE under vacuum, the residue was subject to silica gel column (hexane/ethyl acetate, from 20/1 to 15/1) to afford the pure compound B3 as a yellowish solid (1.25 g, 58%). ¹H NMR (400 MHz, CDCl₃): δ 9.10 (d, J=4.0 Hz, 1H), 8.46 (dd, J=8.0, 4.0 Hz, 1H), 7.72 (d, J=8.0 Hz, 1H), 4.08 (t, J=12 Hz, 2H), 2.82 (t, J=12.0 Hz, 2H), 1.85 (m, 2H), 1.72 (m, 2H), 1.48-1.28 (m, 22H), 0.87 (m, 6H). 13C NMR (75 MHz, CDCl₃): δ 161.1, 160.8, 151.3, 145.1, 128.4, 127.9, 123.5, 120.3, 44.2, 35.2, 31.8, 31.7, 29.4, 29.3, 29.3, 29.2, 29.1, 28.8, 27.3, 26.9, 22.6, 22.6, 14.0, 14.0. HRMS (ESI) m/z: [M+H]⁺ Calcd, for C₂₅H₄₀N₃O₃ 430.3070; Found 430.3063.

Compound 1b: The compound 1b (3.76 g, 99%) as a white solid was prepared from benzoxazinone B2b (2.48 g, 10.0 mmol) and n-octylamine (1.29 g, 10.0 mmol) via the same method as the preparation of compound B3 without any modification. ¹H NMR (500 MHz, CDCl₃): δ 11.79 (s, 1H), 8.92 (d, J=10.0 Hz, 1H), 8.39 (s, 1H), 8.32 (dd, J=10.0 Hz, 1H), 6.33 (broad, 1H), 3.48 (dd, J=15.0 and 10.0 Hz, 2H), 1.67 (m, 2H), 1.39-1.29 (m, 20H), 0.89 (t, J=10.0 Hz, 3H). ¹³C NMR (75 MHz, CDCl₃): δ 178.4, 167.2, 145.8, 141.3, 127.4, 122.5, 121.0, 120.0, 40.4, 31.7, 29.3, 29.2, 29.1, 27.4, 26.9, 22.6, 14.0. HRMS (ESI) m/z: [M+Na]⁺ Calcd, for C₂₀H₃₁N₃NaO₄ 400.2212; Found 400.2197.

Compound 1c: The compound 1c (4.77 g, 99%) as a yellowish oil was prepared from benzoxazinone B2c (3.76 g, 10.0 mmol) and n-hexylamine (1.02 g, 10.0 mmol) via the same method as the preparation of compound B3 without any modification. 1H NMR (400 MHz, CDCl₃): δ 11.62 (s, 1H), 8.95 (d, J=9.2 Hz, 1H), 8.36 (s, 1H), 8.28 (dd, J=9.2 Hz, 6.8 Hz, 1H), 6.36 (broad, 1H), 3.47-3.44 (m, 6H), 1.65 (m, 2H), 1.52 (m, 2H), 1.42-1.38 (m, 2H), 1.31 (m, 10H), 1.25-1.18 (m, 10H), 0.90 (t, J=6.8 Hz, 3H), 0.84 (t, J=7.2 Hz, 3H). ¹³C NMR (75 MHz, CDCl₃): δ 176.6, 167.0, 145.4, 141.3, 127.2, 122.5, 121.3, 120.8, 77.4, 71.7, 45.1, 40.4, 31.8, 31.4, 29.4, 29.3, 29.3, 29.3, 26.6, 26.0, 22.7, 22.6, 22.5, 14.0, 14.0. HRMS (ESI) m/z: [M+H]⁺ Calcd, for C₂₆H₄₄N₃O₅ 478.3281; Found 478.3272.

### Synthesis of Benzoxazinone Dimer 2b′ and 2c′

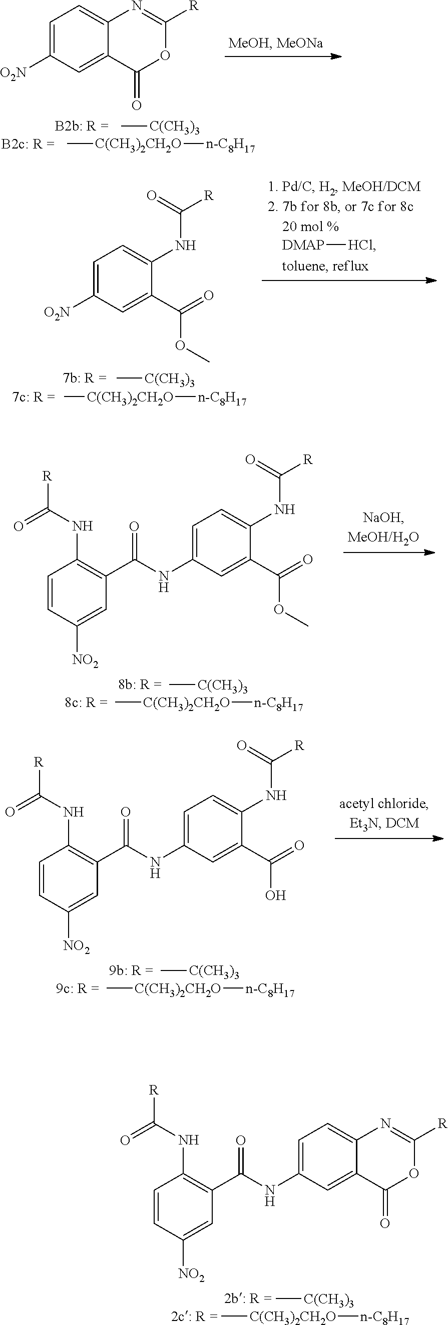

Compound 7b: To a solution of benzoxazinone B2b (4.96 g, 20.0 mmol) in methanol (100 mL) was added 10 mol % of sodium methoxide (0.11 g). The obtained reaction solution was stirred at room temperature overnight. Methanol was removed and the residue was then dissolved in ethyl acetate (100 mL). The organic layer was washed with brine (20 mL×2 times) and dried over anhydrous Na₂SO₄. The methyl ester 7b was obtained as an off-white solid after removing ethyl acetate (5.49 g, 98%). ¹H NMR (500 MHz, CDCl₃): δ 11.66 (s, 1H), 9.01 (d, J=10.0 Hz, 1H), 8.95 (s, 1H), 8.36 (d, J=10.0 Hz, 1H), 4.02 (s, 3H), 1.37 (s, 9H). ¹³C NMR (75 MHz, DMSO-d₆): δ 177.8, 167.0, 146.4, 141.6, 129.7, 126.6, 120.7, 116.3, 53.7, 27.4. HRMS (ESI) m/z: [M+Na]⁺ Calcd, for C₁₃H₁₆N₂NaO₅ 303.0957; Found 303.0952.

Compound 7c: The compound 7c (7.92 g, 97%) as a yellowish oil was prepared from the solution of benzoxazinone B2c (7.52 g, 20.0 mmol) and 10 mol % of sodium methoxide (0.11 g) in methanol (100 mL) via the same method as the preparation of compound 7b without any modification. ¹H NMR (400 MHz, CDCl₃): δ 11.60 (s, 1H), 9.01 (d, J=9.6 Hz, 1H), 8.93 (d, J=2.8 Hz, 1H), 8.36 (dd, J=9.6, 2.8 Hz, 1H), 3.99 (s, 3H), 3.47-3.44 (m, 4H), 1.51 (m, 2H), 1.33 (s, 6H), 1.21-1.17 (m, 10H), 0.83 (t, J=6.8 Hz, 3H). ¹³C NMR (75 MHz, CDCl₃): δ 176.6, 166.8, 146.7, 141.4, 129.1, 126.8, 120.6, 115.0, 77.3, 71.7, 52.9, 45.2, 31.7, 29.3, 29.3, 26.1, 22.7, 22.6, 22.4, 14.0. HRMS (ESI) m/z: [M+Na]⁺ Calcd, for C₂₁H₃₂N₂NaO₆ 431.2158; Found 431.2152.

Compound 8b: The reduction reaction of methyl ester 7b to prepare corresponding aromatic amine was accomplished via the general hydrogenation method. The freshly prepared aromatic amine (0.75 g, 3.0 mmol) and B2b (0.74 g, 3.0 mmol) were dissolved in dry toluene (50 mL) followed by addition of 20 mol % of DMAP. HCl salt. The obtained suspended solution was heated under reflux overnight. The organic layer was then washed with brine (30 mL×2 times) and dried over anhydrous Na₂SO₄. After removal of toluene under vacuum, the obtained residue was dissolved in the hot methanol. The white precipitate then formed and was collected by filtration to give the pure compound 8b as an off-white solid (1.39 g, 93%). ¹H NMR (500 MHz, CDCl₃): δ 11.49 (s, 1H), 11.24 (s, 1H), 9.10 (s, 1H), 8.73 (d, J=9.5 Hz, 1H), 8.63 (d, J=9.5 Hz, 1H), 8.60 (d, J=2.5 Hz, 1H), 8.34 (d, J=2.5 Hz, 1H), 8.20 (dd, J=9.5, 2.5 Hz, 1H), 7.65 (dd, J=9.5, 2.5 Hz, 1H), 3.98 (s, 3H), 1.38 (s, 9H), 1.32 (s, 9H). ¹³C NMR (75 MHz, CDCl₃): δ 177.5, 176.9, 168.0, 166.2, 145.4, 141.8, 137.9, 133.1, 127.9, 127.7, 125.1, 123.9, 121.5, 121.2, 121.0, 116.8, 53.1, 27.6, 27.4. HRMS (ESI) m/z: [M−MeOH+H]⁺ Calcd, for C₂₄H₂₇N₄O₆ 467.1931; Found 467.1921.

Compound 8c: The reduction reaction of methyl ester 7c to prepare corresponding aromatic amine was accomplished via the general hydrogenation method. To a solution of freshly prepared aromatic amine (1.13 g, 3.0 mmol) and B2c (1.13 g, 3.0 mmol) in dry toluene (50 mL) was added 20 mol % of DMAP·HCl salt. The obtained suspended solution was heated under reflux overnight. The organic layer was then washed with brine (30 mL×2 times) and dried over anhydrous Na₂SO₄. After removal of toluene under vacuum, the residue was subject to flash silica gel column (hexane/ethyl acetate, from 5/1 to 2/1) to afford the pure compound 8c as a yellowish oil (2.01 g, 89%). ¹H NMR (400 MHz, CDCl₃): δ 11.35 (s, 1H), 11.21 (s, 1H), 8.92 (s, 1H), 8.73 (d, J=9.2 Hz, 1H), 8.64 (d, J=8.8 Hz, 1H), 8.58 (s, 1H), 8.34 (s, 1H), 8.20 (d, J=9.2 Hz, 1H), 7.66 (d, J=8.8 Hz, 1H), 3.95 (s, 3H), 3.51 (s, 2H), 3.45-3.39 (m, 6H), 1.54 (m, 2H), 1.45 (m, 2H), 1.35 (s, 6H), 1.28 (s, 6H), 1.22-1.12 (m, 20H), 0.84-0.81 (m, 6H). ¹³C NMR (75 MHz, CDCl₃): δ 176.9, 176.4, 167.7, 165.5, 144.8, 141.7, 138.1, 132.2, 127.2, 127.1, 123.6, 123.4, 122.0, 121.8, 121.3, 116.0, 77.5, 77.3, 71.8, 71.7, 52.4, 45.0, 44.9, 31.8, 31.7, 29.3, 29.2, 26.1, 26.0, 25.9, 22.8, 22.7, 22.6, 22.6, 22.4, 14.1, 14.0. HRMS (ESI) m/z: [M+H]⁺ Calcd, for C₄₁H₆₃N₄O₉ 755.4595; Found 755.4627.

Compound 9b: To a solution of methyl ester 8b (1.00 g, 2.0 mmol) in methanol (30 mL) was added an aqueous NaOH solution (0.32 g of NaOH in 1 mL of H₂O). The reaction solution was stirred at room temperature overnight. Then methanol was removed and the residue was acidified with diluted HCl aqueous solution. The resulting aqueous layer was then extracted with ethyl acetate (50 mL×2 times). The organic layer was then washed with brine and dried over anhydrous Na₂SO₄. The removal of ethyl acetate under vacuum afforded the acid 9b as an off-white solid (0.94 g, 97%). ¹H NMR (500 MHz, CDCl₃): δ 11.54 (s, 1H), 11.05 (s, 1H), 8.93 (d, J=11.5 Hz, 1H), 8.79 (d, J=11.5 Hz, 1H), 8.63 (s, 1H), 8.35-8.33 (m, 2H), 8.21 (s, 1H), 7.82 (d, J=11.5 Hz, 1H), 1.35 (s, 9H), 1.32 (s, 9H). ¹³C NMR (75 MHz, DMSO-d₆): δ 177.5, 176.9, 169.9, 166.1, 145.5, 141.7, 138.6, 132.9, 127.9, 127.6, 125.1, 124.4, 121.4, 120.9, 120.4, 116.7, 27.6, 27.4. HRMS (ESI) m/z: [M+Na]⁺ Calcd, for C₂₄H₂₈N₄NaO₇ 507.1856; Found 507.1845.

Compound 9c: The hydrolysis of methyl ester 8c (1.51 g, 2.0 mmol) to prepare acid 9c (1.39 g, 94%) was carried out using the same method as the preparation of compound 9b without any modification. ¹H NMR (400 MHz, CDCl₃): δ 11.58 (s, 1H), 10.82 (s, 1H), 9.24 (s, 1H), 8.89-8.86 (m, 2H), 8.43 (d, J=8.8 Hz, 1H), 8.32 (d, J=9.2 Hz, 1H), 7.87-7.85 (m, 2H), 3.57 (m, 2H), 3.45-3.39 (m, 6H), 1.52 (m, 2H), 1.45 (m, 2H), 1.36 (m, 6H), 1.29 (m, 6H), 1.23 (m, 4H), 1.15-1.11 (m, 16H), 0.81-0.79 (m, 6H). ¹³C NMR (75 MHz, CDCl₃): δ 176.8, 176.3, 165.7, 145.4, 141.7, 138.3, 132.6, 127.5, 124.3, 123.6, 121.9, 121.6, 121.0, 115.3, 77.8, 77.2, 72.0, 71.7, 45.1, 44.8, 31.7, 31.7, 29.3, 29.2, 26.0, 26.0, 23.0, 22.8, 22.6, 22.5, 14.0, 14.0. HRMS (ESI) m/z: [M+Na]⁺ Calcd, for C₄₀H₆₀N₄NaO₉ 763.4258; Found 763.4275.

Compound 2b′: To a solution of acid 9b (0.48 g, 1.0 mmol) and triethyl amine (0.25 g, 2.5 mmol) in dry DCM (10 mL) was added acetyl chloride (0.16 g, 2.0 mmol) dropwise. The reaction solution was stirred at room temperature overnight. Any solid was filtered out and the obtained solution was concentrated under high vacuum. The residue was then subject to flash silica gel column (CH₂Cl₂/ethyl acetate, from 10/1 to 5/1) to afford pure compound 2b′ as an off-white solid (0.33 g, 71%). ¹H NMR (500 MHz, CDCl₃): δ 11.51 (s, 1H), 8.99 (s, 1H), 8.92 (d, J=9.5 Hz, 1H), 8.71 (s, 1H), 8.45 (s, 1H), 8.34 (d, J=9.5 Hz, 1H), 8.28 (d, J=8.5 Hz., 1H), 7.68 (d, J=8.5 Hz., 1H), 1.38 (s, 9H), 1.34 (s, 9H). ¹³C NMR (75 MHz, DMSO-d₆): δ 177.5, 167.3, 166.4, 159.6, 145.4, 142.8, 141.8, 138.3, 129.9, 128.0, 127.7, 125.2, 121.6, 121.2, 119.2, 117.1, 37.9, 27.9, 27.4. MS (ESI) m/z: [M+H]⁺ Calcd. for C₂₄H₂₇N₄O₆ 467.2; Found 467.1.

Compound 2c′: To a solution of acid 9c (0.74 g, 1.0 mmol) and triethyl amine (0.25 g, 2.5 mmol) in dry DCM (10 mL) was added acetyl chloride (0.16 g, 2.0 mmol) dropwise. The reaction solution was stirred at room temperature overnight. Any solid was filtered out and the obtained solution was concentrated under high vacuum. The residue was then subject to flash silica gel column (hexane/ethyl acetate, from 5/1 to 2/1) to afford pure compound 2c′ as an off-white solid (0.48 g, 67%). ¹H NMR (400 MHz, CDCl₃): δ 11.28 (s, 1H), 8.85 (d, J=7.2 Hz, 1H), 8.79 (s, 1H), 8.64 (d, J=2.0 Hz, 1H), 8.43 (d, J=2.0 Hz, 1H), 8.32 (dd, J=7.2, 2.0 Hz, 1H), 8.30 (dd, J=6.8, 2.0 Hz, 1H), 7.66 (d, J=6.8 Hz, 1H), 3.58 (s, 2H), 3.46-3.41 (m, 6H), 1.49 (m, 4H), 1.37 (s, 6H), 1.30 (s, 6H), 1.23-1.13 (m, 20H), 0.84-0.82 (m, 6H). ¹³C NMR (75 MHz, CDCl₃): δ 176.7, 165.8, 165.4, 159.9, 145.3, 143.6, 141.6, 137.0, 129.0, 128.0, 127.8, 123.0, 122.0, 121.1, 118.9, 117.3, 77.2, 71.8, 71.7, 45.0, 42.8, 31.7, 29.3, 29.2, 26.0, 26.0, 23.0, 22.8, 22.6, 14.1. MS (ESI) m/z: [M+H]⁺ Calcd. for C₄₀H₅₉N₄O₈ 723.4; Found 723.1.

### Stepwise Chain Elongation Reaction Via the DMAP·HCl Mediated Ring Opening of Benzoxazinone.

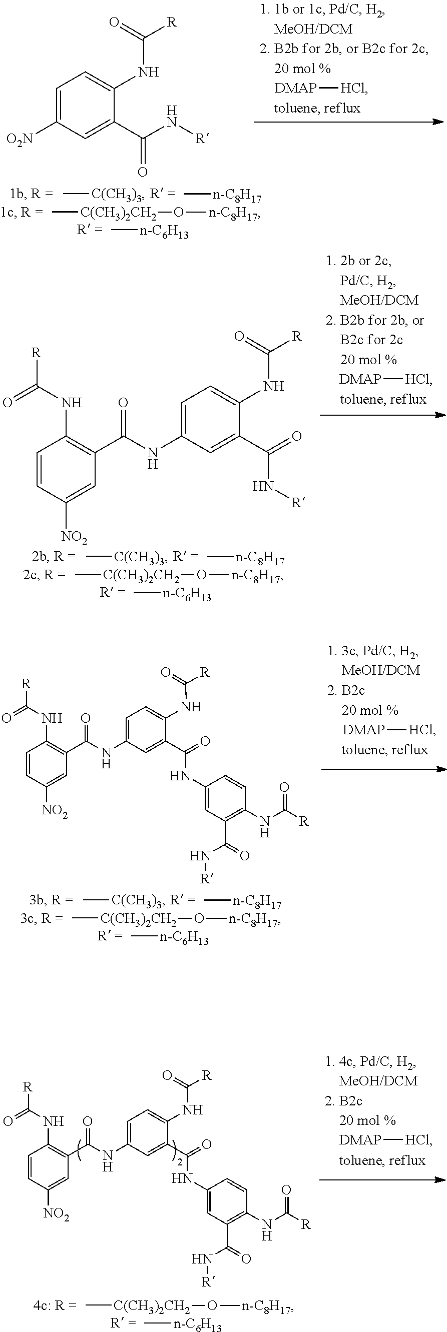

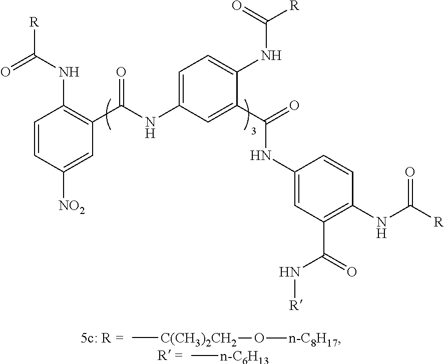

Compound 2b: The reduction of nitro 1b to its corresponding aromatic amine was accomplished via the general hydrogenation method. To a solution of freshly prepared aromatic amine (1.04 g, 3.0 mmol) and B2b (0.74 g, 3.0 mmol) in dry toluene (50 mL) was added 20 mol % of DMAP·HCl salt. The obtained suspended solution was heated under reflux overnight with vigorously stirring. The organic layer was washed with brine (20 mL×2 times) and dried over anhydrous Na₂SO₄. After removal of toluene under vacuum, the obtained residue was dissolved in hot methanol. The white precipitate then formed and was collected by filtration to give pure compound 2b (1.75 g, 98%). ¹H NMR (500 MHz, CDCl₃): δ 11.50 (s, 1H), 11.10 (s, 1H), 9.16 (s, 1H), 8.78 (d, J=9.5 Hz, 1H), 8.64 (d, J=2.5 Hz, 1H), 8.44 (d, J=9.0 Hz, 1H), 8.23 (dd, J=9.5, 3.0 Hz, 1H), 7.99 (d, J=2.5 Hz, 1H), 7.35 (dd, J=9.5, 2.5 Hz, 1H), 6.43 (t, J=6.0 Hz, 1H), 3.48 (m, 2H), 1.65 (m, 2H), 1.42-1.28 (m, 28H), 0.88 (t, J=13.5 Hz, 3H). ¹³C NMR (75 MHz, CDCl₃): δ 178.7, 178.2, 168.4, 165.9, 145.4, 141.5, 136.3, 132.1, 127.4, 124.9, 123.6, 122.1, 122.0, 121.5, 120.7, 119.8, 40.5, 40.2, 40.1, 31.8, 29.4, 29.3, 29.2, 27.5, 27.3, 26.9, 22.6, 14.1. HRMS (ESI) m/z: [M+Na]⁺ Calcd. for C₃₂H₄₅N₅NaO₆ 618.3268; Found 618.3258.

Compound 2c: The reduction of nitro 1c to its corresponding aromatic amine was accomplished via the general hydrogenation method. The coupling reaction between freshly prepared aromatic amine (1.34 g, 3.0 mmol) and B2c (1.13 g, 3.0 mmol) was carried out in the same method as the preparation of compound 2b. After removal of toluene, the obtained residue was subject to flash silica gel column (hexanes/ethyl acetate, from 5/1 to 2/1) to afford the pure compound 2c (2.35 g, 95%) as a yellowish oil. ¹H NMR (400 MHz, CDCl₃): δ 11.42 (s, 1H), 11.07 (s, 1H), 9.10 (s, 1H), 8.80 (d, J=9.2 Hz, 1H), 8.69 (d, J=2.8 Hz, 1H), 8.33 (d, J=9.2 Hz, 1H), 8.26 (dd, J=9.2, 2.8 Hz, 1H), 7.84 (d, J=2.4 Hz, 1H), 7.35 (dd, J=9.2, 2.4 Hz, 1H), 6.55 (t, J=6.0 Hz, 1H), 3.50 (s, 2H), 3.44 (s, 2H), 3.42 (m, 4H), 3.38 (m, 2H), 1.65 (m, 2H), 1.53 (m, 2H), 1.42 (m, 6H), 1.31 (m, 10H), 1.28 (m, 8H), 1.20 (m, 10H), 1.12 (m, 6H), 0.89-0.80 (m, 9H). ¹³C NMR (75 MHz, CDCl₃): δ 176.8, 176.2, 168.2, 165.6, 145.1, 141.6, 135.9, 132.2, 127.3, 125.0, 123.4, 122.4, 122.1, 121.7, 121.4, 119.9, 77.6, 77.3, 71.8, 71.7, 45.0, 44.6, 40.1, 31.8, 31.7, 31.5, 29.4, 29.4, 29.3, 29.2, 29.2, 26.7, 26.0, 26.0, 22.9, 22.7, 22.6, 22.6, 22.6, 14.1, 14.0. HRMS (ESI) m/z: [M+Na]⁺ Calcd. for C₄₆H₇₃N₅NaO₈ 846.5357; Found 846.5340.

Compound 3b: Reduction of nitro 2b to its corresponding amine was accomplished via the general hydrogenation method. To a solution of freshly prepared amine (1.70 g, 3.0 mmol) and benzoxazinone B2b (0.74 g, 3.0 mmol) in dry toluene (100 mL) was added 20 mol % of DMAP·HCl salt. The obtained suspended solution was heated under reflux overnight with vigorous stirring. The organic layer was then washed with brine (20 mL×2 times) and dried over anhydrous Na₂SO₄. After removal of toluene, the obtained crude product was washed with hot ethyl acetate to afford the compound 3b (2.31 g, 95%) as a white solid. ¹H NMR (500 MHz, CDCl₃): δ 11.50 (s, 1H), 11.10 (s, 1H), 9.16 (s, 1H), 8.77 (d, J=10.0 Hz, 1H), 8.64 (s, 1H), 8.43 (d, J=10.0 Hz, 1H), 8.25 (d, J=10.0 Hz, 1H), 7.99 (s, 1H), 7.35 (d, J=5.0 Hz, 1H), 6.43 (t, J=7.5 Hz, 1H), 3.48 (m, 2H), 1.66 (m, 2H) 1.42-1.35 (m, 28H), 0.88 (t, J=7.5 Hz, 3H). ¹³C NMR (75 MHz, CDCl₃): δ 178.7, 178.2, 168.4, 165.9, 145.4, 141.5, 136.3, 132.1, 127.4, 124.9. 123.6, 122.1, 122.0, 121.5, 120.7, 119.8, 40.5, 40.2, 40.1, 31.8, 29.4, 29.3, 29.2, 27.5, 27.3, 26.9, 22.6, 14.1. HRMS (ESI) m/z: [M+Na]⁺ Calcd. for C₄₄H₅₉N₇NaO₈ 836.4323; Found 836.4306.

Compound 3c: The reduction reaction of nitro 2c to its corresponding amine was accomplished via the general hydrogenation method. The coupling reaction between freshly prepared amine (2.38 g, 3.0 mmol) and benzoxazinone B2c (1.13 g, 3.0 mmol) was carried out in the same method as the preparation of compound 3b. After removal of toluene, the obtained residue was then dissolved in the hot acetonitrile. The white solid then formed and was collected by filtration to afford the pure compound 3c as a white solid (3.23 g, 92%). ¹H NMR (400 MHz, 10% DMSO-d₆/90% CDCl₃): δ 11.64 (s, 1H), 11.14 (s, 1H), 10.73 (s, 1H), 10.59 (s, 1H), 9.84 (s, 1H), 8.90 (d, J=9.2 Hz, 1H), 8.85 (d, J=2.4 Hz, 1H), 8.58-8.52 (m, 2H), 8.33 (dd, J=9.6, 2.8 Hz, 1H), 8.11 (m, 2H), 7.93 (dd, J=9.2, 2.4 Hz, 1H), 7.74 (dd, J=9.2, 2.4 Hz, 1H), 7.20 (t, J=5.6 Hz, 1H), 3.48-3.42 (m, 14H), 1.62 (m, 2H), 1.55-1.47 (m, 6H), 1.39 (m, 2H), 1.30-1.28 (m, 26H), 1.23 (m, 10H), 1.19 (m, 10H), 1.15 (m, 6H), 0.89-0.83 (m, 12H). ¹³C NMR (75 MHz, 10% DMSO-d₆/90% CDCl₃): δ 176.3, 175.5, 175.4, 168.4, 166.9, 165.7, 145.5, 141.2, 135.5, 135.4, 133.2, 133.0, 127.1, 124.7, 124.2, 123.7, 123.4, 122.2, 122.1, 121.7, 121.0, 120.8, 119.1, 77.5, 77.1, 77.0, 71.5, 44.9, 44.4, 44.3, 31.6, 31.6, 31.5, 31.3, 29.3, 29.2, 29.1, 29.1, 29.1, 29.0, 26.5, 25.9, 25.8, 22.8, 22.7, 22.7, 22.4, 22.4, 14.0, 13.9. HRMS (ESI) m/z: [M+H]⁺ Calcd, for C₆₆H₁₀₄N₇O₁₁ 1170.7794; Found 1170.7795.

Compound 4c: Reduction of nitro 3c to its corresponding amine was accomplished via the general hydrogenation method. To a solution of freshly prepared amine (3.42 g, 3.00 mmol) and benzoxazinone B2c (1.13 g, 3.00 mmol) in dry toluene (100 mL) was added 20 mol % of DMAP. HCl salt. The obtained suspended solution was heated under reflux for 2 days with vigorous stirring. Upon the complete consumption of starting materials, the organic layer was then washed with brine (20 mL×2 times) and dried over anhydrous Na₂SO₄. After removal of toluene under vacuum, the obtained residue was then dissolved in hot acetonitrile. The white solid formed and was then collected by filtration to afford the pure compound 4c as a white solid (4.23 g, 93%). ¹H NMR (400 MHz, 10% DMSO-d₆/90% CDCl₃): δ 11.60 (s, 1H), 11.15 (s, 1H), 10.72 (m, 2H), 10.60 (s, 1H), 9.96 (s, 1H), 9.77 (s, 1H), 8.91 (d, J=9.6 Hz, 1H), 8.84 (d, J=2.8 Hz, 1H), 8.57-8.49 (m, 3H), 8.33 (dd, J=9.6, 2.8 Hz., 1H), 8.19 (s, 1H), 8.14 (d, J=2.4 H7, 1H), 8.09 (s, 1H), 7.91 (dd, J=8.8, 2.4 Hz, 1H), 7.82 (dd, J=8.8, 2.4 Hz, 1H), 7.73 (dd, J=8.8, 2.4 Hz, 1H), 7.22 (t, J=6.0 Hz, 1H), 3.48-3.41 (m, 18H), 1.61 (m, 2H), 1.50 (m, 8H), 1.29-1.19 (m, 70H), 0.89-0.83 (m, 15H). 13C NMR (75 MHz, 10% DMSO-d₆/90% CDCl₃): δ 176.2, 175.5, 175.4, 168.4, 167.0, 166.9, 165.8, 145.4, 141.2, 135.6, 135.4, 135.1, 133.4, 133.1, 133.0, 128.8, 128.0, 127.1, 124.8, 124.2, 124.1, 123.8, 123.5, 123.4, 122.4, 122.0, 121.7, 121.0, 120.8, 120.0, 119.1, 77.5, 77.1, 77.0, 71.5, 44.9, 44.4, 44.3, 31.6, 31.3, 29.3, 29.2, 29.1, 29.1, 29.0, 26.5, 25.9, 25.8, 22.8, 22.7, 22.6, 22.5, 22.4, 14.0, 13.9. HRMS (ESI) m/z: [M+Na]⁺ Calcd, for C₈₆H₁₃₃N₉NaO₁₄ 1538.9870; Found 1538.9953.

Compound 5c: Reduction of nitro 4c to its corresponding amine was accomplished via the general hydrogenation method. To a solution of freshly prepared amine (4.46 g, 3.00 mmol) and benzoxazinone B2c (1.13 g, 3.00 mmol) in dry toluene (100 mL) was added 20 mol % of DMAP·HCl salt. The obtained suspended solution was heated under reflux for 3 days with vigorous stirring. The organic layer was then washed with brine (20 mL×2 times) and dried over anhydrous Na₂SO₄. After removal of toluene under vacuum, the obtained residue was dissolved in hot acetonitrile. The white solid then formed and was collected by filtration to afford pure compound 5c as a white solid (5.08 g, 91%). ¹H NMR (400 MHz, 10% DMSO-d₆/90% CDCl₃): δ 11.57 (s, 1H), 11.15 (s, 1H), 10.72 (s, 1H), 10.70 (s, 1H), 10.66 (s, 1H), 10.61 (s, 1H), 9.99 (s, 1H), 9.91 (s, 1H), 9.78 (s, 1H), 8.90 (d, J=9.6 Hz, 1H), 8.84 (d, J=2.4 Hz, 1H), 8.57 (d, J=8.8 Hz, 1H), 8.52-8.49 (m, 3H), 8.33 (d, J=8.4 Hz, 1H), 8.25 (s, 1H), 8.19 (s, 1H), 8.15 (s, 1H), 8.10 (s, 1H), 7.90 (d, J=9.2 Hz, 1H), 7.80-7.75 (m, 2H), 7.72 (d, J=10.0 Hz, 1H), 7.19 (t, J=6.8 Hz, 1H), 3.48-3.40 (m, 22H), 1.60 (m, 2H), 1.52-1.45 (m, 10H), 1.29-1.27 (m, 42H), 1.23-1.19 (m, 44H), 0.88-0.83 (m, 18H). ¹³C NMR (75 MHz, 10% DMSO-d₆/90% CDCl₃): δ 176.2, 175.5, 175.3, 168.4, 167.1, 167.0, 166.9, 165.8, 145.4, 141.2, 135.6, 135.4, 135.1, 133.4, 133.4, 133.1, 133.0, 127.2, 124.7, 124.2, 124.1, 123.8, 123.5, 123.5, 122.4, 122.0, 121.6, 121.6, 121.0, 121.0, 120.8, 120.0, 119.1, 77.5, 71.5, 44.3, 31.6, 31.3, 29.3, 29.1, 29.0, 26.5, 25.9, 25.8, 22.8, 22.7, 22.6, 22.4, 22.4, 14.0. HRMS (MALDI) m/z: [M+Na]⁺ Calcd, for C₁₀₆H₁₆₃N₁₁NaO₁₇ 1886.2160; Found 1886.2136.

### [4+2] Chain Elongation Reaction Via the DMAP. HCl Mediated Ring Opening of Benzoxazinone.

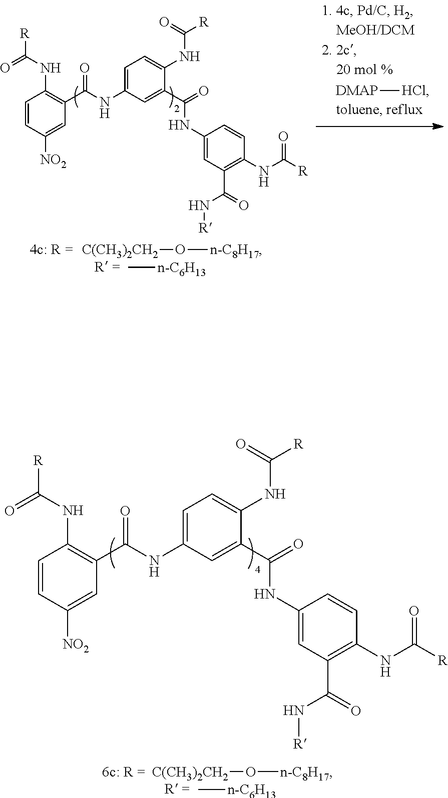

Compound 6c: The reduction reaction of nitro 4c to prepare its corresponding aromatic amine was accomplished via the general hydrogenation method. To a solution of freshly prepared aromatic amine (2.23 g, 1.50 mmol) and benzoxazinone dimer 2c′ (1.09 g, 1.50 mmol) in dry toluene (100 mL) was added 20 mol % of DMAP·HCl salt. The obtained suspended solution was heated under reflux for 3 days. The organic layer was then washed with brine and dried over anhydrous Na₂SO₄. After removal of toluene under vacuum, the residue was dissolved in hot acetonitrile. The white precipitate then formed and was collected by filtration to give pure compound 6c as an off-white solid (3.05 g, 92%). ¹H NMR (400 MHz, 10% DMSO-d₆/90% CDCl₃): δ 11.56 (s, 1H), 11.15 (s, 1H), 10.73 (s, 1H), 10.70 (s, 1H), 10.64 (m, 2H), 10.62 (s, 1H), 10.02 (s, 1H), 9.96 (m, 2H), 9.80 (s, 1H), 8.89 (d, J=9.2 Hz, 1H), 8.83 (d, J=2.8 Hz, 1H), 8.57 (d, J=9.2 Hz, 1H), 8.51 (m, 4H), 8.33 (d, J=8.0 Hz, 1H), 8.26 (m, 2H), 8.22 (s, 1H), 8.15 (s, 1H), 8.11 (s, 1H), 7.89 (d, J=8.8 Hz, 1H), 7.77-7.69 (m, 4H), 7.23 (t, J=7.2 Hz, 1H), 3.47-3.45 (m, 14H), 3.42-3.40 (m, 12H), 1.61 (m, 2H), 1.49 (m, 14H), 1.29-1.26 (m, 52H), 1.23 (m, 12H), 1.18-1.15 (m, 36H), 0.85-0.82 (m, 21H). 13C NMR (75 MHz, 10% DMSO-d₆/90% CDCl₃): δ 176.2, 175.5, 175.4, 168.4, 167.1, 167.1, 167.0, 165.8, 145.4, 141.3, 135.5, 135.3, 135.1, 133.5, 133.4, 133.2, 133.0, 127.2, 124.2, 124.1, 123.8, 123.6, 123.4, 122.4, 122.3, 122.0, 121.6, 121.0, 120.0, 120.0, 119.1, 77.5, 77.2, 71.5, 44.9, 44.4, 44.3, 31.6, 31.3, 29.2, 29.1, 29.0, 26.5, 25.8, 22.8, 22.6, 22.4, 14.0. HRMS (MALDI) m/z: [M+Na]⁺ Calcd, for C₁₂₆H₁₉₃N₁₃NaO₂₀ 2232.4416; Found 2232.4634.

### X-Ray Crystallography Data

Single crystals of all the compounds were obtained from hexane/ethyl acetate (1/2, v/v) by slow evaporation of solvents at room temperature. Single crystal data was collected by the X scan technique at 293 K for B2b and 100 K for 2b′ on an Agilent Super Nova Dual diffractometer with an Atlas S2 detector, using Moka (λ=0.71073 Å). Data of the single crystal structure were solved by direct methods which revealed the positions of all non-hydrogen atoms, and were refined on F² by a full-matrix least squares procedure using anisotropic displacement parameters. All hydrogen atoms were placed in ideal geometry and were refined using a riding model.

TABLE 2
Crystal data and structure refinement for B2b and
2b′. Computer programs: CrysAlisPro 1.171.38.43f
(Rigaku OD, 2015), SHELXTL (Sheldrick, 2015)

| Identification code | B2b | 2b′ |
|---|---|---|
| Empirical formula | C₁₂H₁₂N₂O₄ | C₂₄H₂₆N₄O₆ |
| Formula weight | 248.24 | 466.49 |
| Temperature(K) | 293(2) K | 293(2) |
| Wavelength (Å) | 0.71073 | 0.71073 |
| Crystal system, space | Monoclinic, P2(1)/c | Triclinic, P-1 |
| group | | |
| a, b, c (Å) | 5.9968(14), 23.292(4), | 11.4728(5), 12.3051(6), |
| | 8.5071(14) | 17.1650(8) |
| α, β, γ (°) | 90, 99.92(2), 90 | 80.909(4), 71.174(4), |
| | | 86.760(4) |
| Volume (Å³) | 1170.5(4) | 2264.76(18) |
| Z, Dₓ (Mg · m⁻³) | 4, 1.409 | 4, 1.368 |
| μ (mm⁻¹) | 0.108 | 0.100 |
| F(000) | 520 | 984 |
| Crystal size (mm) | 0.150 × 0.130 × 0.110 | 0.15 × 0.13 × 0.12 |
| Theta range for data | 2.58 to 23.98 | 1.90 to 25.00 |
| collection(deg.) | | |
| Limiting indices | −6 <= h <= 6, | −13 ≤ h ≤ 13, |
| | −26 <= k <= 20, | −14 ≤ k ≤ 14, |
| | −9 <= l <= 9 | −18 ≤ l ≤ 20 |
| Reflections collected/ | 5480/1807 [R(int) = | 15168/7988 [R(int) = |
| unique | 0.0404] | 0.0247] |
| Completeness to | 99.0% | 100.0% |
| theta = 68.17 | | |
| Refinement method | Full-matrix least- | Full-matrix least- |
| | squares on F² | squares on F\hat{ }2 |
| Data/restraints/ | 1807/6/152 | 7988/0/613 |
| parameters | | |
| Goodness-of-fit on F² | 1.122 | 0.928 |
| Final R indices [I > | R₁ = 0.0825, | R₁ = 0.0429, |
| 2σ(I)] | wR₂ = 0.2139 | wR₂ = 0.0989 |
| R indices (all data) | R₁ = 0.1064, | R₁ = 0.0554, |
| | wR₂ = 0.2299 | wR₂ = 0.1068 |
| Δρₘₐₓ, Δρₘᵢₙ (e Å³) | 0.540 and −0.376 | 0.223 and −0.234 |

### Example 2

This example provides a description of anion receptors based on linear and cyclic aromatic oligoamides.

### Design of Non-Cyclic, Anion-Binding Aromatic Oligoamides

5 Foldamers having inner cavities that are electrostatically positive, i.e., those with positive charges or multiple H-bond donors, are very rare. The availability of foldamers with such cavities will address current challenges in the binding, recognition, or transport of species especially various anions.

As shown in FIG. 98A, inverting the orientation of the backbone amide groups of oligoamide AO leads to oligomer OA. To ensure the inversion of each backbone amide group, a highly favorable, six-membered (S(6)-type) intramolecular H-bond is introduced between the oxygen atom of each backbone amide group and the amide proton of an adjacent acylamino side chain. Such an intramolecular H-bond keeps the backbone oxygen atom of the backbone amide group from engaging in additional, effective H-bonding and, at the same time, allows each backbone NH group to participate in intermolecular H-bonding interaction with anionic or polar molecular guests.

Unlike that of 1, the backbone of oligoamide OA is only partially constrained. Around each backbone amide group of OA, the rotation of the aryl-CO single bond is limited while the rotation of the C(O)NH-aryl bond remains relatively unhindered. It is thus expected that, in solution, oligoamide OA alone will adopt multiple conformations, each of which, such as OA′, exits in a small proportion (FIG. 98B). Among all the possibilities, conformation OA has all the amide protons being placed convergently. Upon adding a guest that undergoes H-bonding interactions with the backbone amide and aromatic protons, conformation OA is stabilized and the equilibrium is shifted toward complex OA·G, resulting in anion-induced folding of oligomer OA.

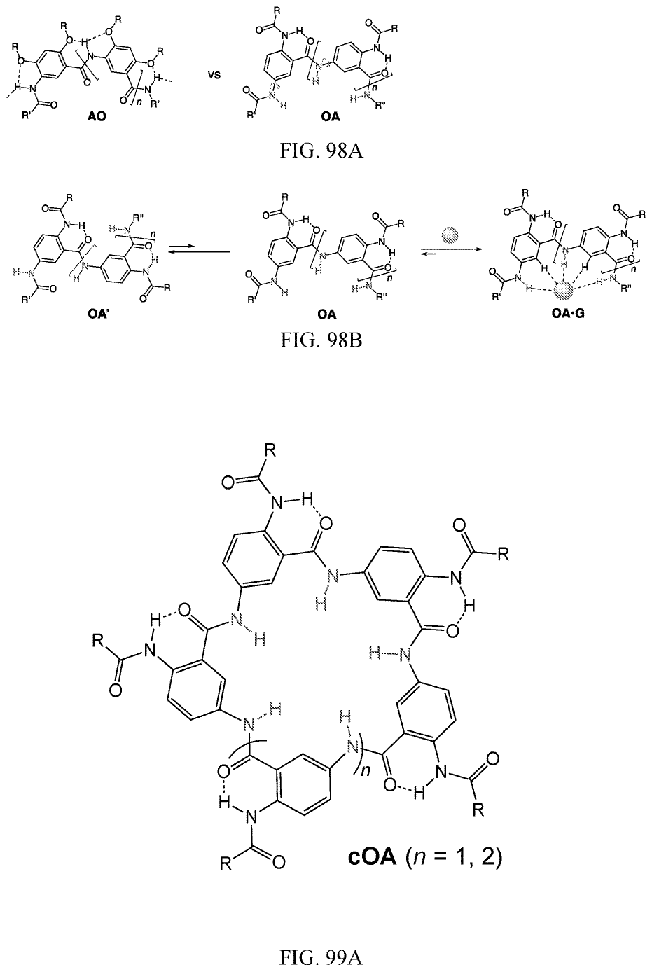

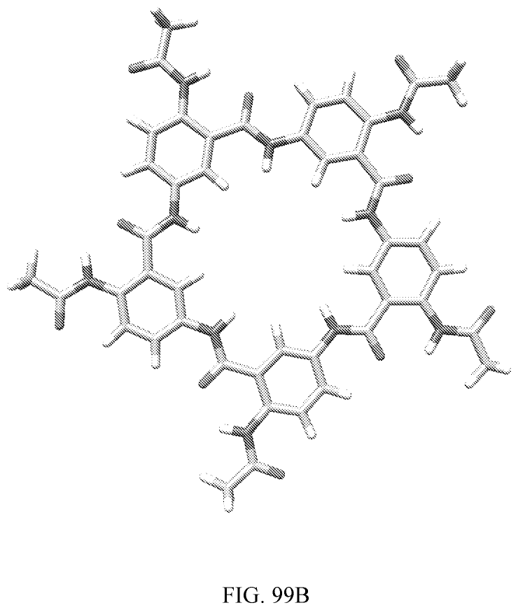

Design of cyclic, anion-binding aromatic oligoamides. Oligomer OA may also be cyclized into the corresponding macrocycle cOA in which the backbone amide NH groups are convergently placed, i.e., being forced to point toward the center of the macrocyclic structure (FIG. 99). Simple computer modeling indicates, due to the bond length and especially bond angles associated with the aromatic rings and the amide groups, macrocycles cOA comprising five or six residues i.e., being cyclic pentamer (5mer) or hexamer (6mer), are mostly likely to be obtained. Results from initial experimental studies (see below) indicate that the pentameric cOA macrocycles can be prepared in high yields. The macrocycles based on cOA are expected to exhibit significantly enhanced binding affinities toward anions since the cyclic structure serves to drastically reduce or even completely remove the entropy cost of organizing multiple NH groups.

### Synthesis.

Synthesis of linear oligoamides: A new synthetic pathway based on a novel, highly efficient amide coupling method.

The synthesis of OA, oligomer of 5-amino-N-acylanthranlic acid, could not be performed based on established amide-coupling chemistry because anthranilic acid or its N-acylated derivative are known to undergo the self-cyclization when being subjected to acylating reagents such as acid chlorides or other amide-coupling agents. Specifically, the intramolecular cyclization involves the carboxyl group and the adjacent amino or acylamino group, giving derivatives having a 4H-3, 1-benzoxazin-4-one (or benzoxazinone) core. Such self-cyclization prevents the intended amide coupling reaction with other amines from happening. Indeed, an attempted coupling of acid A1 (Scheme 1) and an amino building block failed to yield any amide product.

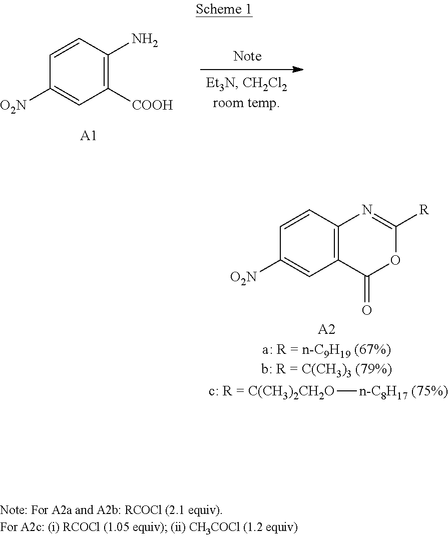

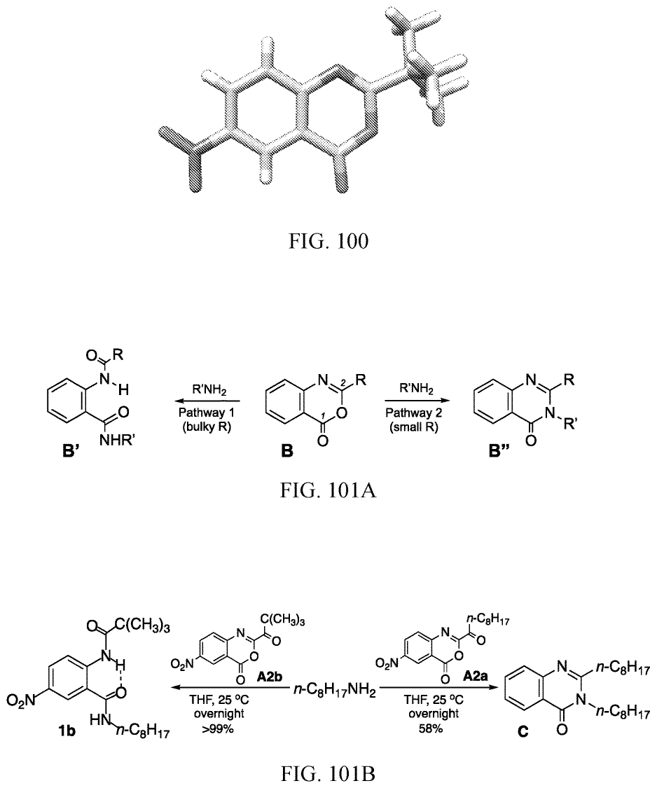

Treating 5-nitro-anthranlic acid A1 with two or more equivalents of decanoyl chloride or trimethylacetyl chloride gives benzoxazinone derivatives A2a or A2b in good yields (Scheme 1). Similarly, compound A2c, which differs with A2b in its side chain, was prepared by converting A1 into the corresponding 5-nitro-N-acylanthranlic acid that was then further treated with acetyl chloride to furnish the heterocycle. The ¹H NMR spectra of A2a, A2b, and A2c indicate that the three compounds share the same benzoxazinone core. The identities of A2a, and A2b, and A2c were further verified by ESI-MS spectra which confirm the corresponding molecular weights of the expected products. In addition, single crystals of A2b were obtained, allowing the determination of its X-ray structure (FIG. 100), which confirms that acid A1 indeed undergoes cyclization to give the benzoxazinone derivative as expected.

The reactions of (4H)-3, 1-benzoxazin-4-one B with amines were reported to follow one of two possible pathways (FIG. 101A). One involves the nucleophilic addition to carbonyl carbon C-1, leading to ring-opening product B′ with the release of an acylamino side chain and the formation of a new amide bond; the other gives quinazolone B″ upon nucleophilic addition to carbon C-2.

Steric factors play an important role in determining which one of the two pathways a reaction follows. When R and/or R′ are bulky and impose steric hindrance, ring-opening of B leads to the acylation of amine R′NH₂, which releases the acyamino (RCONH−) side chain and forms a new amide bond, thus resulting in the desired product B′; when R and/or R′ are small or linear groups that do not impose significant steric hindrance, heterocycle B″ is generated.

Compound A2a or A2b was treated with one equivalent of octylamine to assess the outcome of nucleophilic addition reactions involving these two benzoxazinone derivatives (FIG. 101B). It was observed that the reaction involving A2a afforded quinazolinone C in a yield of 58%, while the reaction of A2b and octylamine proceeded nearly quantitatively to give the ring-opening product, amide 1b. The molecular weights of A3 and 1b were confirmed by mass spectra. ¹H NMR spectra revealed two amide proton signals at 6.30 and 11.70 ppm for 1b while no amide signal can be detected for C. These results demonstrate that, to ensure the formation of the amide bond and the release of the acylamino side chain, an R group with a bulky a-carbon such as the t-butyl group of A2b need to be present.

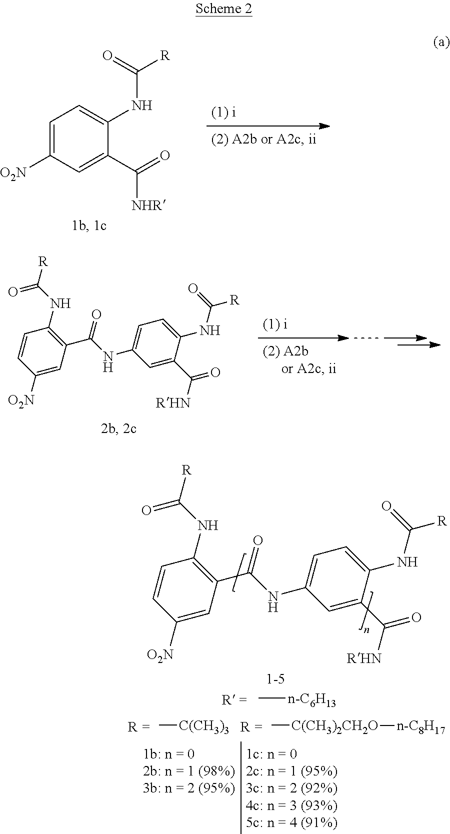

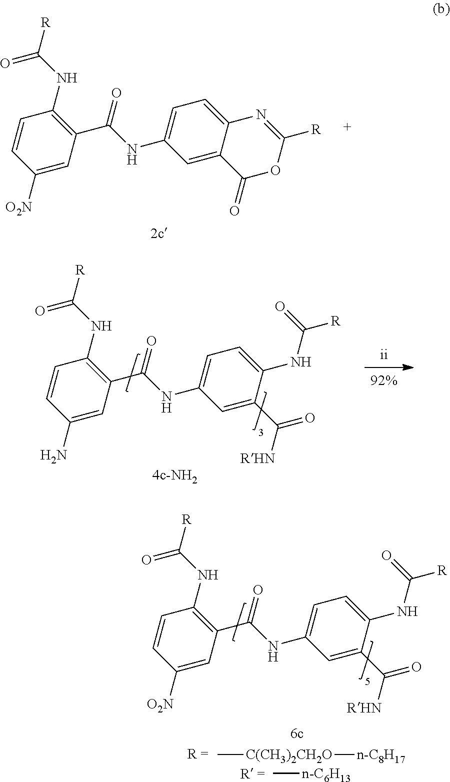

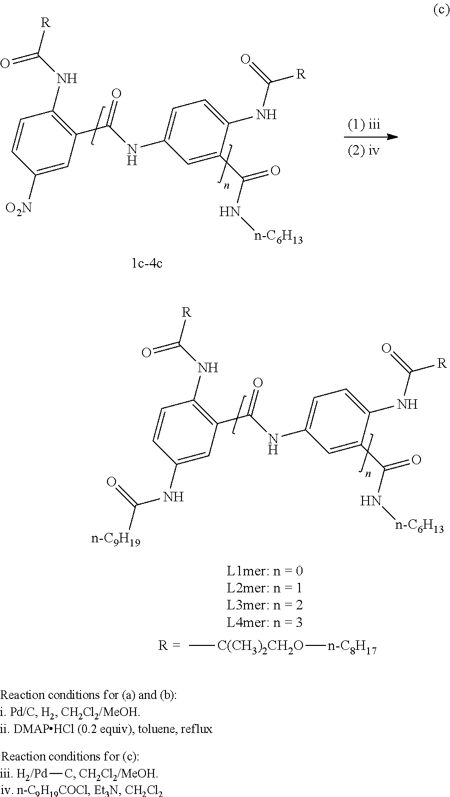

Based on the observation made with the reaction of A2b and n-octylamine, the synthesis of oligoamides represented by general structure OA (Scheme 2) was first probed by refluxing A2b and the amine derived from 1b (one equiv.) in toluene in the presence of 4-dimethylaminopyridine hydrochloride (DMAP, 0.2 equiv), which gave dimer 2b in 98% yield. Reducing 2b to the corresponding amine followed by coupling with A2b under the same condition produced trimer 3b again in very high (95%) yield. Attempts to prepare the tetramer bearing the same side chains of 1b-3b were hampered by the formation of an insoluble product that prevents characterization. Oligoamides 2c-5c, comprising two to five residues that bear side chains with α-quaternary carbons and n-octyloxy tails, are expected to show enhanced solubility. Indeed, starting from 1c by repetitive coupling of monomer A2c based on the step-wise steps and conditions for preparing oligomers 2b and 3b, oligoamides 2c-5c were obtained in very high yields of 95%, 92%, 93%, and 91%.

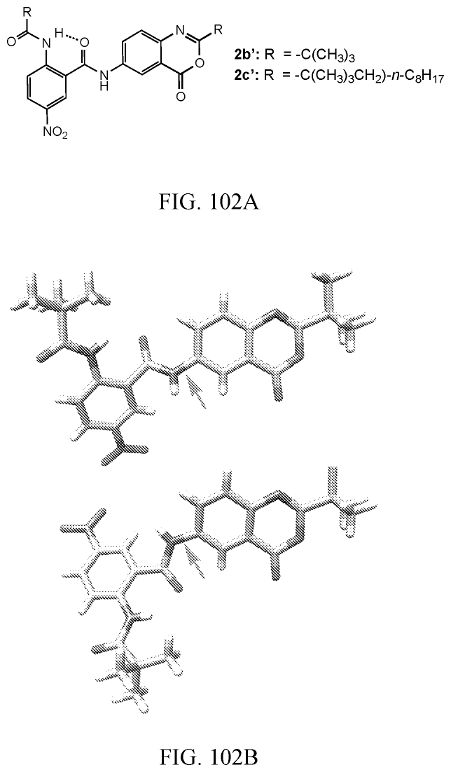

Dimeric 2b′ and 2c′ were prepared. ¹H NMR spectra demonstrate that 2b′ and 2c′ share the same backbone. The presence of the benzoxazinone unit is confirmed by the solid-sate structure of 2b′ (FIG. 102), in which two conformations related by ~180° rotation around the single bond between the benzoxazinone unit and the rest of the molecule are revealed.

Under the same conditions for preparing oligoamides 2-5 based on the repetitive coupling of A2b or A2c, refluxing 2c′ and tetramer amine 4c-NH₂ in toluene gave hexamer 6c in 92% yield (Scheme 2b).

The highly efficient formation of oligoamides 2b, 3b, 2c-5c, and 6c indicates that the amide coupling steps adopted in this work, which is based on the ring-opening of benzoxazinone moiety, involves repetitive coupling of benzoxazinone monomers B2b and B2c to a growing oligomer chain, or the coupling of two oligomeric reactants like 2c′ and 4c-NH₂. This new and highly efficient synthetic method, which involves simple refluxing without the need of adding any coupling reagents, allows the preparation of aromatic oligoamides bearing acyamino side chains. Due to the self-cyclization of anthranlic acid and its N-acyl derivatives, aromatic oligoamides presented here cannot be prepared based on standard amide coupling chemistry and remained unknown until this work.

To ensure the formation of amide bonds, side chains having a bulky (quaternary) α-carbon as shown by A2b and A2c, and the corresponding oligoamides, are required. In addition to the bulky α-carbon, a wide variety of “tails” including alkyl, oligoether, and aliphatic chains bearing various solubilizing terminal groups such as amino, hydroxyl, and carboxyl groups, that are not limited to those of A2b and A2c, can be introduced to tune the solubility of the corresponding oligoamides.

Oligoamides L1mer, L2mer, L3mer, and L4mer were obtained by first reducing the nitro groups of 1c-4c followed by acylation with decanoyl chloride (Scheme 2c). With two, three, four, and five backbone NH groups, oligoamides L1mer-L4mer exhibit very good solubility in organic solvents such as chloroform and DMSO, which facilitates the study of their anion-binding behavior.

### Synthesis of Cyclic Oligoamides Based on One-Pot Macrocyclization

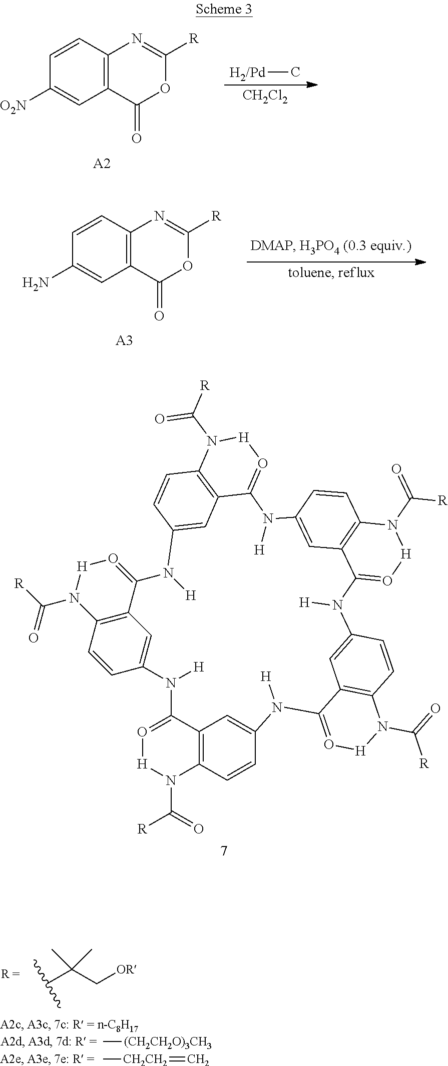

A macrocyclization procedure leading to the one-pot formation of pentameric macrocycles 7 has been discovered. This one-pot macrocyclization involves the self-condensation of monomer A3, prepared by reducing A2 with catalytic hydrogenation, by refluxing in toluene in the presence of DMAP and 0.3 equiv. of phosphoric acid. Macrocycles 7c-e have been obtained in good (60-70%) yields. These macrocycles exhibit good solubility in a variety of nonpolar and polar organic solvents. Besides, macrocycle 7d has a solubility in water in the millimolar concentration range.

Similar to the above discussion on side chains of the linear oligoamides, to ensure the formation of amide bonds and thus the success of the one-pot cyclization, side chains having a bulky (quaternary) α-carbon as shown by A2b and A2c, should be incorporated into the corresponding oligoamide macrocycles. In addition to the bulky α-carbon, a wide variety of “tails” including alkyl, oligoether, and aliphatic chains bearing various solubilizing terminal groups including, amino, hydroxyl, and carboxyl groups, that are not limited to those of A2b and A2c, can be introduced to tune the solubility of the corresponding oligoamide macrocycles.

### Anion Binding.

Anion binding with linear oligoamides: Systematically tunable binding strength.

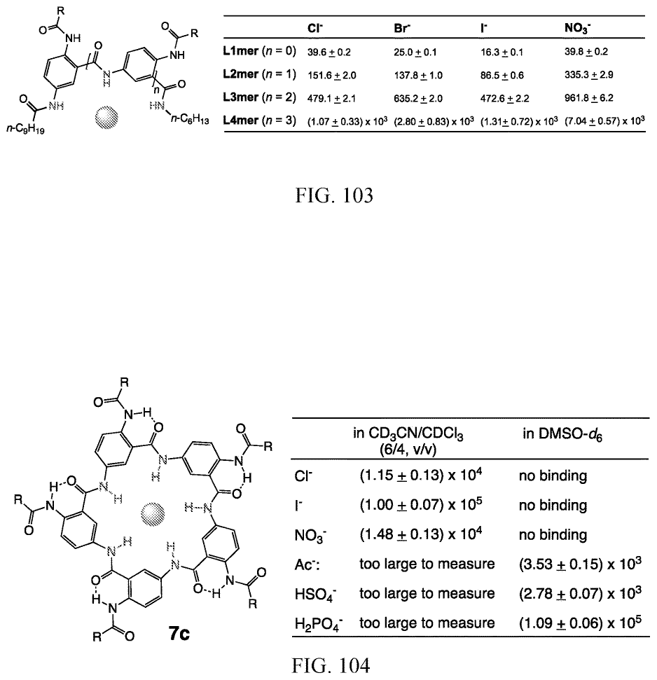

Anion binding with four amides, i.e., L1mer-L4mer, was studied in the mixed solvents of CD₃CN/CDCl₃ (1/9, v/v) by titrating an amide host with 0 to 5 equivalents of tetrabutylammonium (n-Bu₄N⁺) salt of the corresponding anion. The binding of chloride, bromide, iodide, and nitrate was examined with the four amides in CDCl₃ containing 10% CD₃CN. The titration data were collected by monitoring the changes in chemical shifts of the backbone amide protons and the inner aromatic protons with varying ratios of the anion guests. Job plots indicate that these linear oligoamides and their anion guests bind in a 1:1 stoichiometry. As shown in FIG. 103, for each anion, the binding affinities show a linear correlation with the length of the oligoamides, i.e., the number of amide NH groups available for H-bonding with the anion guest. For example, the Kₐ's of chloride range from the very modest 39 M⁻¹ with L1mer to over 1,000 M⁻¹ with tetramer L4mer. Different anions also showed different binding strength with the same oligoamide host. Among the halides, while the shortest L1mer binds chloride most strongly, the longer L2mer, L3mer, and L4mer all bind the bromide ion more strongly than chloride or iodide, suggesting that the bromide ion probably fits the curvature of the longer oligoamides better, which results in effective H-bonding interaction. The binding of the nitrate ion with each of the oligoamides is stronger than any of the halide ion, with the strongest binding (~104 M⁻¹) being found between L4mer and the nitrate ion. These initial results demonstrate that by varying oligoamide lengths, anion binding strength can be systematically adjusted, a very important feature for developing anion carriers capable of efficiently transporting and delivering anions across cell membranes.

Anion binding with macrocyclic oligoamides: Tight binding of anions.

The binding of anions by macrocycle 7c was examined in CH₃CN/CHCl₃ (6/4, v/v) and in DMSO by titrating with various equivalents of the salts of chloride, iodide. nitrate, and dihydrogen phosphate, with tetrabutylammonium (n-Bu₄N⁺) being the counterion ion. Following the changes in the chemical shifts of backbone amide protons and “inner” aromatic protons allowed the determination of binding constants. The obtained titration data indicate that macrocycle 7c binds with these anions in a 1:1 stoichiometry.

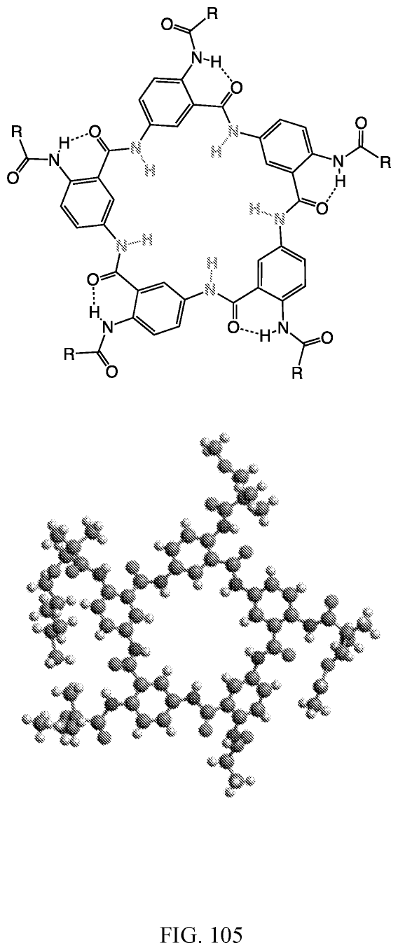

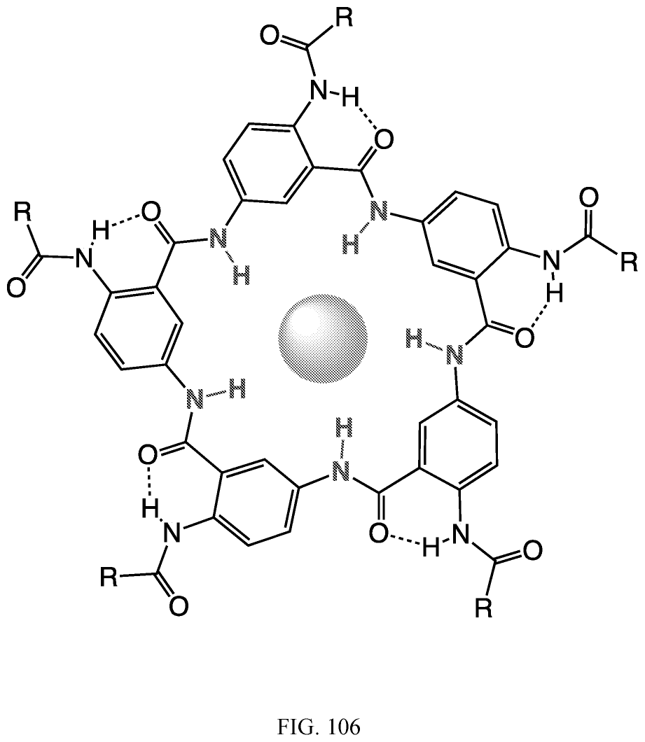

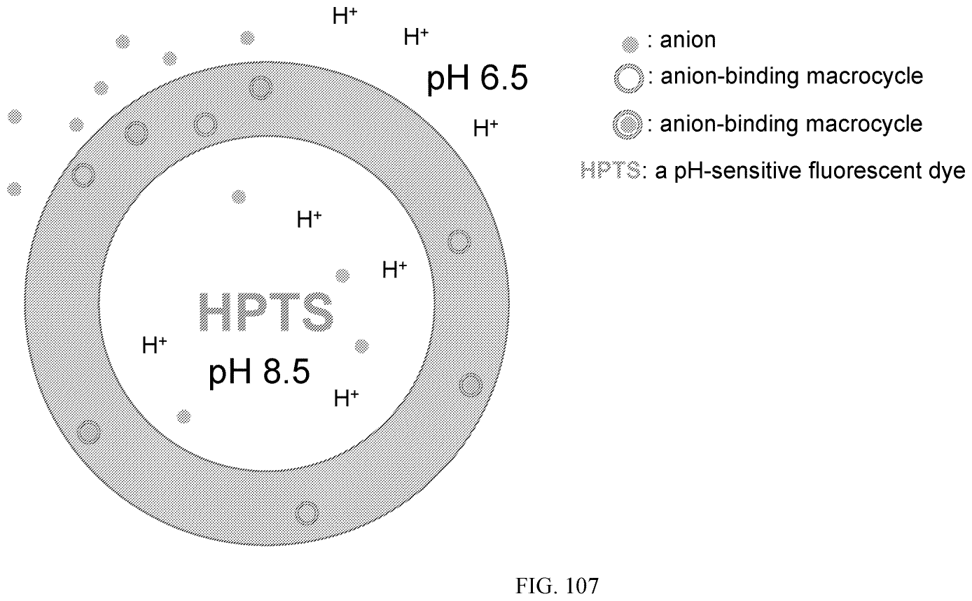

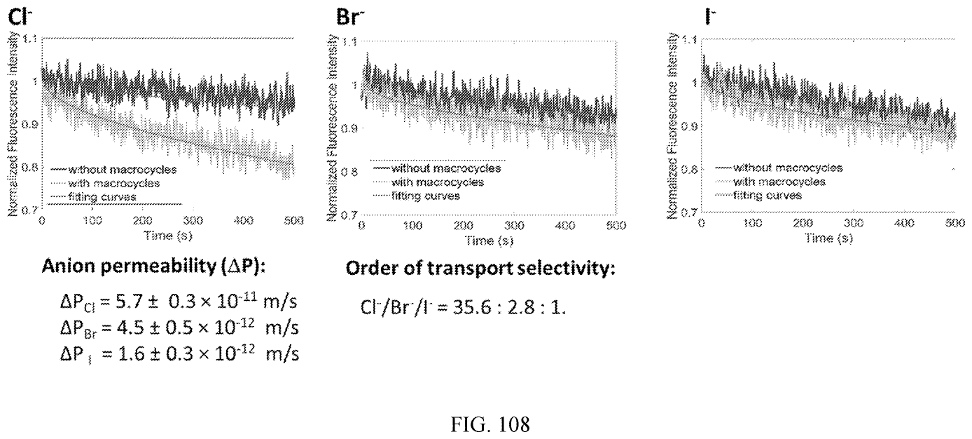

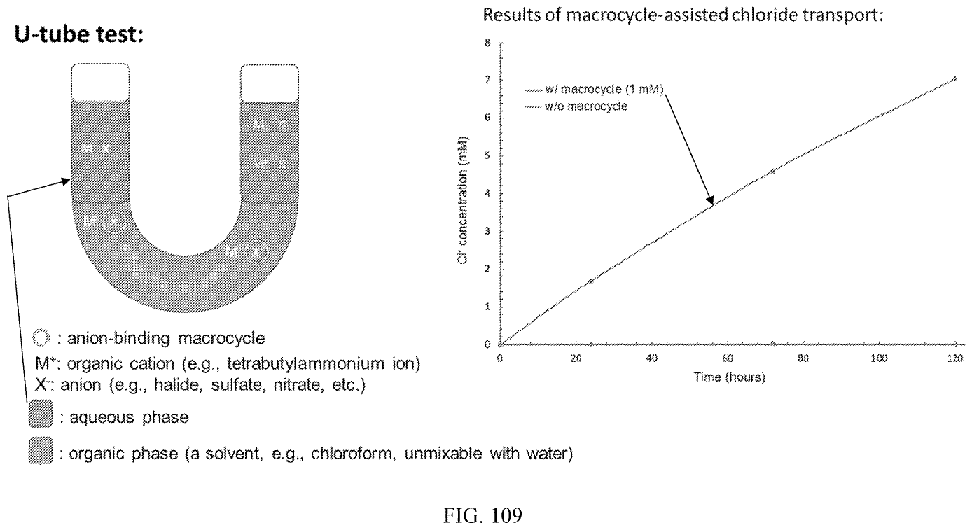

As shown in FIG. 104, even in the rather polar solvent of CD₃CN/CDCl₃ (6/4), macrocycle 7a binds chloride, iodide, and nitrate with much larger (10⁴-10⁵ M⁻¹) Kₐ's than those observed with the linear oligoamides. The binding of acetate, bisulfate, or dihydrogen phosphate with 7c is too strong to be measurable by ¹H NMR. Even in the highly polar DMSO, the binding constants of acetate, bisulfate, or dihydrogen phosphate with 7c are still over 10³ M⁻¹, with that of dihydrogen phosphate being over 10⁵ M⁻¹. The observed strong binding of anions by macrocycle 7c indicate that these oligoamide macrocycles could provide a new series of receptors with high affinities for anions including biologically important ones.

### Expected Biological Activities.

These anion receptors, most of which show desirability solubility in most organic solvents, are expected to facilitate transmembrane anion transport and exert their biological activities such as killing cancer cells by altering the pH inside and outside cells, and treating cystic fibrosis by re-balancing chloride gradient across cell membranes.

### Example 3

This example provides a description of anion receptors based on cyclic aromatic oligoamides.

This example describes the design and synthesis of star-shaped aromatic pentaamide macrocycles c5 featuring a constrained backbone that locks multiple amide NH and CH groups into an unambiguous, convergent placement. Macrocycles c5 represent the newest member of a novel family of cyclic compounds including MacLachlan's camperstarenes, Zeng's pentamers and Flood's cyanostars that have a rare C₅ symmetry. The non-deformable cavity of c5 defined by these NH and CH donors, along with the positive ends of large amide dipoles that also point inward, is highly electropositive and predisposed for anion accommodation. The resultant anion binders could find important applications such as serving as remedies for malfunctioned anion transport found in a variety of diseases.

Materials and instruments. Chemicals were purchased from commercial sources and used as received. Silica gel for analytical thin layer chromatography (TLC) and column chromatography (mesh 230~400) were purchased from Sorbent Technologies Inc. ¹H-NMR spectra were recorded at 400 MHz on Varian Inova-400 and 500 MHz on Varian Inova-500. ¹³C-NMR spectra were recorded at 75 MHz on Varian Mercury-300 spectrometers, at ambient temperature using CDCl₃ or DMSO-d₆ as solvents (Cambridge Isotope Laboratories, Inc.). Chemical shifts are reported in parts per million (ppm) downfield from TMS (tetramethylsilane) or residual of deuterated solvents. Coupling constant in 1H-NMR were expressed in Hertz (Hz). High-resolution electrospray ionization mass spectra (HRMS-ESI) was recorded on a Bruker SolariX 12 T Fourier Transform Mass Spectrometer. Agilent 6530 Q-TOF LC/MS was used to collect mass spectra of the complexes between c5c and anions in negative ion mode.

Unless otherwise specified, all solvents, including high boiling point solvents, were removed under vacuum with a rotary evaporator. Anhydrous toluene was applied for the coupling reactions.

Isothermal titration calorimetry (ITC) experiments were performed using a MicroCal VP-ITC. ITC experiments were conducted by adding ~1.5 mL of a solution containing c5a to the sample cell. Followed by titrating 10 μL aliquots of a solution containing the corresponding TBA·X salt (~10-14× concentration of c5a solution) from the syringe into the sample cell. Heats of dilution were obtained by titrating 10 μL aliquots of the TBA·X solution from the syringe into the sample cell in the absence of c5a. The heats of dilution were subtracted from the raw data. ITC data processing and curve fitting was done using the Origin program (MicroCal).

Preparation of 4-(N,N-dimethylamino)pyridine salt. 4-(N,N-Dimethylamino)pyridine hydrochloride (DMAP·HCl) and hydrobromide (DMAP·HBr) were synthesized via the published method without any modification. For the synthesis of 4-(N, N-Dimethylamino)pyridine hydroiodide (DMAP·Hl), DMAP·HCl and KI (2.0 eq.) were dissolved in acetone. The white solid, KCl formed and was filtered out. The resulting solution was dried under vacuum to afford the crude DMAP·Hl salt. Further purification of DMAP·Hl salt was done by dissolving the crude product in hot toluene and filtering out any insoluble solid. The filtrate was concentrated to afford DMAP·Hl salt.

Hydrogenation reaction. The reduction of nitro compounds to their corresponding amine were carried out in a mixed solvent of 20% methanol and 80% DCM, in the presence of a catalytic amount of Pd/C and pressured hydrogen gas. For the reduction of nitro groups in benzoxazinone residues, CH₂Cl₂ was used as a solvent as methanol could react with benzoxazinone residues to afford corresponding methyl ester. The resulting reaction solution was stirred at room temperature for 2-8 hours. Upon the completion of reduction reactions, developed TLC plates were stained with a solution of ninhydrin in ethanol and the spot of aromatic amines always turned to be from red to purple. Pd/C was then filtered out and the filtrate was concentrated to afford the corresponding amines which were pure enough and applied directly to the coupling reaction without any further purifications. No analytical data is available for aromatic amines, as they were typically considered to be unstable under the ambient environment.

¹H NMR titration methods. Titrations of c5a and c5b with anions as their tetra-n-butylammonium (TBA), tetra-ethylammonium (TEA) and tetra-methylammonium (TMA) salts were carried out by ¹H NMR (400 MHz) at 25° C. 0.4 mL of corresponding hosts solutions were prepared in the rubber-cap NMR tubes. Aliquots of concentrated guest solution containing hosts were added into the NMR tubes using 20 microliter pipette. An initial ¹H NMR spectrum was collected and additional spectra were obtained after each injection of guest solution. All titration ¹H NMR spectra were stacked together and the chemical shifts of the internal aromatic CH proton were then fitted to 1:1 binding model using BindFit v0.5 (http://app.supramolecular.org/bindfit/) to evaluate binding constants to anions.

UV-Vis titration methods. The host solution of c5a (20 μM) was prepared in CH₃OH:CHCl₃ (5:95, v/v). The solution of guests (50 equivalents) being studied was prepared in the host solutions in order to avoid any dilution effect during titration procedure. Aliquots of guest solution were added into the host solution in a spectrometric cell. The UV-Vis spectra was collected by Beckman Coulter DU 800 spectrophotometer at room temperature.

Competition NMR experiments for determining the Kₐ's of slow-exchange oxoanions. Interaction between c5a and acetate, DPP, or MPP (“anion”) is in slow-exchange on the NMR time scale. In the presence of C₁ as the competitor, fast-exchange happened in NMR titrations, which allowed the Kₐ of the complex between c5a and each these anions to be determined.

In NMR titration experiment, the solution of an anion of interest was gradually added to the solution containing c5a (0.5 mM) and TBA·Cl (100 mM), the competitor, resulting in the signal of proton a of c5a to shift in the ¹H NMR spectra. The change in chemical shifts was due to the formation of complex c5a·anion which is more stable than complex c5a·Cl⁻.

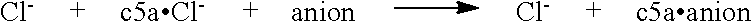

Because the chloride ion is in large excess, the concentration of free chloride, [Cl⁻], can be regarded as being unchanged (constant) before and after titrating with the anion. The titration data could be fitted into a 1:1 binding isotherm to obtain Kₐ (relative).

$${K}_{a} ( relative ) = \frac{[ c 5 a \cdot anion ]}{[ c 5 a \cdot {Cl}^{-} ] \times [ anion ]} = \frac{{K}_{a}}{{K}_{a ( {Cl}^{-} )} \times [ {Cl}^{-} ]}$$

$$where$$

$${K}_{a} = \frac{[ c 5 a \cdot anion ]}{[ c 5 a ] \times [ anion ]}$$

$${K}_{a ( {Cl}^{-} )} = \frac{[ c 5 a \cdot {Cl}^{-} ]}{[ c 5 a ] \times [ Cl - ]} ( association constant betweem c 5 a and {Cl}^{-} )$$

Because the binding constant of c5a with Cl⁻ (K_{a(Cl-)}) is measured by direct NMR titration, the binding constant of c5a with the slow-exchange oxoanions is then calculated based on following equation:

$${K}_{a} = {K}_{a} ( relative ) \times {K}_{a ( {Cl}^{-} )} \times ⌊ {Cl}^{-} ⌋$$

Two-step competition ITC experiments for determining the Kₐ of c5a and iodide. In CH₃CN:CHCl₃ (5:95, v/v), the binding affinity of c5a for I⁻ was too high to be determined with direct NMR or ITC titration. Instead, two-step competition ITC experiments with Cl⁻ as the competitor were performed. Titrating macrocycle c5a (0.5 mM) with TBA·I (5.0 mM) in the presence of TBA·Cl (2.5 mM) gave the K (relative), which, along with K_{(Cl-)}, yielded the Kₐ of c5a and I⁻.

### Synthesis:

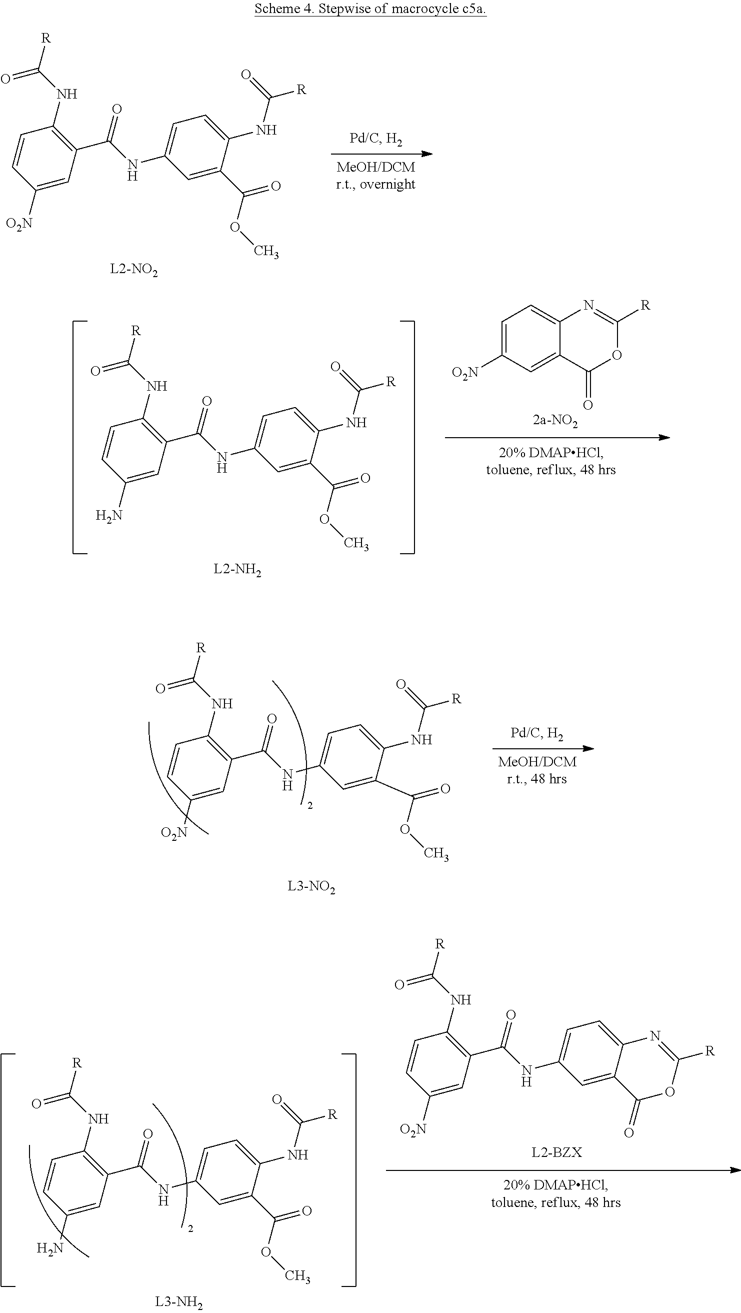

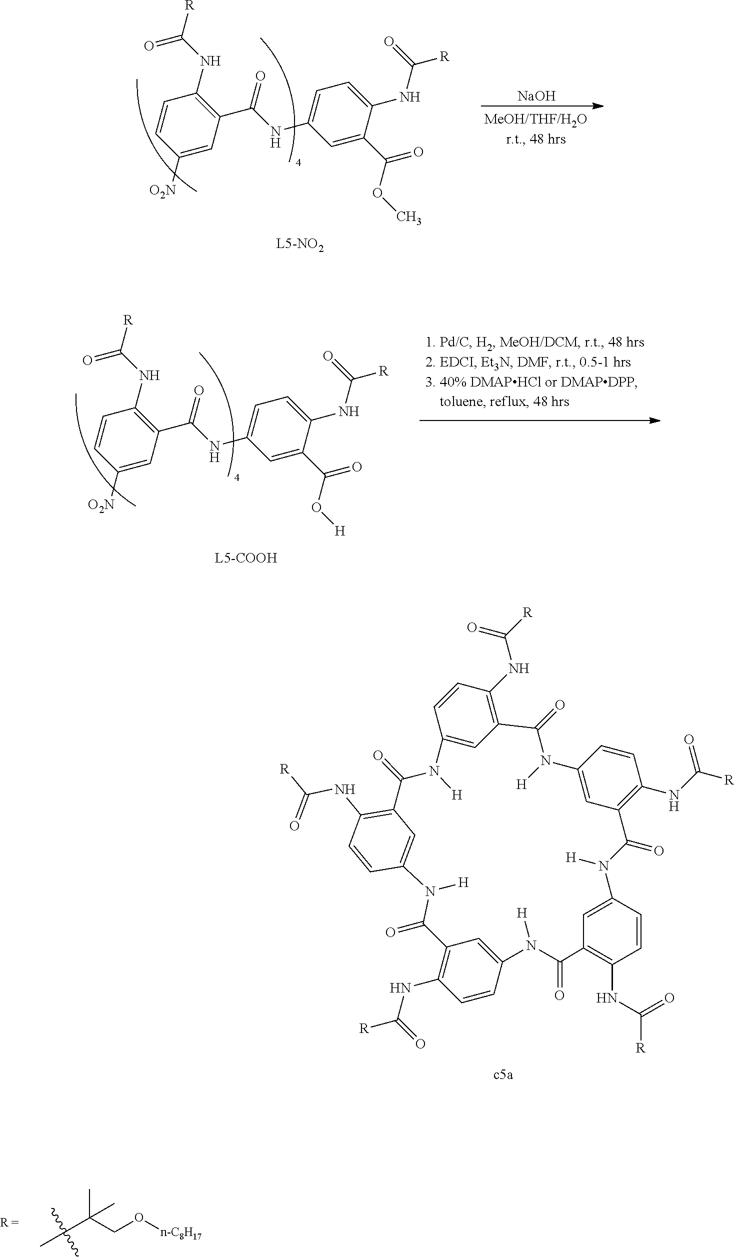

Nitro trimer methyl esterL3-NO₂: The hydrogenation of nitro dimer methyl ester L2-NO₂ to prepare its corresponding amine L2-NH₂ was accomplished via the general method. To a solution of the freshly prepared amine L2-NH₂ (1.85 g, 2.56 mmol) and benzoxazinone monomer 2a-NO₂ (0.96 g, 2.56 mmol) in dry toluene (80 mL) was added 20 mol % of DMAP·HCl. The obtained suspended solution was heated under reflux for 2 days. Then, the organic layer was washed with brine (40 mL×2 times) and dried over anhydrous Na₂SO₄. After removal of toluene under vacuum, the crude product was subjected to flash silica gel column (hexane/ethyl acetate, from 6/1 to 4/1) to afford the pure L3-NO₂ as a yellowish solid (2.56 g, 91%). ¹H NMR (400 MHz, CDCl₃): δ 11.48 (s, 1H), 11.21 (s, 1H), 10.85 (s, 1H), 9.17 (s, 1H), 8.83 (d, J=7.6 Hz, 1H), 8.74-8.71 (m, 2H), 8.65 (s, 1H), 8.35-8.32 (m, 2H), 8.28 (dd, J=7.6, 2.0 Hz, 1H), 7.86 (d, J=2.0 Hz, 1H), 7.75 (dd, J=7.2, 2.0 Hz, 1H), 7.57 (dd, J=7.2, 2.0 Hz, 1H), 3.92 (s, 3H), 3.55-3.50 (m, 5H), 3.47-3.43 (m, 5H), 3.41-3.37 (m, 4H), 1.61 (m, 2H), 1.54 (m, 2H), 1.45 (m, 4H), 1.34 (m, 12H), 1.29 (m, 12H), 1.23 (m, 14H), 1.12 (m, 12H), 0.90-0.80 (m, 10H). ¹³C NMR (75 MHz, 5% DMSO-d₆/95% CDCl₃): δ 176.42, 175.72, 167.91, 166.90, 165.84, 145.43, 141.35, 137.92, 135.77, 132.99, 132.67, 127.16, 126.70, 125.01, 124.18, 123.28, 122.63, 122.37, 121.10, 121.04, 120.84, 115.63, 77.39, 77.16, 77.09, 71.56, 71.54, 52.26, 44.94, 44.72, 44.35, 31.64, 31.60, 29.24, 29.19, 29.18, 29.15, 29.09, 29.10, 29.07, 25.92, 25.86, 25.83, 22.86, 22.76, 22.69, 22.47, 22.46, 13.98, 13.95. HRMS (ESI) m/z: [M+Na]⁺ Calcd, for C₆₁H₉₂N₆O₁₂Na 1123.6671; Found 1123.6687.

Nitro pentamer methyl esterL5-NO₂: The hydrogenation of nitro trimer methyl ester L3-NO₂ to prepare its corresponding amine L3-NH₂ was accomplished via the general method. To a solution of the freshly prepared amine L3-NH₂ (3.3 g, 3.08 mmol) and benzoxazinoneL2-BZX (2.34 g, 3.23 mmol) in dry toluene (70 mL) was added 20 mol % of DMAP·HCl. The obtained suspended solution was heated under reflux for 2 days. Then, the organic layer was washed with brine (50 mL×2 times) and dried over anhydrous Na₂SO₄. After removal of toluene under vacuum, the crude product was dissolved in the hot acetonitrile. The white precipitate formed and was collected via filtration. After drying it in the oven overnight, nitro pentamer methyl ester LS-NO₂ was obtained as a white solid (5.13 g: 93%). ¹H NMR (400 MHz, DMSO-d₆): δ 11.29 (s, 1H), 11.01 (s, 1H), 10.94 (s, 1H), 10.71 (s, 1H), 10.68 (s, 1H), 10.65 (s, 1H), 10.50 (s, 1H), 10.49 (s, 1H), 10.39 (s, 1H), 8.74-8.71 (m, 2H), 8.57 (d, J=7.2 Hz, 1H), 8.44-8.43 (m, 2H), 8.37 (d, J=6.8 Hz, 1H), 8.34-8.29 (m, 2H), 8.25 (s, 1H), 8.18-8.16 (m, 2H), 7.94 (d, J=7.2 Hz, 1H), 7.81-7.77 (m, 2H), 7.72 (d, J=7.6 Hz, 1H), 3.87 (s, 3H), 3.40 (m, 2H), 3.36 (m, 11H), 3.30 (m, 12H), 1.42 (m, 2H), 1.35 (m, 8H), 1.21 (m, 10H), 1.17 (m, 13H), 1.15 (m, 27H), 1.08 (m, 42H), 0.78 (m, 15H). ¹³C NMR (75 MHz, DMSO-d₆): δ 175.99, 174.99, 174.97. 174.89, 174.87, 174.61, 168.08, 167.38, 167.35, 167.34, 167.32, 167.27, 166.03, 145.12, 141.65, 137.50, 135.36, 134.86, 134.69, 134.35, 134.00, 133.99, 133.96, 133.92, 133.31, 129.33, 128.04, 126.41, 124.91, 124.88, 124.22, 124.10, 124.06, 124.00, 123.94, 123.88, 122.05, 122.03, 122.01, 121.95, 121.74, 121.72, 121.68, 121.08, 121.07, 121.05, 120.99, 120.97, 120.95, 120.88, 115.91, 109.99. 77.43, 77.40, 77.37, 77.32, 77.28, 77.19, 77.17, 77.13, 77.07, 77.04, 77.01, 76.95, 76.94, 76.92, 76.89, 76.84, 76.82, 71.23, 71.15, 71.14, 71.11, 71.05, 70.99, 70.92, 52.81, 52.78, 44.80, 44.65, 44.63, 44.13, 31.49, 28.98, 25.92, 25.76, 22.89, 22.84, 22.67, 22.57, 22.33, 14.11. HRMS (ESI) m/z: [M+Na]⁺ Calcd, for C₁₀₁H₁₅₂N₁₀O₁₈Na 1816.1184; Found 1816.1207.

Nitro pentamer acid L5-COOH): To a solution of L5-NO₂ (0.96 g, 0.54 mmol) in 50 mL of methanol/THF (v/v, 4/1) was added NaOH (0.10 g in 1 mL of H₂O). The hydrolysis reaction was carried out at room temperature for 4 days. The reaction solution was concentrated under vacuum, to which 1 M HCl aqueous solution was added to adjust the pH value of the solution to 3-4. Meanwhile, the white precipitate formed and was collected via the filtration. The crude product was then washed with acetonitrile. After drying it in the oven overnight, nitro pentamer acid L5-COOH was obtained as a white solid (0.86 g, 89%). 1H NMR (400 MHz, DMSO-d₆): δ 13.70 (broad, 1H), 11.46 (broad, 1H), 11.31 (s, 1H), 11.02 (s, 1H), 10.73 (s, 1H), 10.69 (s, 1H), 10.61 (s, 1H), 10.52 (m, 2H), 10.44 (s, 1H), 8.75-8.72 (m, 2H), 8.61 (d, J=9.2 Hz, 1H), 8.46-8.43 (m, 2H), 8.40-8.31 (m, 3H), 8.27 (s, 1H), 8.19-8.17 (m, 2H), 7.91 (d, J=9.2 Hz, 1H), 7.82-7.72 (m, 3H), 3.40-3.31 (m, 22H), 1.35 (m, 10H), 1.19-1.08 (m, 80H), 0.77 (m, 15H). ¹³C NMR (75 MHz, DMSO-d₆): δ 176.02, 175.23, 175.01, 174.88, 170.07, 167.35, 167.23, 166.12, 166.04, 145.15, 141.65, 138.06, 135.39, 134.89, 134.75. 133.98, 133.91, 133.68, 133.32, 128.04, 125.97, 124.92, 124.21, 124.05, 123.94, 123.02, 122.03, 121.92, 121.73, 121.08, 120.23, 116.68, 77.51, 77.08, 76.95, 71.18, 70.94, 44.81, 44.70, 44.19, 31.51, 29.05, 28.98, 25.93, 25.77, 22.86, 22.70, 22.57, 22.35, 14.14, HRMS (ESI) m/z: [M+Na]⁺ Calcd, for C₁₀₀H₁₅₀N₁₀O₁₈Na 1802.1027; Found 1802.0988.

Macrocycle c5a: Hydrogenation reaction of nitro pentamer acid L5-COOH to its corresponding amine was carried out via the general procedure. To a solution of the freshly prepared aromatic amine (0.55 g, 0.31 mmol) in 3 mL of dry DMF was added EDCl (89 mg, 0.46 mmol) and triethylamine (68 mg, 0.62 mmol). The obtained reaction solution was stirred at room temperature for 0.5-1 hrs. Then, all solvent was removed under vacuum. The mixture was used for the cyclization reaction without any further purification. To accomplish the intramolecular cyclization, the mixture was suspended in dry toluene (160 mL), followed by addition of 40 mol % DMAP·HCl or a mixture containing DMAP (15 mg, 0.12 mmol) and DPP (30 mg, 0.12 mmol) (40 mol % DMAP·DPP). The obtained suspended solution was heated under reflux for 48 hrs. Then the organic layer was washed with brine (50 mL×2 times) and dried over anhydrous Na₂SO₄. After removal of toluene under vacuum, the crude product was subjected to flash silica gel column (DCM/MeOH, from 50/1 to 30/1) to afford the crude product. The further purification was accomplished by the precipitation of the crude product in hot acetonitrile. Then the white solid was collected via filtration and dried in the oven overnight. The pure c5a was obtained as a white solid (0.17 g (32%) when DMAP·HCl was used as the catalyst; 0.19 g (35%) when DMAP·DPP was used as the catalyst), corresponding to overall yields of 22% and 24%, respectively, for c5a from the stepwise synthesis starting from the benxazinone monomer. ¹H NMR (400 MHz, DMSO-d₆): δ 10.58 (s, 5H), 10.43 (s, 5H), 8.40 (d, J=9.2 Hz, 5H), 8.33 (d, J=8.8 Hz, 5H), 7.61 (s, 5H), 3.41 (s, 10H), 3.37 (t, J=6.4 Hz. 10H), 1.42 (m, 10H), 1.19-1.13 (m, 50H), 1.09 (m, 30H), 0.77 (t, J=6.8 Hz, 15H). ¹³C NMR (75 MHz, DMSO-d₆): δ 174.78, 166.94, 134.87, 133.81, 124.71, 123.91, 122.17, 121.28, 77.23, 71.28, 44.31, 31.67, 29.34, 29.23, 29.17, 26.01, 23.01, 22.52, 14.37. HRMS (ESI) m/z: [M+Na]⁺ Calcd, for C₁₀₀H₁₅₀N₁₀O₁₅Na 1754.1180; Found 1754.1160.

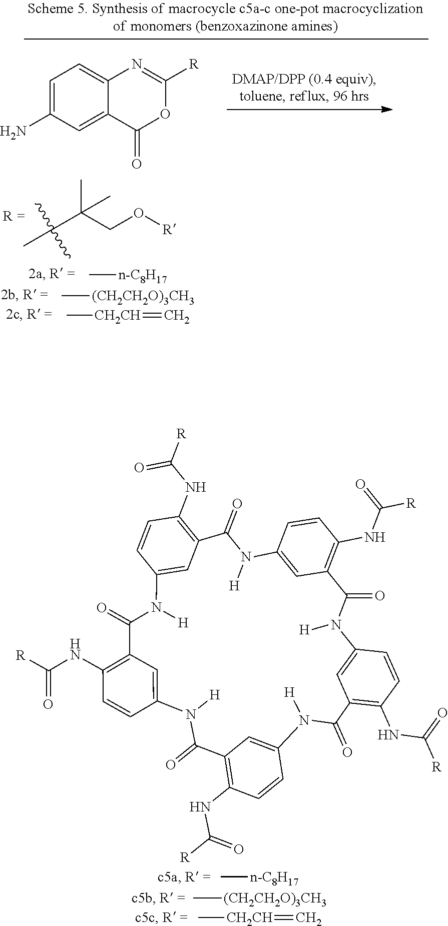

General synthetic procedure for macrocycle c5a-c: To a solution of benzoxazinone amine 2a-c (0.09 mmol) in dry toluene (45 mL) were added DMAP (4.4 mg, 0.036 mmol) and DPP (9.0 mg, 0.036 mmol). The resulting reaction solution was heated under reflux for 96 hrs. Then, the reaction solution was washed with brine (45 mL×2 times) and dried over anhydrous Na₂SO₄. Under high vacuum, the reaction solution was concentrated for further purification.

Macrocycle c5a: The crude product was subject to silica gel column (DCM/MeOH, from 50/1 to 30/1) to offer the pure c5a as a white solid (14.0 mg, 45%). The characterization data of c5a via the one-pot macrocyclization is consistent to the one obtained via the step-wise synthetic method.

Macrocycle c5b: The crude product was subject to silica gel column (DCM/MeOH, from 40/1 to 20/1) to offer the pure c5b as a white solid (13.0 mg, 38%). ¹H NMR (400 MHz, DMSO-d₆): δ 10.65 (s, 5H), 10.45 (s, 5H), 8.37-8.32 (m, 10H), 7.69 (s, 5H), 3.57-3.33 (m, 75H), 3.19 (s, 10H), 1.22 (s, 30H). ¹³C NMR (75 MHz, DMSO-d₆): δ 174.92, 167.09, 134.96. 133.85, 124.72, 124.27, 122.35, 121.61, 77.56, 71.60, 70.79, 70.18, 70.08, 69.91, 69.81, 58.36, 44.23, 22.82. HRMS (ESI) m/z: [M+Na]⁺ Calcd, for C₉₅H₁₄₀N₁₀O₃₀Na 1923.9635; Found 1923.9617.

Macrocycle c5c: The crude product was subject to silica gel column (CH₂Cl₂/MeOH, from 50/1 to 30/1) to offer the pure c5c as a white solid (9.6 mg, 39%). ¹H NMR (400 MHz, DMSO-d₆): δ 10.68 (s, 5H), 10.44 (s, 5H), 8.38 (d, J=8.8 Hz, 5H), 8.34 (d, J=8.8 Hz, 5H), 7.63 (s, 5H), 5.86-5.81 (m, 5H), 5.23 (d, J=17.2 Hz, 5H), 5.05 (d, J=10.4 Hz, 5H), 3.98 (d, J=5.6 Hz, 10H), 3.46 (s, 10H), 1.24 (s, 30H). ¹³C NMR (75 MHz, DMSO-d₆): δ 174.75, 167.00, 147.97, 135.38, 133.75, 124.39, 121.62, 116.84, 84.83, 76.79, 71.88, 44.34, 23.00. HRMS (ESI) m/z: [M+Na]⁺ Calcd, for C₇₅H₉₀N₁₀NaO₁₅ 1393.6485; Found. 1393.6451.

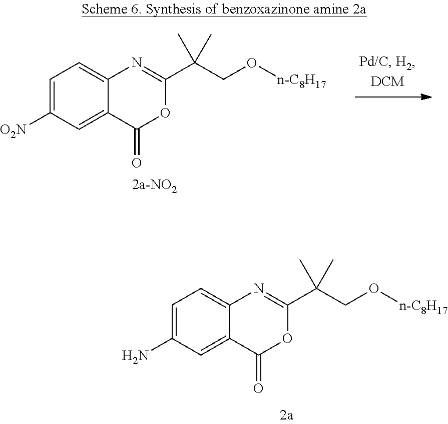

Compound 2a: In the presence of H₂ gas, the solution of 2a-NO₂ (0.75 g, 2.0 mmol) and catalytic amount of Pd/C in DCM was stirred at room temperature overnight. All solid was filtered out and resulting filtrate was concentrated under vacuum. The pure benzoxazinone amine 2a was obtained as a white solid (0.64 g, 93%). ¹H NMR (400 MHz, CDCl₃): δ 7.42 (d, J=8.4 Hz, 1H), 7.35 (d, J=2.8 Hz, 1H), 7.08 (d, J=8.4 and 2.8 Hz, 1H), 4.02 (broad, 2H), 3.58 (s, 2H), 3.41 (t, J=6.4 Hz, 1H), 1.48 (m, 2H), 1.35 (s, 2H), 1.25-1.17 (m, 10H), 0.85 (t, J=6.8 Hz, 3H). ¹³C NMR (75 MHz, CDCl₃): δ 162.59, 160.59, 146.51, 138.49, 128.05, 123.77, 117.65, 110.56, 77.27, 71.62, 42.36, 31.75, 29.35, 29.32, 29.22, 26.03, 23.07, 22.61, 14.07. MS (ESI) m/z: [M+H]⁺ Calcd, for C₂₀H₃₁N₂O₃, 347.23; Found 347.19.

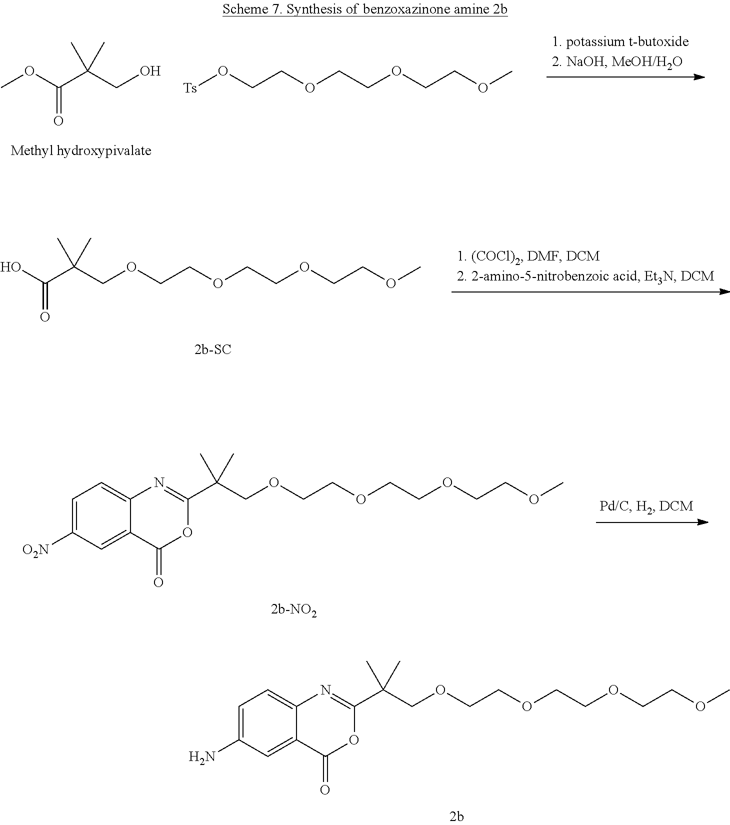

Compound 2b-SC: At 0° C., to the solution of methyl hydroxypivalate (10.37 g, 1.0 eq) and 2-(2-(2-methoxyethoxy) ethoxy)ethyl 4-methyl benzenesulfonate (25.0 g, 1.0 eq) in dry DMF (75 mL) was added potassium t-butoxide (9.69 g, 1.1 eq) portionwise. The reaction mixture was heated to 80° C. for 2 days. The reaction was cooled to room temperature and the solid was filtered off. The reaction solution was concentrated under vacuum and then diluted with ethyl acetate. The resulting solution was washed with 1 M HCl (50 mL×2 times), water (50 mL×2 times) and brine (50 mL×2 times). The organic layer was dried over anhydrous Na₂SO₄ and then removed under vacuum. The crude methyl ester was dissolved in MeOH (100 mL), followed by an addition of NaOH aqueous solution (5.76 g in 75 mL of H₂O). The obtained reaction solution was stirred at room temperature for 2 days. MeOH was removed under vacuum. The aqueous residue was acidified with diluted HCl aqueous solution. Then, the aqueous solution was extracted with ethyl acetate (50 mL×3 times). The organic layer was combined and washed with water (50×2 times) and brine (50×2 times) combined and concentrated to give pure 2b-SC as an oil (6.9 g, 33% for two steps). ¹H NMR (400 MHz, CDCl₃): δ 3.68-3.64 (m, 10H), 3.61-3.59 (m, 2H), 3.50 (s, 2H), 3.40 (s, 3H), 1.22 (s, 6H). ¹³C NMR (75 MHz, CDCl₃): δ 181.14, 77.58, 71.77, 70.93, 70.52, 70.49, 70.21, 70.04, 58.74, 43.19, 22.06. HRMS (ESI) m/z: [M−H]⁻ Calcd, for C₁₂H₂₃O₆ 263.1500; Found 263.1498.

Compound 2b-NO₂: To a solution of 2b-SC (10.56 g, 40 mmol) in dry DCM (50 mL) was added oxalyl chloride (5.59 g, 44 mmol). After addition of one drop of dry DMF as an initiator, the reaction was stirred at room temperature for 5 hours. The corresponding acid chloride was obtained after removal of DCM under vacuum, which was used for the next step without any further purification. To a solution of 2-amino-5-nitrobenzoic acid (2.91 g, 16 mmol) and triethylamine (5.45 g, 50 mmol) in DCM (50 mL) was added freshly prepared acid chloride. The obtained reaction solution was stirred overnight. Then the reaction solution was concentrated under vacuum and the crude product was subject to silica gel column (ethyl acetate/hexanes, from 3/1 to 1/1). The pure 2b-NO₂ was prepared as a yellow oil (3.88 g, 59%). ¹H NMR (400 MHz, CDCl₃): δ 9.03 (d, J=2.8 Hz, 1H), 8.58 (dd, J=9.6 and 2.8 Hz, 1H), 7.74 (d, J=9.6 Hz, 1H), 3.69 (s, 2H), 3.60-3.56 (m, 10H), 3.51 (m, 2H), 3.36 (s, 3H), 1.40 (s, 6H). ¹³C NMR (75 MHz, CDCl): δ 169.67, 158.02, 150.66, 146.42, 130.34, 128.59, 124.30, 117.24, 77.65, 71.82, 70.99. 70.50, 70.47, 70.39, 70.26, 58.89, 43.28, 22.88. MS (ESI) m/z: [M+H]⁺ Calcd, for C₁₉H₂₇N₂O₈, 411.17; Found 411.06.

Compound 2b: Benzoxazinone amine 2b was prepared via the same method as the preparation of 2a without any modification. The 2b (0.68 g, 89%) was obtained as a yellow oil from 2b-NO₂ (0.82 g, 2.0 mmol). ¹H NMR (400 MHz, CDCl₃): δ 7.37 (d, J=8.4 Hz, 1H), 7.32 (d, J=2.8 Hz, 1H), 7.06 (d, J=8.4 and 2.8 Hz, 1H), 4.05 (s, 2H), 3.64 (s, 2H), 3.58-3.51 (m, 12H), 3.35 (s, 3H), 1.35 (s, 6H). ¹³C NMR (75 MHz, CDCl₃): δ 162.44, 160.48, 146.53, 138.45, 128.05, 123.77, 117.68, 110.56, 77.79, 71.87, 71.08, 70.56, 70.42, 70.30, 58.97, 42.35, 23.02. MS (ESI) m/z: [M+H]⁺ Calcd, for C₁₉H₂₉N₂O₆, 381.20; Found 381.17.

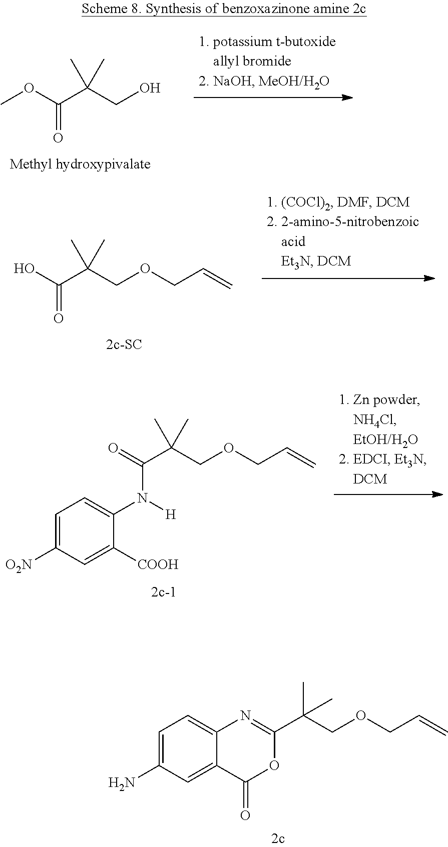

Compound 2c-SC: At 0° C., to the solution of methyl hydroxypivalate (13.2 g, 100 mmol) and allyl bromide (13.3 g, 110 mmol) was added potassium t-butoxide (12.4 g, 110 mmol) portionwise. The reaction mixture was stirred overnight at room temperature. The reaction mixture was diluted with ethyl acetate. The resulting solution was washed with 1 M HCl (50 mL×2 times), water (50 mL×2 times) and brine (50 mL×2 times). The organic layer was dried over anhydrous Na₂SO₄ and then removed under vacuum. The obtained crude methyl ester was dissolved in MeOH (100 mL), followed by an addition of NaOH aqueous solution (4 g in 10 mL of H₂O). The obtained reaction solution was stirred overnight at room temperature. MeOH was removed under vacuum. The aqueous residue was acidified with diluted HCl aqueous solution. Then, the aqueous solution was extracted with ethyl acetate (50 mL×3 times). The organic layer was combined and washed with water (50×2 times) and brine (50×2 times) combined and concentrated to give pure 2c-SC as an oil (6.1 g, 39% for two steps). ¹H NMR (400 MHz, CDCl₃): δ 5.94-5.84 (m, 1H), 5.30-5.17 (m, 2H), 4.02 (d, J=5.6 Hz, 2H), 3.45 (s, 2H), 1.23 (s, 6H). ¹³C NMR (75 MHz, CDCl₃): δ 183.89, 135.20, 117.42, 77.00, 72.85, 43.89, 22.63. HRMS (ESI) m/z: [M−H]⁻ Calcd, for C₈H₁₃O₃ 157.0870; Found. 157.0868.

Compound 2c-1: To a solution of 2c-SC (6.1 g, 39 mmol) in dry DCM (50 mL) was added oxalyl chloride (5.2 g, 41 mmol). After addition of one drop of dry DMF as an initiator, the reaction was stirred at room temperature for 5 hours. The corresponding acid chloride was obtained after removal of DCM under vacuum, which was used for the next step without any further purification. To a solution of 2-amino-5-nitrobenzoic acid (6.5 g, 36 mmol) and triethylamine (6.4 g, 59 mmol) in DCM (50 mL) was added freshly prepared acid chloride. The obtained reaction solution was stirred overnight. Then the reaction solution was concentrated under vacuum and the resulting residue was then dissolved in 100 mL of ethyl acetate. The organic layer was washed with dilute HCl aqueous solution (20 mL×2 times) followed by brine (20 mL×2 times). The organic layer was then dried over anhydrous Na₂SO₄ and removed under vacuum. The crude product was dissolved in hot ethyl acetate/hexane. Then white precipitate formed and was collected via simple filtration to give pure compound 2c-1 (5.5 g. 56%). ¹H NMR (400 MHz, CDCl₃): δ 11.50 (s, 1H), 9.03 (d, J=9.6 Hz, 1H), 8.96 (d, J=2.8 Hz, 1H), 8.40 (dd, J=9.6 and 2.8 Hz, 1H), 5.90-5.83 (m, 1H), 5.27-5.15 (m, 2H), 4.04 (d, J=5.6 Hz, 2H), 3.53 (s, 2H), 1.36 (s, 6H). ¹³C NMR (75 MHz, CDCl₃): δ 177.45, 170.88, 147.64, 142.32, 134.71, 130.64, 128.18, 121.68, 118.19, 77.27, 73.06, 45.75, 23.18. HRMS (ESI) m/z: [M−H]⁻ Calcd, for C₁₅H₁₇N₂O₆ 321.1092; Found. 321.1087.

Compound 2c: To a solution of 2c-1 (2.0 g, 7.2 mmol) and zinc powder (1.9 g, 28.8 mmol) in ethanol (40 mL) was added NH₄Cl (2.4 g in 8 mL of H₂O). The reaction solution was heated under reflux for 5 hrs. Then, the hot solution was filtered to remove any insoluble solid. The obtained solution was concentrated and water had to be removed under vacuum to give the dry amine product, which was used in the next step without further purification. To a suspended solution of crude amine in DCM (20 mL) was added EDCl (1.4 g, 7.2 mmol) and triethylamine (1.6 g, 14.4 mmol). The reaction was stirred overnight at room temperature. After removal of DCM, the reaction residue was subject to silica gel column (hexane/ethyl acetate, from 5/1 to 3/1). The benzoxazinone amine 2c was obtained as a yellowish solid (0.77 g, 39% for two steps). ¹H NMR (400 MHz, CDCl₃): δ 7.42 (d, J=8.4 Hz, 1H), 7.37 (d, J=2.8 Hz, 1H), 7.09 (dd, J=8.4 and 2.8 Hz, 1H), 5.89-5.80 (m, 1H), 5.31-5.12 (m, 2H), 4.00-3.98 (m, 4H), 3.61 (s, 2H), 1.37 (s, 6H). ¹³C NMR (75 MHz, CDCl₃): δ 162.51, 160.54, 146.40, 138.60, 134.77, 128.15, 123.81, 117.74, 116.77, 110.68, 76.64, 72.32, 42.30, 23.11. MS (ESI) m/z: [M+H]⁺ Calcd, for C₁₅H₁₉N₂O₃, 275.32; Found 275.68.

Vesicle Preparation. A portion (200 μL) of a solution of 1-palmitoyl-2-oleoyl-sn-glycero-3-phosphocholine (POPC) or dipalmitoylphosphatidylcholine (DPPC) (from Avanti Polar Lipids, US) in chloroform (25 mg/mL) was first diluted into 10 mL of chloroform. A thin lipid film was prepared by evaporating the solution on a rotary evaporator in vacuo for at least 3 hours.

For POPC-LUVs with encapsulated HPTS and an intravesicular pH of 11.5, the POPC thin film was hydrated with a buffer (10 mM HEPES, 100 mM NaCl, pH 11.5) containing 8-hydroxypyrene-1,3,6-trisulfonic acid trisodium salt hydrate (HPTS, from Sigma-Aldrich, US) (0.03 mM) at 37° C. for >30 min, with the final concentration of the lipid being 0.1 mg/mL. The resultant lipid suspension was subjected to 5 freeze-thaw cycles (liquid nitrogen −37° C. water bath), followed by extrusion through a 0.1-μm polycarbonate membrane (Sigma-Aldrich, US) to produce a solution of large unilamellar vesicles (LUVs). The LUVs were separated from the extravesicular HPTS and other components by size exclusion chromatography using Sephadex G-50 (GE Healthcare, US), pre-equilibrated with the buffer of 10 mM HEPES, 100 mM NaCl, pH 11.5.

For POPC-LUVs with encapsulated HPTS and an intravesicular pH of 7.0, the POPC thin film was hydrated with a buffer (10 mM HEPES, 100 mM NaCl, pH 7.0) containing 1 mM of HPTS (from Sigma-Aldrich, US) at 37° C. for >30 min, with the final concentration of the lipid being 0.1 mg/mL. The resultant lipid suspension was subjected to 5 freeze-thaw cycles (liquid nitrogen −37° C. water bath), followed by extrusion through a 0.1-μm polycarbonate membrane (Sigma-Aldrich, US) to produce a solution of large unilamellar vesicles (LUVs). The LUVs were separated from the extravesicular HPTS and other components by size exclusion chromatography using Sephadex G-50 (GE Healthcare, US), pre-equilibrated with the buffer of 10 mM HEPES, 100 mM NaCl, pH 7.0.

For POPC-LUVs with encapsulated lucigenin, the thin film was hydrated with a buffer (10 mM HEPES, 100 mM NaNO₃, pH 7) containing with 1 mM of 1 mM N,N′-dimethyl-9,9′-biacridinium dinitrate (lucigenin, from TCI, Japan) at 37° C. for >30 min, with the final concentration of the lipid being 1 mg/mL. The resultant lipid suspension was subjected to 5 freeze-thaw cycles (liquid nitrogen −37° C. water bath), followed by extrusion through a 0.1-μm polycarbonate membrane (Sigma-Aldrich, US) to produce a solution of LUVs. The LUVs were separated from the extravesicular lucigenin and other components by size exclusion chromatography using Sephadex G-50 (GE Healthcare, US), pre-equilibrated with the buffer of 10 mM HEPES, 100 mM NaCl, pH 7.0.

For DPPC-LUVs with encapsulated HPTS, the thin film was hydrated with a buffer (10 mM HEPES, 100 mM NaCl, pH 7) containing 0.03 mM of 8-hydroxypyrene-1,3,6-trisulfonic acid trisodium salt hydrate (HPTS, from Sigma-Aldrich, US) at 45° C. for >30 min, with the final concentration of the lipid being 0.1 mg/mL. The resultant lipid suspension was subjected to 5 freeze-thaw cycles (liquid nitrogen −45° C. water bath), followed by extrusion through a 0.1-μm polycarbonate membrane (Sigma-Aldrich, US) at 45° C. to produce a solution of LUVs. The LUVs were separated from the extravesicular lucigenin and other components by size exclusion chromatography using Sephadex G-50 (GE Healthcare, US), pre-equilibrated with the buffer of 10 mM HEPES, 100 mM NaCl, pH 7.0.

Determination of the transmembrane permeability of halide transport mediated by c5a with the HPTS assay. An aliquot (10 μL) of macrocycle c5a (0.5 mM) in DMF was added to 1.8 mL of a buffer comprising of 10 mM HEPES, 100 mM NaX (X═Cl, Br or I) at pH 6.5, to which 200 μL POPC LUVs with intravesicular components of 10 mM HEPES, 100 mM NaCl, pH 11.5, was added. The fluorescence emission of the intravesicular HPTS was monitored with a fluorescence spectrometer (Cary Eclipse, Agilent, US) at the excitation wavelengths of 405 nm and 450 nm simultaneously, based on which the ratio of emission intensities corresponding to the 405-nm and 450-nm excitation was obtained. After 400 s, the vesicles were lysed by adding Triton X-100 (20 μL, 10% v/v) to obtain the minimal fluorescence intensity for normalization.

Each of the normalized time-lapse fluorescence emission curves was fitted with the quantitative model (black lines) according to the Goldman-Hodgkin-Katz flux theory⁴ and the carrier-mediated transport model developed by Behr et al⁵, based on which the anion permeability of vesicles with or without c5a was determined. The total anion permeability (ΔP_{X-}) of each halide ion elicited by c5a was calculated by subtracting the anion permeability measured with vesicles in the absence of c5a.

Quantitative model for vesicle-based halide transport HPTS assay, to describe the process of cross-membrane transport of c5a-halide, a quantitative flux model with a combining of the Goldman-Hodgkin-Katz theory and carrier-mediated transport model was developed here. According to the Goldman-Hodgkin-Katz theory, the ion flux across the vesicle membrane can be calculated as:

$$j = μ nP \frac{{[ A ]}_{out} - {[ A ]}_{i n} {e}^{n μ}}{1 - {e}^{n μ}}$$

where

$$μ = \frac{{FE}_{m}}{R T} .$$

F is the Faraday constant, Eₘ is the membrane potential, R is the molar gas constant. T is the temperature, n is the charge valence, P is the permeability and [A]ₒᵤₜ is the concentration of ion-carrier complex at the outer membrane-solution interface, [A]ᵢₙ is the concentration at the inner membrane-solution interface. According to the carrier-mediated transport model developed by Behr et. al, [A]ₒᵤₜ and [A]ᵢₙ can be calculated as follow:

$${[ A ]}_{o u t} = {K}_{a} [ C ] \frac{{[ S ]}_{out}}{1 + {{K}_{a} [ S ]}_{out}}$$

$${[ A ]}_{i n} = {K}_{a} [ C ] \frac{{[ S ]}_{i n}}{1 + {{K}_{a} [ S ]}_{in}}$$

where Kₐ is the association constants for the binding of carrier with ion, [C] is the concentration of carriers in the membrane, [S]ₒᵤₜ and [S]ᵢₙ is the extra-vesicular and intra-vesicular ion concertation. So the ion flux j can be re-written as:

$$j = μ nP {K}_{a} [ C ] \frac{\frac{{[ S ]}_{out}}{1 + {{K}_{a} [ S ]}_{out}} - \frac{{[ S ]}_{i n}}{1 + {{K}_{a} [ S ]}_{i n}} {e}^{n μ}}{1 - {e}^{n μ}}$$

Based on this formula, the change in intra-vesicular halide ion and further resulting pH variation were numerically calculated iteratively with a given time-step (10-7 s) for the whole duration of experimental measurements. The calculations were carried out with Matlab (Math Work, Natick, USA). All the codes are available upon request.

Determination of the EC50 values of halide transport mediated by c5a with the HPTS assay. An aliquot (20 μL) POPC LUVs (intravesicular components: 10 mM HEPES, 100 mM NaCl, pH 11.5) was added to 1.98 mL of a buffer comprised of 10 mM HEPES, 100 mM NaCl, pH 7, followed by adding 20 μL of c5a in DMF. The emission of the intravesicular HPTS was monitored at 510 nm with a fluorescence spectrometer (Cary Eclipse, Agilent, US) at the excitation wavelengths of 405 nm and 450 nm. After 200 s, the vesicles were lysed by adding Triton X-100 (20 μL, 10% v/v) to obtain the maximum fluorescence intensity for normalization.

The time course of the emission intensity of intravesicular HPTS, Iₜ, was obtained first by ratiometric analysis (Rₜ=I_{t,450}/I_{t,405}), followed by normalization according to equation (S1),

$$Nomalized intensity ratio = \frac{{R}_{t} - {R}_{0}}{{R}_{∞} - {R}_{0}} \qquad ( S 1 )$$

where R₀=Rₜ at t=100 s, before the addition of c5a; R_{∞}=Rₜ at t=350 s upon the addition of Triton X-100. Normalized intensity ratio at 300 s just before addition of Triton X-100 was defined as the transmembrane activity (Y) which was analyzed with the Hill equation (S2) to give effective concentration EC50 and the Hill coefficient n, (S2)

$$Y = {Y}_{∞} + \frac{{Y}_{0} - {Y}_{∞}}{{( 1 + \frac{c}{EC 50} )}^{n}} \qquad ( S 2 )$$

where Y₀ is Y in absence of carrier, Y₀ is Y with excess carrier, and c is the carrier concentration in a cuvette.

Determination of halide transport with the lucigenin assay. An aliquot (20 μL) POPC LUVs (intravesicular components: 10 mM HEPES, 100 mM NaNO₃, pH 7.0) was added to 1.98 mL of each of the buffers comprised of 10 mM HEPES, 100 mM NaX (X═Cl, Br, or I), pH 7, followed by adding 20 μL of c5a (0.2 mM) in DMF, with the final concentration of c5a being 10 μM. The emission of the intravesicular lucigenin was monitored at 505 nm with a fluorescence spectrometer (Cary Eclipse, Agilent, US) at the excitation wavelength of 430 nm. After 200 s, the vesicles were lysed by adding Triton X-100 (20 μL, 10% v/v) to obtain the minimal fluorescence intensity for normalization.

Differentiation of carrier vs channel chloride transport with DPPC LUVs. An aliquot (20 μL) DPPC LUVs (intravesicular components: 10 mM HEPES, 100 mM NaCl, pH 7.0) was added to 1.98 mL of a buffer comprised of 10 mM HEPES, 100 mM NaCl, pH 7, followed by incubating at 45° C. for 3 min. An aliquot (20 μL) of c5a (0.2 mM) in DMF was added to the LUVs, with the final concentration of c5a being 5 μM, and the solution was incubated for an additional 3 min at 45° C. LUVs with c5a were incubated either at 45° C. or 25° C. for 3 minutes. The emission of the intravesicular HPTS was monitored at 510 nm with a fluorescence spectrometer (Cary Eclipse, Agilent, US) at the excitation wavelengths of 405 and 450 nm, followed by applying a base pulse (20 μL of 0.5 mM NaOH) at 50 s. After 200 s, the vesicles were lysed by adding Triton X-100 (20 μL, 10% v/v) to obtain the maximum fluorescence intensity for normalization.

### Cell Culture and Confocal Imaging:

Primary Cell Culture. Human bronchial epithelial (HBE) cells from cystic fibrosis (CF) patients (DF508 homozygous) were obtained from the UNC-CF Tissue Culture Core under the auspices of protocols approved by the UNC Institutional Review Board using previously reported methodologies. Cultures on 12-mm permeable supports (Transwell-Clear; Costar) were maintained in a well-humidified (>95%) incubator (5% CO₂) at 37° C. during cellular differentiation (3-4 weeks after plating).

Confocal imaging studies. CF HBE cultures were treated with apically with c5a (5 μM in 1% DMSO) or vehicle control (1% DMSO in PBS) daily for 5 days prior to imaging. The height of the luminal airway surface layer (ASL) was visualized by adding a cell-impermeant dye (Texas Red-dextran 70 kDa; 0.1%) to the c5a and vehicle control on the evening prior to imaging. The cultures were mounted on the stage of a scanning confocal microscope (SP5, Leica) outfitted with an environmental chamber that regulated temperature to 37° C., humidity at >50%, and CO₂ at 5%. The thickness of the ASL layer in c5a and control cultures were measured by high-speed X-Z confocal scanning. A custom Matlab script was used to make thickness measurements from the resulting images.

Computational Studies. The density functional theory calculations were performed with the ADF software package. The revPBE-D3 functional and dispersion correction, along with a TZP basis set, were used to geometry optimize all structures involving the macrocycle c5, amide molecular units based on macrocycle c5, linear pentamer L5, and macrocycle c6. The core electrons were frozen for the oxygen, nitrogen, carbon, sulfur, phosphorus, chlorine, bromine, and iodine atoms. Models detailing the host-guest interaction of chloride, bromide, iodide, nitrate, perchlorate, bisulfate, methyl phenylphosphonate, and diphenyl phosphate with the macrocycle c5 were treated with a-1 overall charge on the system. The Hirshfeld charges, dipole moments, and energy decomposition analyses (EDA) were obtained from relaxed structures of macrocycle c5, linear pentamer L5, and/or amide molecular units based on the macrocycle c5.

X-ray crystallography. Single crystals of c5c were obtained from CHCl₃/acetonitrile (1/1, v/v) by slow evaporation of solvents at room temperature. X-ray diffraction data were collected on a Rigaku XtaLAB Synergy diffractometer coupled to a Rigaku Hypix detector with Cu Kα radiation (λ=1.54184 Å), from a PhotonJet micro-focus X-ray source at 100 K. Data of the single-crystal structure were solved by direct methods which revealed the positions of all non-hydrogen atoms, and were refined on F² by a full-matrix least squares procedure using anisotropic displacement parameters. All hydrogen atoms were placed in ideal geometry and were refined using a riding model.

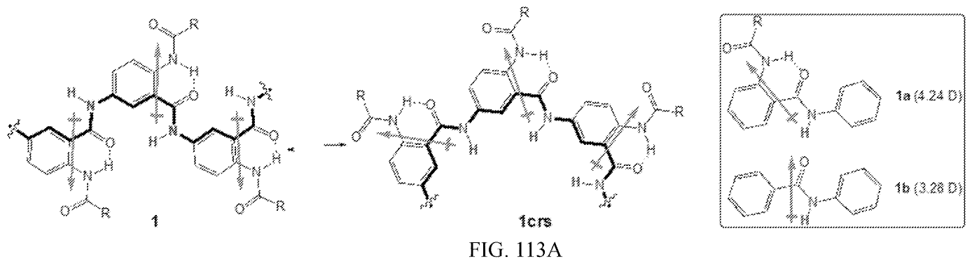

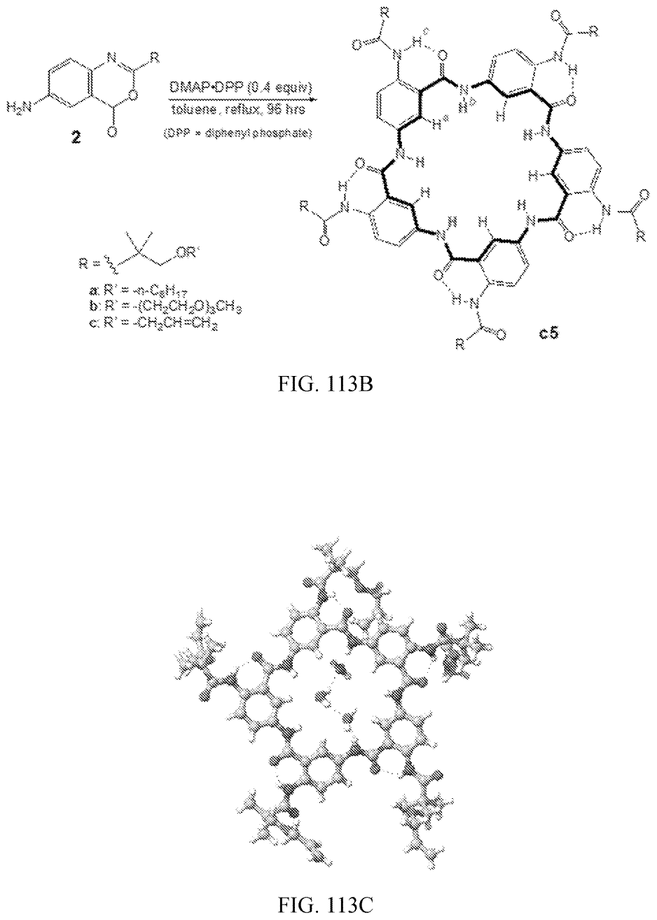

Design of cyclic aromatic pentaamides c5 featuring a constrained cyclic backbone with convergently placed endocyclic CH and NH donors. Anion-binding aromatic oligoamide foldamers share general structure 1 (FIG. 113a). The intramolecular hydrogen bonds incorporated into 1 serve to (1) partially rigidify the oligoamide backbone, (2) prevent the backbone amide C—O groups from engaging in additional H-bonding interaction, and (3) free the backbone amide NH groups to form hydrogen bonds with guest species. As shown in FIG. 113a, amide 1a, i.e., the basic unit of 1, has a large dipole moment (4.24 D) contributed by the two hydrogen-bonded amide groups, while amide 1b, which lacks a sidechain amide group, has a smaller dipole moment (3.28 D). Thus, oligoamide 1 adopts a conformation in which the adjacent amide dipoles align in an energetically favorable antiparallel orientation. Similar to the anion-binding foldamers, upon binding an anion, oligoamide 1 folds into conformation 1crs in which the backbone NH groups, along with the positive ends of the amide dipoles, are oriented to define an electropositive cavity. The entropic barrier for 1 to adopt conformation 1crs leads to modest anion binding affinities that increase with the number of NH groups.

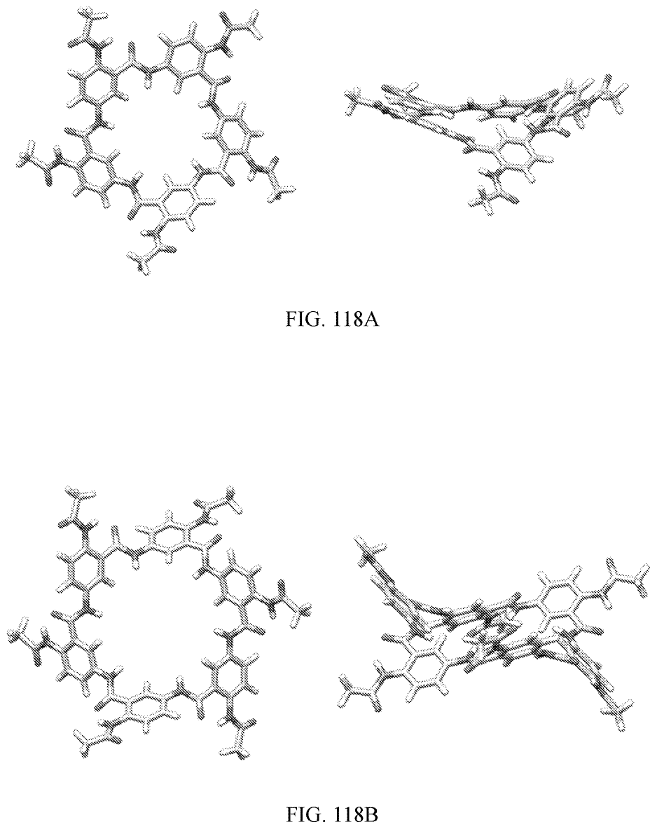

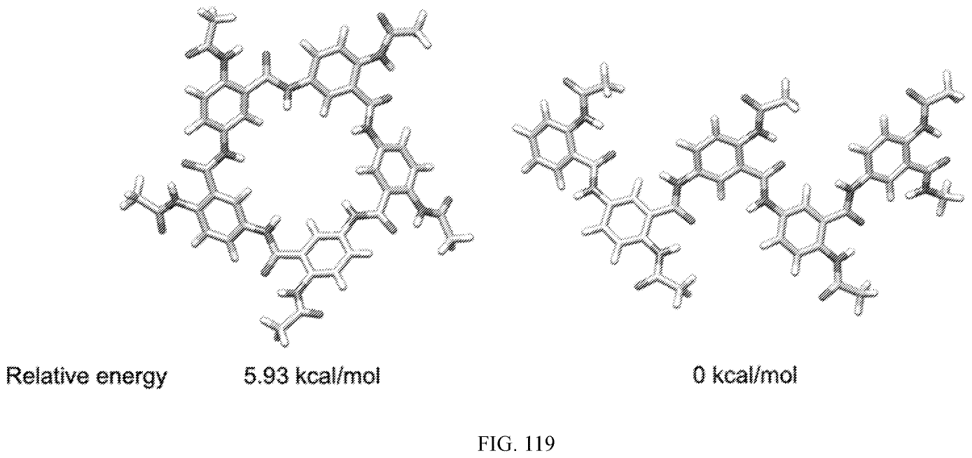

Cyclizing oligoamide 1 yields the corresponding macrocycle. Based on the bond angles defined by the sp2-hybridized C and N atoms constituting the oligoamide backbone, macrocycles with five or six residues are most likely to form. Other smaller or larger macrocycles will be more strained and thus be more difficult to form. Optimization of the five-residue macrocycle c5 (FIG. 113b) with density functional theory (DFT) calculations reveals a slightly puckered conformation (FIG. 118a). In contrast, the analogous six-residue macrocycle has a significantly crinkled shape, indicating a strained structure (FIG. 118b). The cyclic backbone of c5, along with the intramolecular hydrogen bonds, hinders the rotation of the aryl-amide single bonds, which greatly reduces the conformational flexibility of the backbone. The intramolecular hydrogen bonds also prevent the amide groups from flipping 180°, forcing the amide NH and phenyl CH groups to point to the interior of the macrocycle (FIGS. 113b, c and 118a). The formation of c5 is calculated to be 5.93 kcal/mol endothermic with respect to its acyclic counterpart (FIG. 119), which is a result of the repulsion between amide dipoles, and between the partially charged endocyclic NH and CH protons brought into close proximity by the cyclized backbone. As confirmation, an energy decomposition analysis (EDA) reveals that, compared to the linear pentamer, c5 is 16.9 kcal/mol less stable, but gains stability through greater orbital interactions (−9.6 kcal/mol). Consequently, macrocycle c5, with its highly electropositive cavity, as shown with DFT computed Hirshfeld charges of +0.12 e and +0.04 e for each of the NH and CH protons, is in a high-energy unbound state³¹ and is predisposed for binding anions.

One-pot synthesis of macrocycles c5. Pentaamides c5 are new star-shaped macrocycles that cannot be synthesized based on established amide-coupling chemistry. To synthesize c5a, a stepwise approach was first attempted. Using a recently developed amide-coupling strategy, the linear precursor of c5a, prepared by starting from monomer 2a in multiple steps, was cyclized under high-dilution conditions to give c5a in an overall yield of ~24% (Scheme 4). Subsequently, a much more efficient one-pot synthesis of c5a was developed by probing the conditions for the ring-opening reaction of benzoxazinone monomer 2a (FIG. 113b and Scheme 5). In toluene, while heating 2a (2 mM) and 4-(N, N-dimethylamino)pyridine hydrochloride (DMAP·HCl) and DMAP·Hl under reflux led to c5a in 5% and 0% yields, respectively, treating 2a and DMAP·HBr (40 mol %) under the same condition gave c5a in 27% yield, indicating that Br served as a template for one-pot cyclization. Replacing DMAP·HBr with the 1:1 mixture of diphenyl phosphate (DPP) and DMAP (40 mol %) significantly improved the yield (~45%) of c5a, suggesting that DPP is a more effective template than Br. Based on the same one-pot procedure, macrocycles c5b and c5c, which carry sidechains having tri (ethylene glycol) (Tg) and allyl tails, for enhanced solubility and further modification, respectively, were synthesized from monomers 2b and 2c in yields of 38% and 39%. The one-pot macrocyclization process has provided a simple and scalable route allowing further structural tuning and optimization, for instance, by adjusting the side chains and by screening additional anions as templates, based on which a variety of c5 derivatives can be synthesized in sufficient quantities.

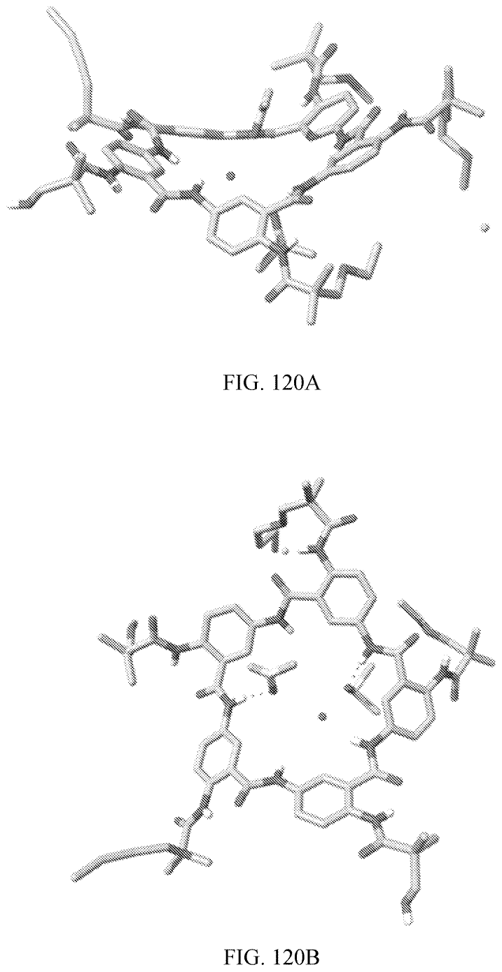

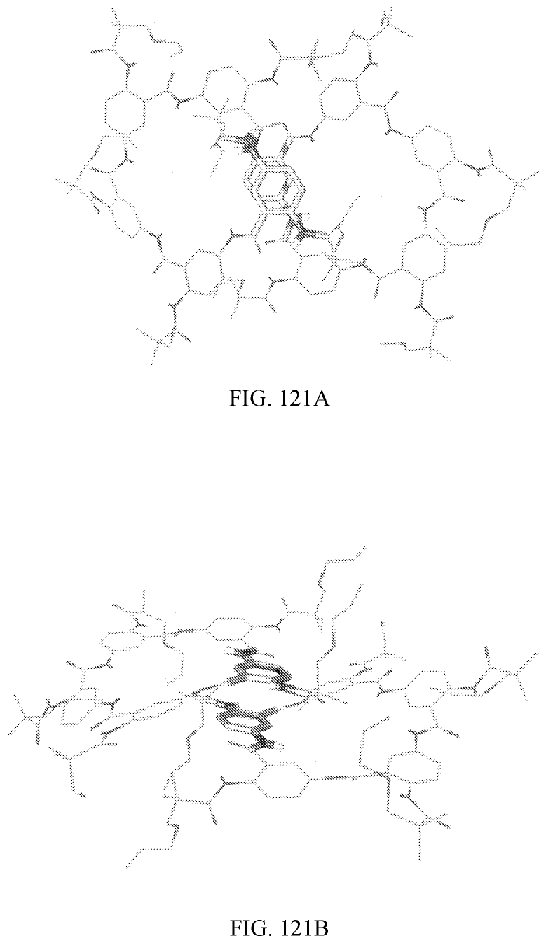

Crystal structures of c5a and c5c, and solution properties of c5a-c. Single crystals of c5a were obtained from DMSO by slow cooling and those of c5c from CHCl₃:CH₃CN (1:1, v/v) by slow evaporation of solvent. The crystal structures of c5a (FIG. 120) and c5c (FIG. 113c) validate our design. Both c5a and c5c have overall planar conformations that resemble the energy-minimized structure of c5 (FIG. 118a). Similar to the CH and NH groups constituting the selectivity filter of CIC Cl⁻ channels, all of the endocyclic NH and CH groups of c5c are oriented toward the center of the macrocycle (FIG. 113c) and defines a cavity of ~6.8 Å (H to H) or ~4.6 Å (van der Waals). Dihedral angles of 0° to 40° are observed between the backbone amide groups and their flanking benzene rings, leading to a slightly puckered conformation that presumably alleviates the repulsion between the amide dipoles and between the partially positive endocyclic hydrogens. The intramolecular hydrogen bonds also enforce the coplanar alignment of the side chain amide groups and the benzene rings to which they are attached. In addition, three water molecules are hydrogen-bonded to amide protons b in the cavity. The crystal structure of c5a (FIG. 120) shows the similar features with all of its endocyclic CH and NH groups being convergently placed. The backbone of c5a is more puckered than that of c5c, which is probably caused by the different crystal packing due to the sidechains. In the cavity of c5a, two DMSO molecules are hydrogen-bonded to two NH groups. Examining the packing of c5c in the solid state indicates that any two adjacent macrocycles have only a small portion, about one residue, of their backbones being stacked at ~3.4 Å (FIG. 121). Such a small contact surface is likely caused by the need to avoid the repulsive interactions between the amide dipoles that would arise should the macrocycles stack face-to-face.

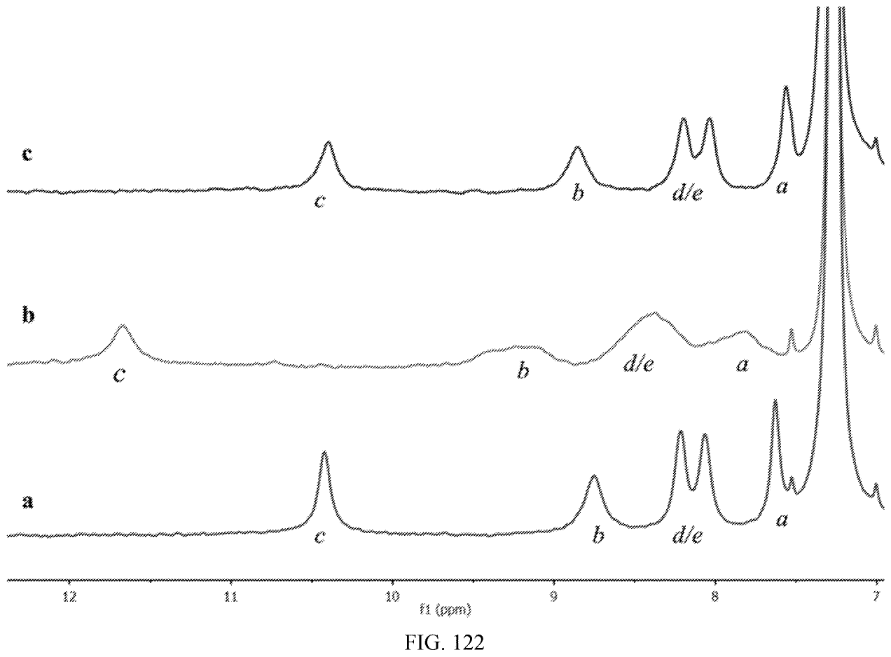

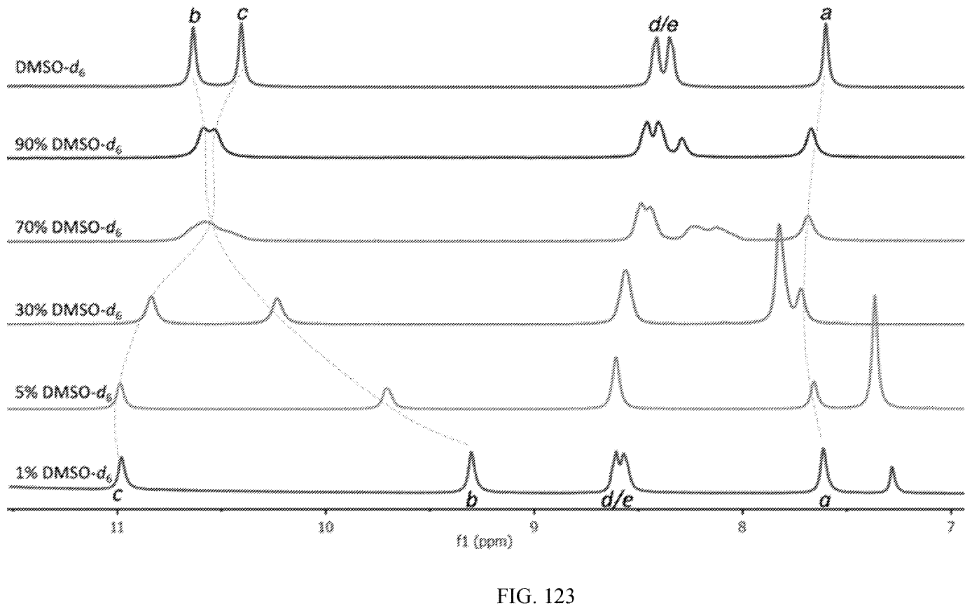

The lack of strong stacking interactions between the macrocyclic molecules is reflected by the overall good solubility of macrocycles c5a,b in solvents such as chloroform. which contrasts with the poor solubilities typical of most acyclic and cyclic aromatic oligoamides. In fact, macrocycle c5a is soluble in CHCl₃, CH₂Cl₂, DMF, and DMSO. With its polar side chains having the tri (ethylene glycol) tails, macrocycle c5b is not only soluble in many organic solvents, but also in water with a reasonable solubility (~1 mM). The 1H NMR spectra of macrocycles c5a-c in CDCl₃ (0.2 mM) reveal dispersed or discernible resonances (FIG. 122), lending further support for the limited self-aggregation of these compounds as indicated by the crystal structures. In CDCl₃ with 1% to 100% (v/v) DMSO-d₆, the NMR signals of c5a (1 mM) are well dispersed, with the resonance of sidechain amide protons c showing an upfield shift of 0.5 ppm, backbone amide protons b exhibiting a large downfield shift of 1.41 ppm, and that of the endocyclic CH proton a experiencing a very small downfield shift of 0.06 ppm (FIG. 123). These observations demonstrate that amide protons b are exposed to solvent while endocyclic phenyl protons a have minimal, if any, interaction with DMSO.

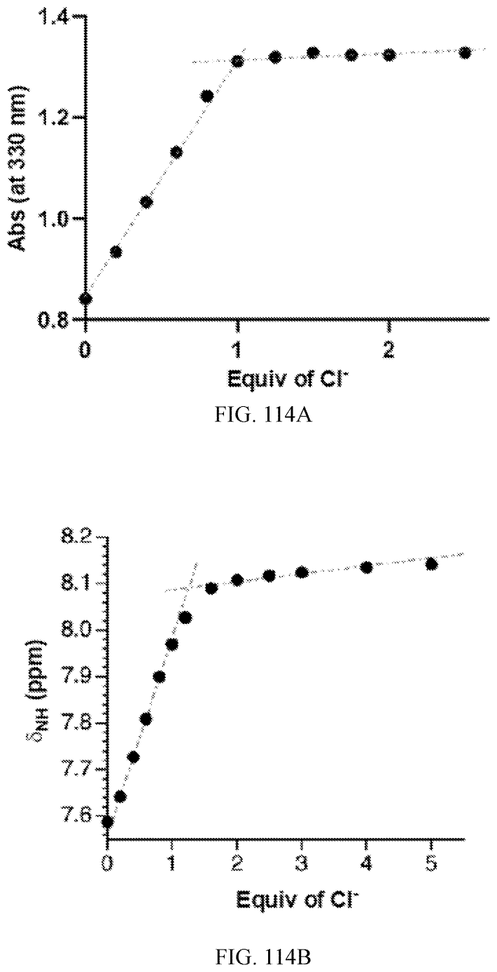

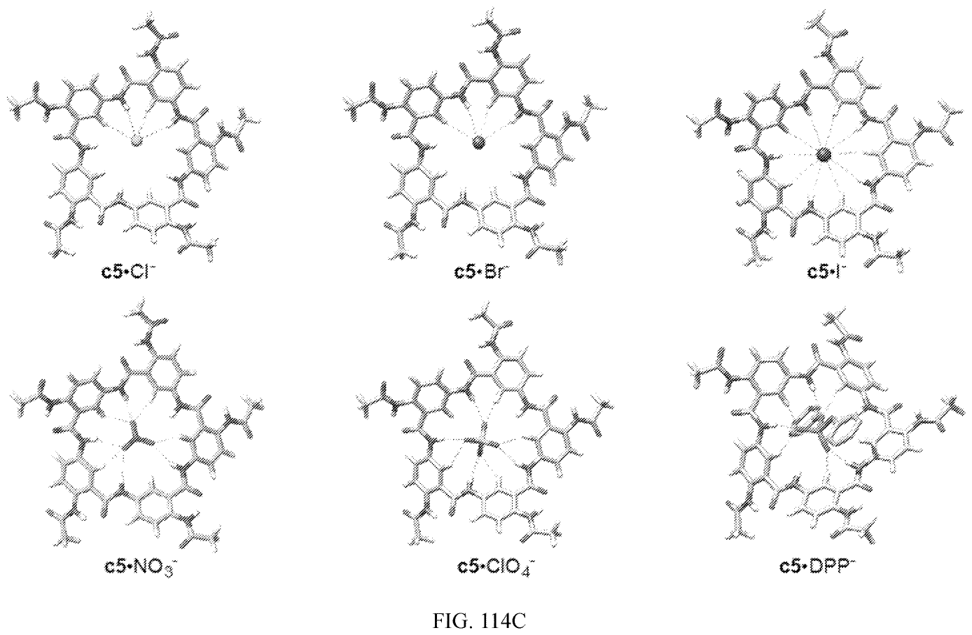

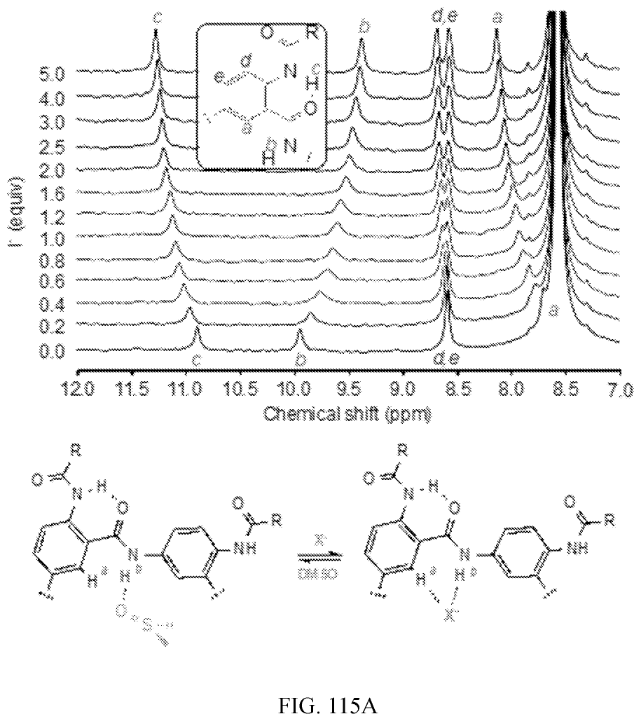

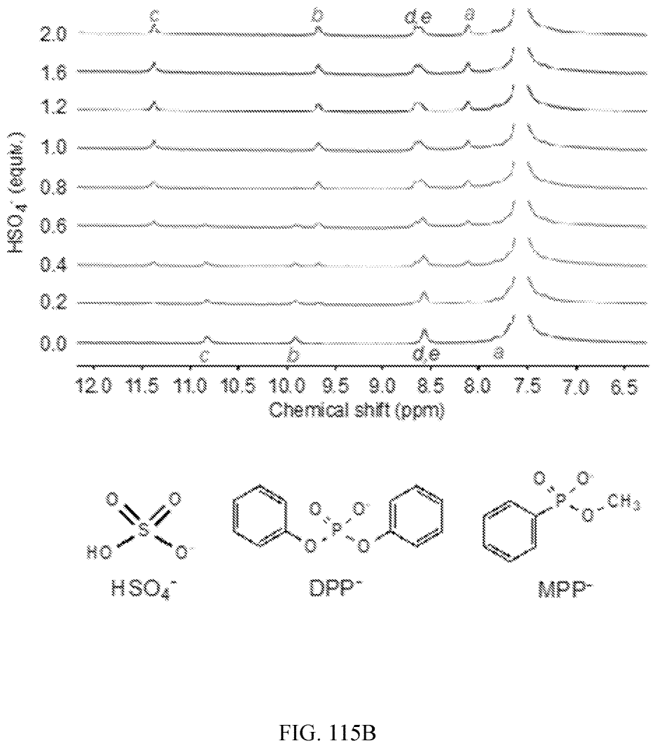

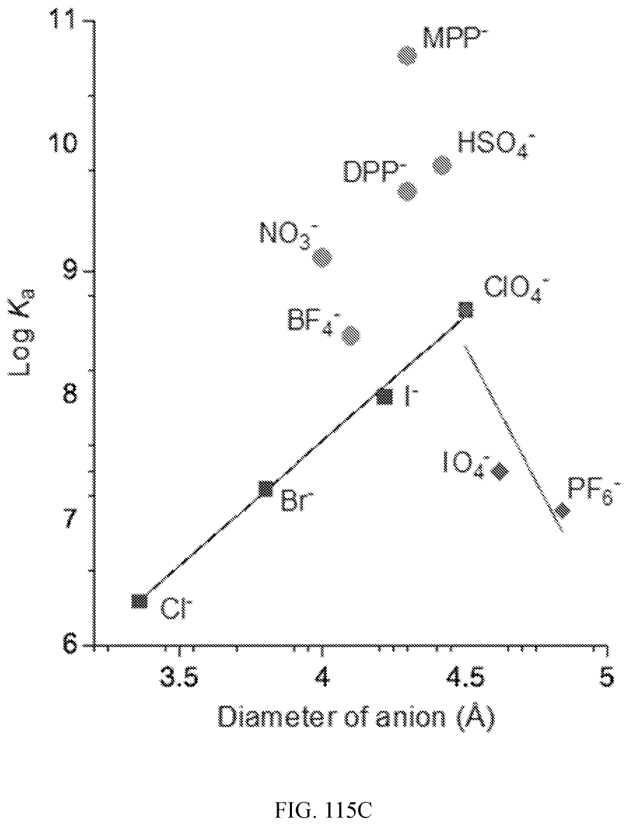

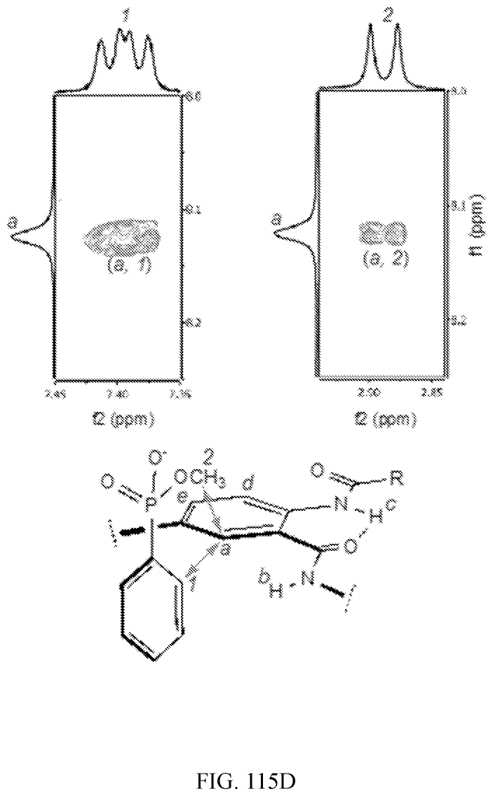

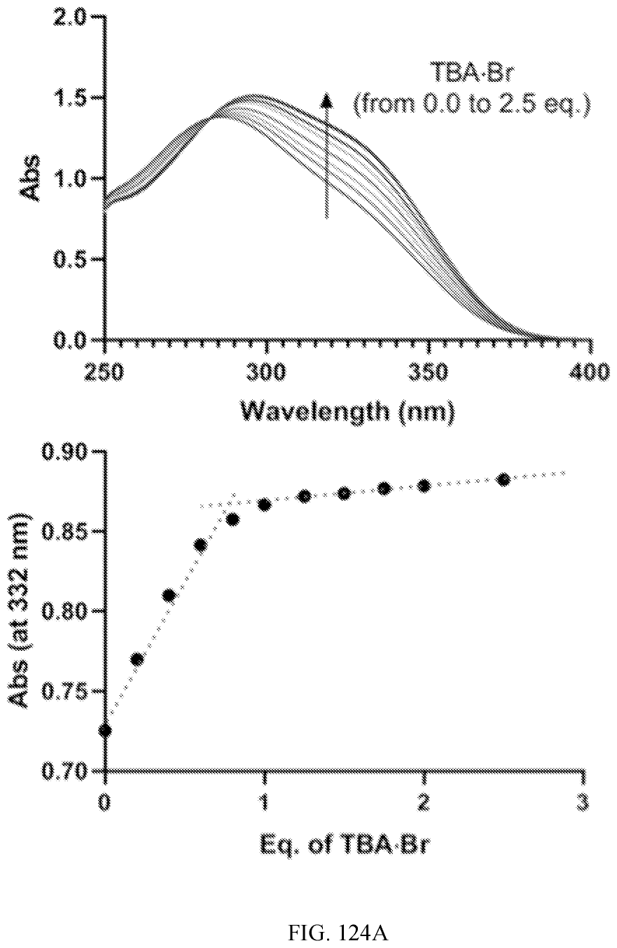

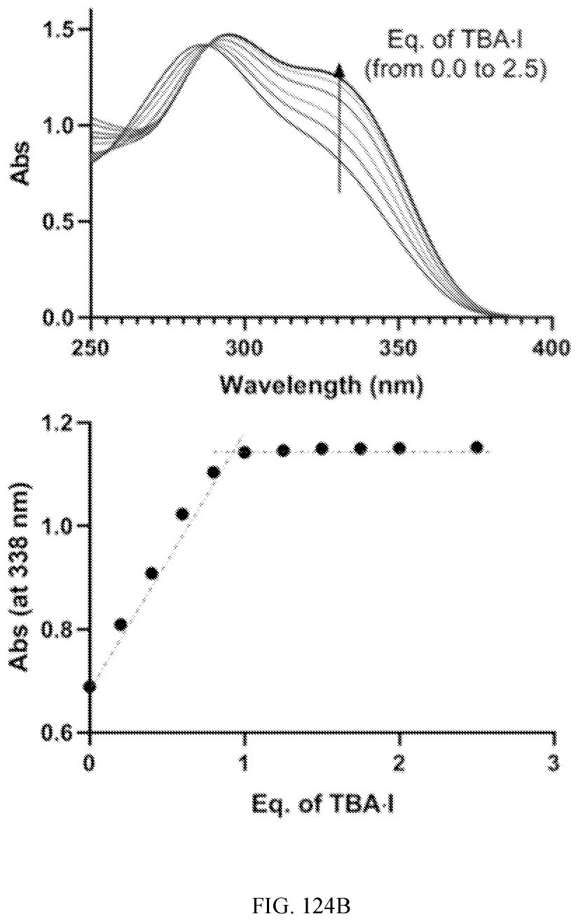

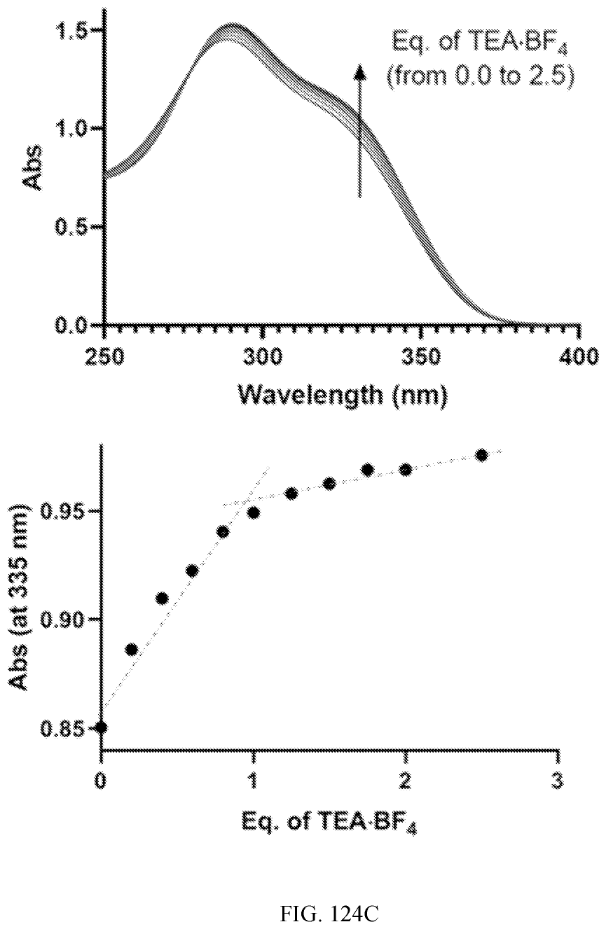

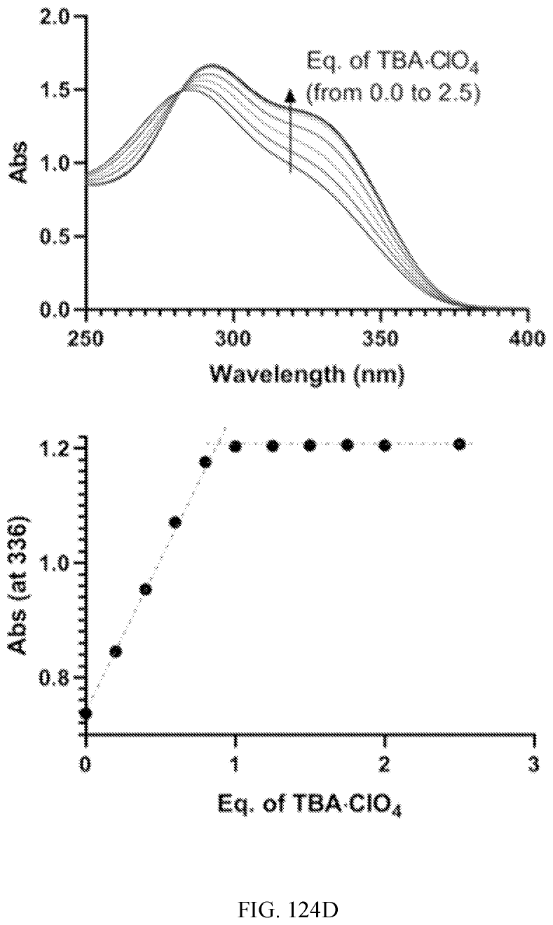

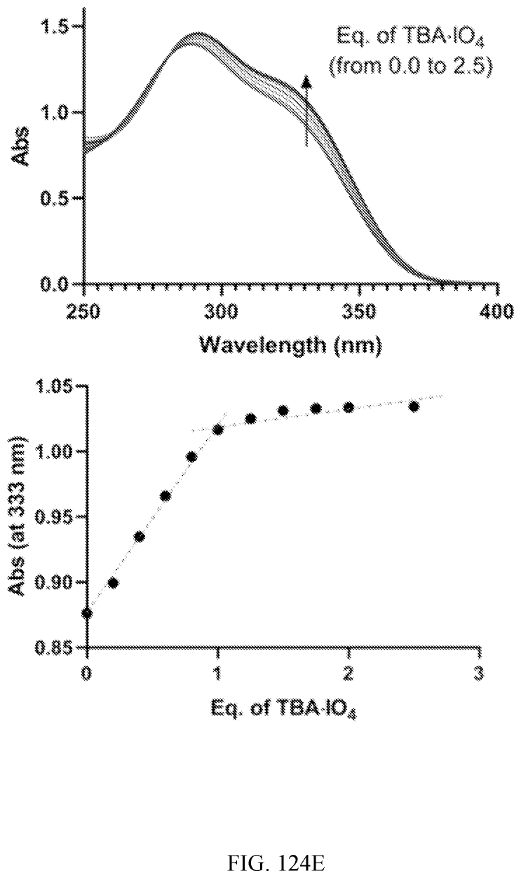

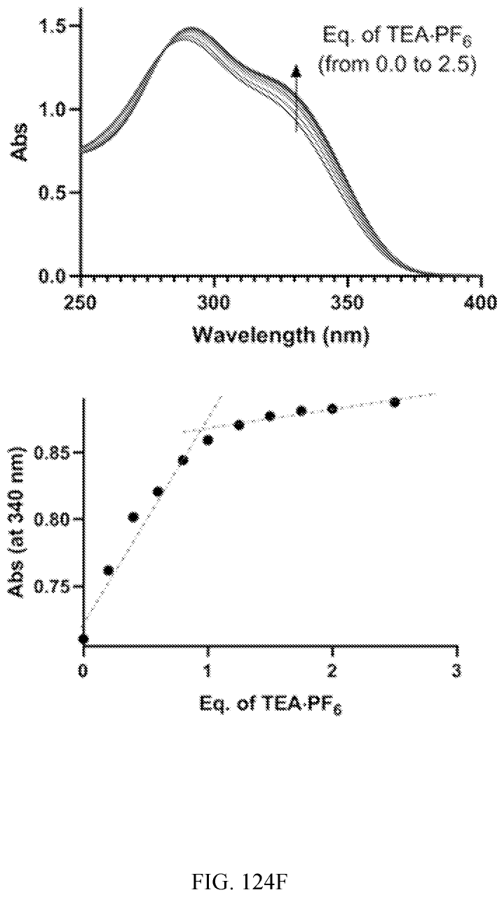

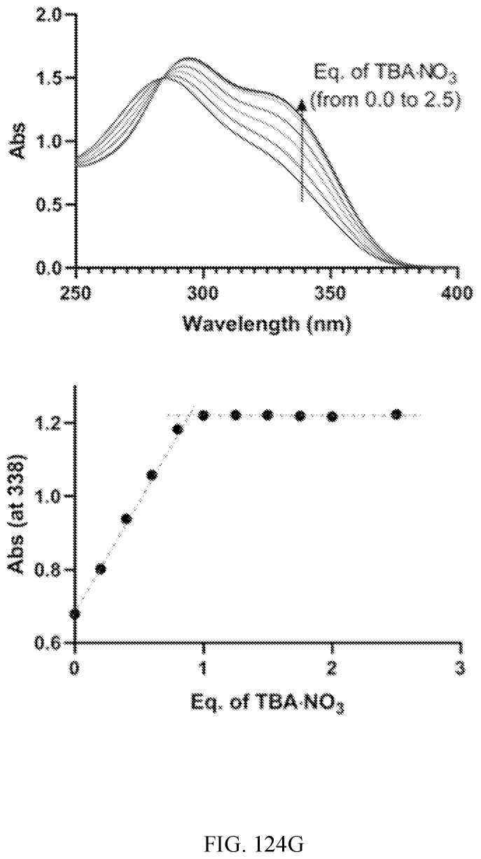

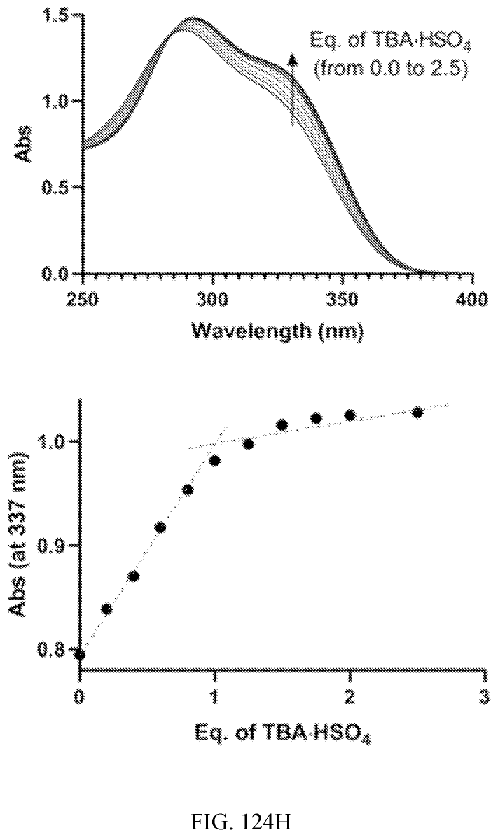

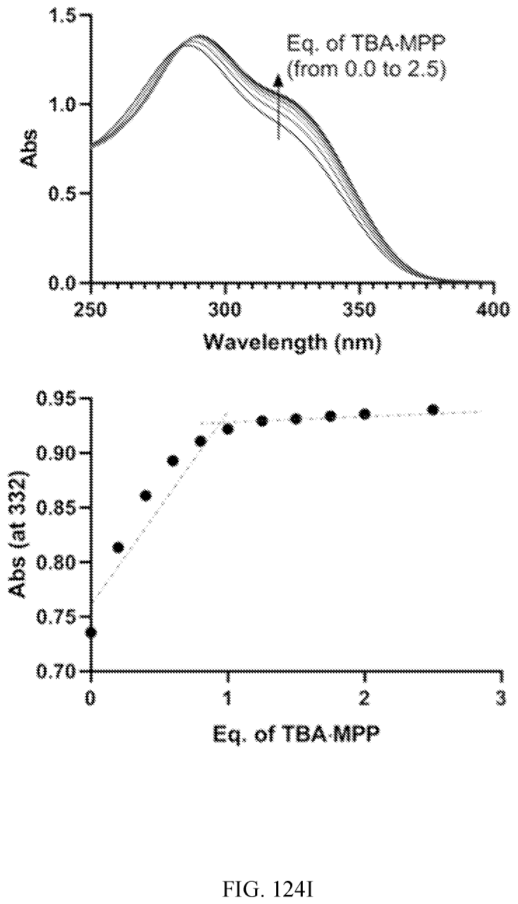

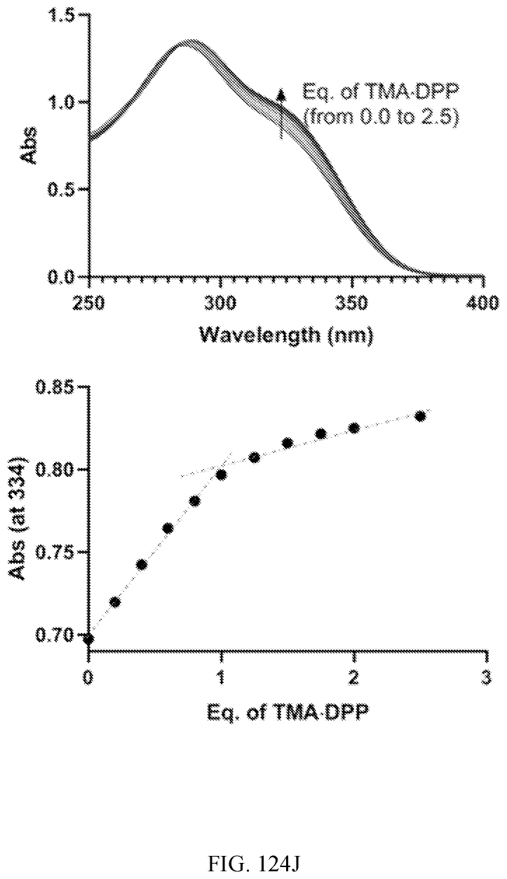

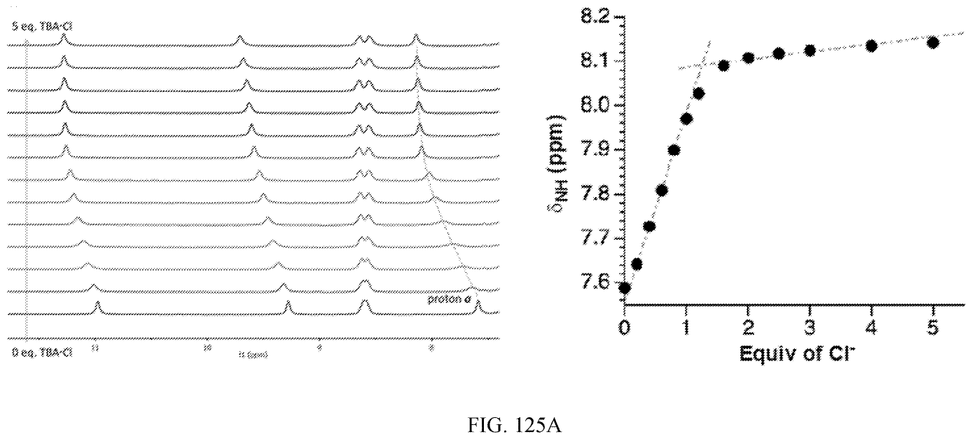

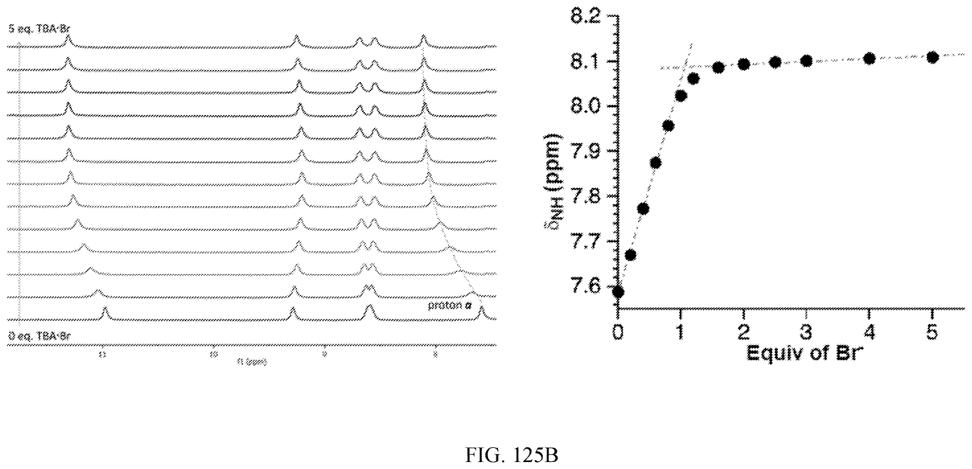

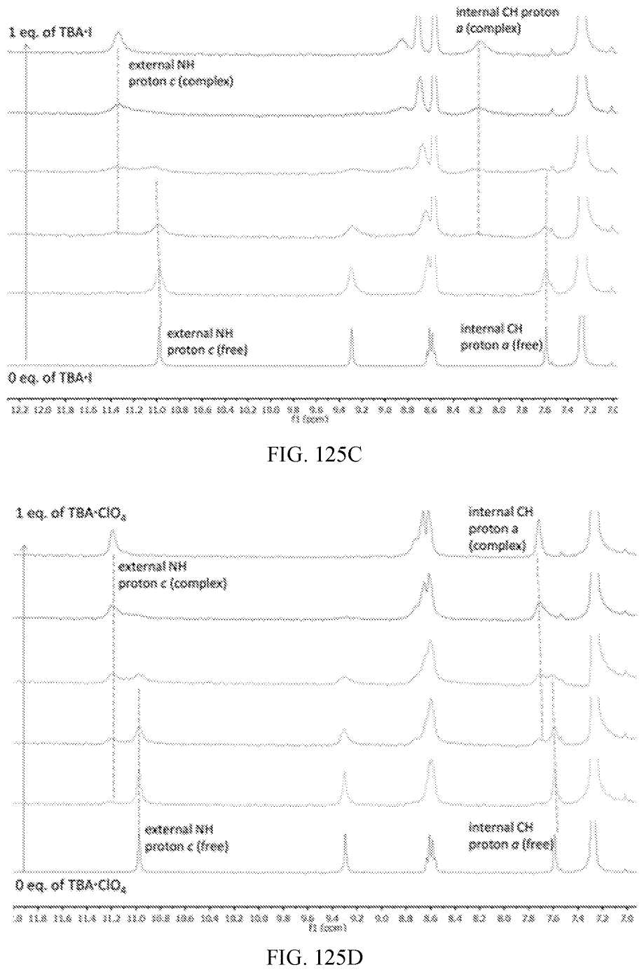

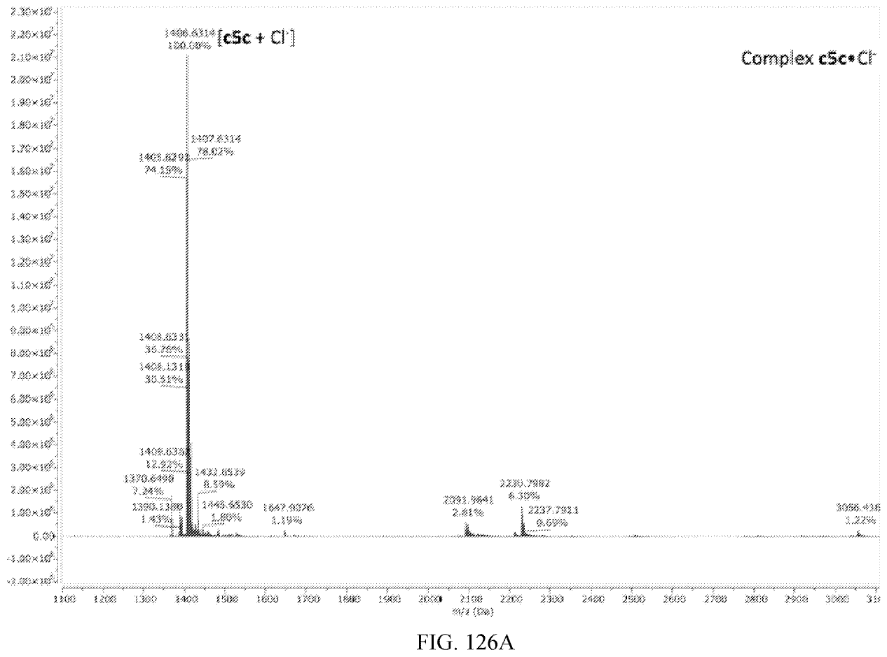

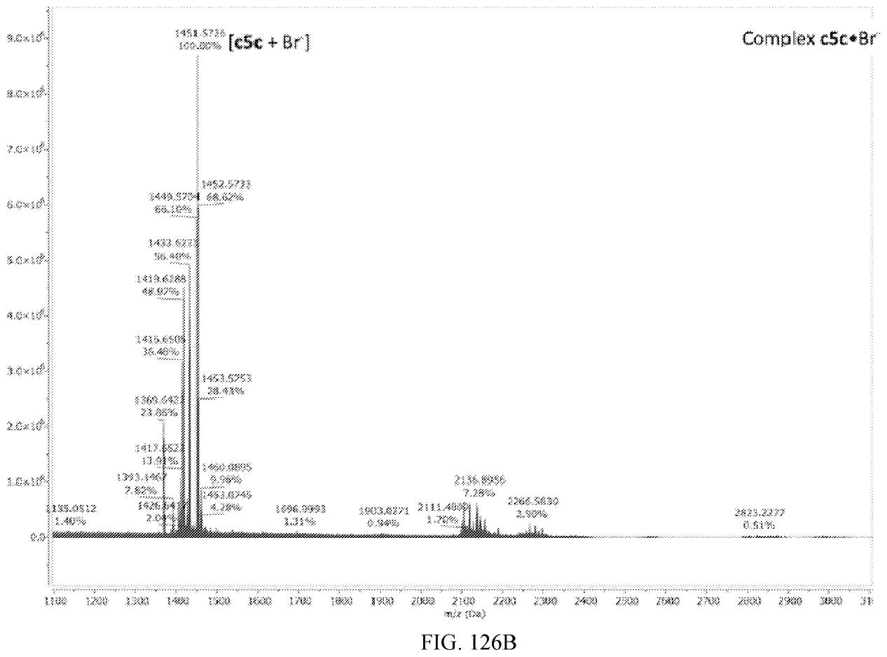

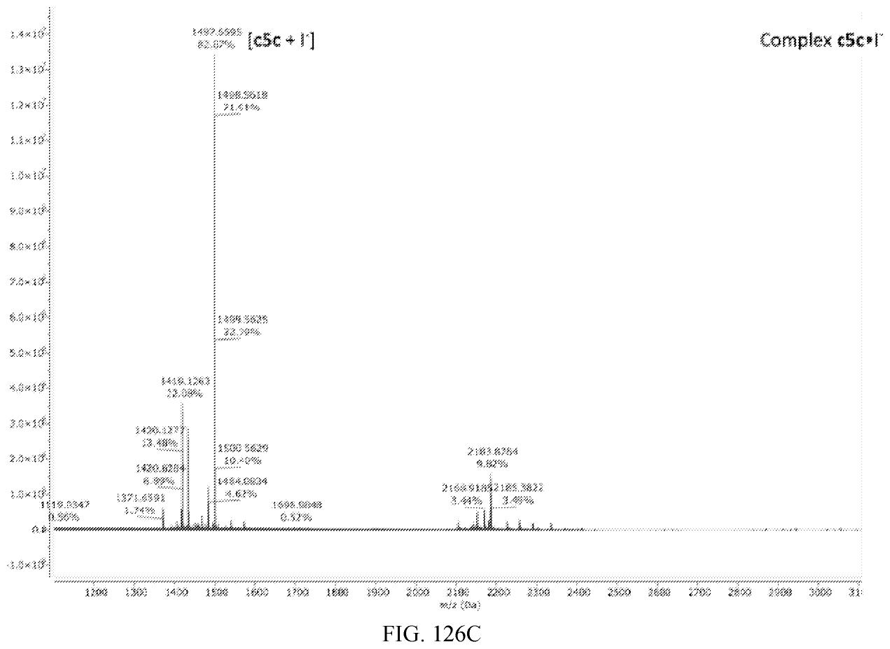

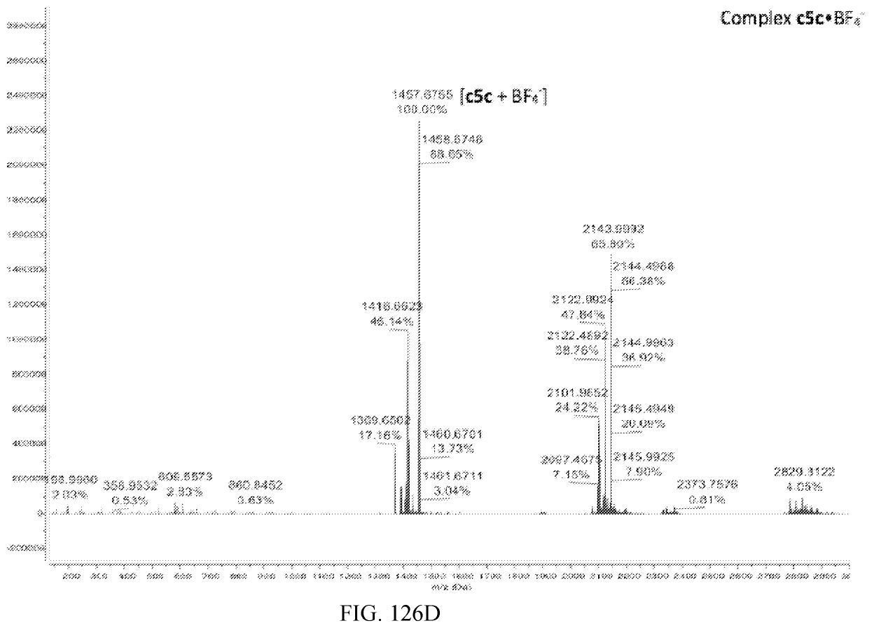

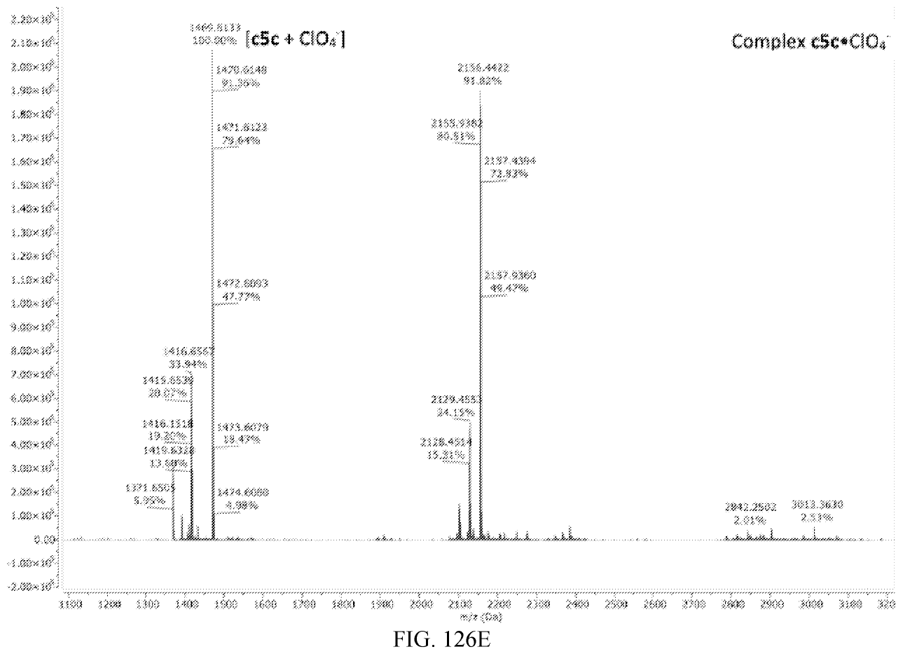

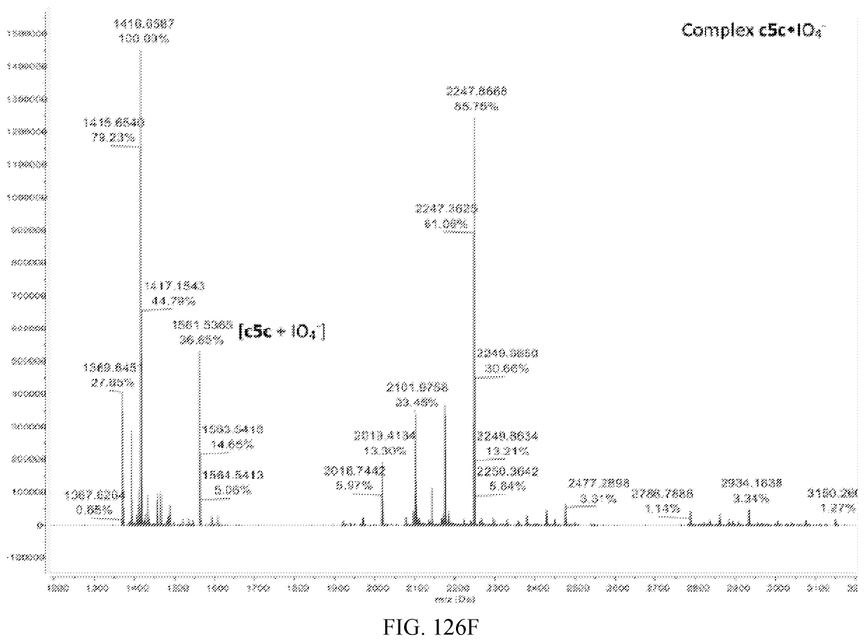

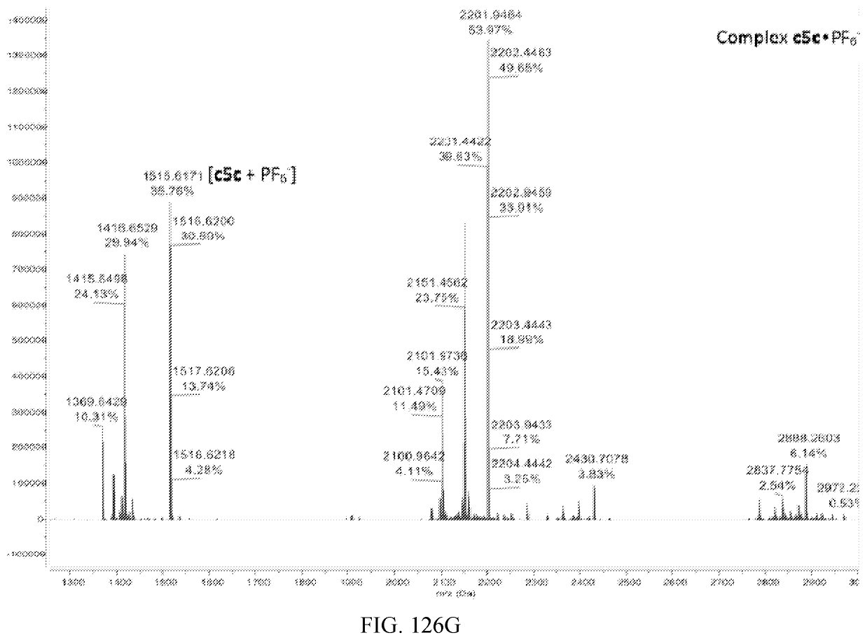

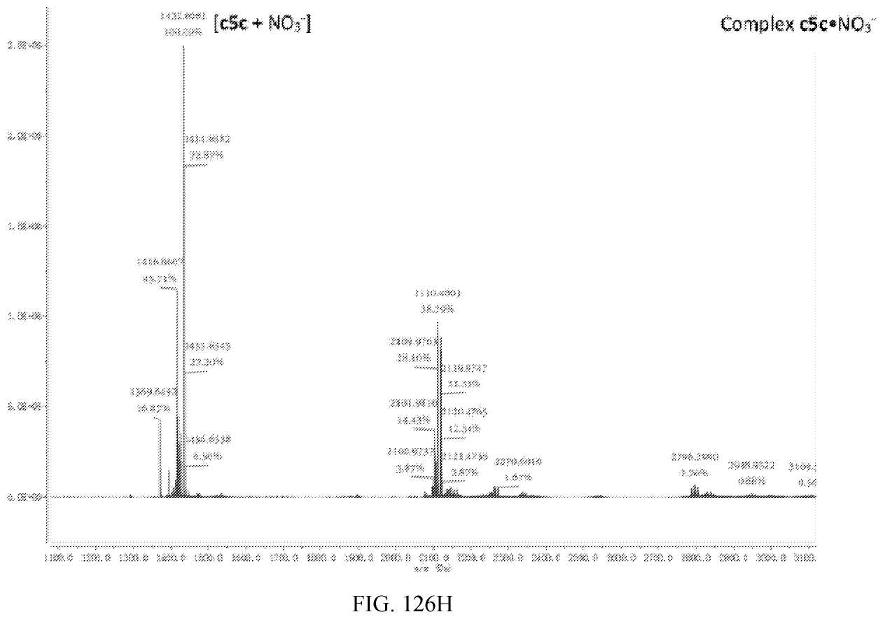

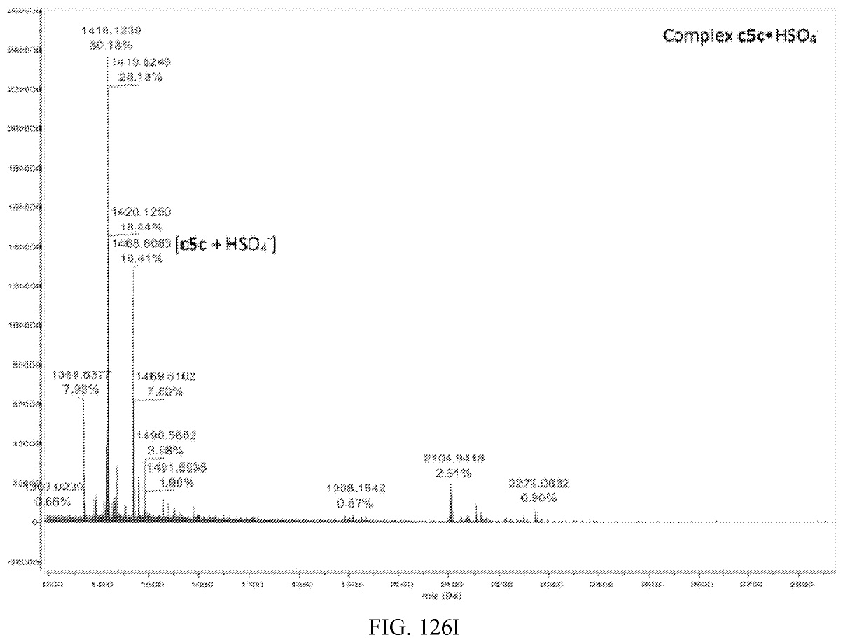

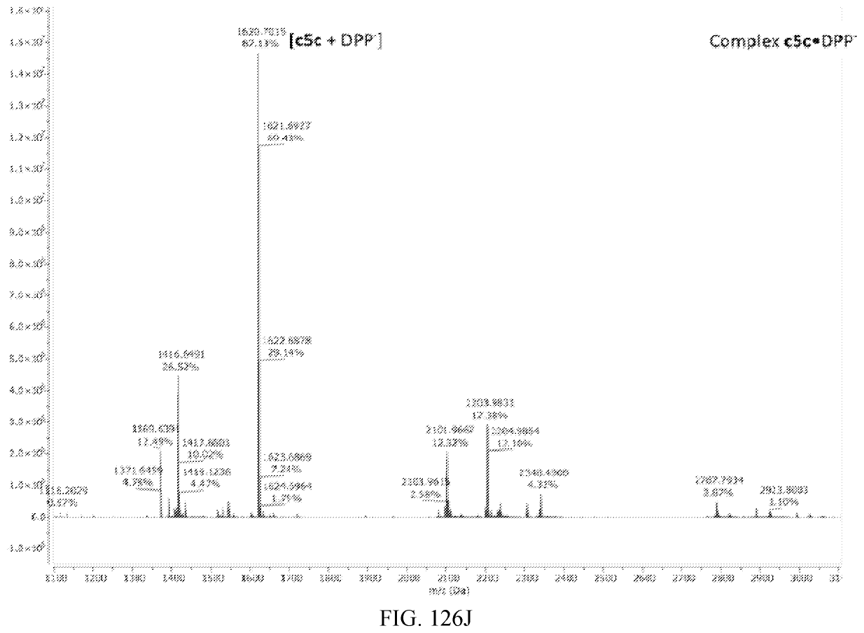

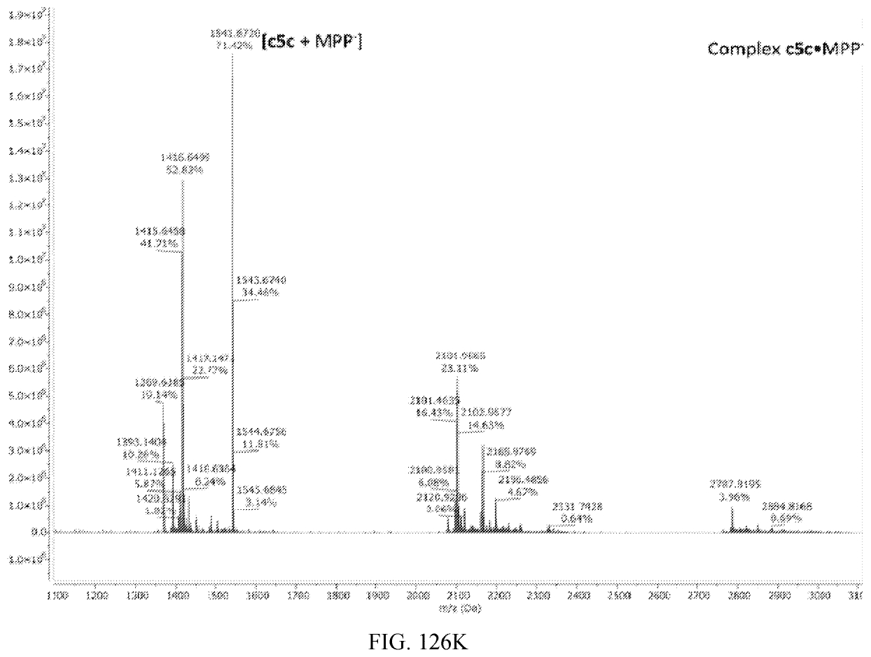

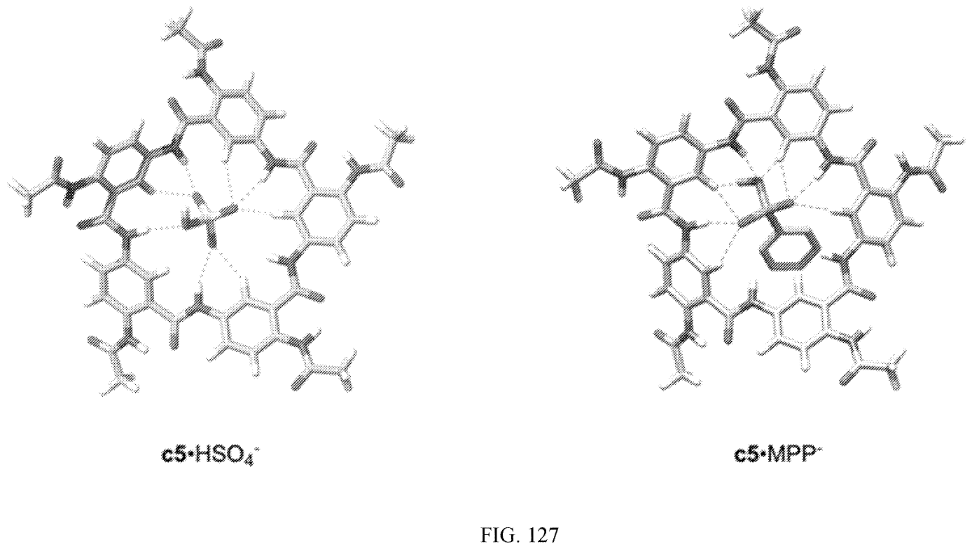

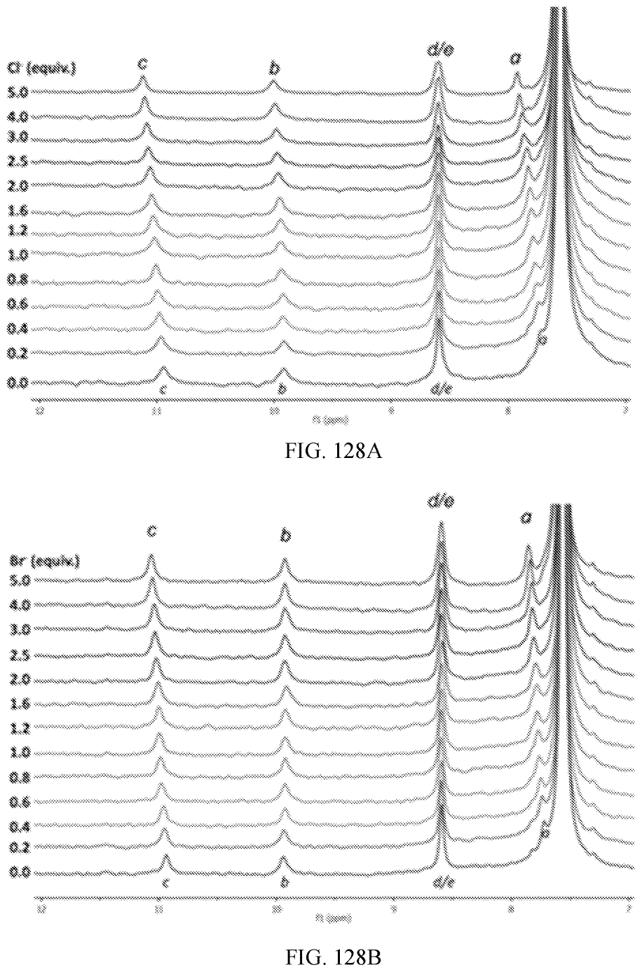

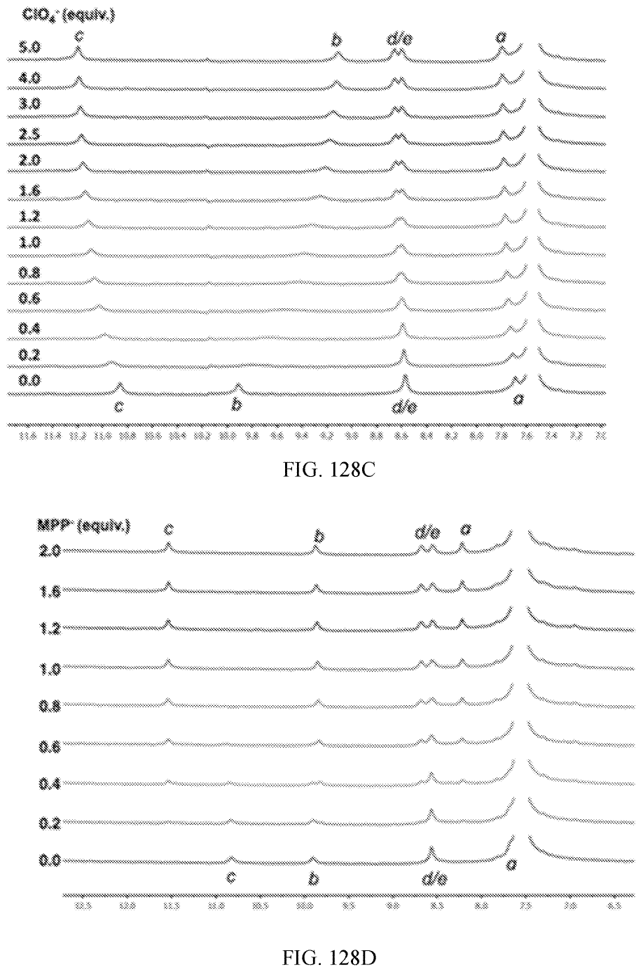

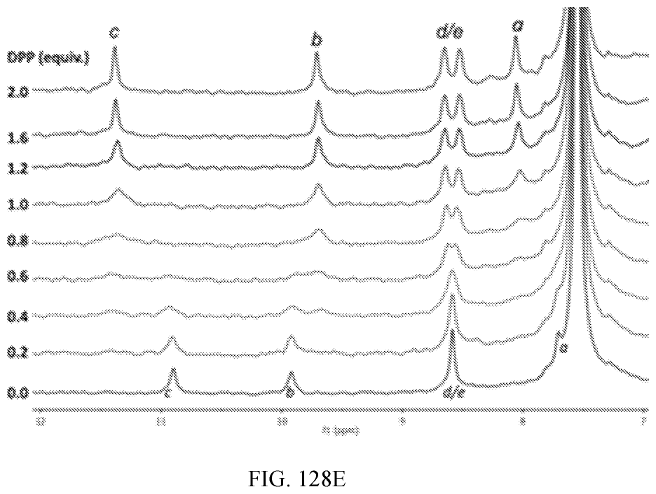

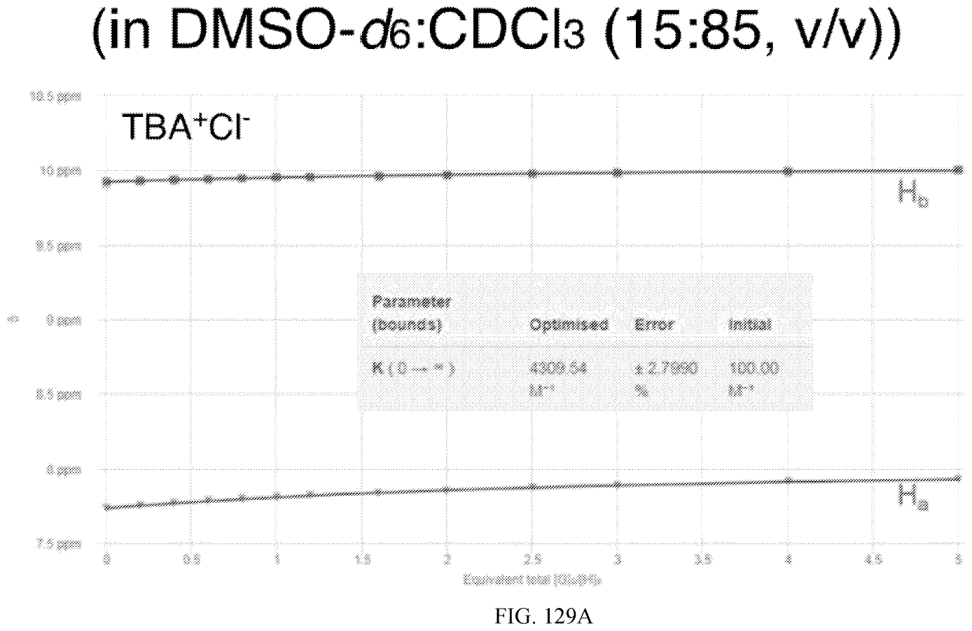

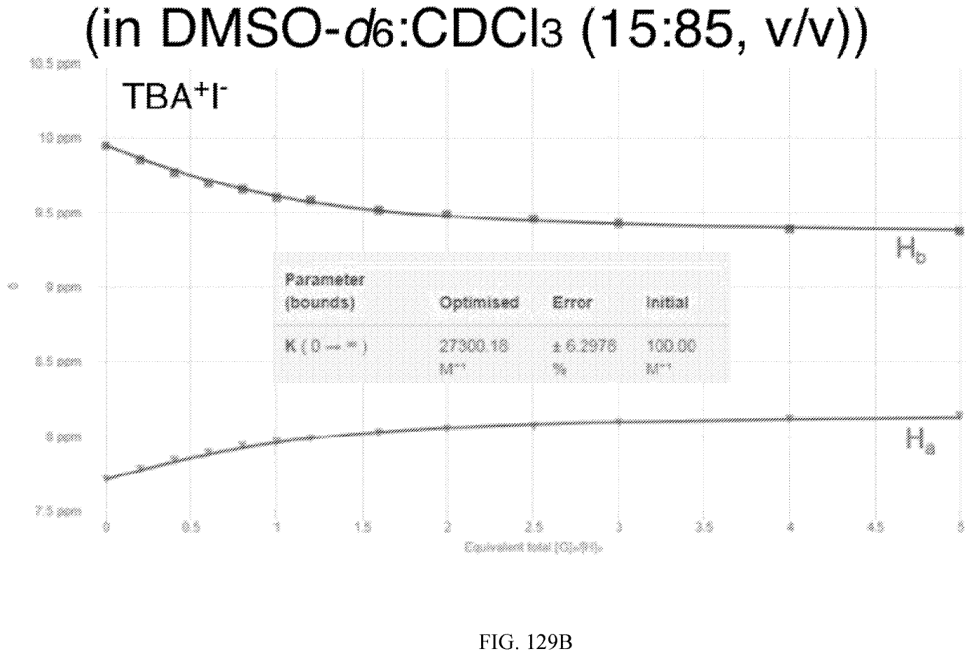

High-affinity anion recognition by macrocycles c5. Macrocycles c5, with their backbone NH and CH groups being highly preorganized, may serve as anion receptors capable of accommodating anions of markedly different properties. Eleven anions including halides Cl⁻, Br⁻, and I⁻, the weakly coordinating BF₄ and octahedral PF₆⁻, oxoanions including NO₃⁻, ClO₄, 104, HSO₄, methyl phenylphosphonate (MPP⁻), and diphenyl phosphate (DPP⁻), with a variety of sizes, shapes, charge densities and hydrogen-bonding capabilities, were examined for their interaction with macrocycles c5.
(i) Binding stoichiometry of macrocycles c5 with anions. The binding stoichiometry of macrocycles c5a and c5c with the anions were analyzed with UV-vis titrations and ¹H NMR titrations in solvents in which macrocycles c5 strongly bind the anions, and with high-resolution mass spectra (HRMS) recorded in negative ion mode. FIG. 114A shows the UV-vis absorbance (at 330 nm) of c5a titrated with tetrabutylammonium chloride (TBA Cl⁻). The linear dependence of the absorbance of c5a changes abruptly at one equiv of TBA⁺Cl⁻, consistent with the 1:1 binding between c5a and C₁. Titrating c5a with the other anions revealed the same abrupt change in absorbance around 330-nm at one equiv of each anion (FIG. 124), indicating that c5a binds with the anions in a 1:1 ratio. In CDCl₃ containing 5% DMSO-d₆, the changes in the chemical shifts of phenyl CH protons a of c5a upon titrating with 0-5 equiv of TBA⁺Cl⁻ exhibit the same abrupt change around one equiv of TBA⁺Cl⁻ (FIGS. 114b and 125a) or TBA⁺Br⁻ (FIG. 125b), confirming the 1:1 binding of c5a and Cl⁻ or Br⁻. In the same solvent, titrating c5a with I⁻ or ClO₄⁻ gave two separate sets of 1H NMR signals indicating slow exchange between the free and bound forms of c5a (FIGS. 125c, d). Consistent with the 1:1 binding between c5a and I⁻ or ClO₄⁻, with ≥1 equiv of the anion salt, the signals of free c5a completely disappear, while those of the bound c5a remain. The 1:1 binding ratio is further demonstrated by the HRMS of the mixtures of macrocycle c5c and all eleven anions (FIG. 126). Macrocycle c5c was used since it has a molecular weight lower than c5a, which allows HRMS to be recorded in the detection range of m/z values of the mass spectrometer. In each case, the HRMS reveals the 1:1 complex as the only or dominant species. In contrast, the 2:1 complex was not detected with any of the anions.
(ii) Computationally optimized structures of 1:1 complexes. The 1:1 complexes of c5 with the spherical halides, planar NO₃⁻, tetrahedral ClO₄⁻ and the substituted phosphate ion DPP⁻ (FIG. 114c), along with HSO₄ and MPP⁻ (FIG. 127), were optimized. Iodide locates at the center of the macrocycle and is within van der Waals contact distances with all endocyclic CH and NH hydrogens. In contrast, C₁ or Br occupies only part of the cavity and is within the van der Waals contact distances with two CH hydrogens and two NH hydrogens. With its slightly flexible backbone, macrocycle c5 is also able to accommodate the different shapes of NO₃⁻, ClO₄⁻, DPP⁻, HSO₄ and MPP⁻ by having multiple contacts between each anion and the endocyclic NH and CH groups, with each bound anion engaging in multiple NH . . . X⁻ and four CH . . . X⁻ interactions.
(iii) Anion-binding affinities of macrocycles c5. The binding of c5a with eight anions shown in Table 3 was first examined. In DMSO-d₆:CDCl₃ (15:85, v/v), titrating c5a with each of the three halides caused phenyl protons a to shift downfield by 0.20 ppm with Cl⁻, 0.13 ppm with Br, or 0.43 ppm with I⁻ (FIG. 115a, top and 128). Compared with protons a, the signal of amide protons b shifts very differently (FIGS. 115a and 128). C₁ induces a small (0.08 ppm) downfield shift, while Br causes a minuscule (−0.02 ppm) and I⁻ a much larger (−0.57 ppm) upfield shift. The shifts of protons b induced by different halides are likely owing to the competition between DMSO and the halides for hydrogen bonding. Binding a halide to c5a expels the DMSO molecules from the cavity (FIG. 115A, bottom). Binding Cl⁻ leads to stronger hydrogen-bonding strength “felt” by protons b, causing the 1H resonance to shift further downfield. On the other hand, displacing DMSO by Br and especially I⁻, as weaker hydrogen-bond acceptors, results in reduced hydrogen-bonding strengths sensed by protons b, leading to upfield shifts.

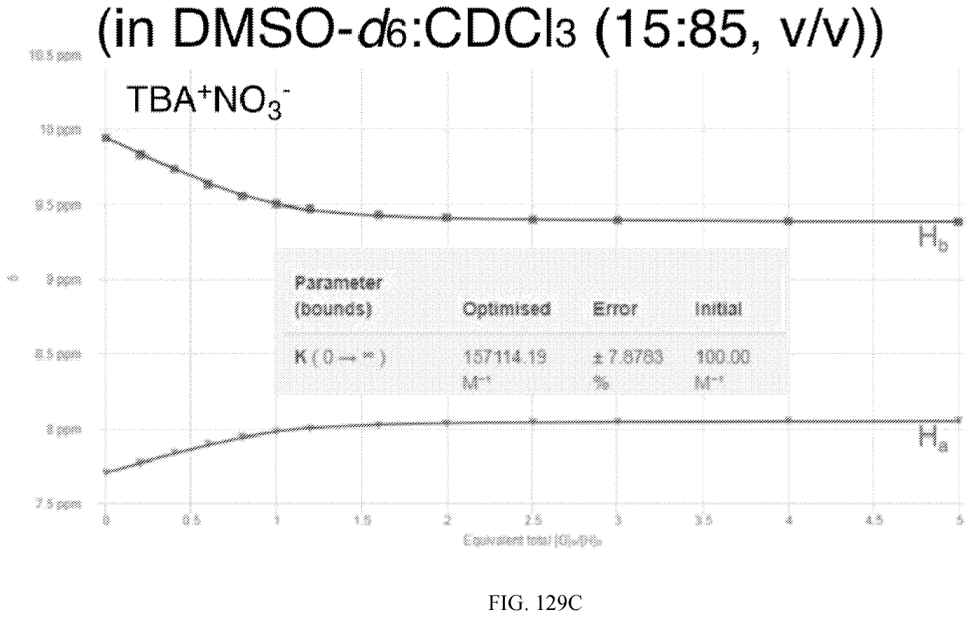

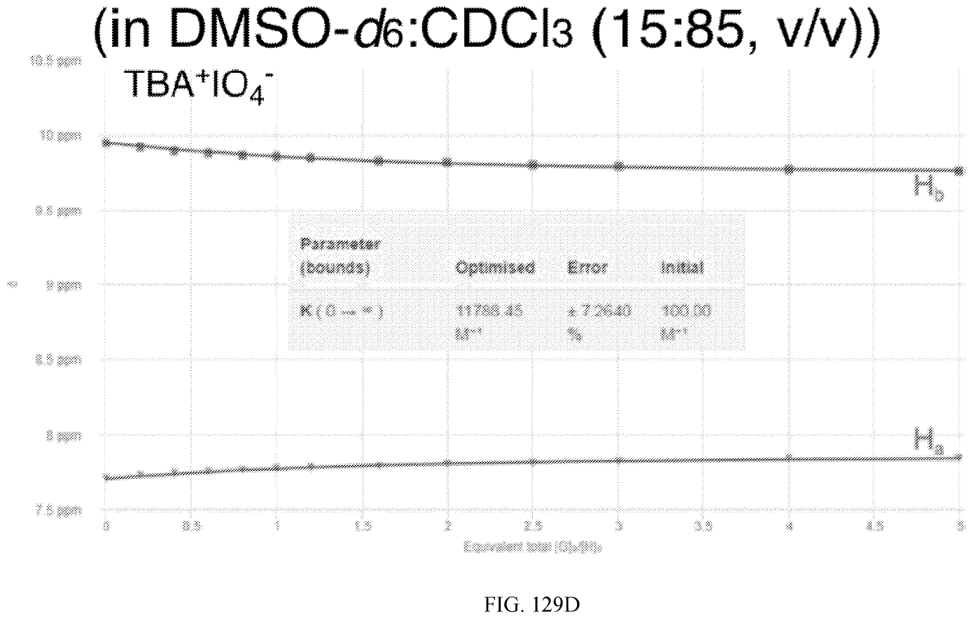

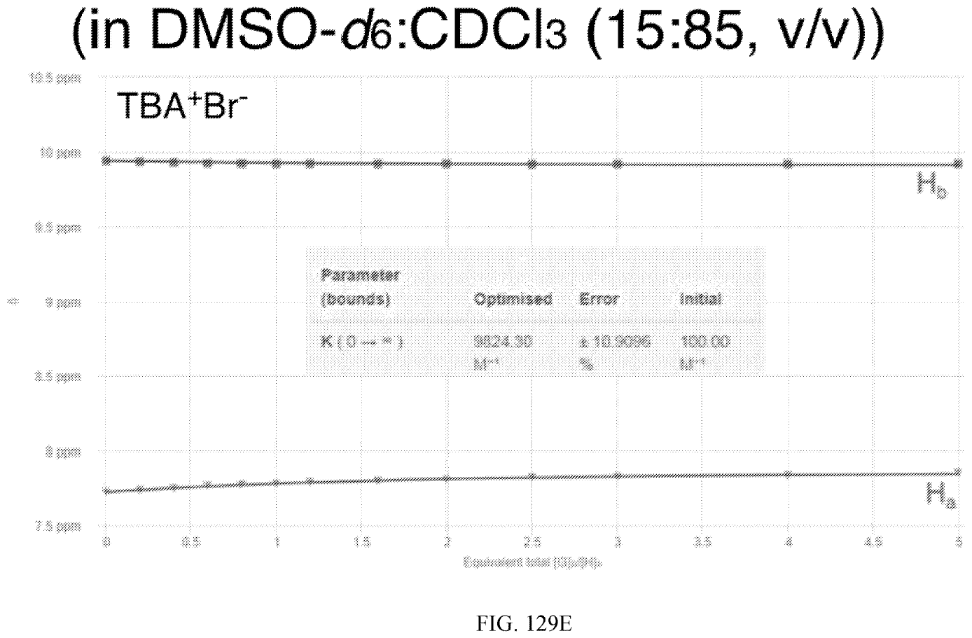

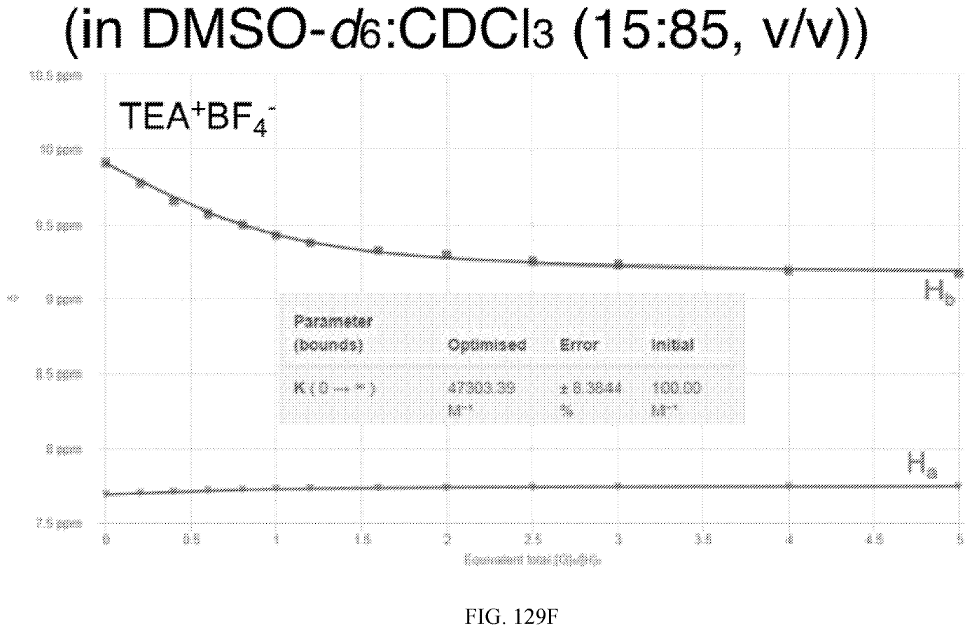

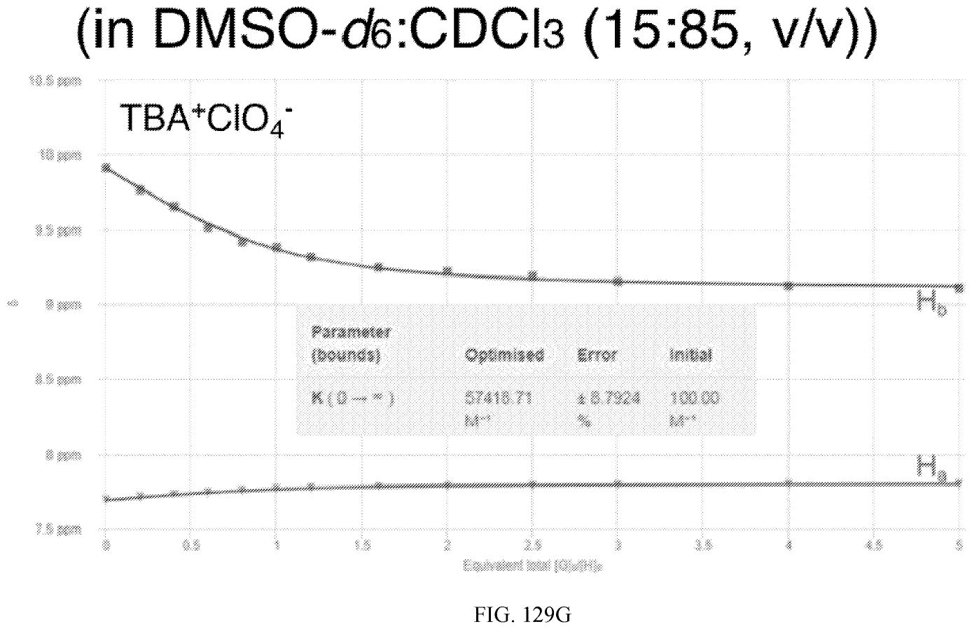

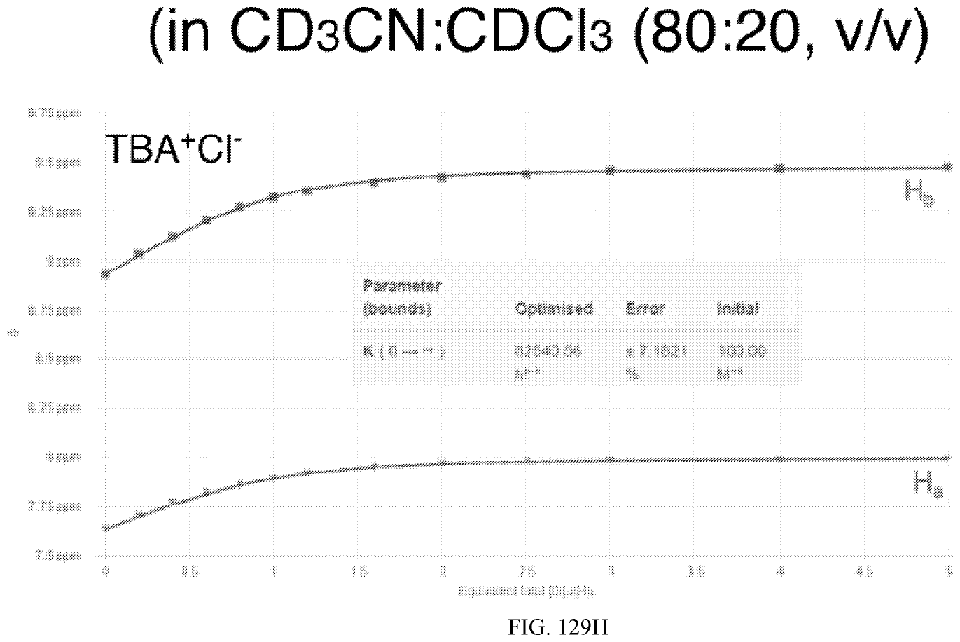

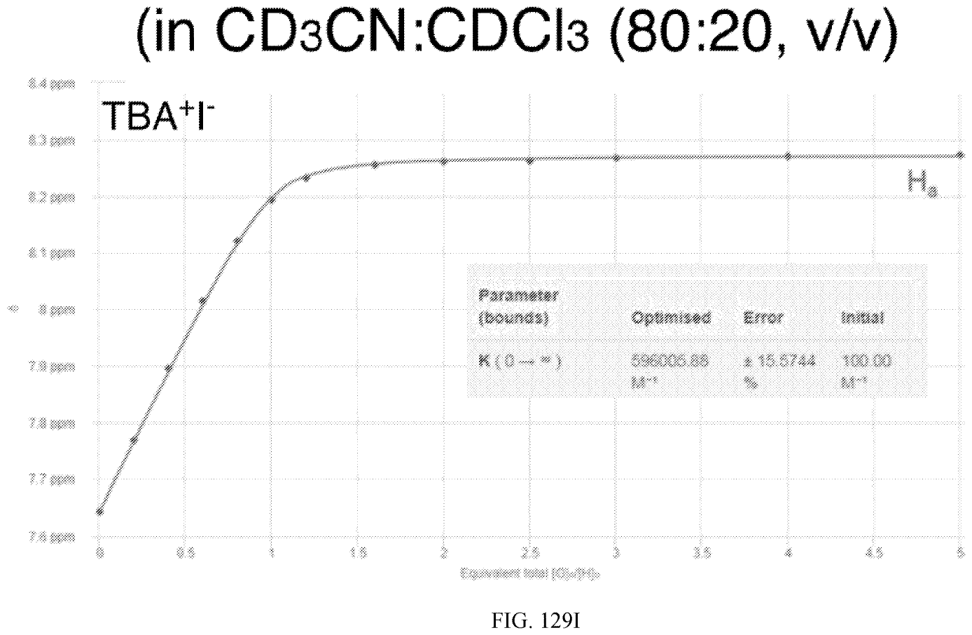

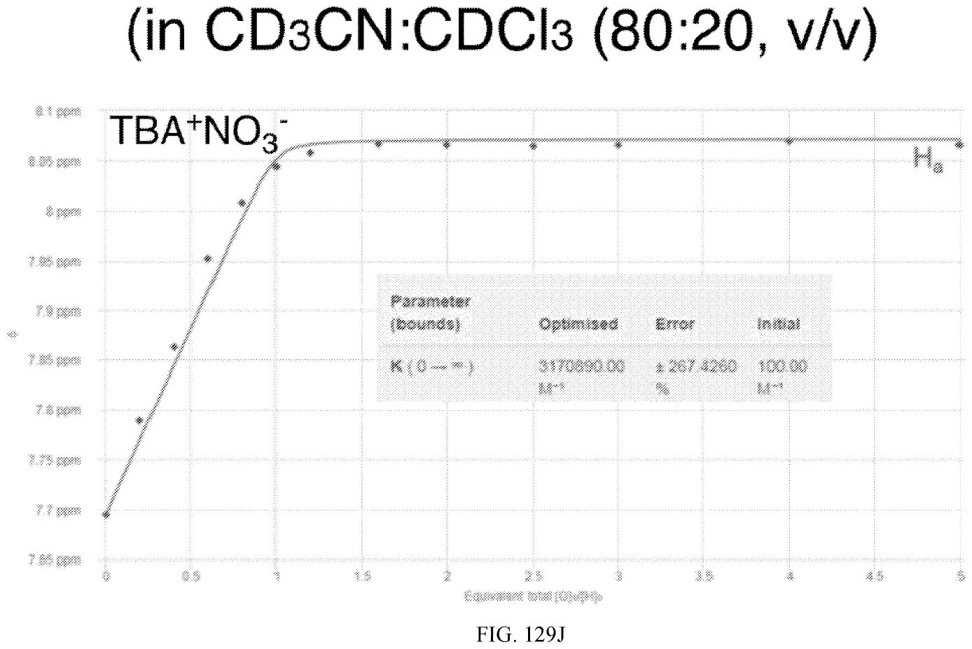

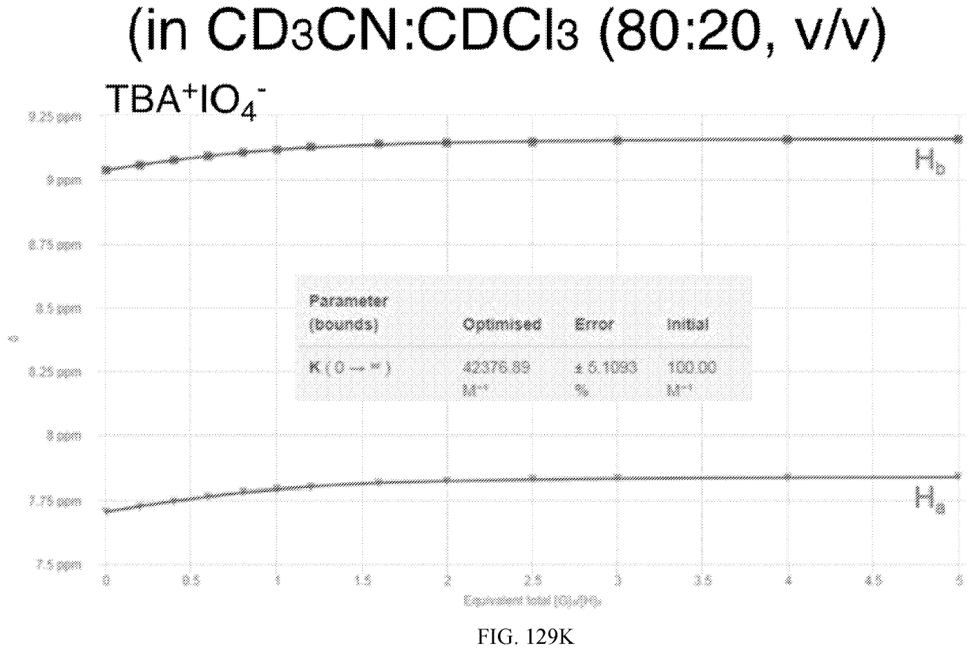

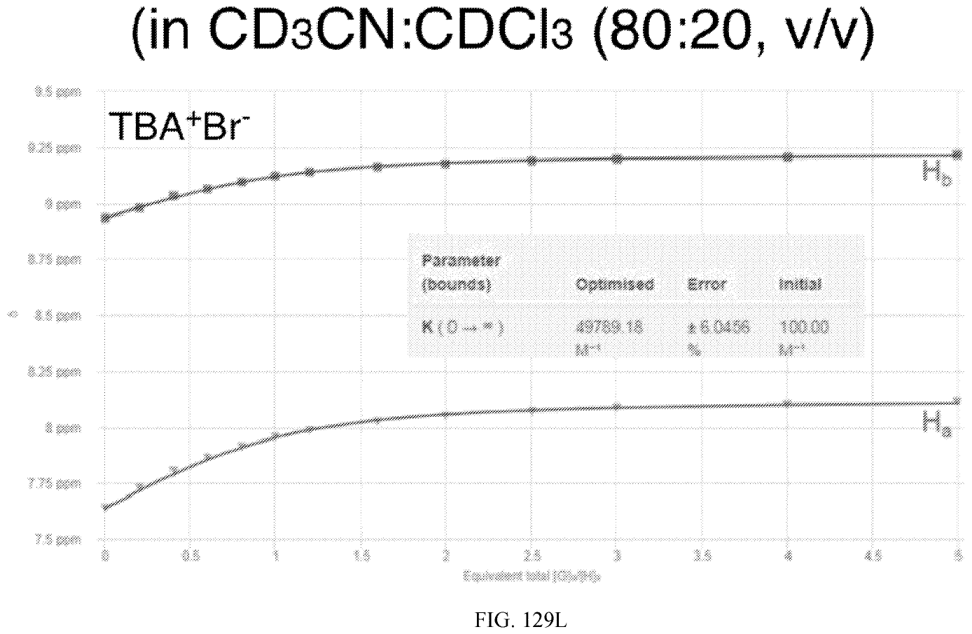

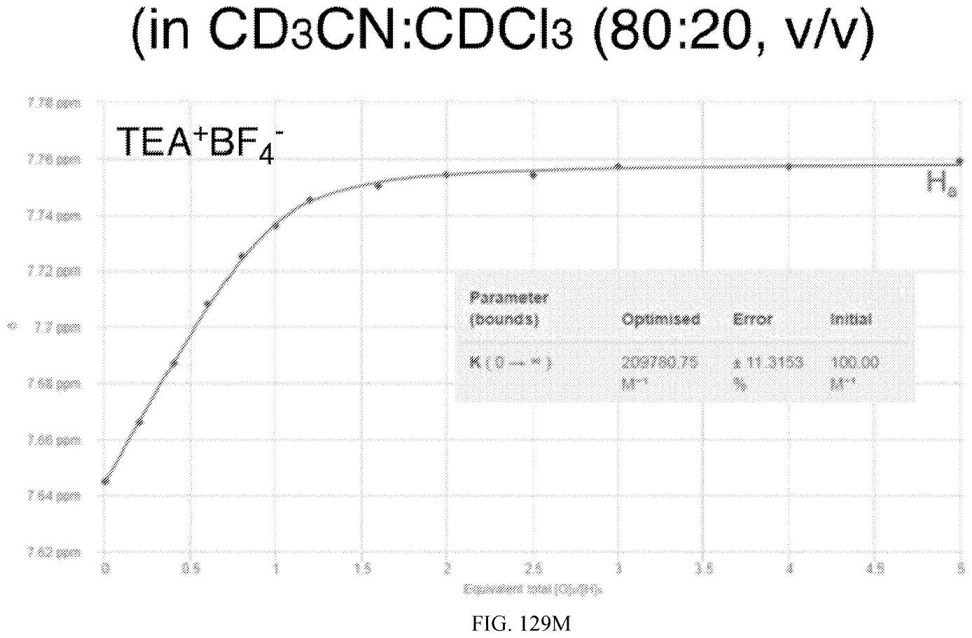

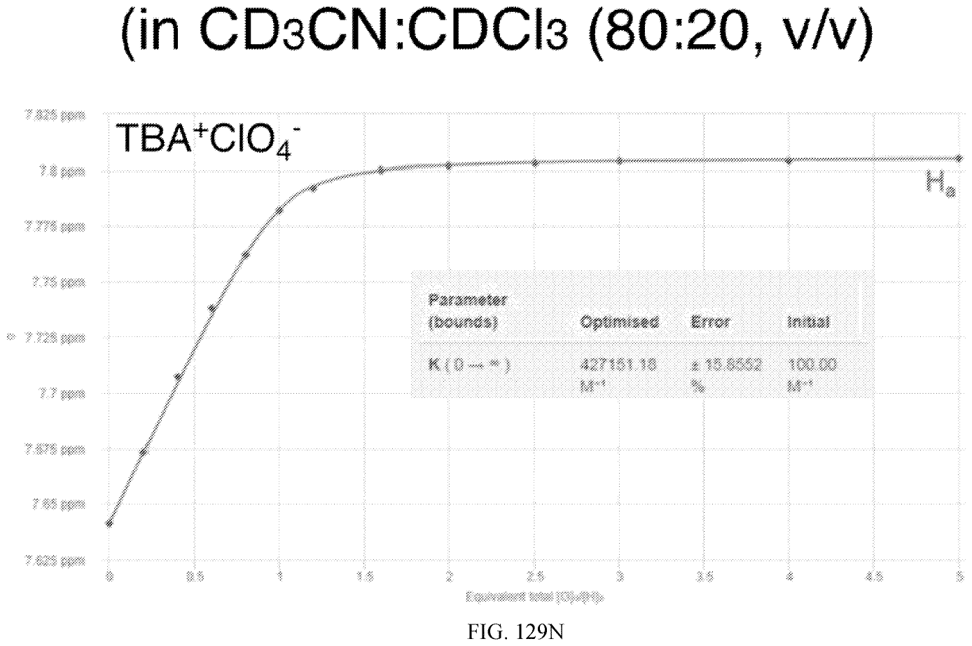

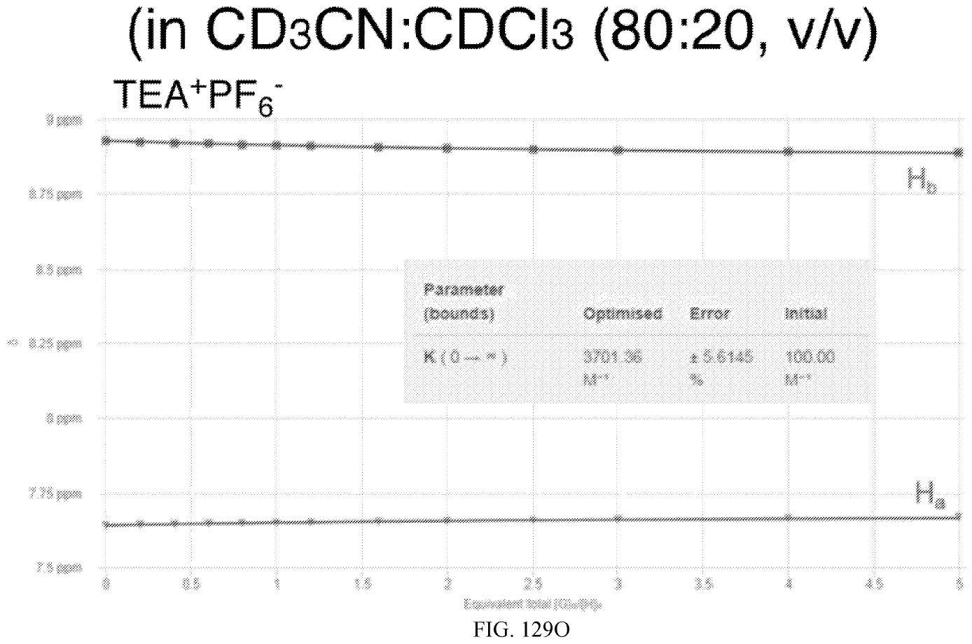

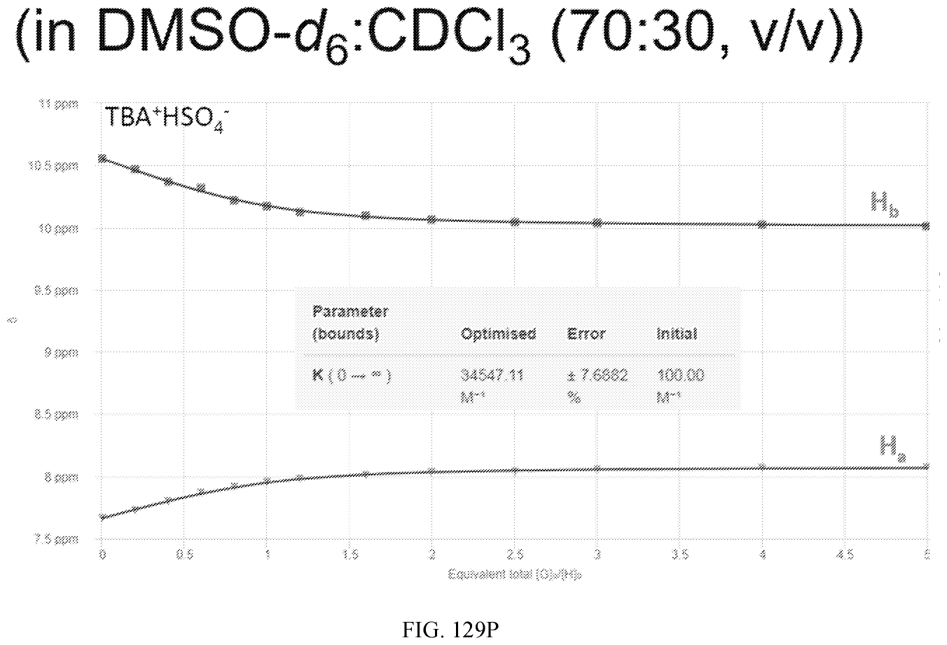

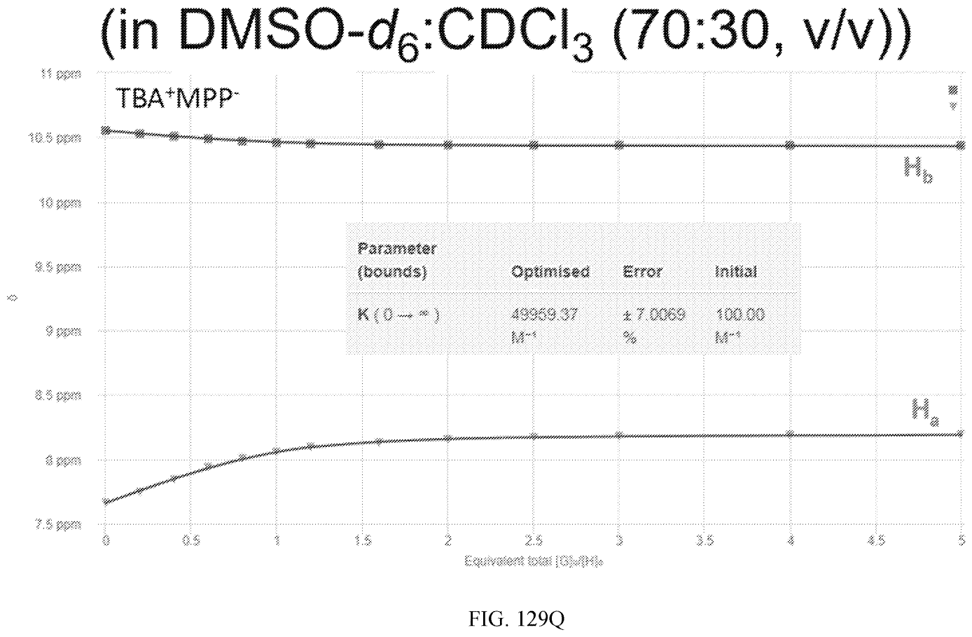

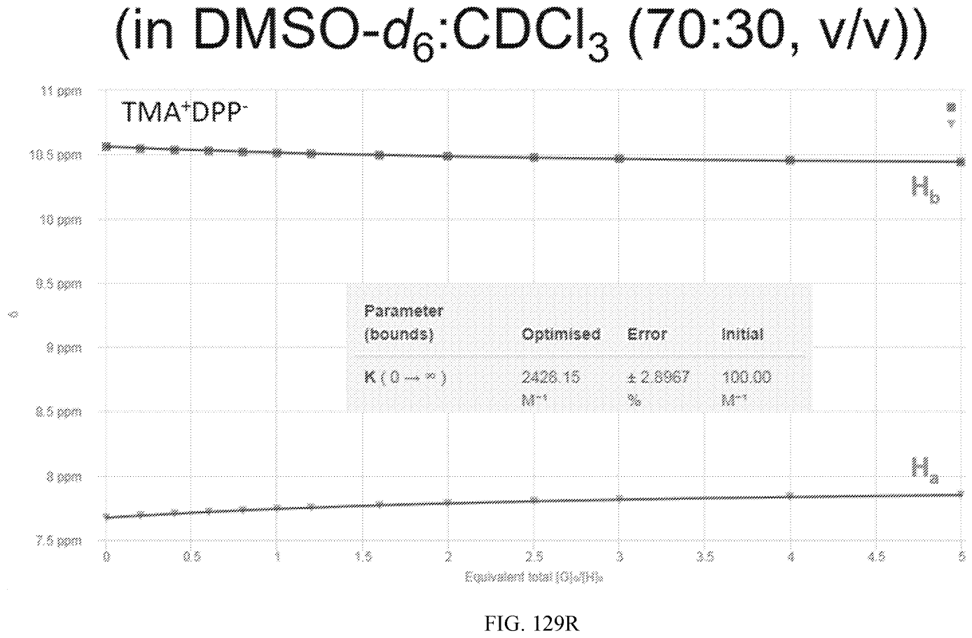

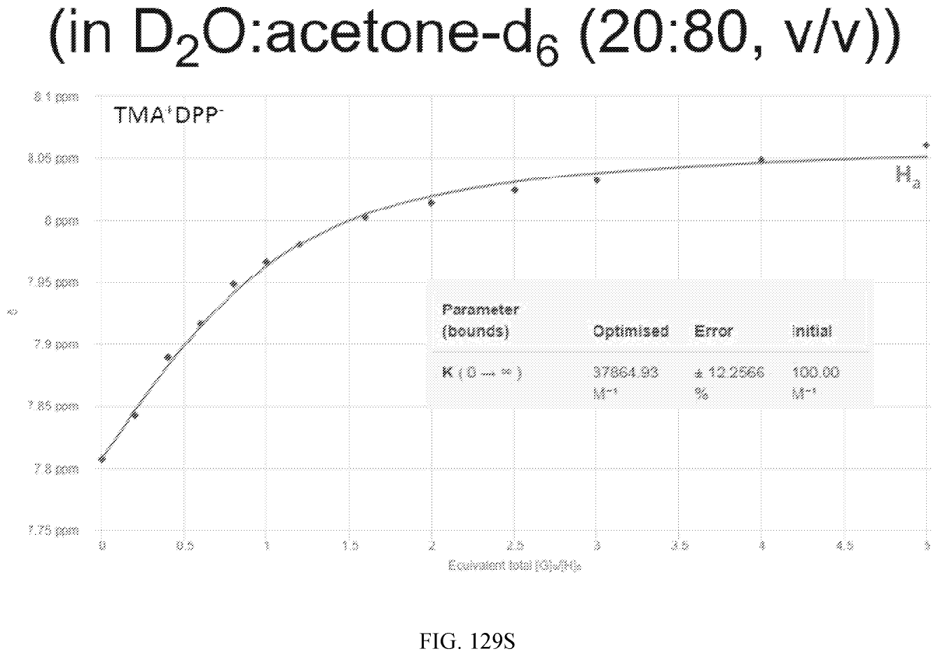

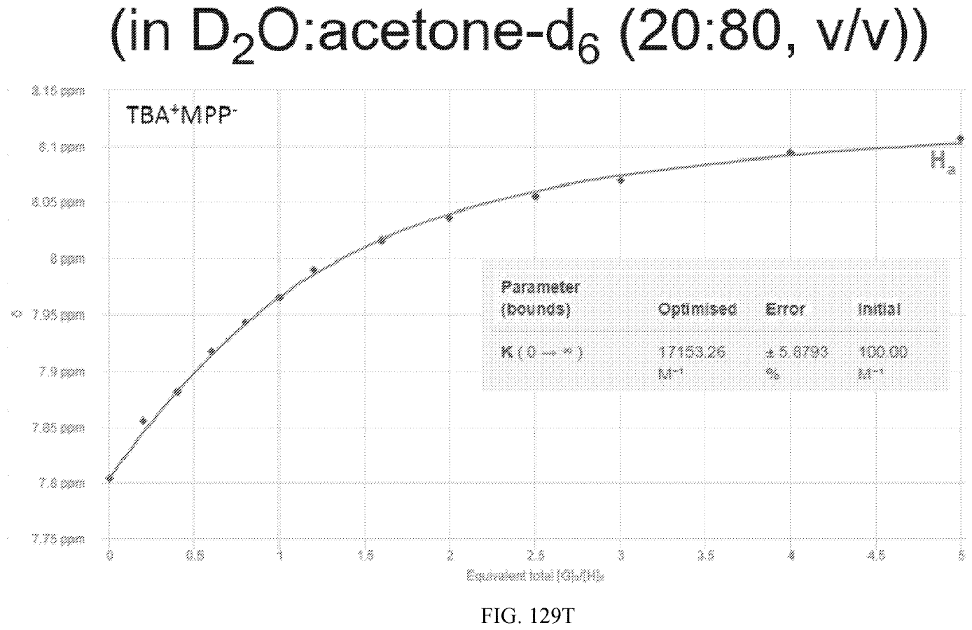

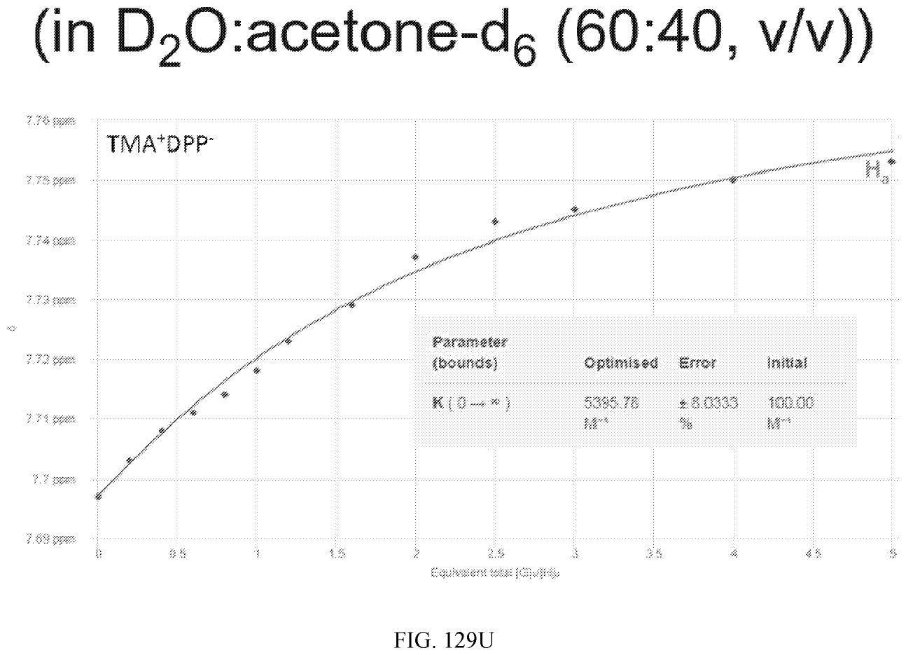

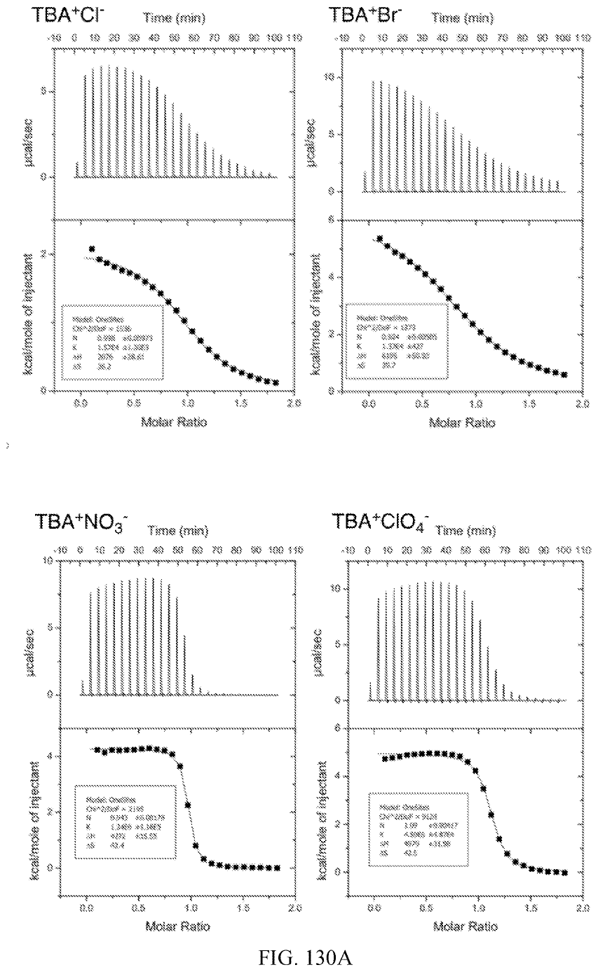

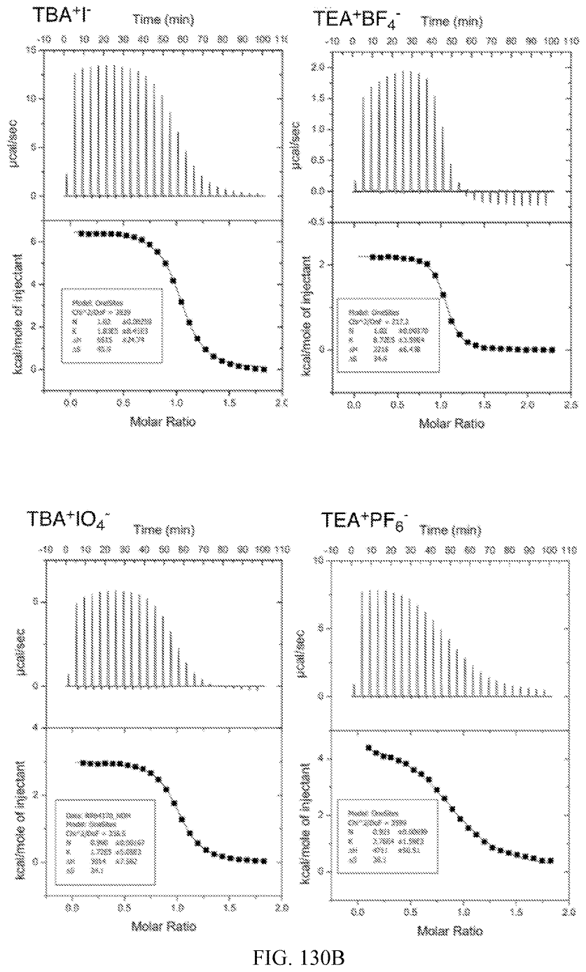

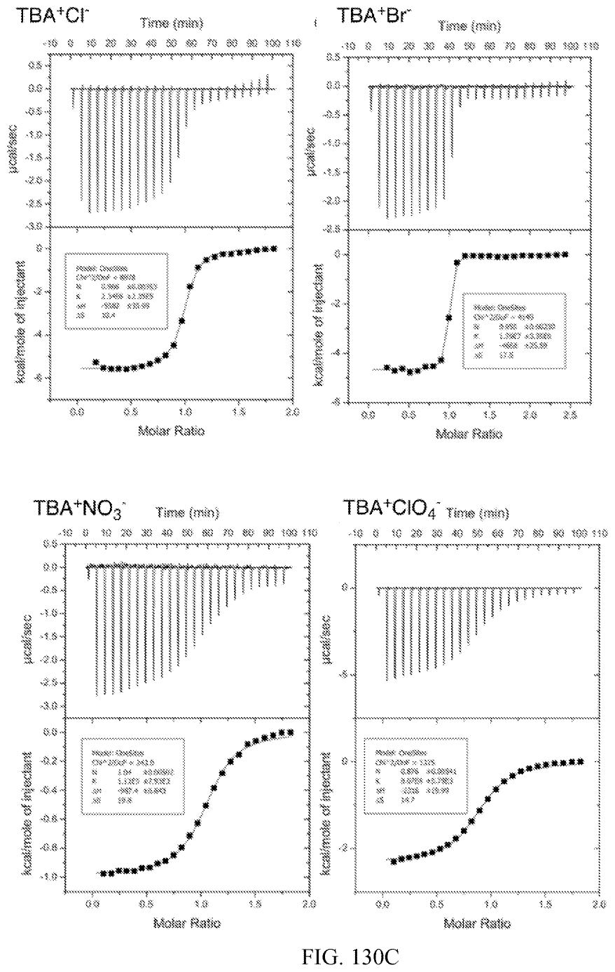

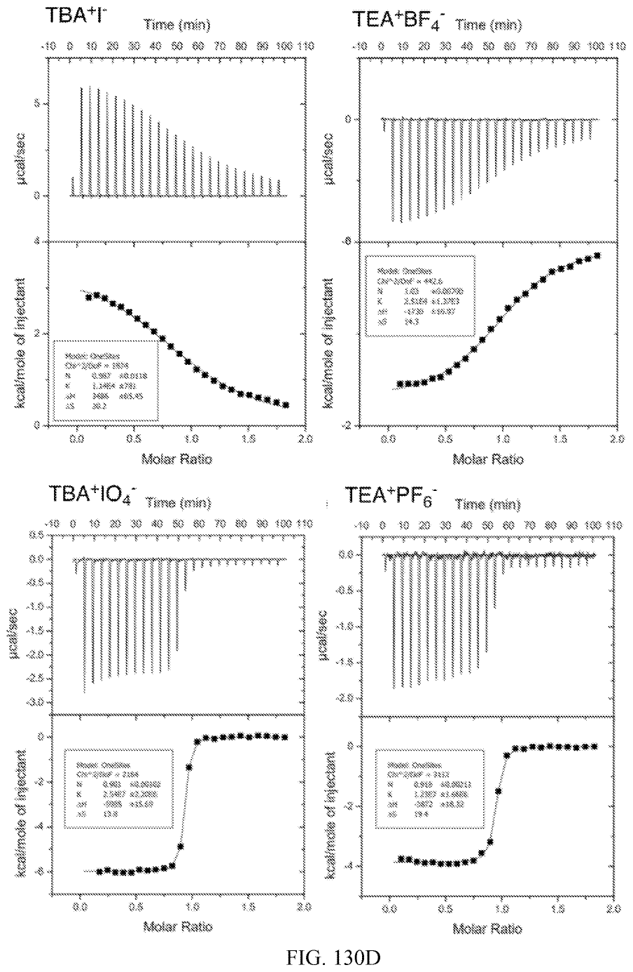

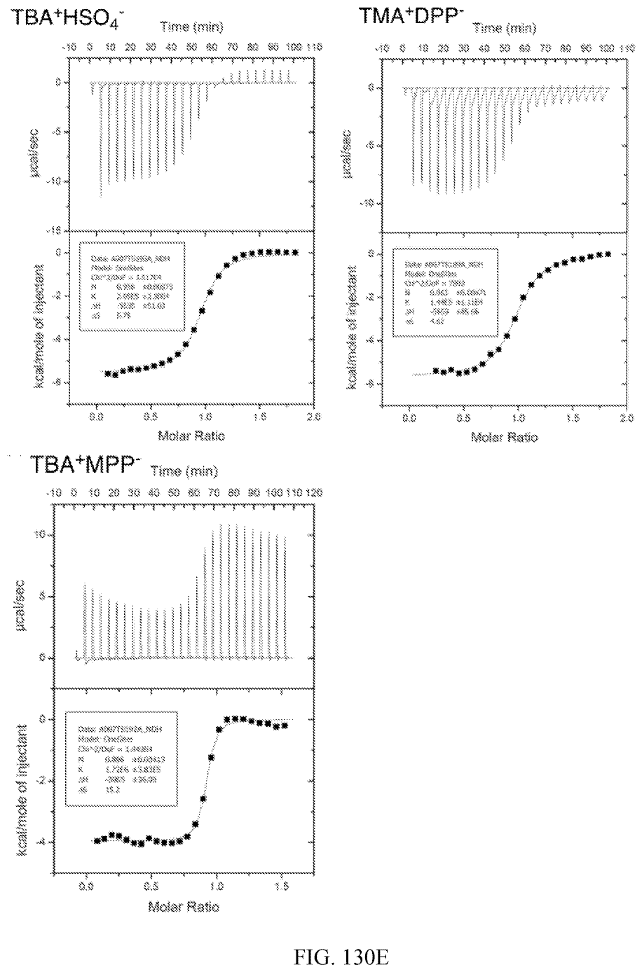

Fitting the NMR titration data in DMSO-d₆:CDCl₃ (15:85, v/v, ε=11) into a 1:1 binding model using the online fitting tool Bindfit³⁵ (FIG. 129) gave the association constants (Kₐ) for the binding of c5a with the anions (Table 3). Triplicate NMR titrations, along with ITC measurements, were performed with I⁻ and ClO₄⁻ in the same solvent, providing Kₐ values that are in the same range and thus corroborate those from single NMR titrations (Table 3). In the much more polar CD₃CN:CDCl₃ (80:20, v/v, ε=31), the Kₐ values determined by ¹H NMR titrations increase by about tenfold. The enhanced affinities of c5a for the anions in the solvent mixture of CD₃CN and CDCl₃ can be explained by the fact that, compared to DMSO, acetonitrile is a much weaker hydrogen-bond acceptor³⁴ and can be replaced by an anion from the cavity of c5 at reduced energy costs. Consistent with the strong solvation of the cavity of c5a by DMSO, the Kₐ values in the considerably less polar DMSO:CHCl ₃ (5:95, v/v, ε=6.9) measured with isothermal titration (ITC) experiments are in fact comparable to those determined in CD₃CN:CDCl₃ (80:20, v/v). The Kₐ values in CH₃CN:CHCl₃ (5:95, v/v, ε=6.4), also obtained with ITC measurements (FIG. 130), increase from two to four orders of magnitudes in comparison to those in CD₃CN:CDCl₃ (80:20, v/v, ε=31), with the Kₐ's of ClO₄⁻ and BF₄⁻ being over 108 M⁻¹ and that of NO₃ being in the 10⁹ M⁻¹ range. The strong binding of large, soft anions such as I⁻ and ClO₄⁻, which have low charge density and high polarizability, with c5a is favored by the fact that these large anions are able to interact with all the endocyclic CH and NH groups while smaller ions such as Cl⁻ and Br⁻ cannot. Besides, the binding of these weakly coordinating and polarizable anions is probably promoted by the five preorganized phenyl CH donors that are featured by their hydrophobicity and moderate polarizability.

The above results indicate that macrocycle c5a binds anions in overall lower affinities in the DMSO-containing solvents than those in the acetonitrile-containing solvents due to the fact that DMSO, as a much stronger hydrogen-bond acceptor, solvates the cavity of macrocycles c5 more effectively than acetonitrile, i.e., desolvating the binding cavity in DMSO-containing solvents is much more energetically demanding than that in acetonitrile-containing solvents.

In comparison with cyanostar iPrCS that forms 1:1 complexes with anions in CD₃CN:CD₂Cl₂ (1:1, v/v, ε=23), 37 in the more polar CD₃CN:CDCl₃ (80:20, v/v, 8=31), macrocycle c5a binds Cl⁻, Br⁻, BF₄⁻, ClO₄⁻, and IO₄⁻ with affinities (10⁴ to 10⁵ M⁻¹) that are 1 to 2 orders of magnitude higher; the Kₐ's of c5a for I⁻ and PF₆⁻ in the more polar solvent, being in the 10⁵ M⁻¹ and 10³ M⁻¹ ranges, respectively, are similar to those of cyanostar iPrCS for these two anions.

TABLE 3
Association constants Kₐ (M⁻¹) for the 1:1 complexes of c5a with various anions at 25° C. ^{a}

| | Diameter | DMSO-d₆:CDCl₃ | DMSO:CHCl₃ | CD₃CN:CDCl₃ | CH₃CN:CHCl₃ |
|---|---|---|---|---|---|
| | (Å) | (15:85, v/v) ^{b} | (5:95, v/v) ^{c} | (80:20, v/v) ^{b} | (5:95, v/v) ^{c} |
| Cl⁻ | 3.36 | (4.31 ± 0.12) × 10³ | (1.60 ± 0.10) × 10⁴ | (8.25 ± 0.59) × 10⁴ | (2.34 ± 0.24) × 10⁶ |
| Br⁻ | 3.80 | (9.82 ± 1.08) × 10³ | (1.37 ± 0.04) × 10⁴ | (4.98 ± 0.30) × 10⁴ | (1.79 ± 0.34) × 10⁷ |
| NO₃⁻ | 4.00 | (1.57 ± 0.13) × 10⁵ | (1.30 ± 0.10) × 10⁶ | >10⁶ | (1.38 ± 0.21) × 10⁹ ^{e} |
| BF₄⁻ | 4.10 | (4.73 ± 0.38) × 10⁴ | (8.70 ± 0.40) × 10⁵ | (2.10 ± 0.23) × 10⁵ ^{g} | (3.09 ± 0.45) × 10⁸ ^{e} |
| I | 4.22 | (2.73 ± 0.17) × 10⁴ | (1.83 ± 0.08) × 10⁵ | (5.96 ± 0.89) × 10⁵ ^{g} | (7.78 ± 4.26) × 10⁷ |
| | | (3.77 ± 0.25) × 10⁴ ^{c} | | | (6.67 ± 0.81) × 10⁷ ^{f} |
| | | (1.18 ± 0.03) × 10⁴ ^{d} | | | |
| ClO₄⁻ | 4.50 | (5.74 ± 0.51) × 10⁴ | (4.50 ± 0.50) × 10⁵ | (4.27 ± 0.67) × 10⁵ ^{g} | (4.96 ± 0.76) × 10⁸ ^{e} |
| | | (9.60 ± 0.76) × 10⁴ ^{c} | | | |
| | | (1.90 ± 0.02) × 10⁴ ^{d} | | | |
| IO₄⁻ | 4.62 | (1.18 ± 0.08) × 10⁴ | (1.72 ± 0.05) × 10⁵ | (4.24 ± 0.21) × 10⁴ | (2.54 ± 0.22) × 10⁷ |
| PF₆⁻ | 4.84 | not detected | (2.80 ± 0.20) × 10⁴ | (3.70 ± 0.22) × 10³ | (1.23 ± 0.17) × 10⁷ |

^{a} Measured at 298 K.
^{b} Determined by NMR titrations by fitting the chemical shift changes of both protons a and b unless indicated otherwise.
^{c} Determined by triplicate NMR titrations.
^{d} Determined by ITC measurements.
^{e} Determined by two-step competition ITC experiments with PF₆⁻ as the competitor.
^{f} Determined by two-step competition ITC experiments with Cl⁻ as the competitor.
^{g} Value based on chemical shift changes of CH protons a only due to large errors involving NH protons b.

Three oxoanions, HSO₄⁻, DPP⁻, and MPP (FIG. 115B, bottom), that are strong hydrogen-bond acceptors, were examined for their binding. In DMSO-d₆:CDCl₃ (15:85, v/v), titrating c5a with the TBA⁺ salt of HSO₄⁻ or MPP⁻, or the tetramethylammonium (TMA⁺) salt of DPP⁻, gave two sets of peaks for protons a, b, or c in the ¹H NMR spectrum (FIG. 115b top, and 128), indicating that the free and bound forms of c5a undergo slow exchange on the NMR time scale. In solvents of high polarity, Kₐ's could be determined by direct ¹H NMR titrations (Table 4). In DMSO-d₆:CDCl₃ (70:30, v/v, ε=34), Kₐ's over 10⁴ M⁻¹ for HSO₄ and MPP⁻, and over 10³ M⁻¹ for DPP were obtained. In acetone-do with 20% D₂O (ε=33), both DPP⁻ and MPP-show binding affinities for c5a in the 10⁴ M⁻¹ range. Even in acetone-de with 60% D₂O (ε=56), DPP still binds with c5b with a Kₐ over 10³ M⁻¹ (FIG. 129). In the much less polar CH₃CN:CHCl₃ (5:95, v/v, ε=6.4), the Kₐ values for the binding of c5a with the three oxoanions had to be determined with two-step competition ITC experiments. The Kₐ's for HSO₄ and DPP are over 10⁹ M⁻¹ and that of MPP is over 10¹⁰ M⁻¹ (Table 4). The high affinities of c5a for the oxoanions demonstrate that the combination of strong hydrogen-bonding capabilities of the anion guests and multiple hydrogen-bonding interactions, especially those with the convergently placed NH groups, can result in remarkable binding affinity and selectivity.

TABLE 4
Association constants Kₐ (M⁻¹) for the 1:1 complexes of macrocycles c5a with three oxoanions.

| | Diameter | CH₃CN:CHCl₃ | CD₃CN:CDCl₃ | DMSO-d₆:CDCl₃ | D₂O:acetone-d₆ | D₂O:acetone-d₆ |
|---|---|---|---|---|---|---|
| | (Å) | (5:95, v/v) ^{a} | (40:60, v/v) ^{b} | (70:30, v/v) ^{c} | (20:80, v/v) ^{c} | (60:40, v/v) ^{c} |
| HSO₄⁻ ^{d} | 4.42 | (7.0 ± 1.4) × | — | (3.52 ± 0.35) × | — | — |
| | | 10⁹ | | 10⁴ | | |
| MPP⁻ ^{d} | ~4.3 | (5.2 ± 1.4) × | (3.11 ± 0.57) × | (4.66 ± 0.55) × | (1.72 ± 0.10) × | — |
| | | 10¹⁰ | 10⁸ | 10⁴ | 10⁴ | |
| DPP⁻ ^{e} | ~4.3 | (4.3 ± 0.8) × | (2.99 ± 0.36) × | (2.43 ± 0.07) × | (3.79 ± 0.45) × | (5.40 ± 0.43) × |
| | | 10⁹ | 10⁷ | 10³ | 10⁴ | 10³ |

^{a} Determined by two-step competition ITC experiments with PF₆⁻ as the competitor.
^{b} Determined by two-step competition NMR titration experiments with Cl⁻ as the competitor at 25° C.
^{c} Determined by NMR titrations at 25° C.
^{d} As the TBA⁺ salts (TBA⁺ = tetrabutylammonium).
^{e} As the TMA⁺ salt (TMA⁺ = tetramethylammonium).

(iv) Binding strength following or deviating from a size-dependent trend. The overall binding strength of macrocycles c5 for an anion is a reflection of the global binding events involving the interactions with all endocyclic NH and CH protons, along with other factors such as the attractive ion-dipole interactions between the anion and the inwardly pointing amide dipoles, and the entropically favorable release of solvent molecules upon binding.

The binding of c5a with the three halides and large, weakly coordinating anions including ClO₄⁻, IO₄⁻, and PF₆ follows a size-dependent trend, with Kₐ's increasing from Cl⁻ to ClO₄⁻ and then declining from ClO₄⁻ to the larger IO₄⁻ and PF₆⁻ (Table 3 and FIG. 115c). Such size-dependence was also observed with previously reported cyanostar iPrCS and bambusurils. In contrast, NO₃⁻ or BF₄⁻ does not follow such a size-dependent trend when binding with c5a. Being smaller than I⁻, NO₃⁻ binds to c5a in the highest affinities among the anions shown in Table 3. BF₄⁻, which is also smaller than I⁻, binds with c5a in affinities that are only lower than NO₃⁻, similar to I⁻ or ClO₄⁻, but higher than the other anions. The observed deviation from size-dependence in the binding of c5a with NO₃⁻ or BF₄⁻ is different from cyanostar and bambusurils. While the mechanism remains to be elucidated, the “abnormal” behavior of NO₃⁻ or BF₄⁻ indicates the adaptability of macrocycle c5a when binding anions. NO₃⁻, with its planar shape and symmetrically distributed O atoms, is compatible with the cavity of c5a in both shape and hydrogen bonding pattern; BF₄, being smaller than I⁻ and other larger weakly coordinating anions, may engage in more effective ion-dipole interactions with the amide dipoles of c5a because of its higher charge density.

The binding of c5a with oxoanions HSO₄⁻, DPP⁻, and MPP⁻ deviates even more significantly from the size-dependent trend shown by halide ions, ClO₄⁻, IO₄⁻ and PF₆⁻ (FIG. 115c), with drastically stronger binding affinities in the same solvents and being less sensitive to the hydrogen-bonding capability of the solvents involved. The stronger, but less size-dependent binding of c5a with oxoanions than with other anions is not observed with known anion binders such as the bambusurils which favor large, weakly coordinating anions like I⁻ and ClO₄⁻.

The exceptional anion binding capabilities of macrocycles c5 can be attributed to the presence of the preorganized endocyclic amide NH and phenyl CH groups. As powerful hydrogen-bond donors, the NH donors form multiple, strong hydrogen bonds with oxoanions that are known to be strong hydrogen-bond acceptors. The five convergently placed, hydrophobic and moderately polarizable phenyl CH groups favor large, polarizable anions capable of interacting with most or all CH and NH groups. These distinct interactions, along with the five inwardly pointed amide dipoles, result in the unique, versatile anion binding behavior shown by macrocycles c5.

(iv) Two-dimensional (ROESY) spectrum of complex c5a·MPP. The 2D (ROESY) NMR spectrum of the 1:1 mixture of c5a and MPP recorded in DMSO-d₆ confirms the formation of complex c5a·MPP. ROEs between protons a of c5a and protons 1 and 2 of MPP were clearly detected (FIG. 115d). In contrast, no significant ROE could be found between the protons/and 2 of MPP⁻ and exterior protons d and e of c5a, suggesting that MPP⁻ indeed is located inside the cavity of c5a.

Selective halide transport across lipid membranes. The promotion of transmembrane anion transport has become an important goal for supramolecular chemistry. Although numerous cation carriers, i.e., cationophores, have been created, effective anionophores are only being developed recently. Anion transporters have potential as tools for biophysical research and may reveal new modes of biological activity. In particular, several genetic disorders which involve defects in natural anion channels, the best-known being cystic fibrosis (CF), may benefit from synthetic anionophores that could be used to treat these conditions via “channel replacement therapy”. Compared to charged anion carriers that may lead to various complications, a more promising approach for channel replacement therapy is arguably the use of uncharged, i.e., neutral anion carriers.

Macrocycle c5a, a neutral compound with a conformationally defined lipophilic exterior and hydrophilic interior, may partition into biomembranes to facilitate transmembrane anion transport. To examine this possibility, unilamellar liposomes were used to characterize the membrane transport properties of halides with c5a. Three independent assays, including the ratiometric measurements of halide-induced fluorescence changes of entrapped trisodium 8-hydroxypyrene-1,3,6-trisulfonate (HPTS), a pH-sensitive dye, the detection of the fluorescence quenching of entrapped lucigenin by transported halides, and the determination of EC50 values of transmembrane halide transport, were performed.

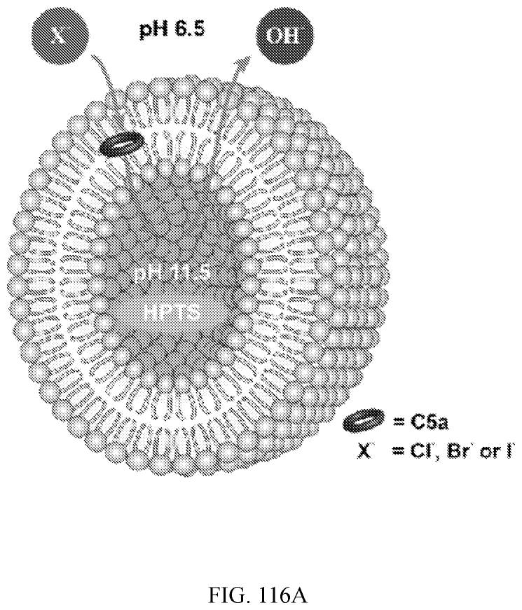

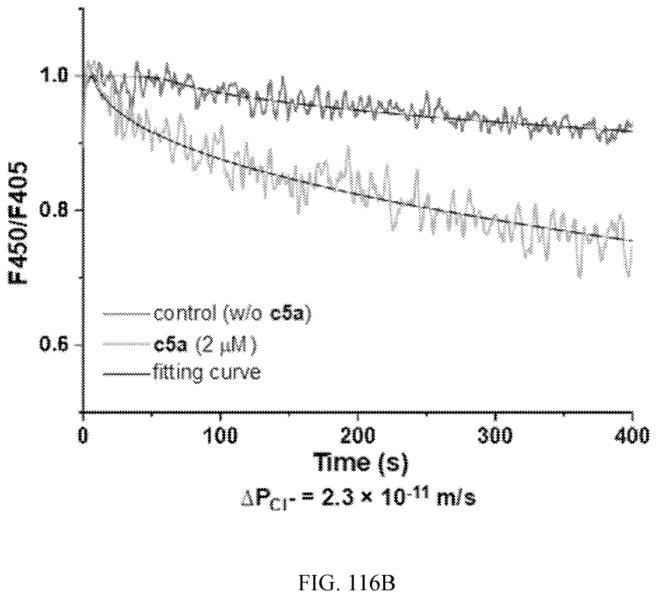

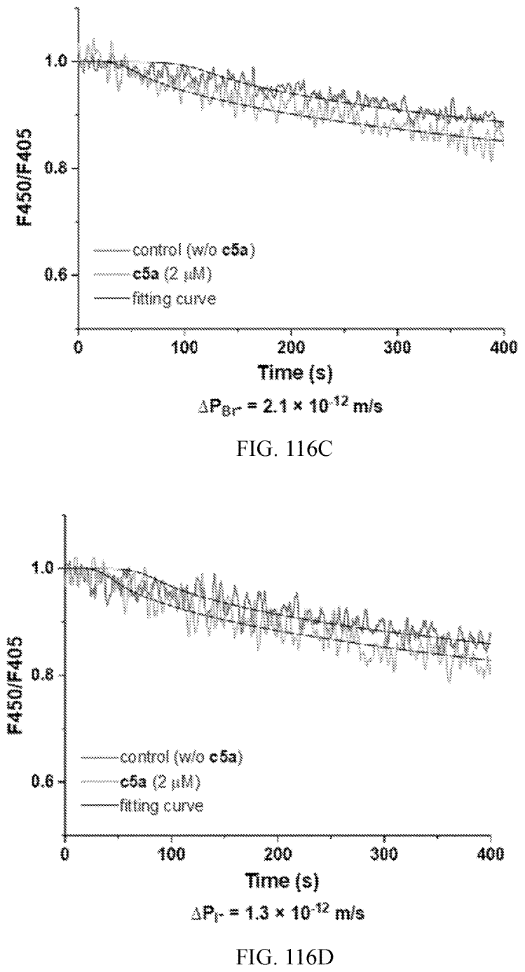

Assessing ion transport by monitoring the fluorescence of HPTS entrapped in large unilamellar vesicles (LUVs) is a well-established, widely used method that allows the transport of different anions to be directly compared under the same conditions. As shown in FIG. 116a, upon the addition of c5a, the collapse of an applied pH gradient was followed with ratiometric fluorescence kinetics. Macrocycle c5a can indeed mediate the transmembrane transport of Cl⁻, Br⁻, and I⁻, with the largest enhancement in C₁ transport (FIG. 116b), followed by Br transport (FIG. 116c), and the smallest increase in I⁻ transport, being observed (FIG. 116d). The ion permeability of vesicles with or without c5a was further determined by fitting these time-lapse ratiometric fluorescence curves with a quantitative model taken into the contributions of association constants. The total anion permeability elicited by c5a (ΔP_{X-}) was calculated by subtracting the ion permeability of the vesicles and that with c5a (see the SI). The corresponding permeability reveals a high transport selectivity for chloride ions with the ratio of Cl⁻:Br⁻:I⁻=17.8:1.6:1, a sequence inversely related to the binding affinities of c5a for these three halides. Such a high transport selectivity, which outperforms those of synthetic Cl⁻ carriers and native biological channels such as the ClC-1 C₁ channel with a transport selectivity of Cl⁻:Br⁻:I⁻=5:2:1, is quite remarkable.

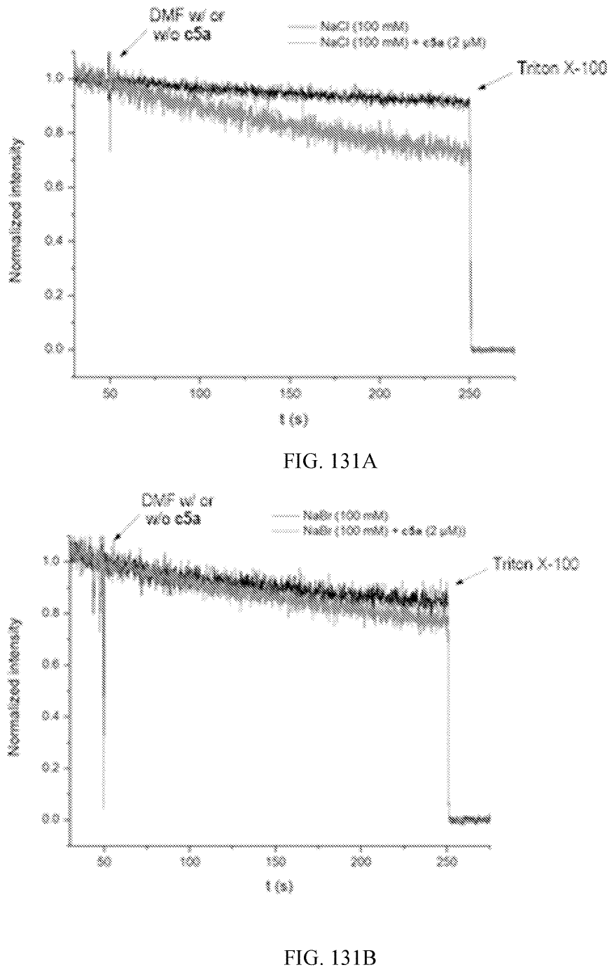

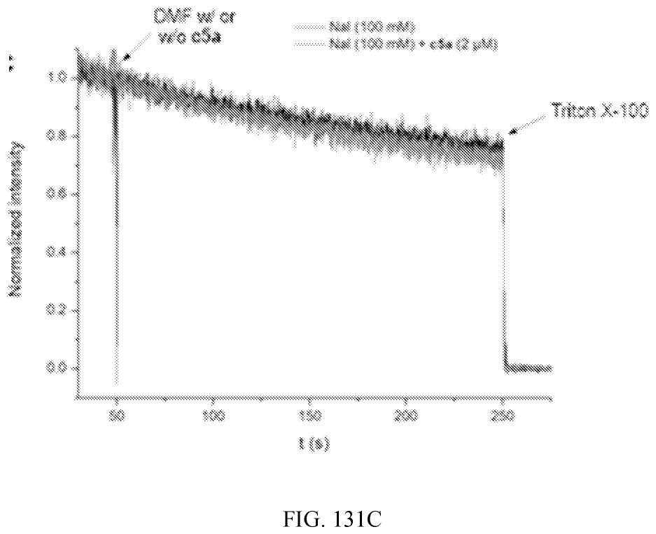

Since HPTS assay indirectly measures anion transport, the fluorescence of lucigenin encapsulated within POPC LUVs in a halide-free medium at pH 7.0 was monitored. The emission of lucigenin is directly quenched by halides at various efficiencies with I⁻ being the most and C₁ the least effective quencher. When halide ions are added to the external medium, any transport of halides into the vesicles would lead to a decrease of lucigenin fluorescence. In the presence of c5a, C₁ results in the most reduction of lucigenin fluorescence, followed by Br⁻, and then I⁻ (FIG. 131), which indicate that Cl⁻ is transported much more efficiently than the other halides with a sequence of Cl⁻>Br⁻>I⁻, in agreement with the HPTS assay.

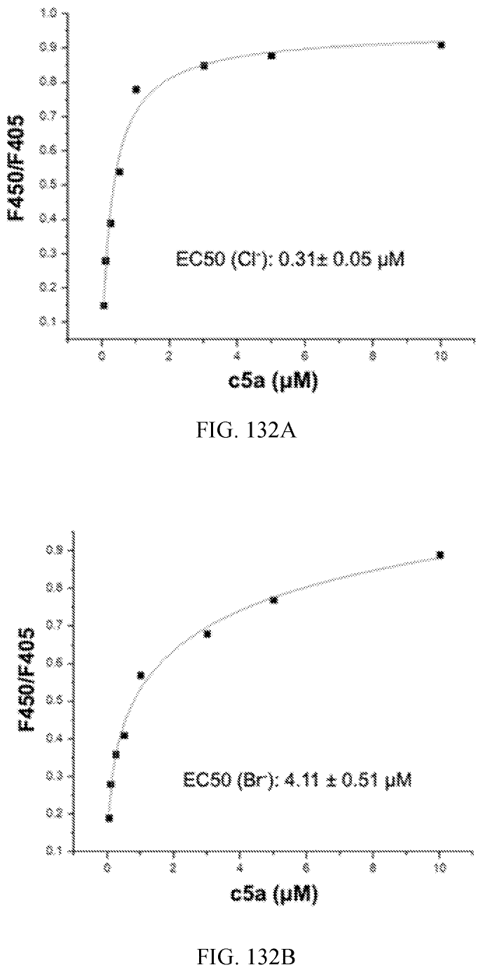

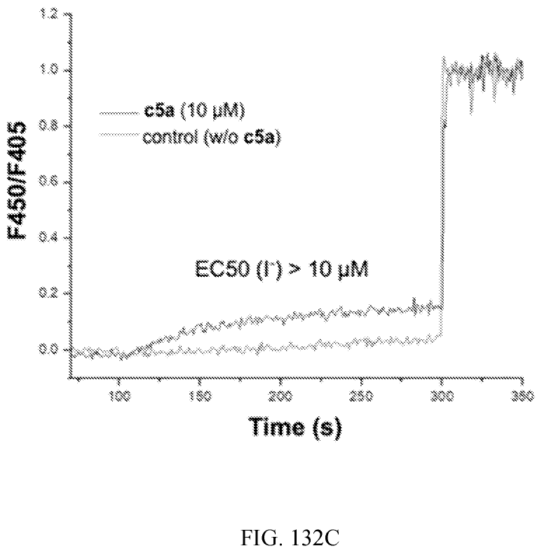

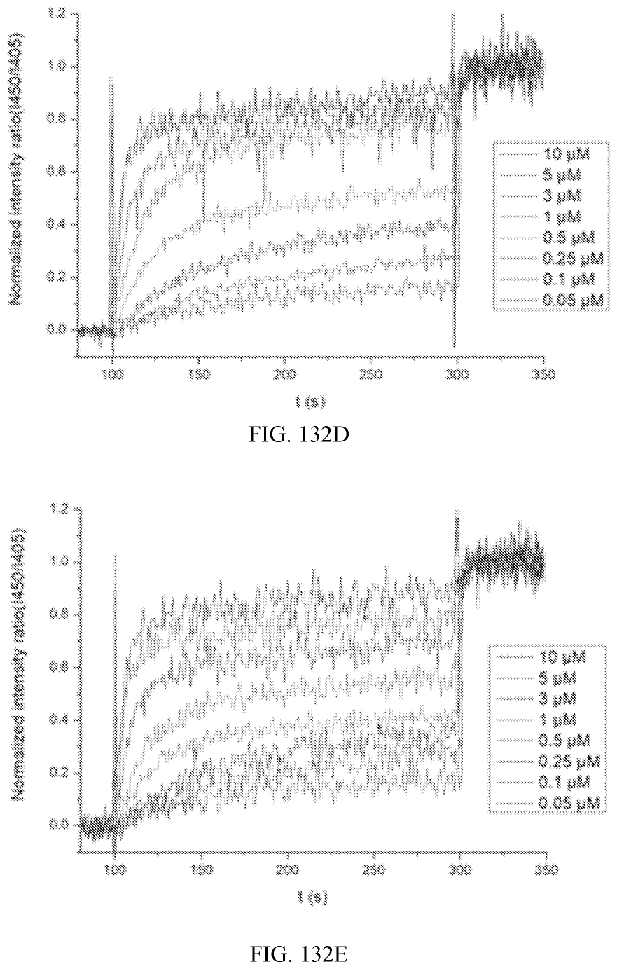

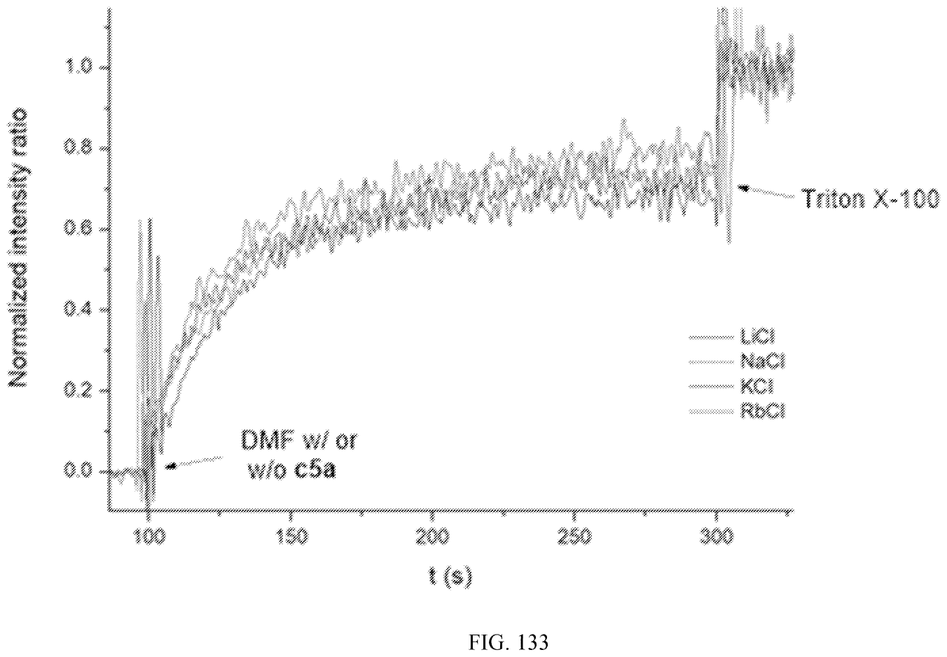

Further examination of halide transport mediated by c5a (0 to 10 μM) was achieved by following the fluorescence of intravesicular HPTS with a transmembrane pH gradient created by an extravesicular base (HO⁻) pulse. The dose-response curves led to EC50 values of 0.31 μM for Cl⁻, 4.11 μM for Br′, and >>10 μM for I⁻ (too large to be measured due to the low transport activity of I⁻) (FIG. 132). The EC50 values provide another set of evidence demonstrating the highly efficient transport of Cl⁻ and confirming the transport selectivity of halides. The transmembrane ion transport mediated by c5a in the presence of LiCl, NaCl, KCl, or RbCl shows insignificant difference, demonstrating that c5a is an anion transporter (FIG. 133).

The measured selectivity sequence for halide transport does not follow the Hofmeister order, which is remarkable but does have precedence. Anti-Hofmeister selectivity in ion transport has been documented with some other synthetic transporters. Although the resolution of the underlying mechanism for this phenomenon requires detailed theoretical and experimental studies, the observed selectivity, which seems to be proportional to the hydrogen-bonding ability of the halides to c5a, may be qualitatively rationalized based on the notion that transport efficiency may follow the “Goldilocks principle”, i.e., the strongest binders may not be the best transporter. Among the three halides, iodide binds with the highest affinity with c5a, which could impede its release from the complex, leading to the least transport efficiency in comparison to that of Cl⁻ with a lower binding affinity.

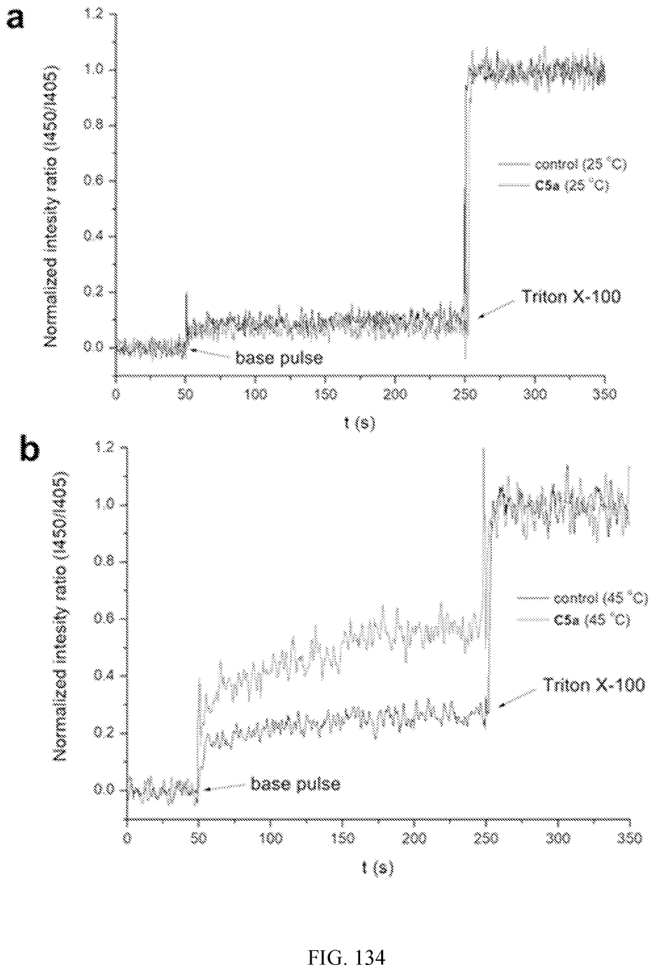

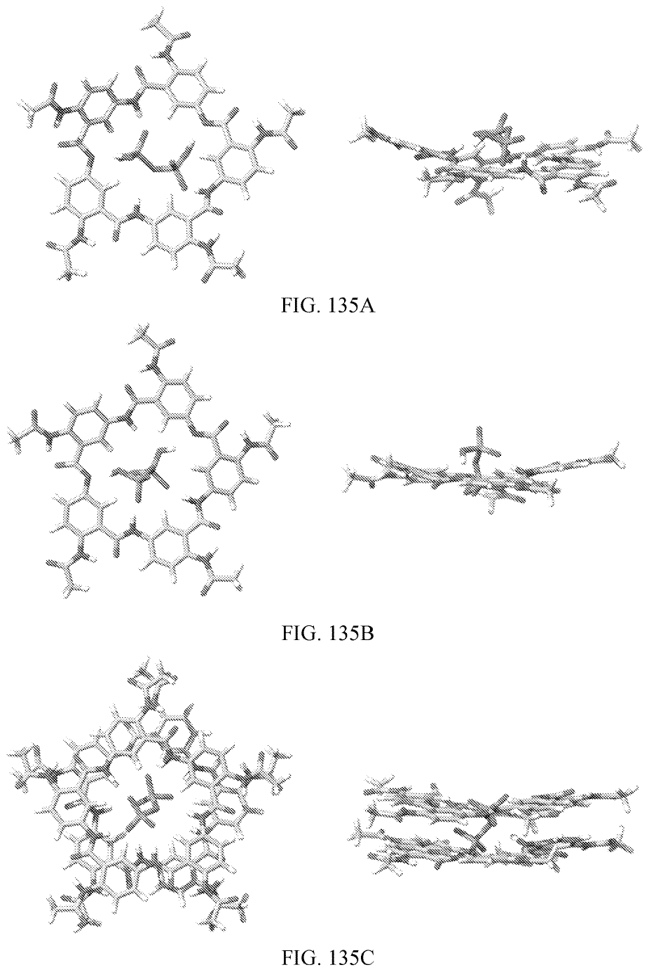

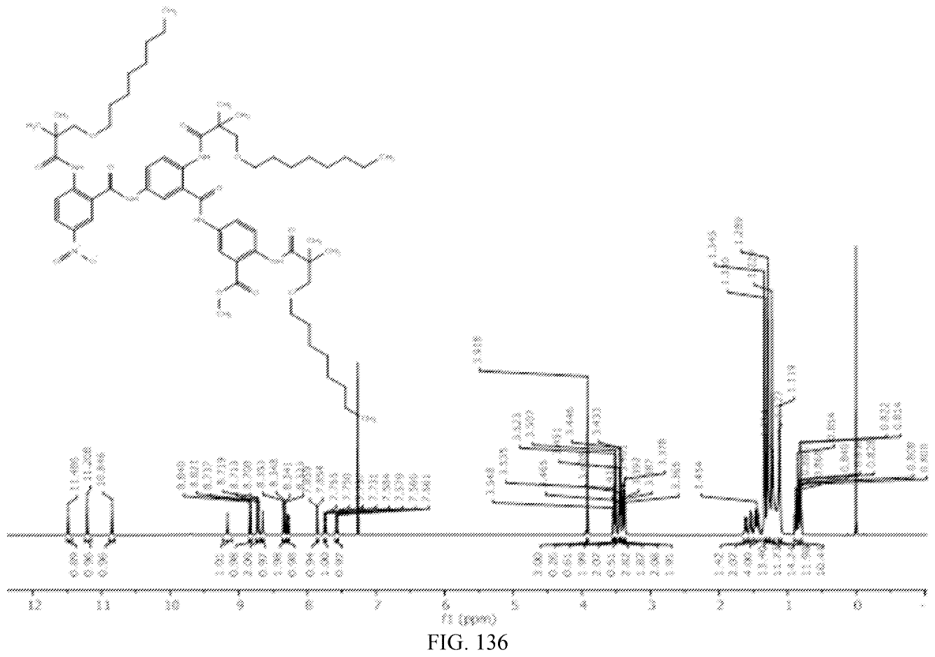

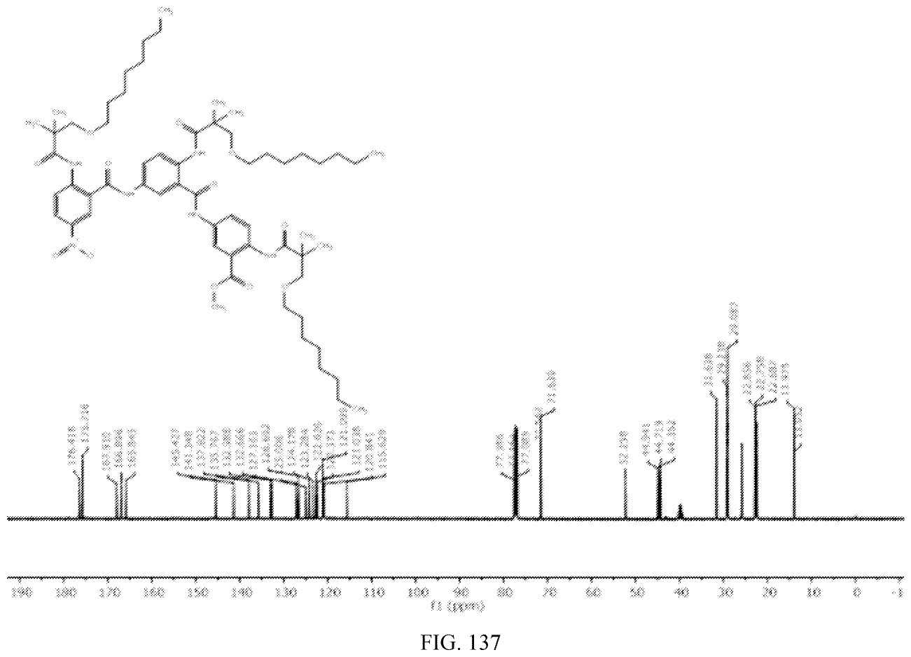

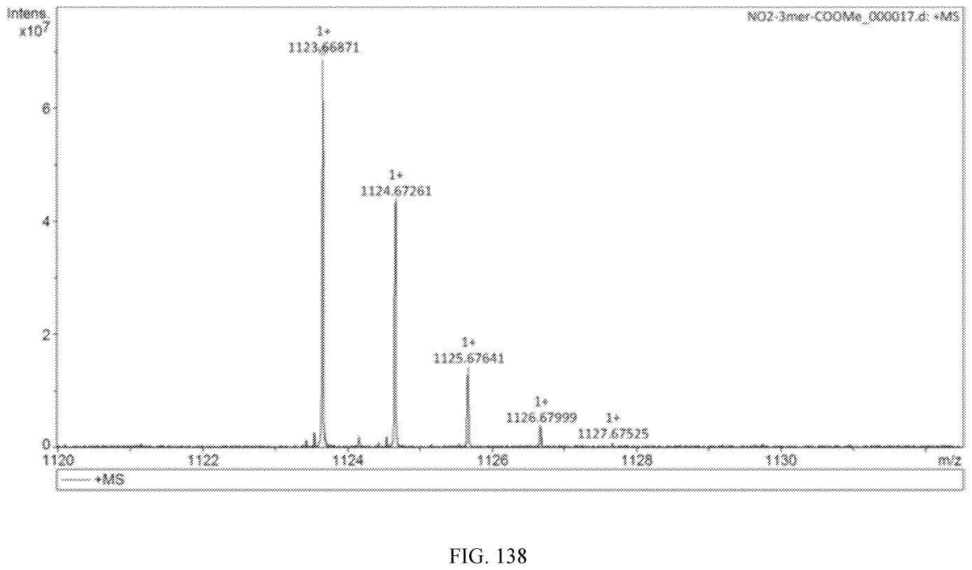

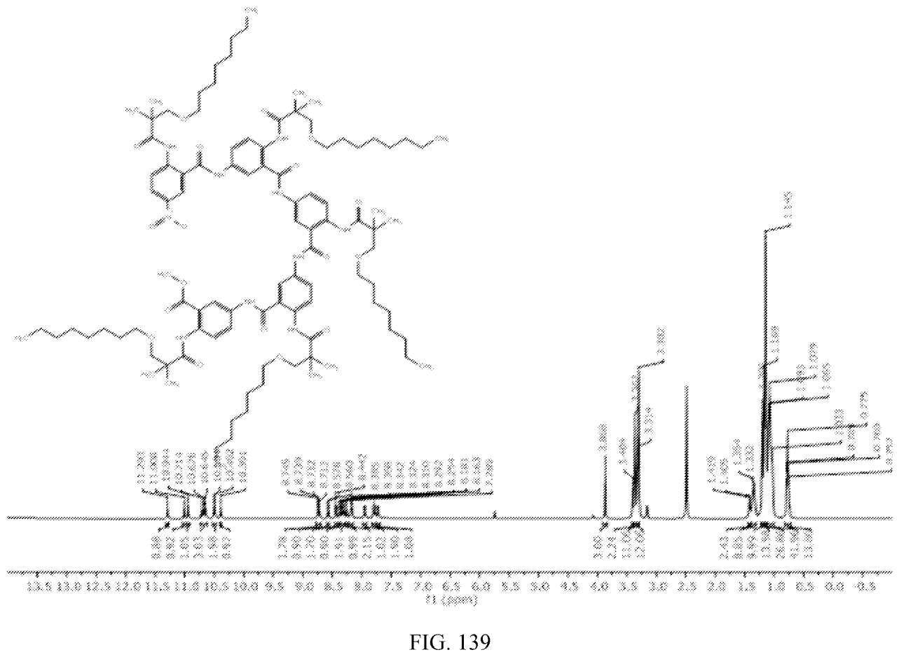

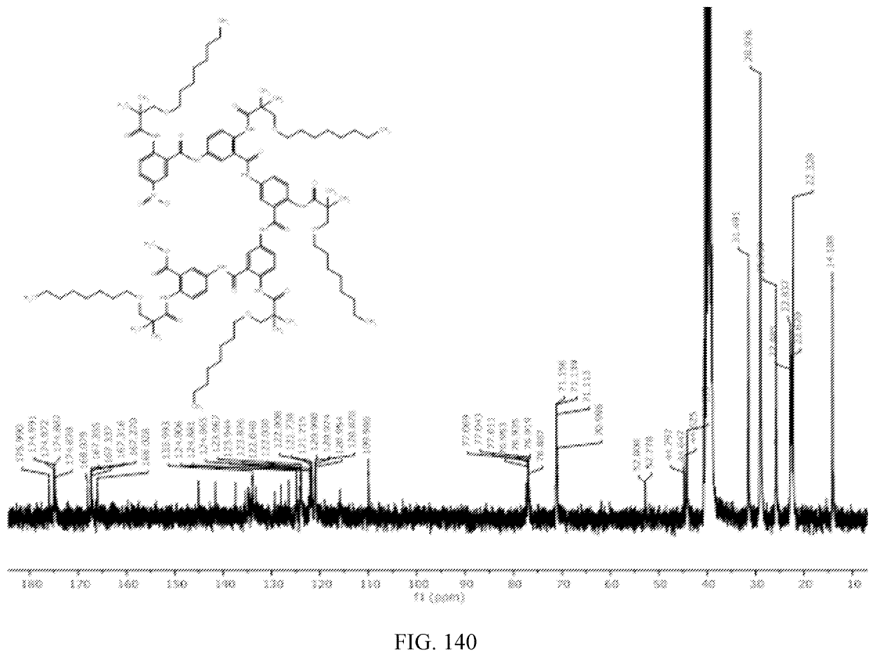

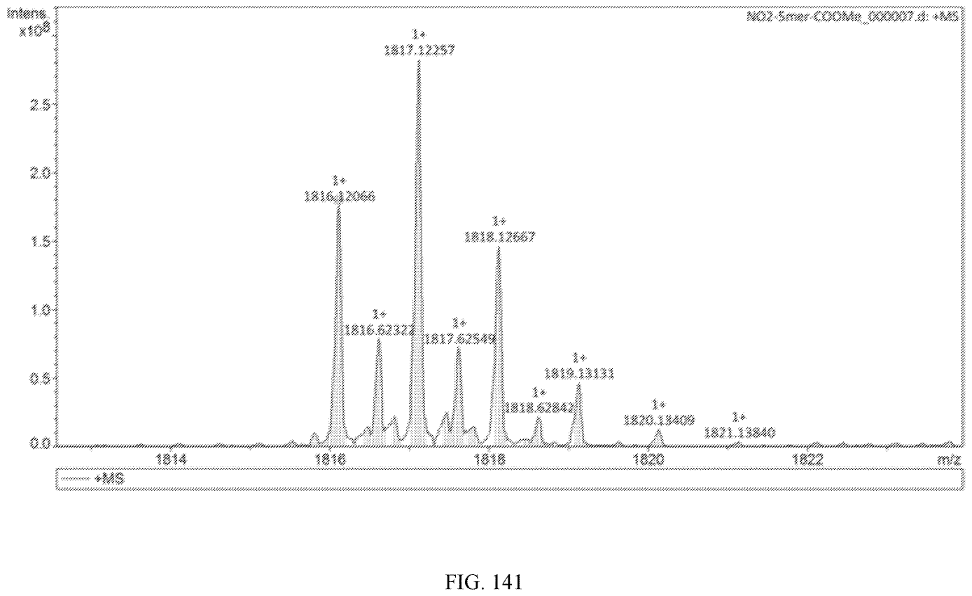

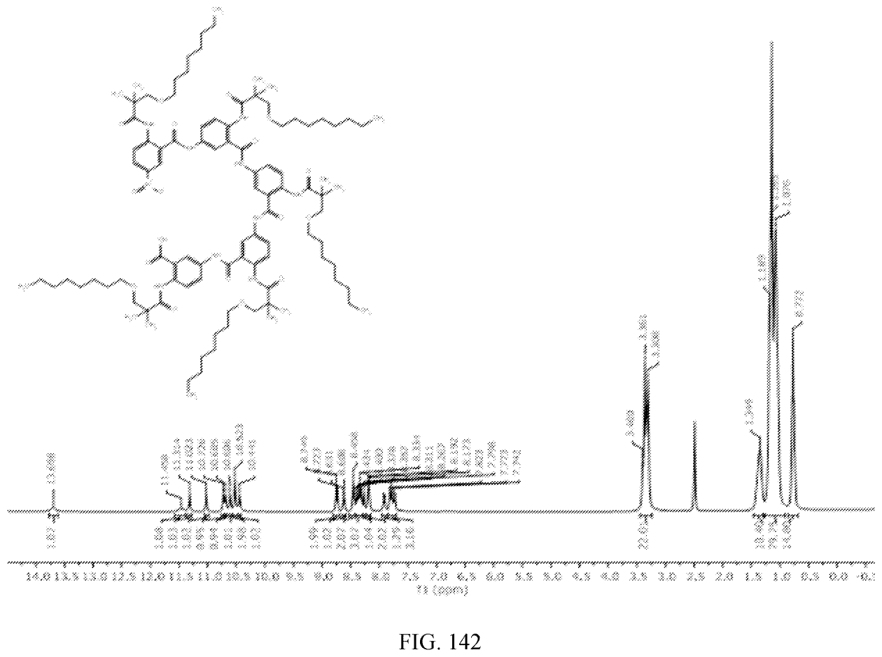

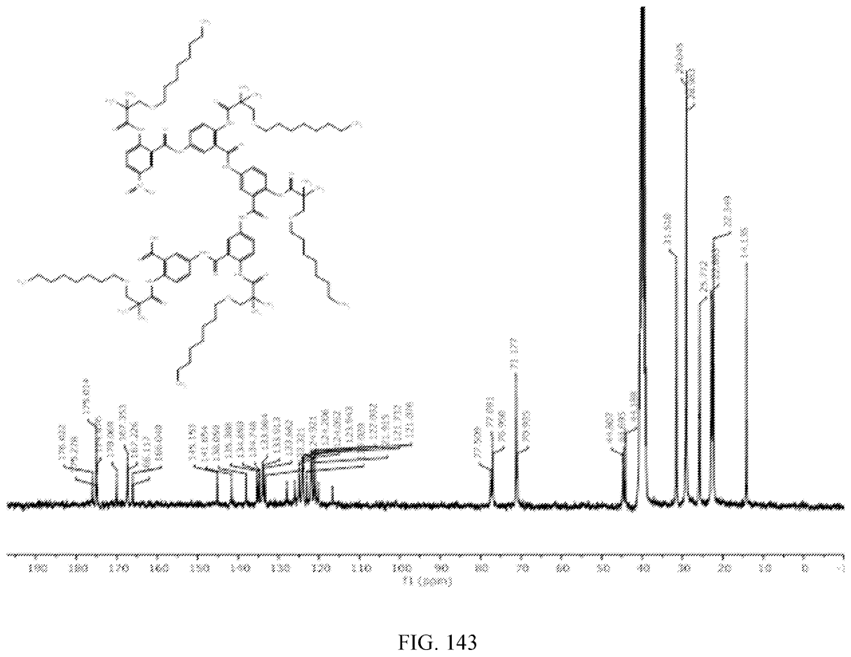

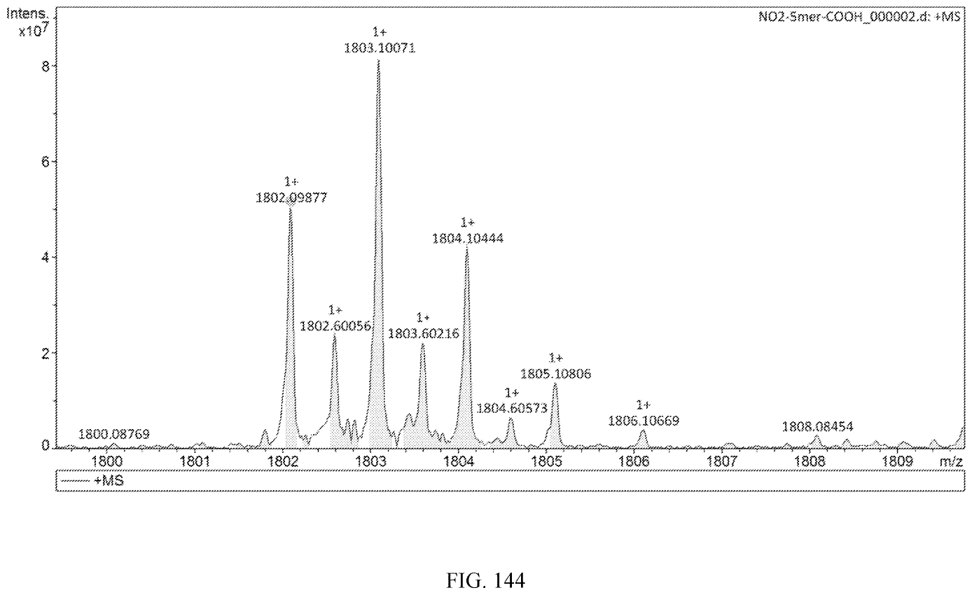

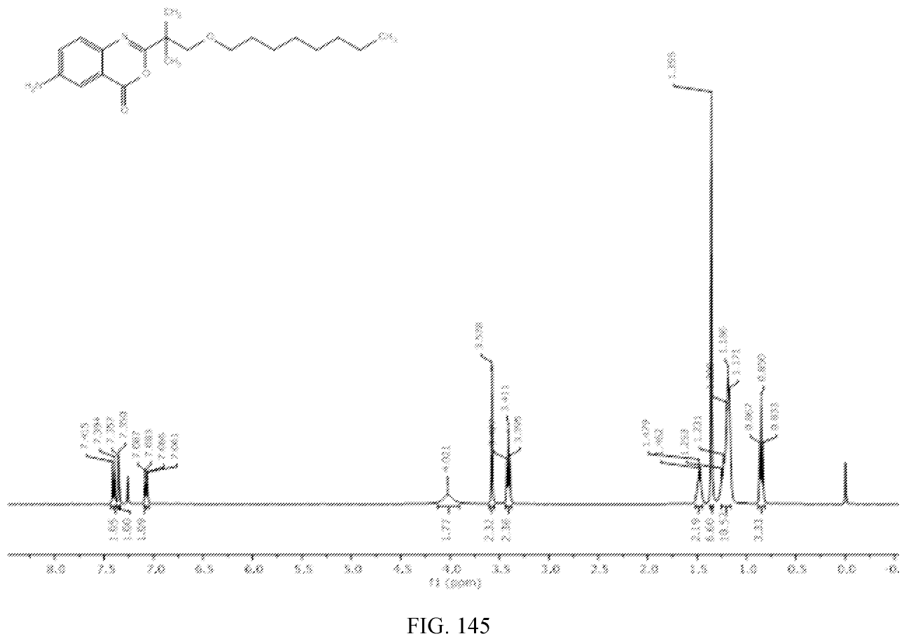

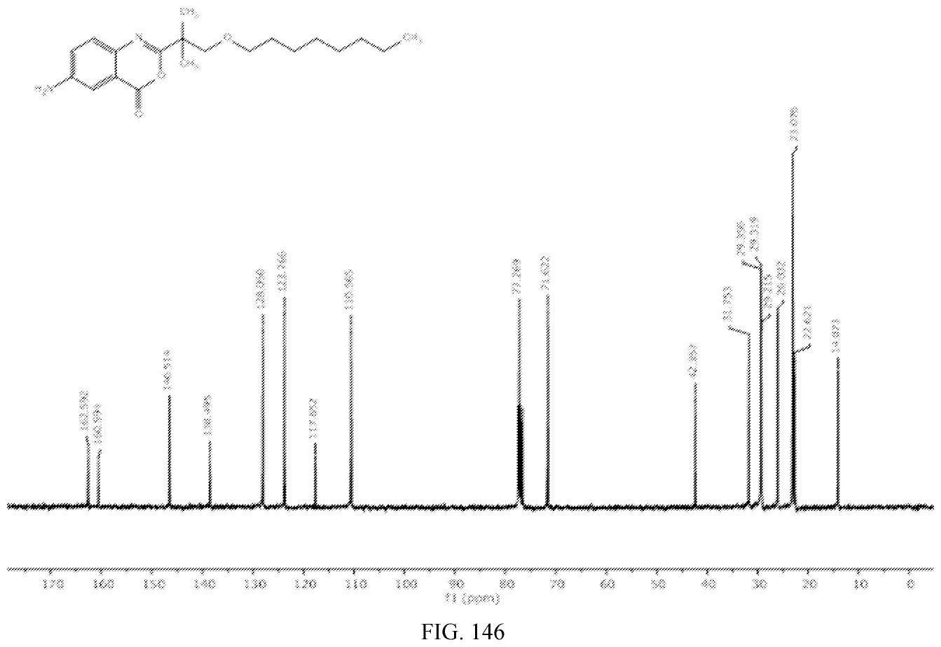

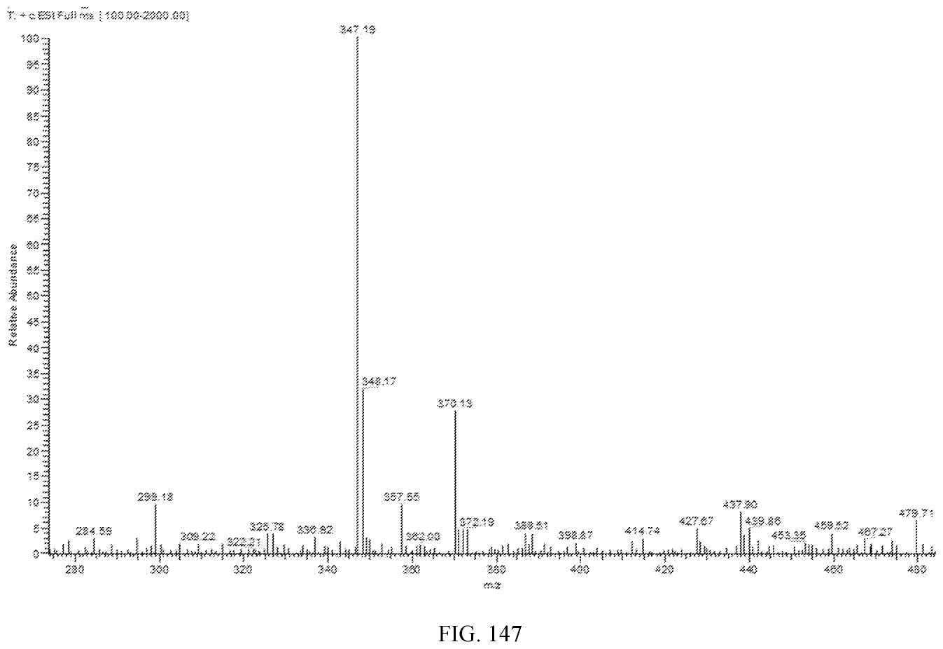

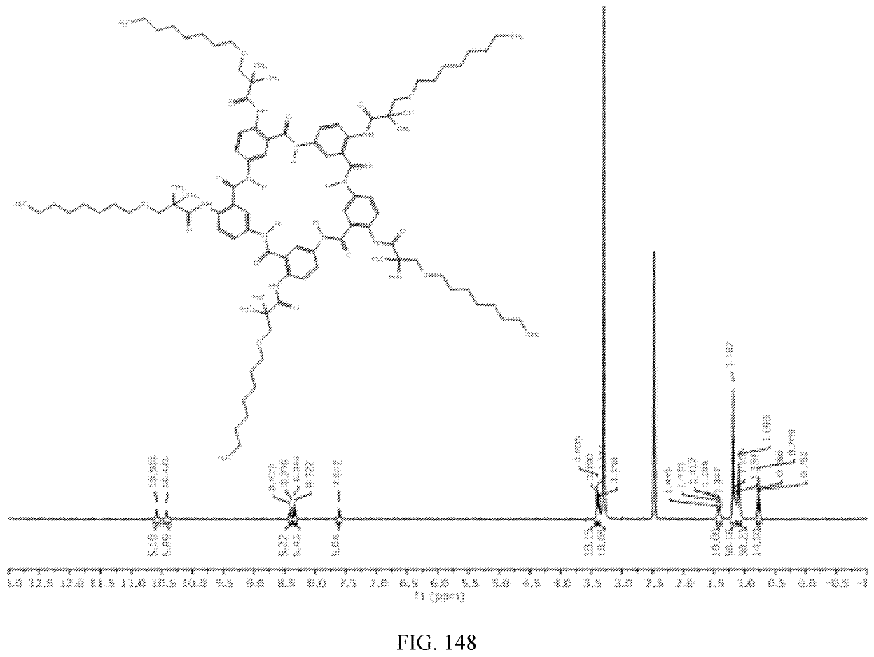

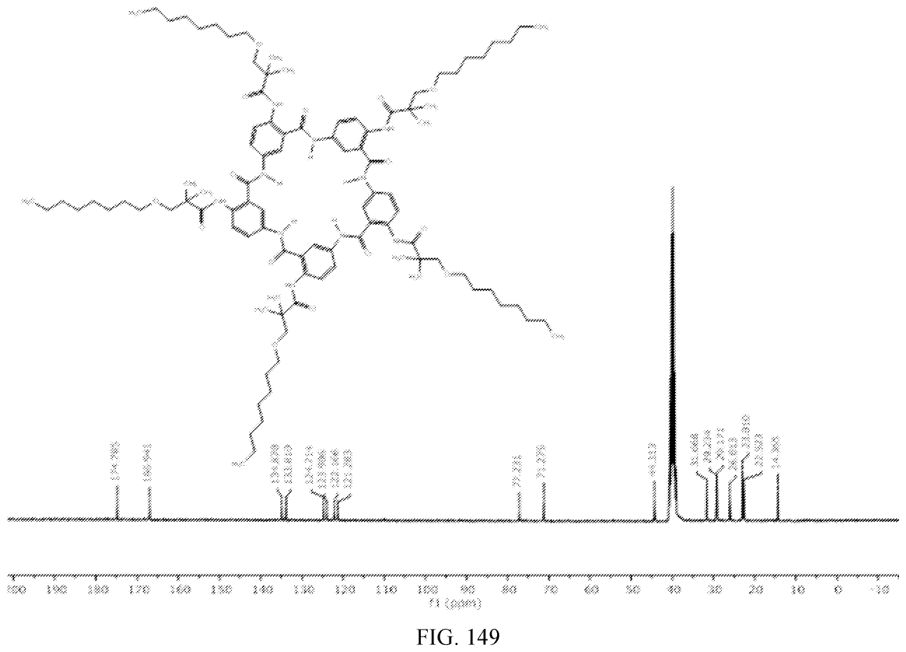

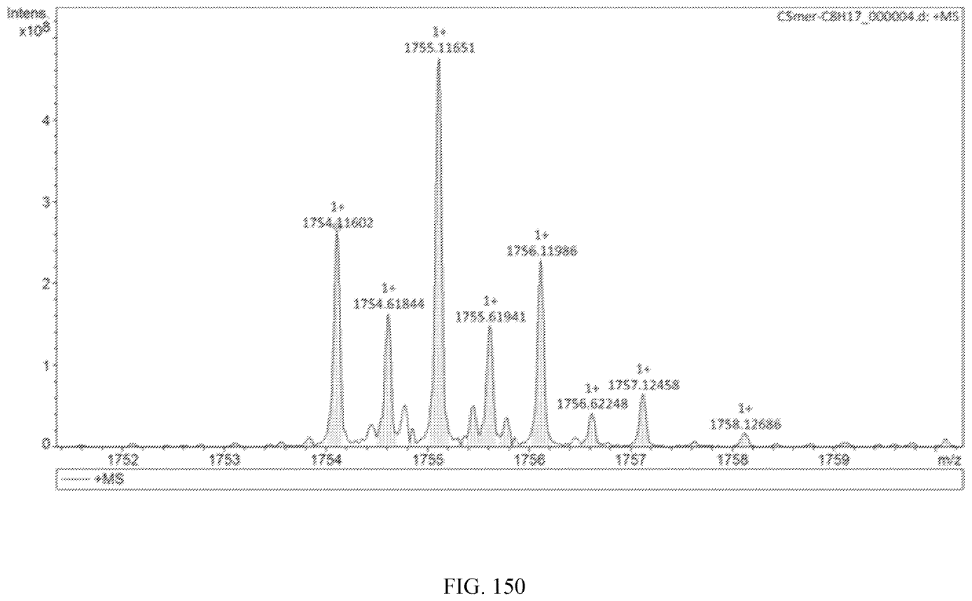

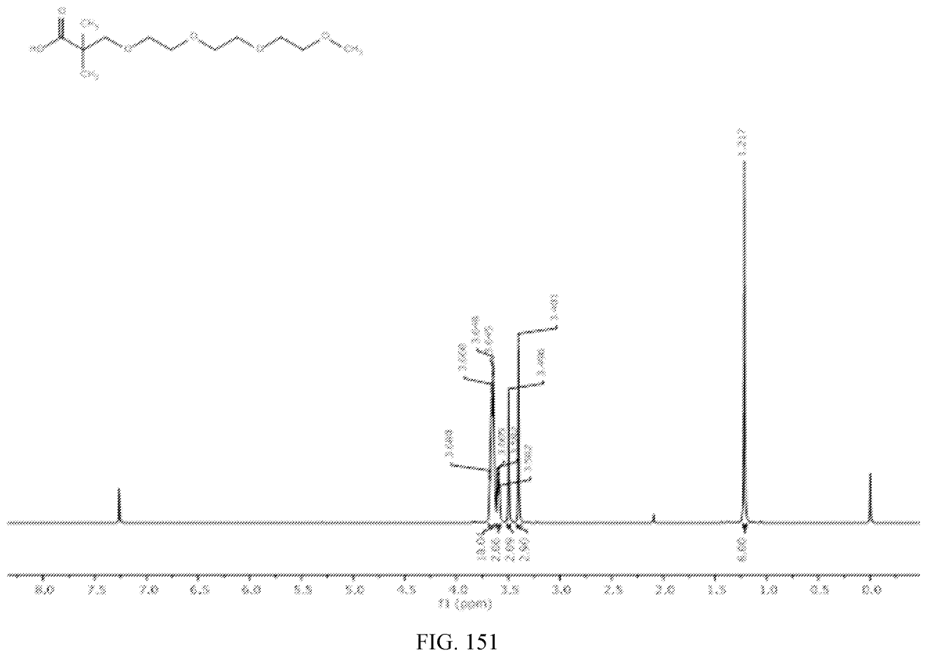

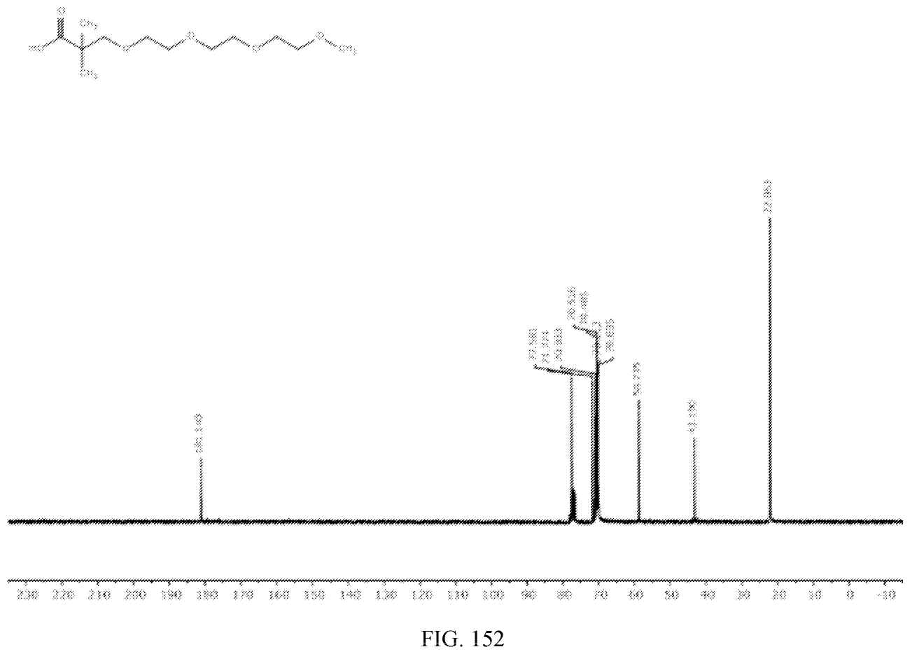

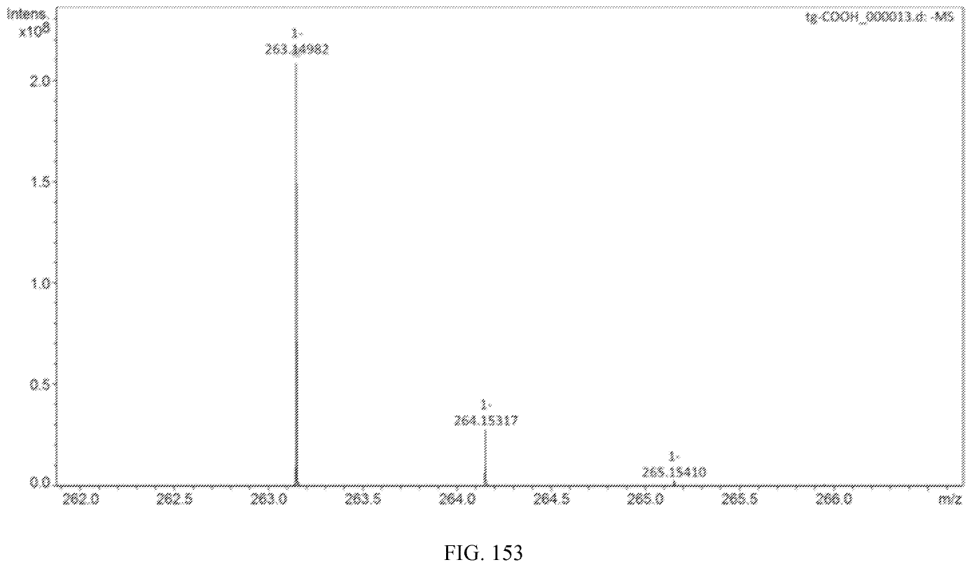

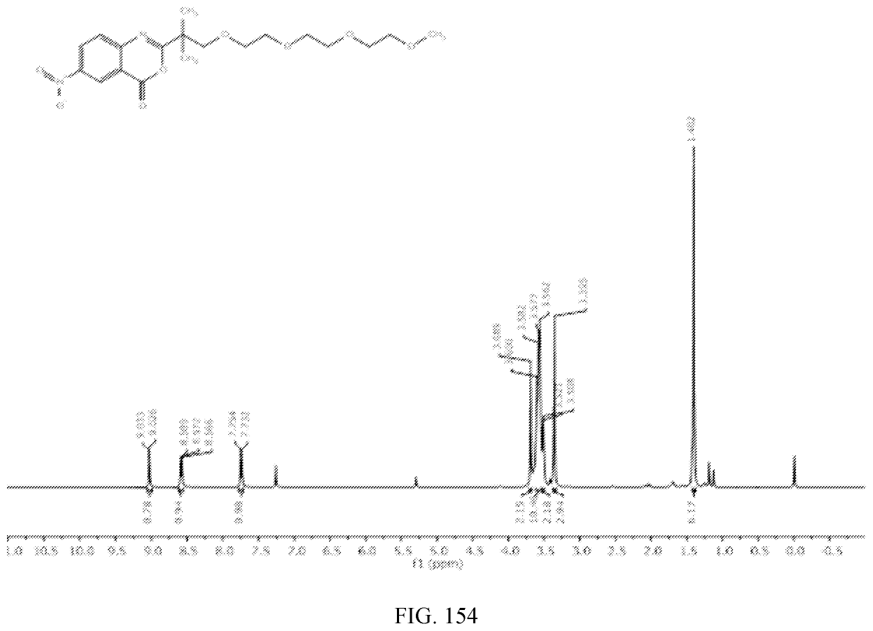

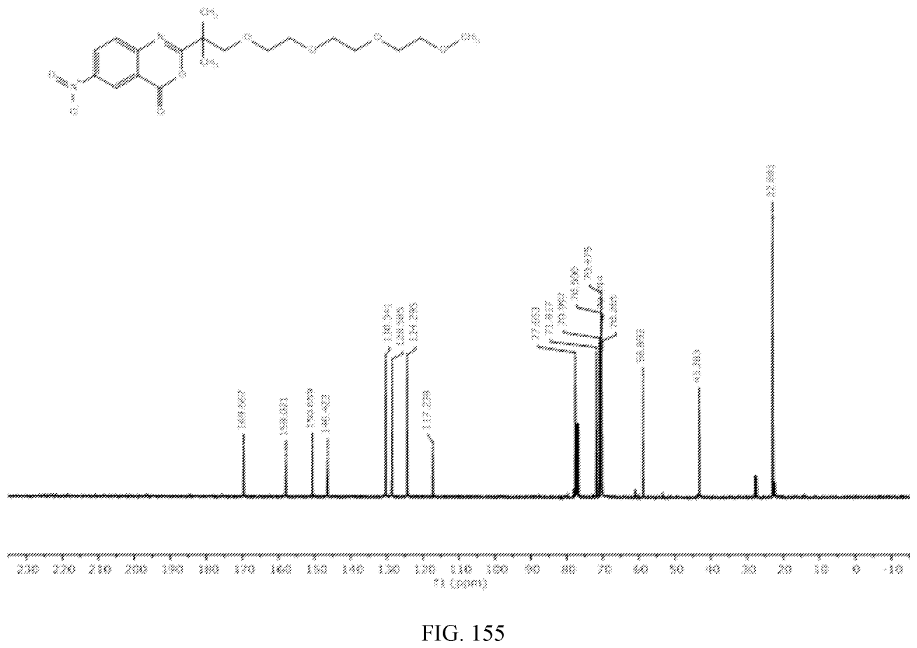

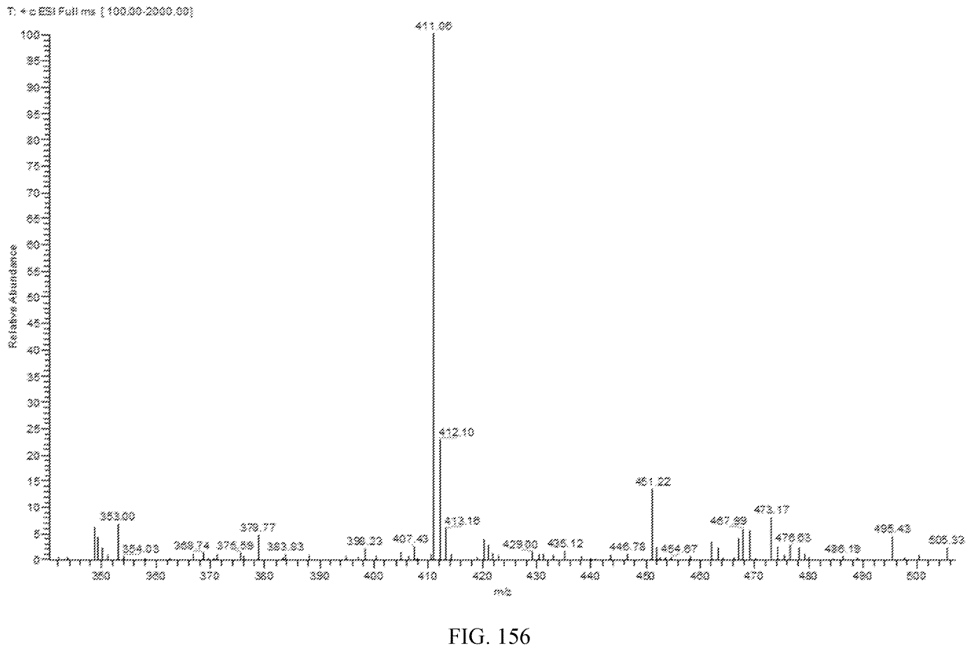

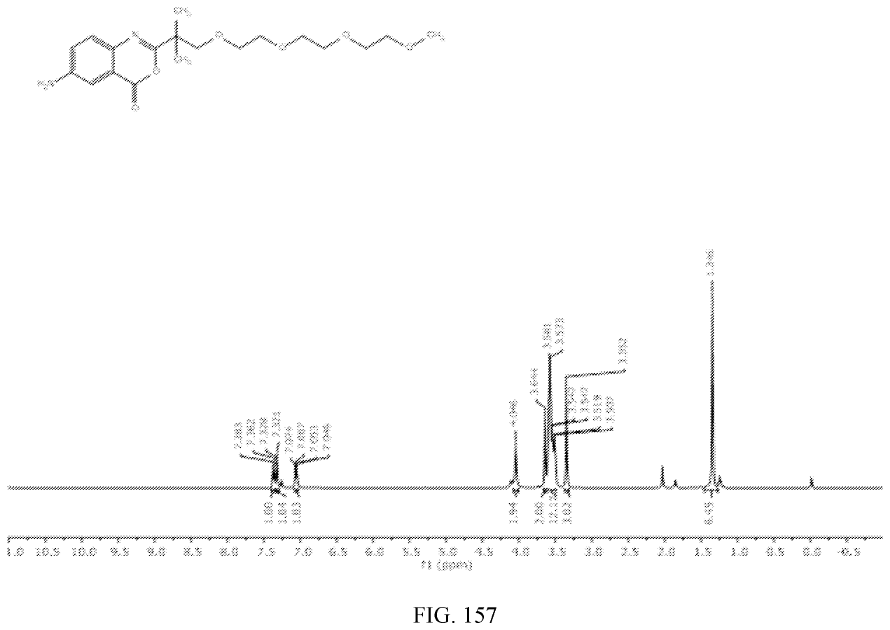

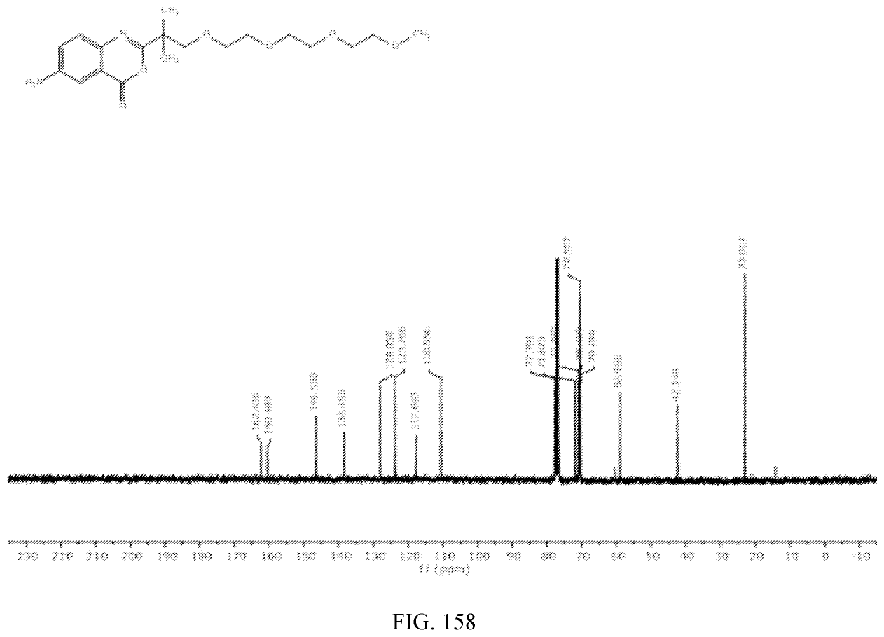

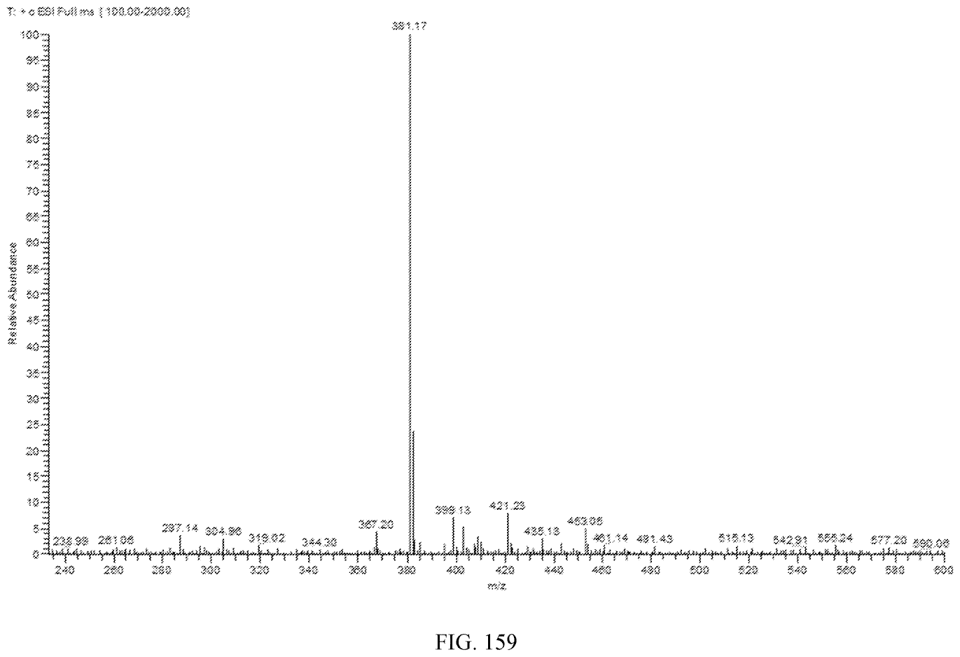

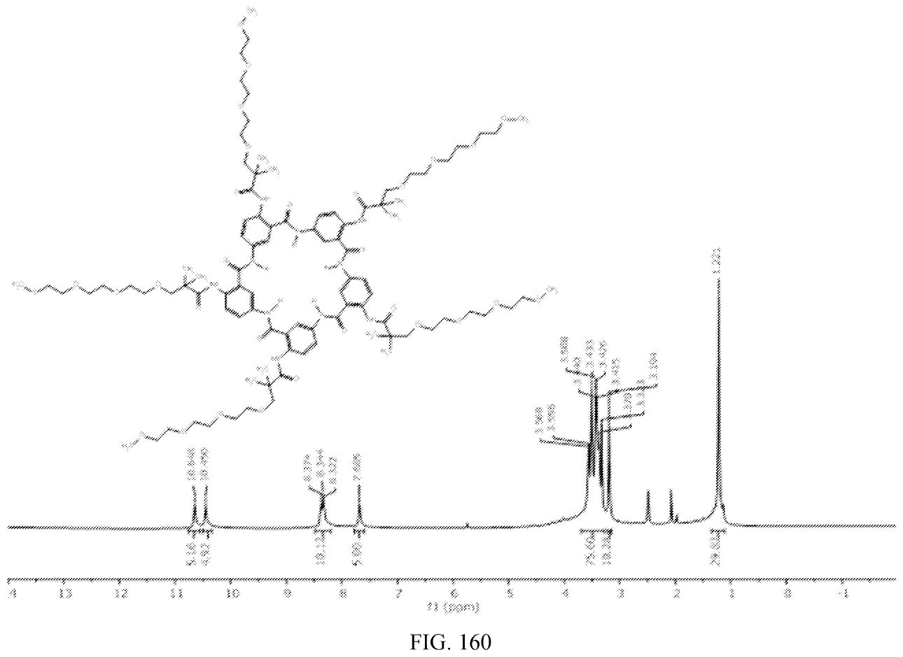

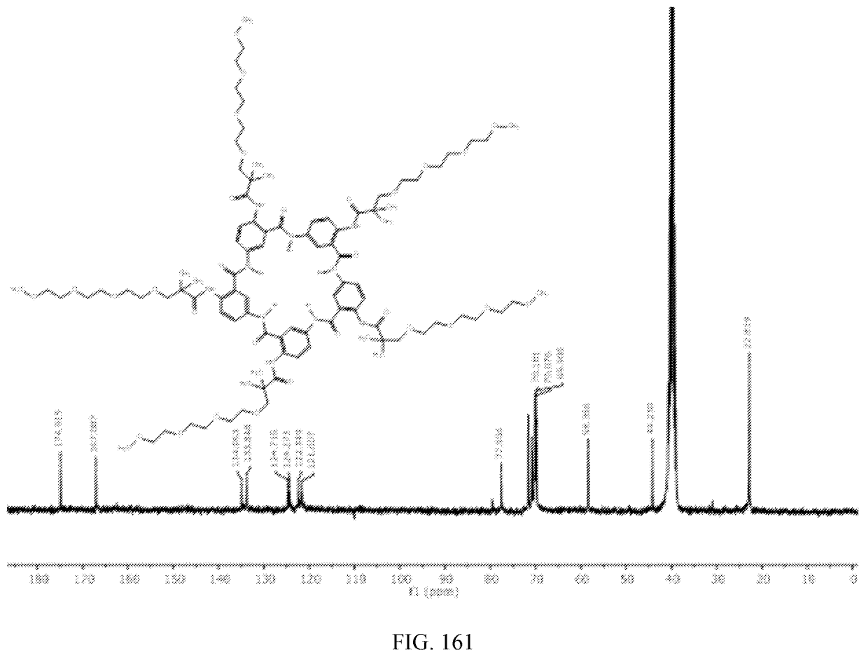

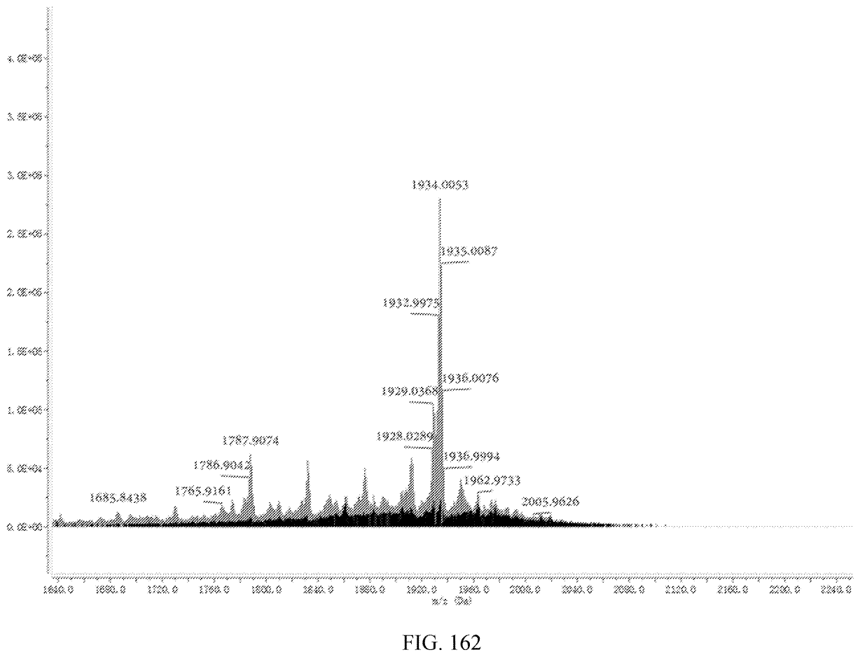

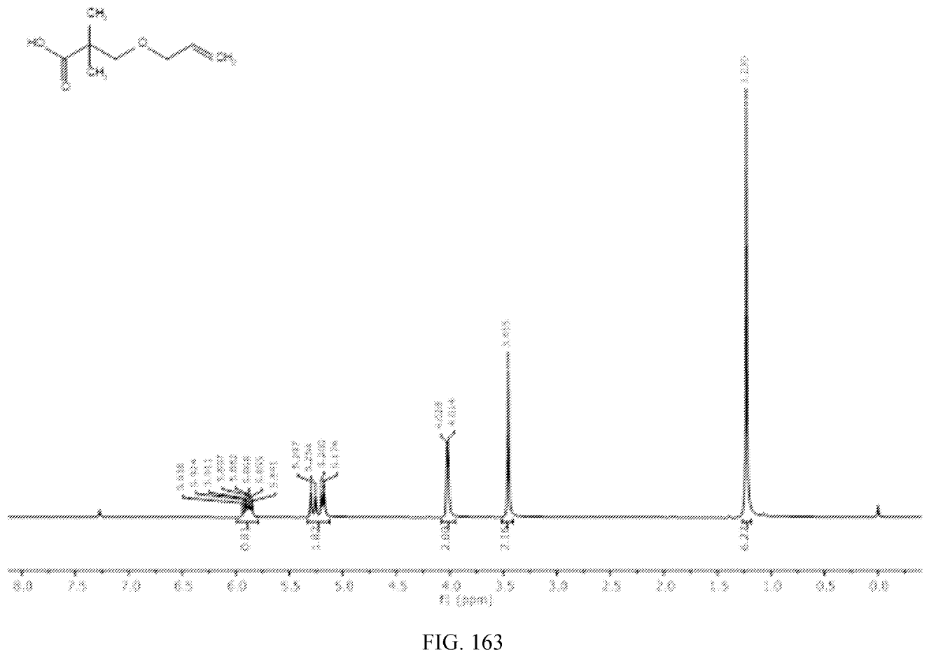

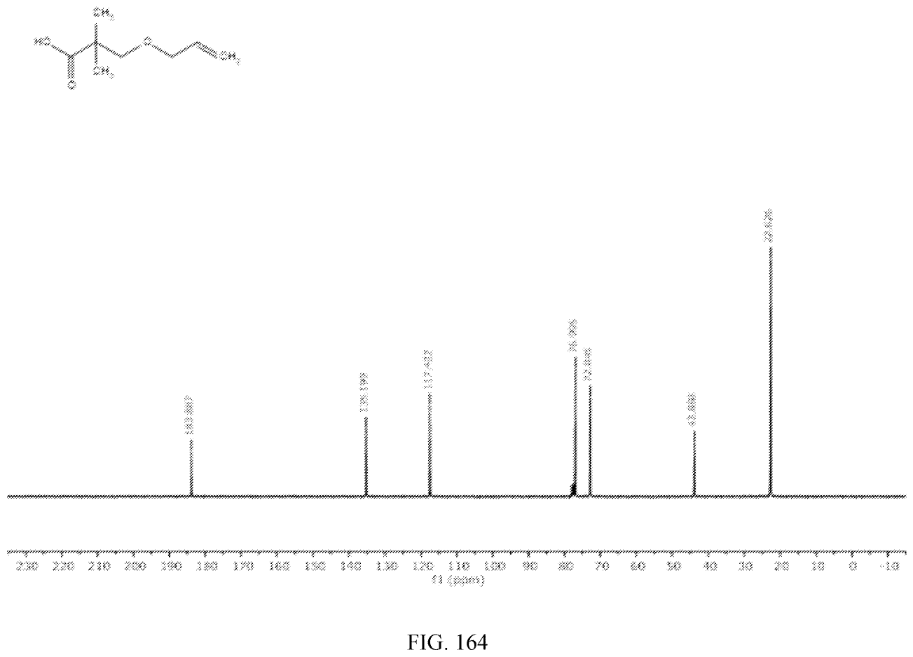

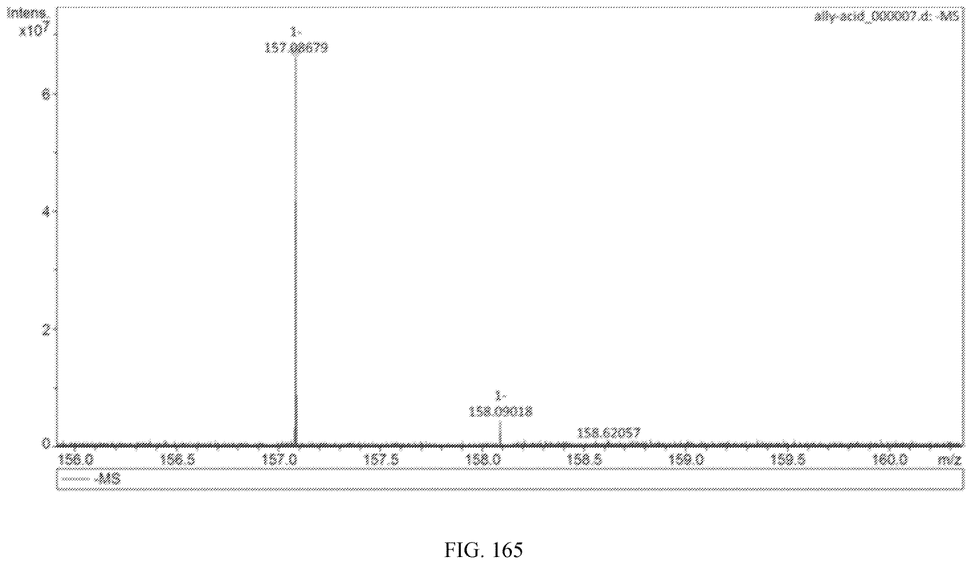

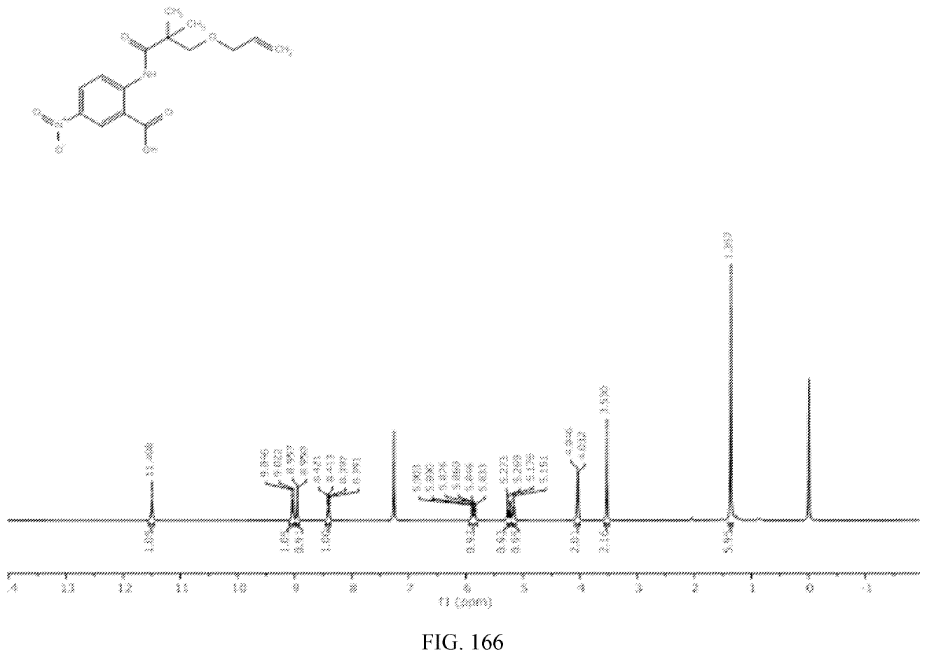

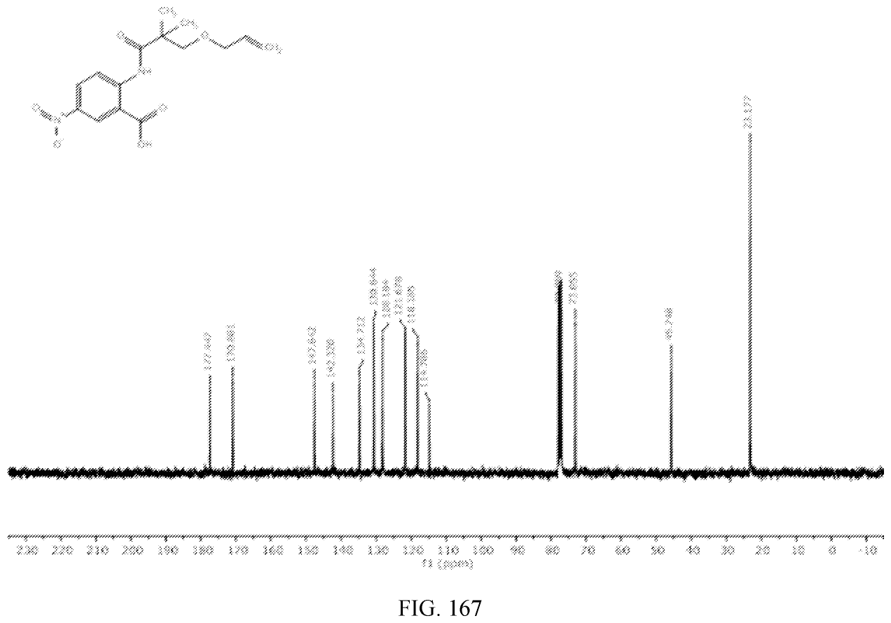

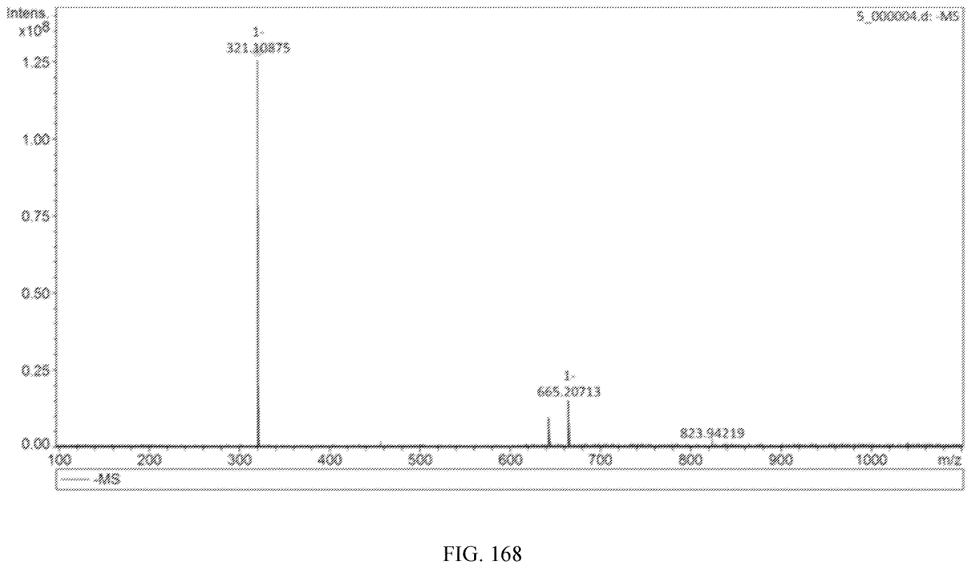

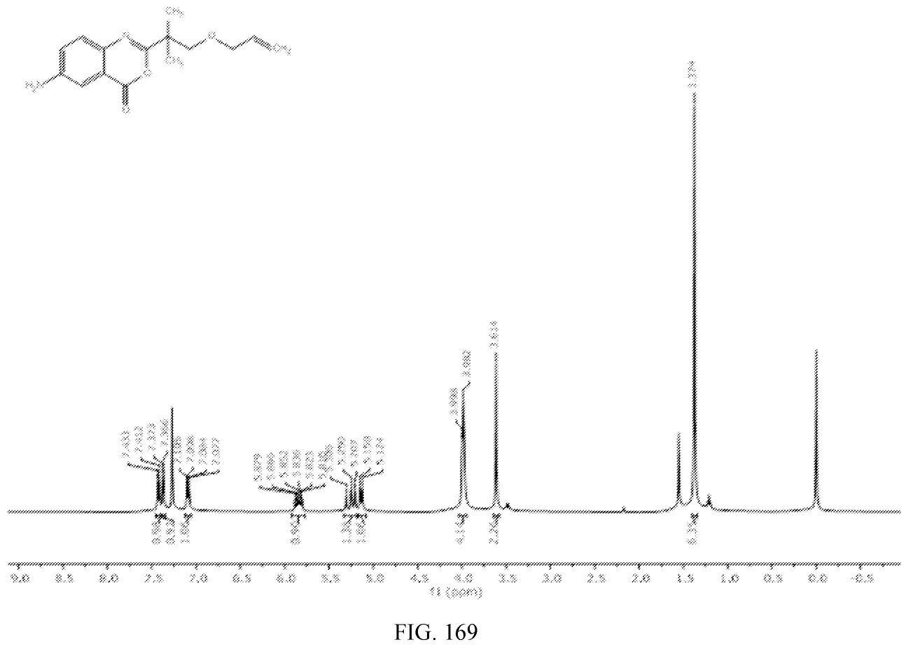

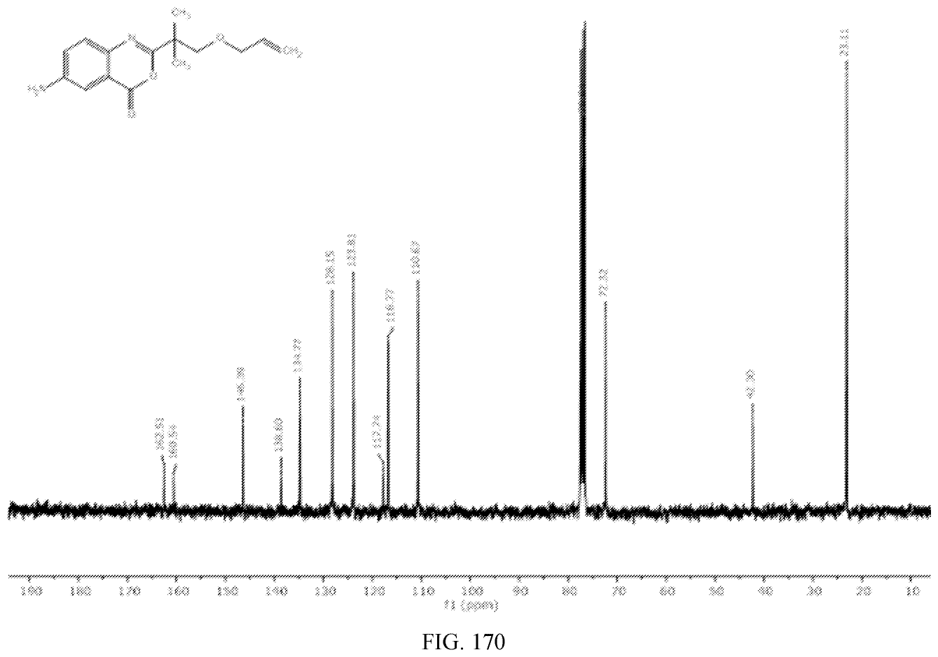

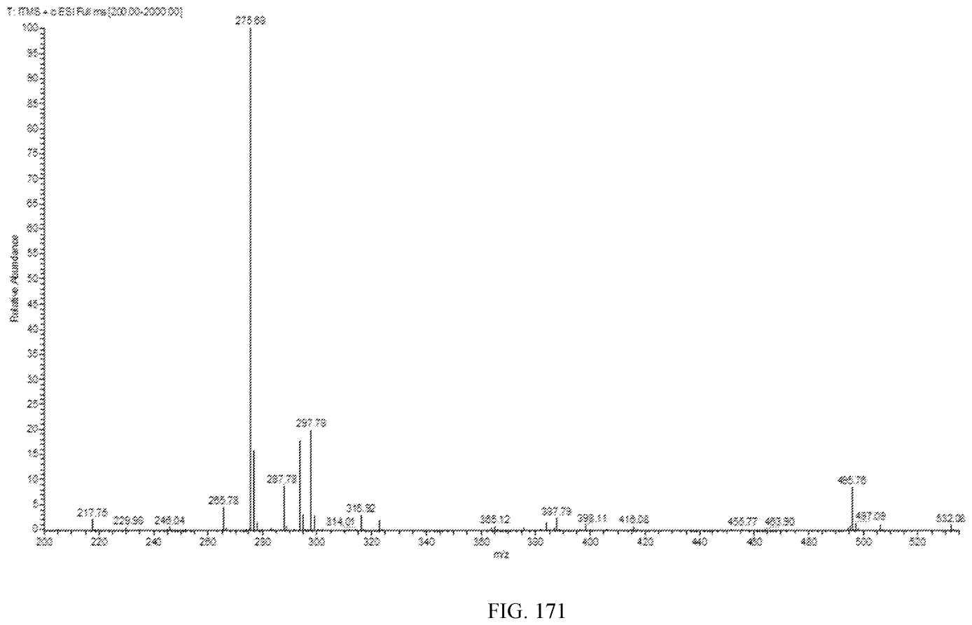

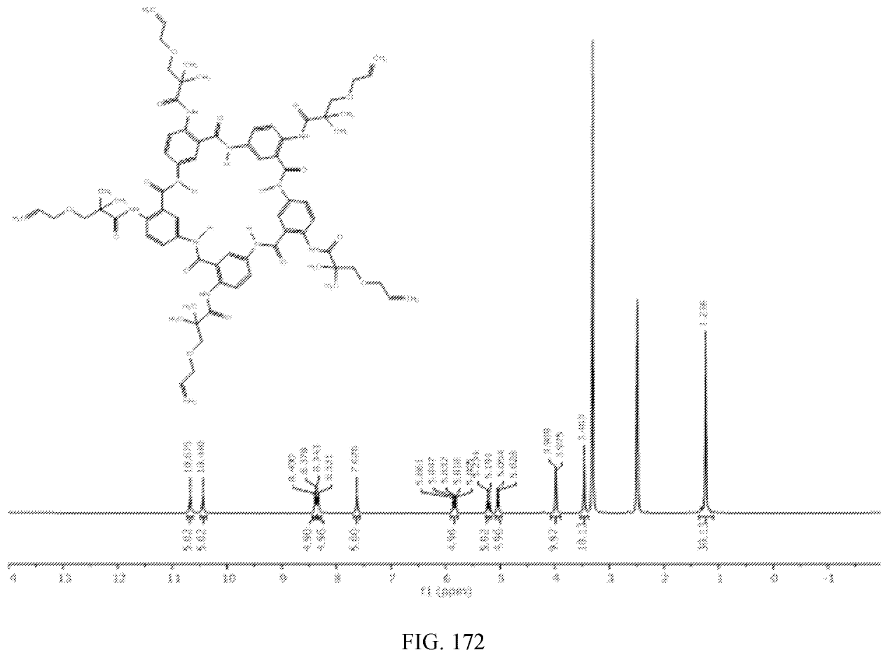

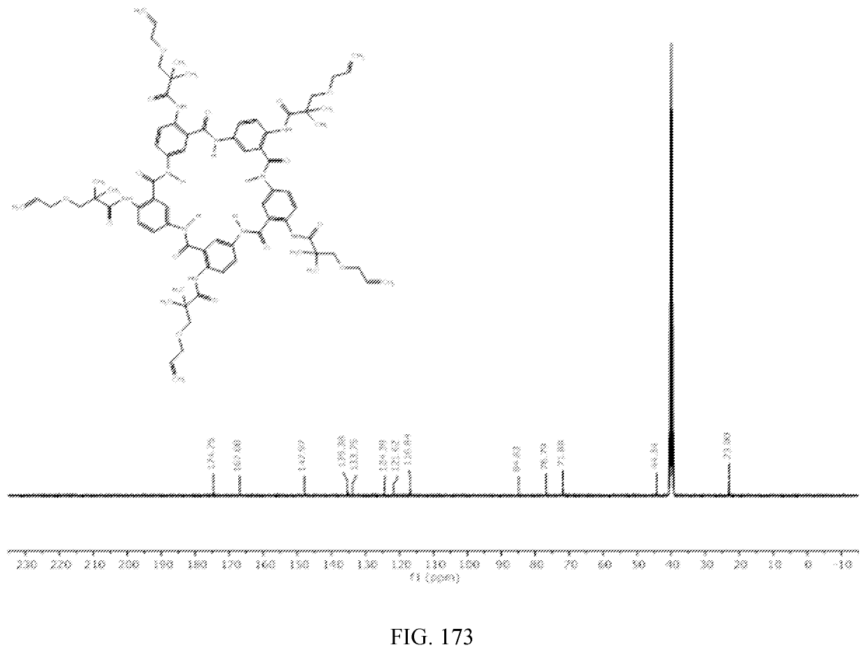

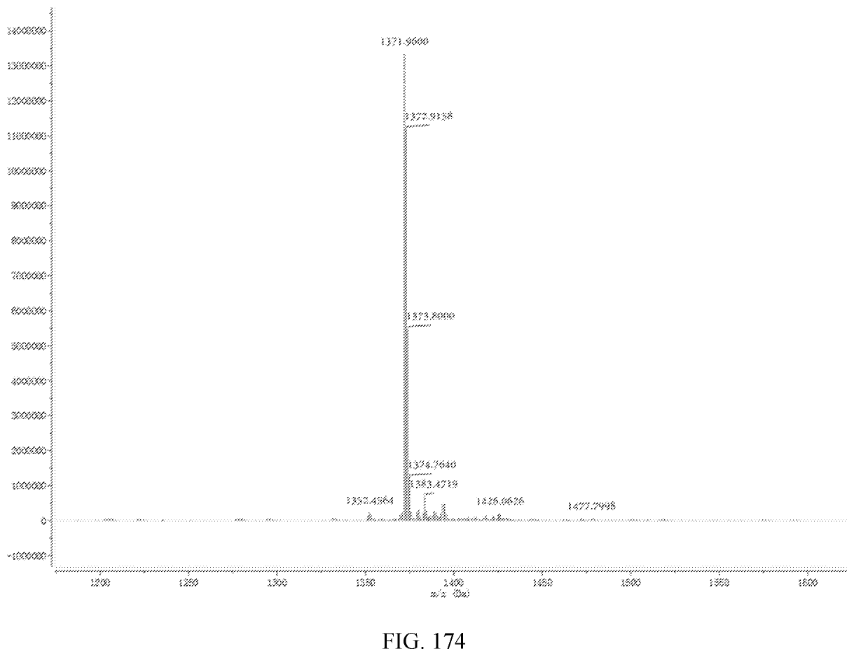

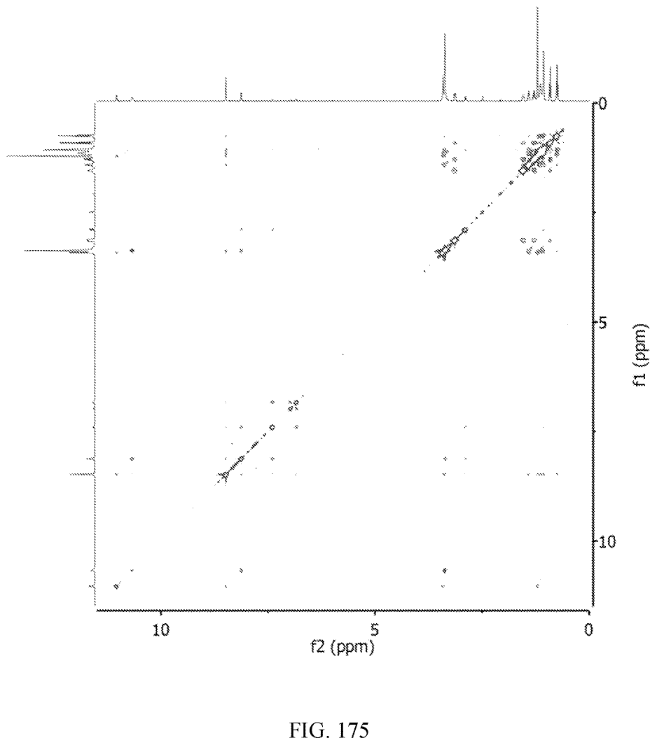

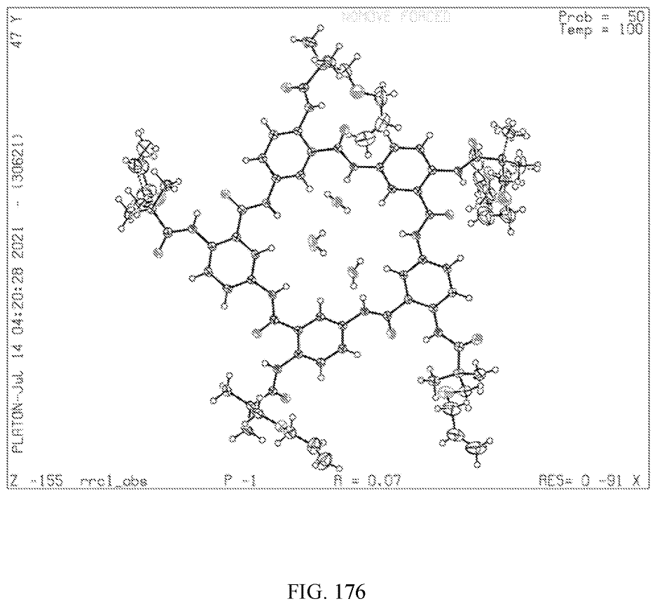

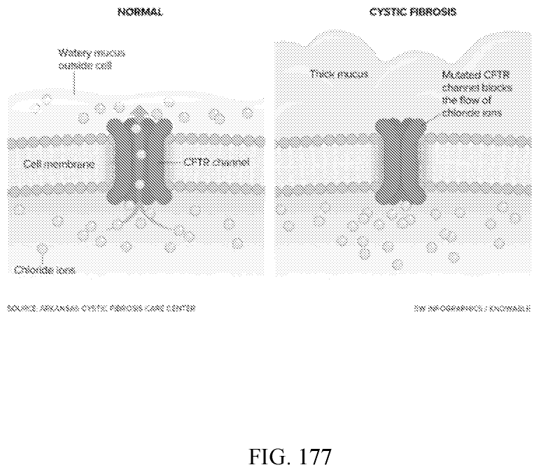

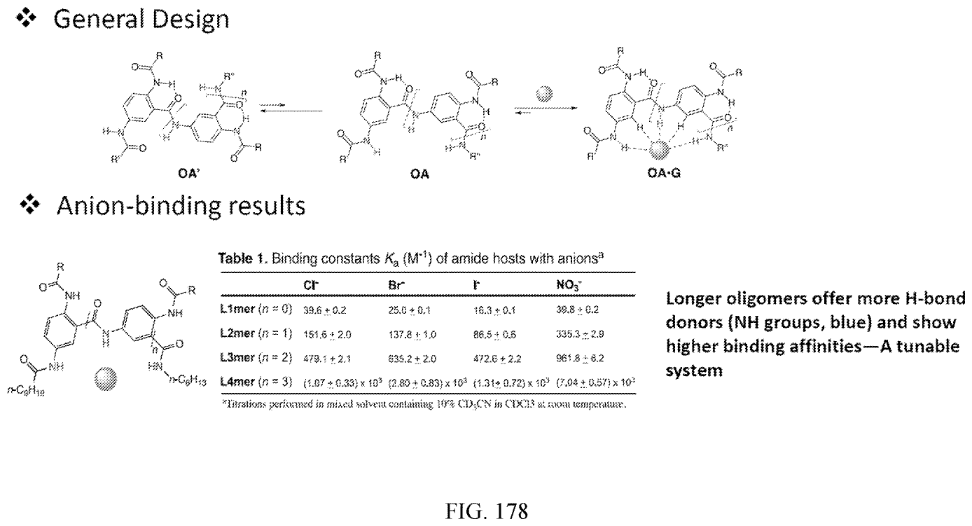

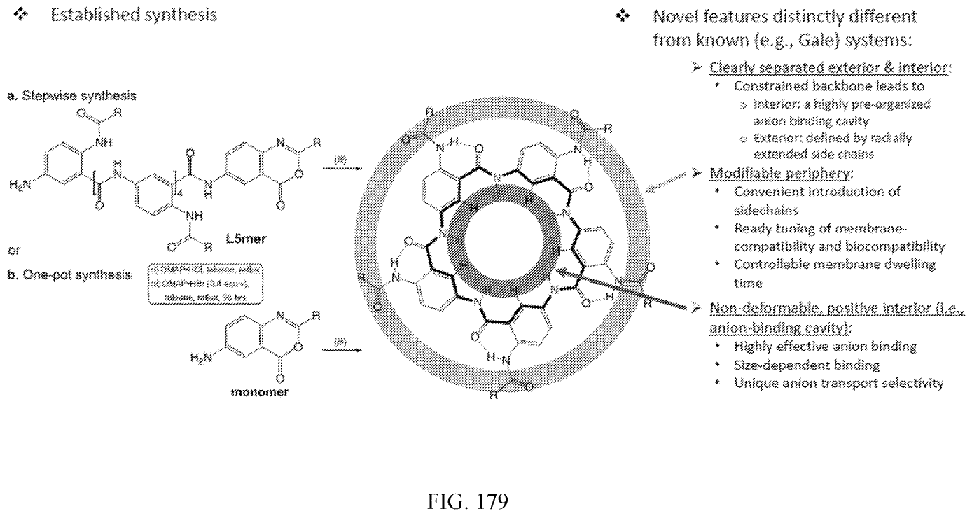

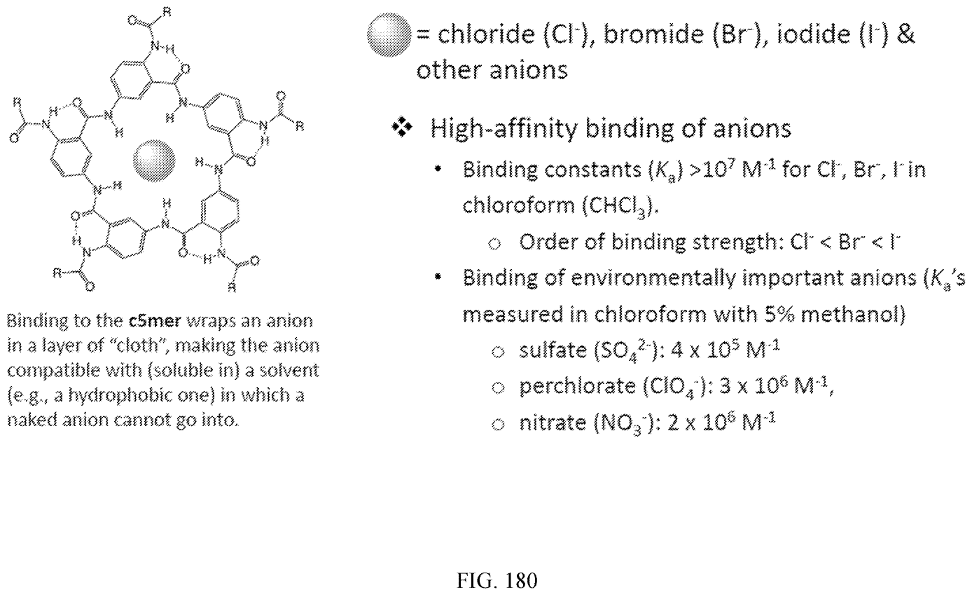

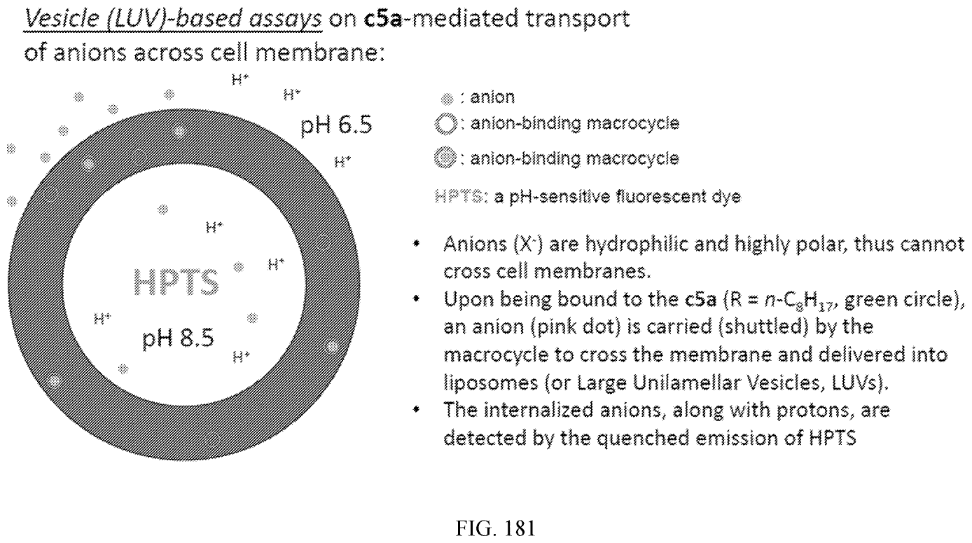

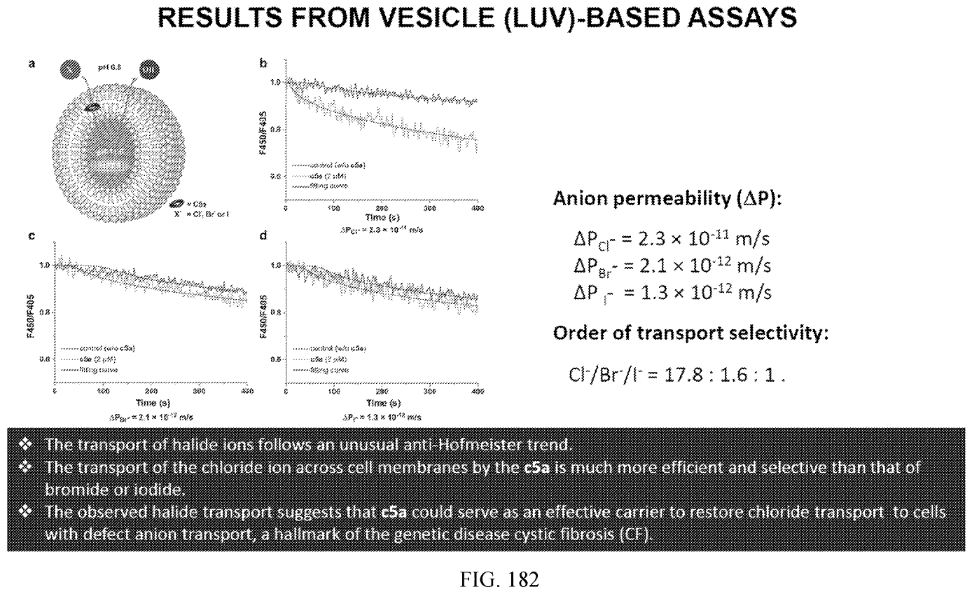

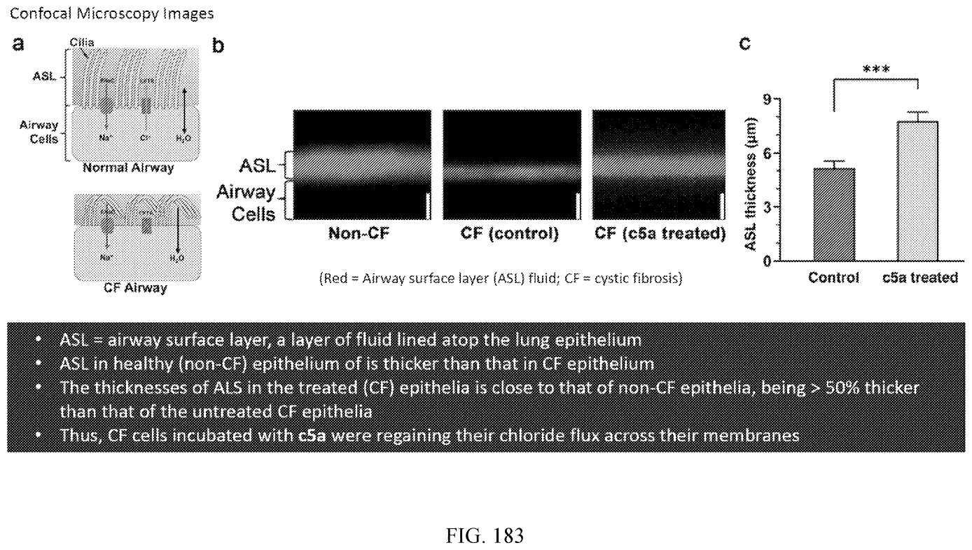

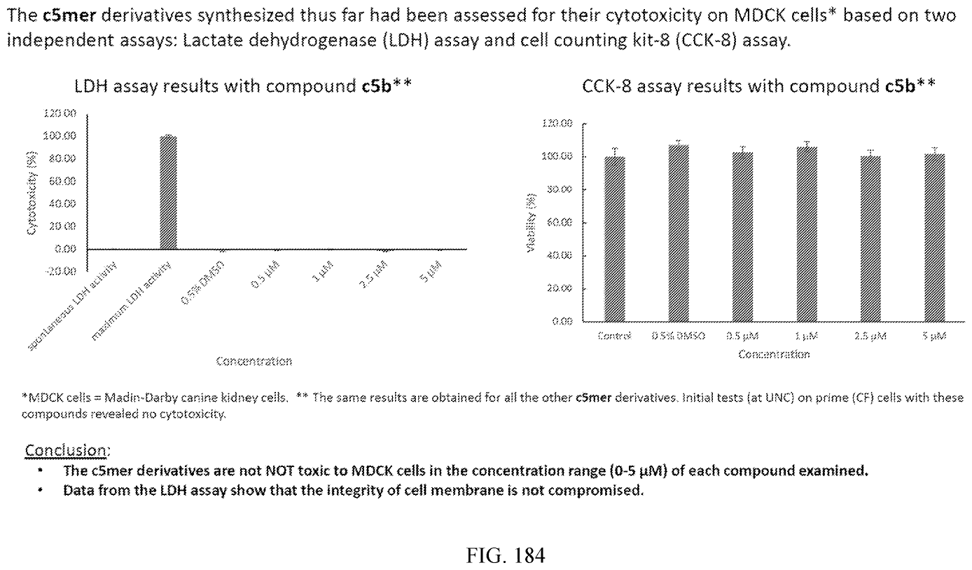

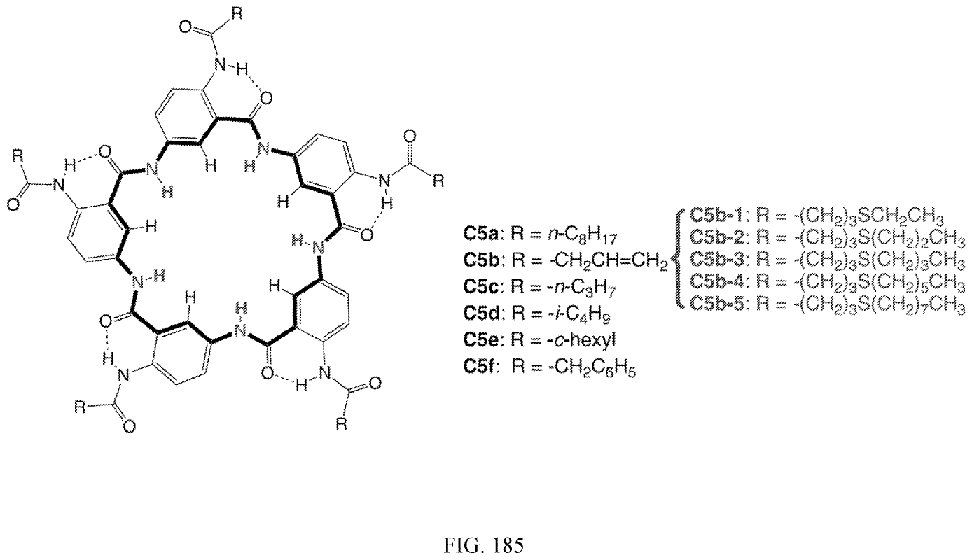

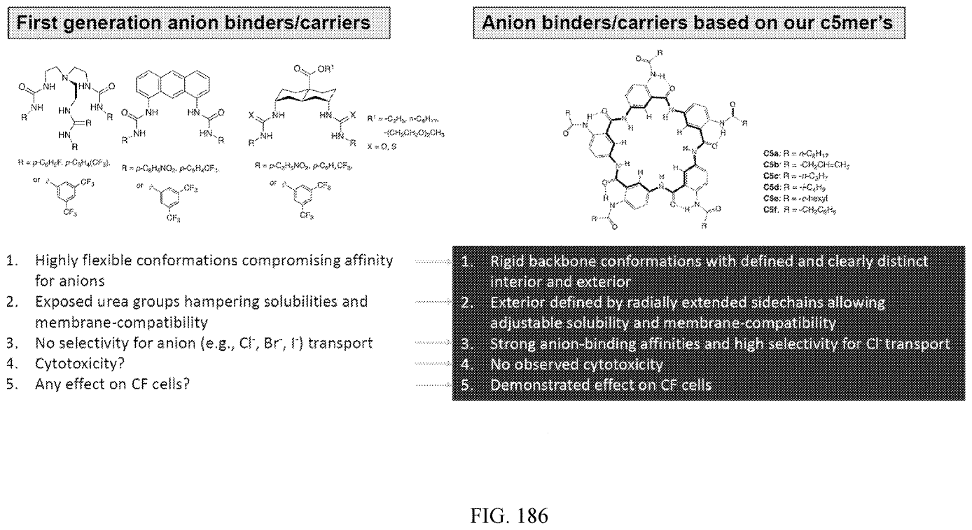

Since macrocycle c5a has a low propensity for self-aggregation, the cross-bilayer anion transport is unlikely mediated by membrane-spanning pores or channels that would require the tubular stacking of multiple molecules of c5a. It is therefore reasonable to hypothesize that c5a mediates halide transport as a carrier. This hypothesis was validated by using a saturated phospholipid, dipalmitoylphosphatidylcholine (DPPC), to form the unilamellar vesicles. DPPC bilayers undergo gel-to-fluid phase transitions at 41° C. Below this temperature, the bilayer is in the gel phase in which c5a could not operate as a carrier. Again using the HPTS fluorescence as an indicator, chloride transport was completely prohibited at 25° C., and when the temperature was raised to 45° C., enhanced halide transport was measured in the presence of c5a (FIG. 134). In these experiments, macrocycle c5a was pre-incubated with the DPPC vesicle in the fluid phase to ensure proper ca insertion (FIG. 134). These results indicate that the mobility of c5a within the lipid bilayer must have played the critical role in transmembrane transport, and the different rates of releasing the bound halides provides a reason for the observed selectivity order of transport.

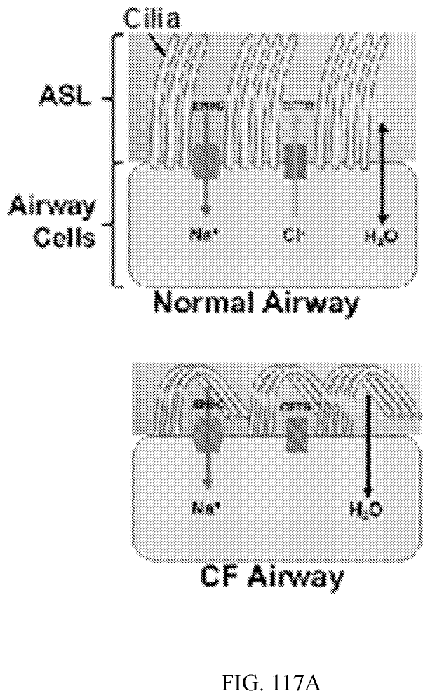

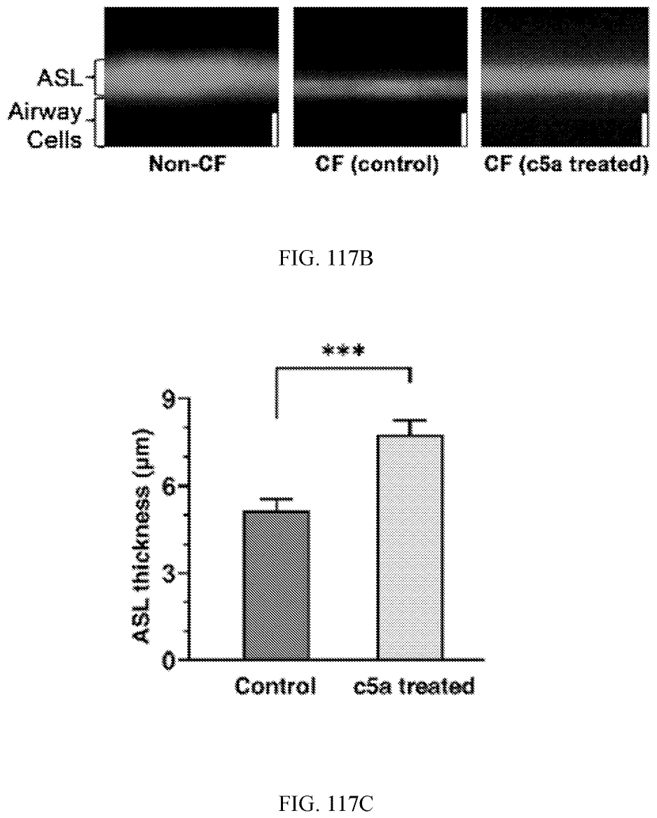

Restoration of depleted airway surface liquid (ASL) of cystic fibrosis cells. The highly efficient and selective transport of Cl⁻ across the cell membrane is biologically and medically significant. Natural chloride channels are known to be responsible for a broad range of functions, with a large spectrum of diseases being caused by defective Cl⁻ channels. With the observed efficient and selective transport of Cl⁻ across lipid bilayers, macrocycles c5 offer the possibility to remedy malfunctioned anion transport found in a variety of diseases. Among these, cystic fibrosis (CF) is an extensively studied life-shortening disease caused by defective anion transport due to the impaired functions of cystic fibrosis transmembrane conductance regulator protein (CFTR) owing to various mutations in the CFTR gene. In the lung, this leads to the dehydration and acidification of the airway surface liquid (ASL), a thin layer of fluid covering the luminal (apical) surface of the airway epithelium (FIG. 117a), causing hyperconcentration of mucin biopolymers, and increased mucus viscoelasticity which impairs cilia beat frequency and reduces mucociliary transport rates.

Human bronchial epithelial (HBE) cells isolated from CF patients were cultured at the time of lung transplant as a model system for diseased airways. These cells were cultured on permeable supports and studied after full differentiation (~21 days). The c5a (5 μM, with 1% DMSO in PBS; 20 μL volume) was then applied apically to the culture daily for five days and compared the changes in the ASL with the control culture under the same condition where a mock solution was applied (1% DMSO in PBS). A small volume (100 nL) of a membrane-impermeable dye, Texas Red-dextran (70 kDa), was nebulized on to the luminal surface of treated and control CF culutres 1 hour prior to visualizing with a scanning confocal microscope. The thickness of the ASL layer on the surface of the HBE cell layer can be conveniently measured by determining the thickness of the fluorescently-labeled fluid layer on the airway surface using X-Z confocal scanning microscopy (FIG. 117b). Compared with the reduced ASL thickness associated with CF, the thickness of the ASL layer, when the CF cells are treated with c5a, is visibly thickened when the CF cells were treated with c5a. Quantitative measurements (FIG. 117c) show that the ASL thickness is increased by more than 50%: 7.8±1.7 μm when treated with c5a, vs 5.1±1.4 μm with the control CF. Therefore, c5a can indeed restore the hydration and the volume of the ASL by rectifying the malfunctioned chloride transport in these cells, which is a key objective of therapeutic interventions of CF. The performance of macrocycle c5a is comparable, if not more so, to the landmark work reported by Burke et al. under similar conditions.

Star-shaped pentaamide macrocycles c5a-c are synthesized from the one-pot reaction of the corresponding monomers based on a unique amide coupling strategy. The efficient, scalable one-pot synthetic procedure allows various derivatives of macrocycles c5 to be prepared in ample quantity with ready structural modification, enabling efficient structural tuning and functional optimization. These novel macrocycles share a fully constrained backbone that locks endocyclic NH and CH groups into a convergent arrangement, leading to high-affinity binding of halides, large anions, and oxoanions. Examining the binding affinities of c5 for halide ions and weakly coordinating anions reveal size-dependent recognition favoring large anions, while the complexation of oxoanions show that anions with strong hydrogen-bonding capabilities achieved remarkable binding affinities, even in solvents containing high proportions of water. The ready insertion of these macrocycles equipped with hydrophobic side chains into lipid bilayers enables efficient transmembrane chloride transport with a selectivity even higher than synthetic and natural chloride transporters. The apical application of c5a to CF cell cultures also successfully restored the ASL, demonstrating the potential of these compounds as therapeutic agents to treat diseases related to anion transport. As these molecules are expected to be immune tolerant, more effective therapeutics might be developed which may circumvent some of the limitations with those based on biomacromolecules may be developed. Based on this structurally tunable platform, the development of the next-generation anion binders, transporters, and potential therapeutics for channel-related diseases can be envisioned.

TABLE 5
Crystal data and structure refinement for c5c.

| Bond precision: | C—C = | Wavelength = |
|---|---|---|
| | 0.0042 A | 1.54184 |
| Cell: | a = 12.3108(1) b = | |
| | 17.9164(1) c = 18.8197(1) | |
| | alpha = 109.473(1) beta = | |
| | 101.931(1) gamma = 102.210(1) | |
| Temperature: | 100 K | |
| | Calculated | Reported |
| Volume | 3647.78(6) | 3647.77(5) |
| Space group | P −1 | P −1 |
| Hall group | −P 1 | −P 1 |
| Moiety formula | C75H90N10O15, | C75H90N10O15, |
| | 3 (H2O) | 3 (H2O) |
| Sum formula | C75H96N10O18 | C75H96N10O18 |
| Mr | 1425.62 | 1425.61 |
| Dx, g cm − 3 | 1.298 | 1.298 |
| Z | 2 | 2 |
| Mu (mm − 1) | 0.768 | 0.768 |
| F000 | 1520.0 | 1520.0 |
| F000′ | 1524.85 | |
| h, k, lmax | 15, 21, 22 | 15, 21, 22 |
| Nref | 13832 | 13812 |
| Tmin, Tmax | 0.894, 0.956 | 0.564, 1.000 |
| Tmin′ | 0.858 | |

Correction method = # Reported T Limits: Tmin = 0.564 Tmax = 1.000
AbsCorr = GAUSSIAN
Data completeness = 0.999 Theta(max) = 70.072
R(reflections) = 0.0734(12452) wR2(reflections) = 0.2269(13812)
S = 1.041 Npar = 1040

Although the present disclosure has been described with respect to one or more particular embodiments and/or examples, it will be understood that other embodiments and/or examples of the present disclosure may be made without departing from the scope of the present disclosure.

1. A compound having the following structure: 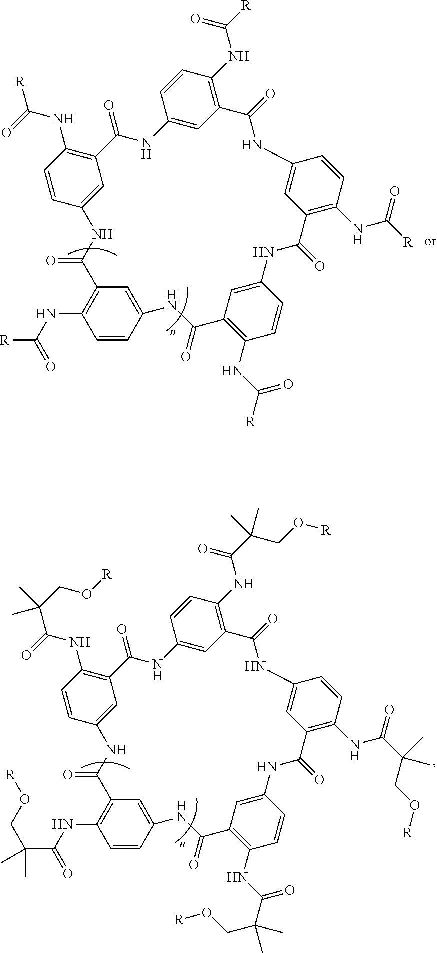
wherein n is 1 or 2, and
wherein R is independently at each occurrence chosen from substituted or unsubstituted aliphatic groups, substituted or unsubstituted cyclic aliphatic groups, and substituted or unsubstituted aryl groups.

2. The compound of claim 1, wherein R is independently at each occurrence chosen from

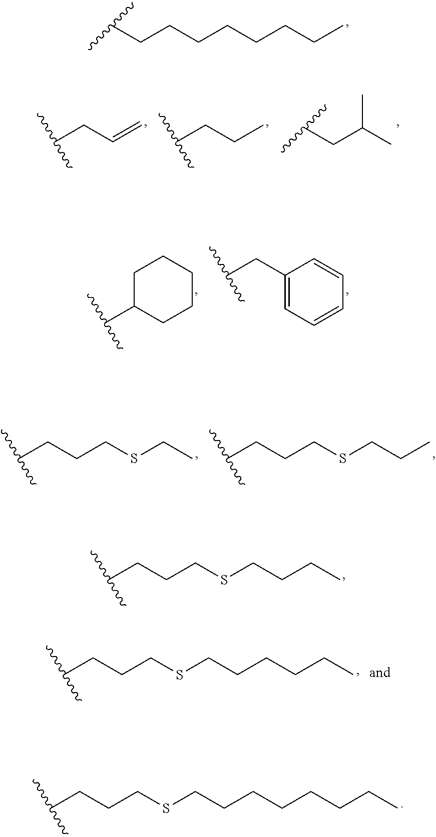

3.-5. (canceled)

6. The compound of claim 1, wherein the compound has the following structure: 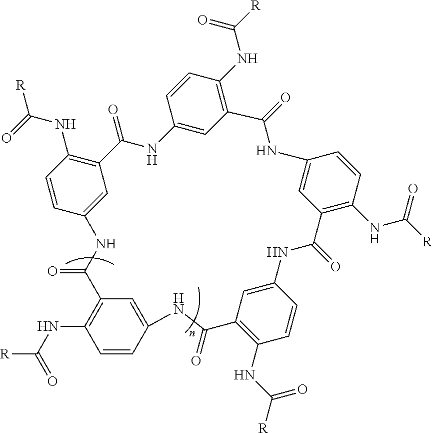
and each R is 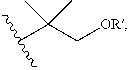
wherein each R′ is independently chosen from:

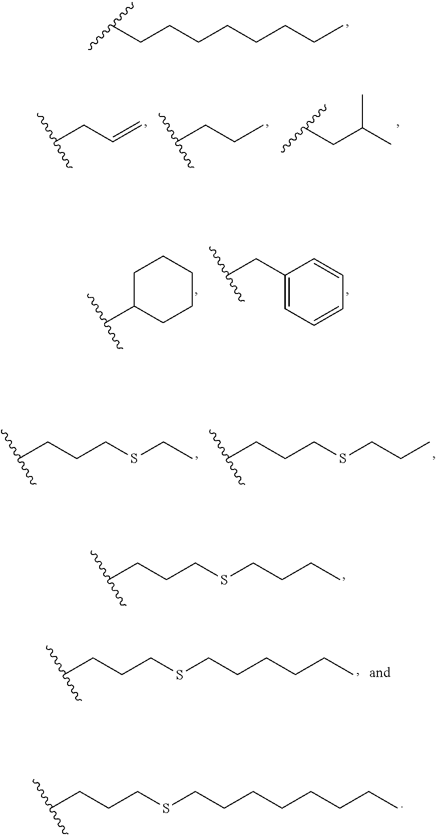

7. A composition comprising one or more compound(s) of claim 1.

8. The composition of claim 7, further comprising one or more hydrogen-bond acceptors and/or ions.

9. The composition of claim 8, wherein the hydrogen-bond acceptors and/or ions are polar guest molecules, anions, cations, or a combination thereof.

10. The composition of claim 9, wherein the anion is chosen from halide ions, nitrate ions, carbonate ions, phosphate ions, sulfate ions, oxo anions, and any combination thereof.

11. The composition of claim 7, wherein the composition comprises a plurality of compounds that all have the same structure or a plurality of compounds wherein at least one of the compounds has a different structure.

12. A method of sequestering one or more hydrogen-bond acceptors and/or ions comprising:
contacting the one or more hydrogen-bond acceptors and/or ions with one or more compound(s) of claim 1, wherein at least a portion or all of the one or more hydrogen-bond acceptors and/or ions are sequestered by the compound(s).

13. The method of claim 12, wherein a complex is formed from the compound(s) and one or more hydrogen-bond acceptors and/or ions.

14. The method of claim 12, wherein the sequestered one or more hydrogen-bond acceptors and/or ions is/are transported across a membrane.

15. The method of claim 14, wherein the membrane is a cell membrane.

16. A method of treating an individual diagnosed with or suspected of having an extracellular and/or intracellular anion imbalance comprising:
administering to the individual one or more compound(s) of claim 1,
such that the extracellular and/or intracellular anion imbalance is adjusted.

17. The method of claim 16, wherein the individual has been diagnosed with cystic fibrosis or cancers.

18. The method of claim 17, wherein the physiological gradient of anion concentration in the individual is at least partially or completely restored.

19. The method of claim 18, wherein one or more symptom(s) related to the extracellular and/or intracellular anion imbalance in the individual is at least partially or completely alleviated.

20. A method of treating an individual diagnosed with or suspected of having cystic fibrosis comprising:
administering to the individual one or more compound(s) of claim 1,
such that one or more symptom(s) related to the cystic fibrosis is at least partially or completely alleviated.

21. The method of claim 20, wherein the individual is a human or a non-human animal.

22. The method of claim 20, wherein the compound forms a complex with a chloride anion.

23. A method for making a compound of claim 1, comprising:
forming a reaction mixture comprising nucleophilic catalyst, an acid, a solvent, and a compound having the following structure: 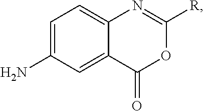
wherein R is independently at each occurrence chosen from substituted or unsubstituted aliphatic groups, substituted or unsubstituted cyclic aliphatic groups, and substituted or unsubstituted aryl groups,
refluxing the reaction mixture,
wherein following refluxing, the compound of claim 1 is formed.

24.-30. (canceled)
